(19) United States
(12) Patent Application Publication
(10) Pub. No.: US 2026/0297588 A1
(43) Pub. Date: Oct. 1, 2026

(54) ALK7 RNA INTERFERENCE AGENTS

(71) Applicant: ELI LILLY AND COMPANY, Indianapolis, IN (US)

(72) Inventors: Ricardo J. SAMMS, Carmel, IN (US); Eitaro AIHARA, Carmel, IN (US); Jesline T. ALEXANDER-CHACKO, Carmel, IN (US); Jorge ALSINA-FERNANDEZ, Indianapolis, IN (US); Robert Andrew BROWN, Carmel, IN (US); Suntara CAHYA, San Diego, CA (US); Christine Chih-Tao CHENG, Carmel, IN (US); Guillermo S. CORTEZ, Indianapolis, IN (US); Isabel Cristina GONZALEZ VALCARCEL, Indianapolis, IN (US); William Howard JACKSON, Indianapolis, IN (US); Xiaoyong LU, Bethesda, MD (US); Ruben Martin MARTINEZ, Indianapolis, IN (US); Fonny Kartawinata MERCHANT, New Palestine, IN (US); James Warren PERFIELD, II, Indianapolis, IN (US); Francisco Alcides VALENZUELA, Indianapolis, IN (US); Jibo WANG, Carmel, IN (US); Aaron David WROBLESKI, Indianapolis, IN (US); Hugo AVILA, Indianapolis, IN (US)

(21) Appl. No.: 19/391,782

(22) Filed: Nov. 17, 2025

Related U.S. Application Data
(60) Provisional application No. 63/723,892, filed on Nov. 22, 2024.
(60) Provisional application No. 63/909,146, filed on Oct. 31, 2025.

(51) Int. Cl. C12N 15/113; A61K 9/00; A61K 31/712; A61K 31/7125; A61K 31/713; A61K 38/00; A61P 3/04; C07K 7/06

(52) U.S. Cl. CPC C12N 15/1137; A61K 9/0019; A61K 31/712; A61K 31/7125; A61K 31/713; A61P 3/04; C07K 7/06; A61K 38/00; C12N 2310/11; C12N 2310/14; C12N 2310/312; C12N 2310/315; C12N 2310/321; C12N 2310/323; C12N 2310/3513; C12N 2310/3515

(57) ABSTRACT

Provided herein are ALK7 RNAi agents, conjugates thereof, and compositions comprising an ALK7 RNAi agent. Also provided herein are methods of using the ALK7 RNAi agents or compositions comprising an ALK7 RNAi agent for reducing ACVR1C expression, and/or treating ALK7 associated metabolic disorder in a subject.

### SEQUENCE LISTING

The present application is being filed along with a Sequence Listing in ST.26 XML format. The Sequence Listing is provided as a file titled “31170_US” created Oct. 28, 2025 and is 4,099,099 bytes in size. The Sequence Listing information in the ST.26 XML format is incorporated herein by reference in its entirety.

### FIELD OF INVENTION

The present disclosure relates to the delivery of therapeutics, in some instances oligonucleotides, to adipose tissue, and more particularly, to peptide conjugates of such therapeutics that are targeted to adipose tissue to modulate genes expression.

### BACKGROUND

The human ACVR1C gene encodes the protein activin receptor-like kinase 7 (ALK7), which is involved in regulatory processes including TGF-β type signaling and tumor suppression. ALK7 is highly expressed in adipose tissue (Carlsson et al., Biochem Biophys Res Commun 2009 May 1; 382(2): 309-314), where it aids in signaling such that lipids stored in adipocytes are maintained. It has been found that loss of ALK7 leads to lipolysis in mice (Yogasawa and Izumi, Adipocyte 2009, 2(4), 246-250).

No direct modulators of ALK7 expression or activity have been approved for therapeutic use. Specific modulation of ALK7 activity or expression levels may be useful for treatment of metabolic disorders, including conditions like obesity and diabetes.

RNA interference (RNAi) is a highly conserved regulatory mechanism in which RNA molecules are involved in sequence-specific suppression of gene expression by double-stranded RNA molecules (dsRNA) (Fire et al., Nature 391:806-811, 1998).

There remains a need for therapeutic agents that can inhibit or adjust the expression of ALK7 for treating ALK7 associated metabolic disorders, e.g., by utilizing RNAi.

Adipose tissue is a connective tissue and an endocrine organ, participating in various physiological processes, including energy homeostasis, glucose metabolism, and inflammation. Dysregulation of adipose tissue function, including accumulation of excess adipose, can lead to metabolic disorders, such as obesity, diabetes, and cardiovascular diseases. Therefore, modulation of adipose tissue gene expression can be a potential strategy for the treatment of these disorders.

### SUMMARY OF INVENTION

Provided herein are ALK7 RNAi agents including a sense strand and an antisense strand, wherein the sense strand and the antisense strand form a duplex, comprising a sense strand and an antisense strand, wherein the sense strand and the antisense strand form a duplex, wherein the duplex comprises one of dsRNA 1-76 or 163-230 of Table 4, wherein optionally one or more nucleotides of the sense strand and the antisense strand are independently modified nucleotides, and wherein optionally one or more internucleotide linkages of the sense strand and the antisense strand are modified internucleotide linkages. For example, the duplex may include the sense strand having SEQ ID NO: 78 and the antisense may be the corresponding antisense strand with SEQ ID NO: 154, and so forth.

In certain embodiments, at least one of the sense strand and the antisense strand comprises one or more independently modified nucleotides.

In certain embodiments, at least one of the sense strand and the antisense strand comprises one or more modified internucleotide linkages.

In some embodiments, at least one of the sense strand and the antisense strand comprises one or more independently modified oligonucleotides and one or more modified internucleotide linkages.

In some embodiments, one or more nucleotides of the sense strand are modified nucleotides. In certain embodiments, each nucleotide of the sense strand is a modified nucleotide. In some embodiments, one or more nucleotides of the antisense strand are modified nucleotides. In some embodiments, each nucleotide of the antisense strand is a modified nucleotide.

In some embodiments, the modified nucleotide is a 2′-fluoro modified nucleotide, 2′-O-methyl modified nucleotide, 2′ deoxy nucleotide (DNA), or 2′-O-alkyl modified nucleotide.

In some embodiments, the sense strand has four 2′-fluoro modified nucleotides at positions 7, 9, 10, and 11 from the 5′ end of the sense strand.

In some embodiments, nucleotides at positions other than positions 7, 9, 10, and 11 of the sense strand are 2′-O-methyl modified nucleotides.

In some embodiments, the antisense strand has four 2′-fluoro modified nucleotides at positions 2, 6, 14, and 16 from the 5′ end of the antisense strand.

In some embodiments, nucleotides at positions other than positions 2, 6, 14 and 16 of the antisense strand are 2′-O-methyl modified nucleotides.

In some embodiments, the sense strand has three 2′-fluoro modified nucleotides at positions 9, 10, and 11 from the 5′ end of the sense strand.

In some embodiments, nucleotides at positions other than positions 9, 10, and 11 of the sense strand are 2′-O-methyl modified nucleotides.

In some embodiments, the antisense strand has five 2′-fluoro modified nucleotides at positions 2, 5, 7, 14, and 16 from the 5′ end of the antisense strand.

In some embodiments, nucleotides at positions other than positions 2, 5, 7, 14, and 16 of the antisense strand are 2′-O-methyl modified nucleotides.

In some embodiments, the antisense strand has five 2′-fluoro modified nucleotides at positions 2, 5, 8, 14, and 16 from the 5′ end of the antisense strand.

In some embodiments, the sense strand has three 2′-fluoro modified nucleotides at positions 9, 10, and 11 from the 5′ end of the sense strand.

In some embodiments, nucleotides at positions other than positions 2, 5, 8, 14, and 16 of the antisense strand are 2′-O-methyl modified nucleotides.

In some embodiments, the antisense strand has five 2′-fluoro modified nucleotides at positions 2, 3, 7, 14, and 16 from the 5′ end of the antisense strand.

In some embodiments, nucleotides at positions other than positions 2, 3, 7, 14, and 16 of the antisense strand are 2′-O-methyl modified nucleotides.

In some embodiments, the antisense strand has three 2′-fluoro modified nucleotides at positions 2, 14, and 16 from the 5′ end of the antisense strand.

In some embodiments, nucleotides at positions other than positions 2, 14, and 16 of the antisense strand are 2′-O-methyl modified nucleotides.

In some embodiments, the sense strand and the antisense strand have one or more modified internucleotide linkages. The modified internucleotide linkage may be a phosphorothioate linkage. In some embodiments, the sense strand has four or five phosphorothioate linkages. In some embodiments, the antisense strand has four or five phosphorothioate linkages.

In some embodiments, the antisense strand has 5′ phosphate or 5′ vinylphosphonate.

In some embodiments, the sense strand comprises an abasic moiety or inverted abasic moiety.

In one embodiment of the ALK7 RNAi agent, the sense strand and the antisense strand comprise a pair of nucleic acid sequences selected from the group consisting of dsRNA 77-162 or 231-326 of Table 6. For instance, the sense strand may be of SEQ ID NO: 230, and the corresponding antisense strand may include SEQ ID NO: 316.

In one embodiment, an ALK7 RNAi agent is provided. The RNAi agent is of the Formula:

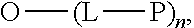

wherein O is a double stranded RNA (dsRNA) comprising a sense stand and an antisense strand, wherein the antisense strand is complementary to ALK7 mRNA;
wherein L is a linker or a bond;
wherein P is an NPR-C-binding protein or an NPR-C binding peptide comprising SEQ ID NO: 1 (GX₁₁IDX₁₄I), wherein XII is arginine, proline, or hydroxyproline, and X₁₄ is arginine or N-methylarginine; and
wherein n is 1, 2, 3, or 4.

In some embodiments, X₁₁ is R. In some embodiments, X₁₄ is R.

In some embodiments, P comprises SEQ ID NO: 2 (GRIDRI).

In some embodiments, P comprises SEQ ID NO: 3 (SX₇X₈X₉GX₁₁IDX₁₄I), wherein:
X₇ is glycine, alanine, proline, hydroxyproline, serine, or cysteine;
X₈ is phenylalanine, or cyclohexylalanine, and
X₉ is glycine, alanine, or serine.

In some embodiments, X₇ is C. In some embodiments, X₉ is G.

In some embodiments, P comprises SEQ ID NO: 4 (SCFGGRIDRI).

In some embodiments, P comprises SEQ ID NO: 5 (FGGRIDRIGA).

In some embodiments, P comprises SEQ ID NO: 6 (RSSX₇FGGRIDRI), wherein X₇ is serine or cysteine.

In some embodiments, P comprises SEQ ID NO: 7 (RSSX₇FGGRIDRIGA). In some embodiments, X₇ is cysteine. In other embodiments, X₇ is serine.

In some embodiments, P comprises SEQ ID NO: 8 (X₇-Cha-X₉GX₁₁IDX₁₄I), wherein:
X₇ is proline, hydroxyproline, glycine, cysteine, or alanine;
X₉ is alanine, serine, or glycine;
X₁₁ is proline, hydroxyproline, or arginine; and
X₁₄ is arginine or N-methylarginine.

In some embodiments, P comprises SEQ ID NO: 9 (fSp-Cha-aGPIDRI).

In some embodiments, P comprises a sequence selected from any one of SEQ ID NOs: 11-57.

In some embodiments, P is a linear peptide. In other embodiments, P is a cyclic peptide. In such embodiments, P can be cyclized by covalent attachment of a side chain of a first cysteine residue to a side chain of a second cysteine residue. The covalent attachment may include a disulfide bond, or may include a thioacetal moiety.

In some embodiments, P comprises a C-terminal hydroxyl.

In some embodiments, P comprises a C-terminal amide.

In some embodiments, P is an NPR-C-binding antibody, or a fragment thereof.

In some embodiments, L comprises a linker core and one or more spacers. In specific embodiments, L comprises Spacer1-Linker Core-Spacer2. In some embodiments, the Linker Core is selected from Table 7. In some embodiments, Spacer1 and Spacer 2 are selected from Table 8. In some embodiments, L is selected from Table 9.

In some embodiments, n is 1 or 2.

In some embodiments, L is attached to the 5′ end of the sense strand and to the N-terminal end of the peptide. In other embodiments, L is attached to the 5′ end of the sense strand and to the C-terminal end of the peptide. In other embodiments, L is attached to the 3′ end of the sense strand and to the N-terminal end of the peptide. In other embodiments, L is attached to the 3′ end of the sense strand and to the C-terminal end of the peptide.

In some embodiments, the RNAi agent includes a fatty acid (FA). In some embodiments, FA is attached to O. In some embodiments, FA is attached to P.

In some embodiments, the RNAi agent comprises (FA)m-O-L-P or O-L-P-(FA)ₘ, wherein m is an integer of 1 to 4.

In some embodiments, the RNAi agent further comprises a Spacer3. In such embodiments, the RNAi agent may be of the formula (FA-Spacer3)ₘ-O-L-P or O-L-P-(Spacer3-FA)ₘ, wherein m is an integer of 1 to 4. In other embodiments, the RNAi agent may be of the formula O-L-(FA-Spacer3)ₘ-P or 0-(Spacer3-FA)ₘ-L-P, wherein m is an integer of 1 to 4. In such embodiments, m may be 1 or 2.

In an RNAi agent comprising formula O-(L-P)ₙ, the sense strand and the antisense strand may form a duplex, selected from the group consisting of: including a sense strand and an antisense strand, wherein the sense strand and the antisense strand form a duplex, comprising a sense strand and an antisense strand, wherein the sense strand and the antisense strand form a duplex, wherein the duplex comprises one of dsRNA 1-76 or 163-230 of Table 4, wherein optionally one or more nucleotides of the sense strand and the antisense strand are independently modified nucleotides, and wherein optionally one or more internucleotide linkages of the sense strand and the antisense strand are modified internucleotide linkages.

In specific embodiments, the sense strand and the antisense strand comprise a pair of nucleic acid sequences selected from the group consisting of dsRNA 77-162 or 231-326 of Table 6.

In one embodiment, the present disclosure provides an ALK7 RNAi agent of Formula:

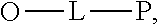

wherein O comprises a comprising a sense stand and an antisense strand, wherein
theantisense strand is complementary to ALK7 mRNA;
wherein L is a linker comprising the formula:

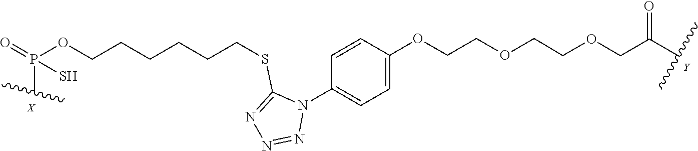

wherein P is an NPR-C-binding peptide comprising SEQ ID NO: 395.

In such an embodiment of the ALK7 RNAi agent the sense strand comprises a first nucleic acid sequence of SEQ ID NO: 115, and the antisense strand comprises a second nucleic acid sequence of SEQ ID NO: 191; or the sense strand comprises a first nucleic acid sequence of SEQ ID NO: 117, and the antisense strand comprises a second nucleic acid sequence of SEQ ID NO: 193; or the sense strand comprises a first nucleic acid sequence of SEQ ID NO: 133, and the antisense strand comprises a second nucleic acid sequence of SEQ ID NO: 209; or the sense strand comprises a first nucleic acid sequence of SEQ ID NO: 134, and the antisense strand comprises a second nucleic acid sequence of SEQ ID NO: 210; or the sense strand comprises a first nucleic acid sequence of SEQ ID NO: 135, and the antisense strand comprises a second nucleic acid sequence of SEQ ID NO: 211.

In one embodiment, the antisense strand is modified with 2′-fluoro at each of positions 2, 5, 7, 14, and 16. In one embodiment, all other positions of the antisense strand are modified with 2′-OMe.

In one embodiment of the ALK7 RNAi agent, the antisense strand comprises phosphorothioate linkages between positions 1 and 2, 2 and 3, 20 and 21, 21 and 22, and 22 and 23. In one embodiment, the ALK7 RNAi agent of any one of claims 47-51, wherein the antisense comprises a vinylphosphonate moiety conjugated to the 5′ end. In one embodiment of the ALK7 RNAi agent, the sense strand is modified with 2′-fluoro at each of positions 9, 10, and 11. In one embodiment of the ALK7 RNAi agent, all other positions of the sense strand are modified with 2′-OMe. In one embodiment of the ALK7 RNAi agent, wherein the sense strand comprises phosphorothioate linkages between positions 1 and 2, 19 and 20, and 20 and 21. In one embodiment, the sense strand comprises the inverted abasic moiety conjugated at the 5′ end, wherein the inverted abasic moiety is conjugated to the sense strand by a phosphorothioate linkage. In another embodiment, the linker L is attached to a 3′ end of the sense strand.

In one embodiment of the ALK7 RNAi agent, the sense strand comprises a first nucleic acid sequence of SEQ ID NO: 568, and the antisense strand comprises a second nucleic acid sequence of SEQ ID NO: 648; or the sense strand comprises a first nucleic acid sequence of SEQ ID NO: 569, and the antisense strand comprises a second nucleic acid sequence of SEQ ID NO: 649; or the sense strand comprises a first nucleic acid sequence of SEQ ID NO: 570, and the antisense strand comprises a second nucleic acid sequence of SEQ ID NO: 650; or the sense strand comprises a first nucleic acid sequence of SEQ ID NO: 571, and the antisense strand comprises a second nucleic acid sequence of SEQ ID NO: 651; or the sense strand comprises a first nucleic acid sequence of SEQ ID NO: 572, and the antisense strand comprises a second nucleic acid sequence of SEQ ID NO: 652; or the sense strand comprises a first nucleic acid sequence of SEQ ID NO: 573, and the antisense strand comprises a second nucleic acid sequence of SEQ ID NO: 649; or the sense strand comprises a first nucleic acid sequence of SEQ ID NO: 574, and the antisense strand comprises a second nucleic acid sequence of SEQ ID NO: 650; or the sense strand comprises a first nucleic acid sequence of SEQ ID NO: 575, and the antisense strand comprises a second nucleic acid sequence of SEQ ID NO: 652; or the sense strand comprises a first nucleic acid sequence of SEQ ID NO: 568, and the antisense strand comprises a second nucleic acid sequence of SEQ ID NO: 648; or the sense strand comprises a first nucleic acid sequence of SEQ ID NO: 576, and the antisense strand comprises a second nucleic acid sequence of SEQ ID NO: 648; or the sense strand comprises a first nucleic acid sequence of SEQ ID NO: 577, and the antisense strand comprises a second nucleic acid sequence of SEQ ID NO: 651.

The present disclosure also provides for a pharmaceutical composition comprising the ALK7 RNAi agent, and a pharmaceutically acceptable carrier.

The present disclosure also provides a method of treating a disease or condition of adipose tissue in a patient in need thereof, comprising administering to the patient an effective amount of the ALK7 RNAi agent or the pharmaceutical composition. The disease or condition may be obesity or obesity-related comorbidity. The RNAi agent or pharmaceutical composition may be administered intravenously or subcutaneously.

The present disclosure also provides for the ALK7 RNAi agent, or the pharmaceutical composition, for use in a therapy.

The present disclosure provides the ALK7 RNAi agent, or the pharmaceutical composition thereof, for use in the treatment of a disease or condition of adipose tissue. The disease or condition may be obesity or an obesity-related comorbidity.

The present disclosure provides for use of ALK7 RNAi agent, or the pharmaceutical composition thereof, in the manufacture of a medicament for treating a disease or condition of adipose tissue. The disease or condition may be obesity or an obesity-related comorbidity.

The present disclosure provides a method of delivering an oligonucleotide to an adipose tissue, comprising administering to a subject ALK7 RNAi agent or a pharmaceutical composition thereof.

### DETAILED DESCRIPTION

Provided herein are ALK7 RNAi agents and compositions comprising an ALK7 RNAi agent. Also provided herein are methods of using the ALK7 RNAi agents or compositions comprising an ALK7 RNAi agent for treating ALK7 associated metabolic disorder, such as obesity and/or an obesity-associated co-morbidity, in a subject.

In one aspect, provided herein are ALK7 RNAi agent comprising a sense strand and an antisense strand, wherein the sense strand and the antisense strand form a duplex, and wherein the antisense strand is complementary to a region of ALK7 mRNA. In some embodiments, provided herein are ALK7 RNAi agent comprising a sense strand and an antisense strand, wherein the sense strand and the antisense strand form a duplex, wherein the sense strand comprises a nucleic acid sequence selected from any one of SEQ ID NOs:78-153 and 230-315, and the antisense strand comprises a nucleic acid sequence selected from any one of SEQ ID NOs: 154-229 and 316-391, wherein optionally one or more nucleotides of the sense strand and the antisense strand are independently modified nucleotides, and wherein optionally one or more internucleotide linkages of the sense strand and the antisense strand are modified internucleotide linkages.

In another aspect, the ALK7 RNAi agent may be a conjugate including a nucleic acid component. In one instance, the conjugate may include an NPR-C binding peptide and a therapeutic agent, such as an oligonucleotide. The NPR-C binding peptide can deliver the therapeutic agent such as an oligonucleotide to adipose tissue with good selectivity. The conjugates optionally include a linker between the NPR-C binding peptide and the therapeutic agent (e.g., an oligonucleotide). The conjugates can also include one or more fatty acids. The present disclosure also includes methods and uses of treating diseases and conditions associated with adipose tissue (e.g., obesity or obesity-related comorbidity) by the conjugates or pharmaceutical composition described herein.

In one aspect, provided herein are conjugates comprising Formula (I): O-(L-P)., wherein O comprises an oligonucleotide; wherein L is a linker or a bond; wherein P is an NPR-C-binding peptide, wherein n is an integer of 1 to 4. In some embodiments, O is an antisense oligonucleotide (ASO), a double stranded RNA (dsRNA), such as a dsRNA including a sense strand and an antisense strand, or a guide RNA. In some embodiments, P is any NPR-C-binding peptide described herein, e.g., an NPR-C-binding peptide in Tables 1-3. In some embodiments, P is an NPR-C-binding antibody or a fragment thereof. In some embodiments, n is 1. In some embodiments, n is 2.

In some embodiments, provided herein are conjugates comprising Formula (I): 0-(L-P)., wherein O comprises a ALK7 RNAi agent; wherein L is a linker or a bond; wherein P is an NPR-C-binding antibody or a fragment thereof, and wherein n is an integer of 1 to 4. In some embodiments, P is a heteromab that binds NPR-C. In some embodiments, P is a Fab, VHH or scFv that binds NPR-C.

In some embodiments, L comprises a linker core and one or more spacers. In some embodiments, L comprises Spacer1-Linker Core-Spacer2.

In some embodiments, the conjugate further comprises a fatty acid (FA) or lipid. In some embodiments, FA is attached to O. In some embodiments, FA is attached to P. In some embodiments, FA is attached to L.

In some embodiments, the conjugate comprises (FA)m-O-L-P or O-L-P-(FA)m, wherein m is an integer of 1 to 4. In some embodiments, m is 1 or 2.

In some embodiments, the conjugate further comprises a Spacer3. In some embodiments, the conjugate comprises (FA-Spacer3)m-O-L-P or O-L-P-(Spacer3-FA)m, wherein m is an integer of 1 to 4. In some embodiments, m is 1 or 2. In some embodiments, the conjugate comprises FA-Spacer3-O-L-P or O-L-P-Spacer3-FA. In some embodiments, the conjugate comprises O-L-(FA-Spacer3)m-P or 0-(Spacer3-FA)m-L-P, wherein m is an integer of 1 to 4. In some embodiments, m is 1 or 2. In some embodiments, the conjugate comprises O-L-FA-Spacer3-P or O-Spacer3-FA-L-P.

### NPR-C Binding Peptide or Protein (P)

Natriuretic peptides (NPs) are a class of endogenous hormones which confer cardiovascular protection through regulation of body fluid homeostasis. They include several structurally related peptide hormones: Atrial Natriuretic Peptide (ANP) and variants such as Urodilatin and mANP, Brain Natriuretic Peptide (BNP), C-type Natriuretic Peptide (CNP) and Dendroaspis Natriuretic Peptide (DNP). Three subtypes of natriuretic peptide receptors (NPR) have been described and include NPR-A, NPR-B and NPR-C. Of these, NPR-C is enriched in adipose tissue, where it acts in the natriuretic peptide system to bind and clear NPs, removing them from circulating blood (Maack et al., Science 238:675-678 (1987)).

Nucleic acid therapeutics, including RNA interference (RNAi) agents such as small interfering RNA (siRNA) and antisense oligonucleotides (ASO) have the potential to selectively target and modulate individual genes related to a disease state. The effectiveness of such treatment can be related to how much of the therapeutic is delivered to the organ, tissue, or even cell of interest.

The conjugates provided herein include a peptide or protein that binds a human NPR-C receptor (“NPR-C binding peptide” or “NPR-C binding protein”). In some embodiments, the peptide binds NPR-C receptor with good affinity and selectivity.

In some embodiments, P is an NPR-C-binding peptide. In some embodiments, the NPR-C binding peptide is derived from ANP. Wild-type human ANP is a 28 amino acid peptide having a 17 amino acid loop formed by an intramolecular disulfide linkage between two cysteine residues present at positions 7 and 23 (SEQ ID NO: 10). It is a cardiac hormone that generally acts to maintain the cardiovascular system. It is part of the body's natural defense against hypoxia and pathological cardiac wall stress. Wild type ANP exhibits similar binding affinity for the receptors NPR-A and NPR-C, but a synthetic peptide C-ANP₄₋₂₃ has high selectivity for NPR-C, not binding to NPR-A at detectable levels. Both linear and cyclic C-ANP₄₋₂₃ bind NPR-C with high potency.

In some embodiments, the NPR-C binding peptide comprises a consensus sequence GX₁₁IDX₁₄I (SEQ ID NO: 1), wherein X₁₁ is arginine, proline, or hydroxyproline, and X₁₄ is arginine or N-methylarginine. In some embodiments, X₁₁ is R. In some embodiments, X₁₄ is R.

In some embodiments, the NPR-C binding peptide comprises GRIDRI (SEQ ID NO: 2).

In some embodiments, the NPR-C binding peptide comprises SX₇X₈X₉GX₁₁IDX₁₄I (SEQ ID NO: 3), wherein:
X₇ is glycine, alanine, proline, hydroxyproline, serine, or cysteine;
X₈ is phenylalanine, or cyclohexylalanine, and
X₉ is glycine, alanine, or serine.

In some embodiments, X₇ is C. In some embodiments, X₉ is G.

In some embodiments, the NPR-C binding peptide comprises SCFGGRIDRI (SEQ ID NO: 4).

In other embodiments, the NPR-C binding peptide comprises FGGRIDRIGA (SEQ ID NO: 5).

In some embodiments, the NPR-C binding peptide comprises RSSX₇FGGRIDRI (SEQ ID NO: 6), wherein X₇ is serine or cysteine.

In some embodiments, the NPR-C binding peptide comprises SEQ ID NO: 7 (RSSX₇FGGRIDRIGA). In certain embodiments, X₇ is cysteine. In other embodiments, X₇ is serine.

In some embodiments, the NPR-C binding peptide comprises SEQ ID NO: 8 (X₇-Cha-X₉GX₁₁IDX₁₄I), wherein:
X₇ is cysteine, proline, hydroxyproline, glycine, or alanine;
X₉ is alanine, serine, or glycine;
X₁₁ is proline, hydroxyproline, or arginine; and
X₁₄ is arginine or N-methylarginine.

In certain embodiments, the NPR-C binding peptide comprises SEQ ID NO: 9 (fSp-Cha-aGPIDRI).

Exemplary NPR-C binding peptides that can be used in conjugates described herein are provided in Tables 1-3. In some embodiments, the NPR-C binding peptide comprises a sequence selected from any one of SEQ ID NO: 11-57.

TABLE 1
Exemplary NPR-C binding peptides

| SEQ ID NO | Peptide Sequence |
|---|---|
| 11 | RSS[CFGGRIDRIGAQSGLGC]-NH₂ |
| 12 | RSSCFGGRIDRIGAQSGLGC-NH₂ |
| 13 | RSSSFGGRIDRIGAQSGLGS-NH₂ |
| 14 | RSS[CFGGRIDRIGAQSGLGC]-OH |
| 15 | RSSCFGGRIDRIGAQSGLGC-OH |
| 16 | RSSSFGGRIDRIGAQSGLGS-OH |
| 17 | RSSSFGGRIDRIGAQSGLGS-NH₂ |
| 18 | RSS[CFGGRIDRIGAC]-NH₂ |
| 19 | RSS[CFGGRIDRIGAQSGLGC]^{CH2}-OH |
| 20 | RSS[CFGGRIDRIGAQSGLGC]^{CH2}-NH₂ |
| 21 | fSp-Cha-aGPIDRI-NH₂ |
| 22 | fS-(D-Hyp)-Cha-sG-Hyp-ID-Arg(Me)- |
| | I-NHCH3 |
| 23 | fSp-Cha-aGPIDRIGSPSSGAPPPS-NH₂ |
| 24 | fSp-Cha-aGPIDRI-OH |
| 395 | GGGGEGGGGEGGGGEKEKEKGGGGSGGGGS- |
| | fSp-Cha-aGPIDRI-NH₂ |

TABLE 2
Exemplary NPR-C binding peptides

| SEQ ID NO | Peptide Sequence |
|---|---|
| 25 | Orn-fSp-Cha-aGPIDSI-NH₂ |
| 26 | fSp-Cha-aGPIDSI-Dap-NH₂ |
| 27 | fS-(D-Pip)-Cha-aGPIDSI-NH₂ |
| 28 | FSG-Cha-GGRIDRI-NH₂ |
| 29 | FSG-Cha-Aib-GRIDRI-NH₂ |
| 30 | fSp-Cha-aGPIERI-NH₂ |
| 31 | fSp-Cha-aGPID-(NMe-Arg)-I-NH₂ |
| 32 | RSFSGFGGRIDRI-NH₂ |
| 33 | RSFSGFGGRIDRIGAQSGLGS-NH₂ |
| 34 | RSFSGFGGRIDRIG-NH₂ |

TABLE 3
Exemplary NPR-C binding peptides

| SEQ ID NO | Peptide Sequence |
|---|---|
| 35 | C-Cha-GGRIDRIG-NH₂ |
| 36 | Ac-C-Cha-GGRIDRIG-NH₂ |
| 37 | S-Cha-GGRIDRIG-NH₂ |
| 38 | A-Cha-GGRIDRIG-NH₂ |
| 39 | Cha-GGRIDRIG-NH₂ |
| 40 | Ac-Cha-GGRIDRIG-NH₂ |
| 41 | G-Cha-GGRIDRIG-NH₂ |
| 42 | SG-Cha-GGRIDRIG-NH₂ |
| 43 | FSG-Cha-GGRIDRIG-NH₂ |
| 44 | SFSG-Cha-GGRIDRIG-NH₂ |
| 45 | RSFSG-Cha-GGRIDRIG-NH₂ |
| 46 | RSSG-Cha-GGRIDRIG-NH₂ |
| 47 | fSG-Cha-GGRIDRIG-NH₂ |
| 48 | Ac-fSG-Cha-GGRIDRIG-NH₂ |
| 49 | fSp-Cha-GGRIDRIG-NH₂ |
| 50 | fSG-Cha-aGRIDRIG-NH₂ |
| 51 | fSG-Cha-GGPIDRIG-NH₂ |
| 52 | Ac-fSp-Cha-aGPIDRIG-NH₂ |
| 53 | Ac-fSp-Cha-aGPIDRI-NH₂ |
| 54 | Ac-fSp-Cha-aGPIDRI-NHCH₃ |
| 55 | Ac-fSp-Cha-aGPID-Arg(Me)-I-NHCH₃ |
| 56 | Ac-fS-(D-Hyp)-Cha-aG-Hyp-ID-Arg(Me)- |
| | I-NHCH₃ |
| 57 | (HOCH2CO)-fS-(D-Hyp)-Cha-sG-Hyp-ID- |
| | Arg(Me-)I-NHCH₃ |

Tables 1-3 do not represent an exhaustive listing of all NPR-C binding peptides which may be utilized in a conjugate disclosed herein. In some instances, one or more amino acids may be substituted for an equivalent number of amino acids representing conservative mutations to the sequence, as are known in the art. For example, in some instances an isoleucine may be substituted where a leucine is indicated, and vice versa.

Suitable NPR-C binding peptides may be of a variety of lengths. In one aspect, the length of the peptide may be from 6 to 30 amino acids. In another aspect, the length of the peptide may be from 7 to 28 amino acids. In another aspect, the length of the peptide may be from 8 to 26 amino acids. In another aspect, the length of the peptide may be from 9 to 24 amino acids. In another aspect, the length of the peptide may be from 10 to 22 amino acids. The peptide may be 6 amino acids long, or 7 amino acids, or 8 amino acids, or 9 amino acids, or 10 amino acids, or 11 amino acids, or 12 amino acids, or 13 amino acids, or 14 amino acids, or 15 amino acids, or 16 amino acids, or 17 amino acids, or 18 amino acids, or 19 amino acids, or 20 amino acids, or 21 amino acids, or 22 amino acids, or 23 amino acids, or 24 amino acids, or 25 amino acids, or 26 amino acids, or 27 amino acids, or 28 amino acids, or 29 amino acids, or 30 amino acids in length.

As mentioned above, the NPR-C binding peptide may be modified. It may be modified at the N-terminal end, at the C-terminal end, at an internal position, or any combination of these. For example, the C-terminal carboxylic acid moiety of a peptide may be converted to an amide to generate a different peptide having the same amino acid sequence (see for example SEQ ID NO: 16 and SEQ ID NO: 17). In some embodiments, NPR-C binding peptide comprises a C-terminal hydroxyl. In some embodiments, NPR-C binding peptide comprises a C-terminal amide.

In some instances, the NPR-C binding peptide may be a linear peptide; that is, one in which there is no intramolecular bond made between any of the N-terminal end, the C-terminal end, and any of the side chains of the amino acids which constitute the peptide.

In other instances, the NPR-C binding peptide may be a cyclic peptide, in which there is exactly one or at least one intramolecular bond made between any of the N-terminal end, the C-terminal end, and any of the side chains of the amino acids which constitute the peptide. The bond which cyclizes the peptide may in one embodiment include atoms only derived from the parent peptide itself, for example a disulfide bond between cysteine side chains, as shown in Formula II below. As illustrated, Formula II shows the cysteine residues separated by 9 amino acids, with each X representing an amino acid residue. It will be appreciated that the amino acid chain may be of any length:

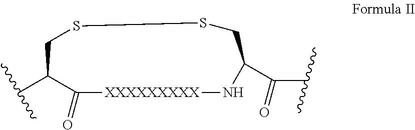

In another embodiment, the peptide may be cyclized using additional atoms in the intramolecular bond, such as an intervening alkyl group between the side chains of two cysteine residues. As illustrated, Formula III shows the cysteine residues separated by 9 amino acids, with each X representing an amino acid residue. It will be appreciated that the amino acid chain may be of any length. The value of z can be any integer from 1 to 20 inclusive.

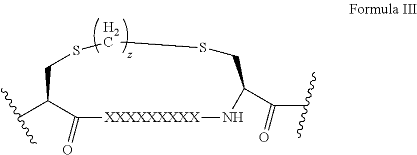

In the formula above, when z=1, the peptide contains a thioacetal moiety, defined by the side chains of the cysteine residues and the methylene group bridging the same.

In some embodiments, the NPR-C binding peptide is cyclized by covalent attachment of a side chain of a first cysteine residue to a side chain of a second cysteine residue. In some embodiments, the covalent attachment comprises a disulfide bond. In some embodiments, the covalent attachment comprises a thioacetal moiety.

In some instances, the NPR-C binding peptide may be a single copy of one of the sequences specified herein. Also envisaged are delivery moieties containing multiple copies of peptides disclosed herein, such as duplications of certain sequences, the use of multiple distinct peptides to generate a single construct, and so forth.

In some embodiments, P is an NPR-C-binding antibody or a fragment thereof. In some embodiments, P is a heteromab that binds NPR-C. In some embodiments, P is a Fab, VHH or scFv that binds NPR-C.

### Oligonucleotide

The conjugates described herein comprise an oligonucleotide. In some embodiments, O is an antisense oligonucleotide, a double stranded RNA (dsRNA), or a guide RNA.

In some embodiments, O is a double stranded RNA (dsRNA) comprising a sense stand and an antisense strand. In some embodiments, at least one nucleotide of the sense strand is a modified nucleotide. In some embodiments, at least one nucleotide of the antisense strand is a modified nucleotide. In some embodiments, at least one internucleotide linkage of the sense strand is a modified internucleotide linkage. In some embodiments, at least one internucleotide linkage of the antisense strand is a modified internucleotide linkage. In some embodiments, the dsRNA comprises a sense strand and an antisense stand, wherein the antisense strand is complementary to a target mRNA, that is, ALK7 mRNA.

Exemplary unmodified sense strand and antisense strand sequences of dsRNA targeting ALK7 mRNA are provided in Table 4.

TABLE 4
Unmodified Nucleic Acid Sequences of dsRNA targeting human ALK7 mRNA
(ALK7 siRNA)

| | | | | | Starting target |
|---|---|---|---|---|---|
| | | SEQ | | SEQ | Position on |
| dsRNA | Unmodified Sense | ID | Unmodified Antisense | ID | gene transcript |
| NO | 5′ to 3′ | NO | 5′ to 3′ | NO | (NM_145259.3) |
| 1 | UUUUAUUUUACUU | 78 | UUUGGUUCAAGUAAA | 154 | 2014 |
| | GAACCAAA | | AUAAAAAC | | |
| 2 | CCCGCCUAACUGC | 79 | UACGAAGAGCAGUUA | 155 | 1632 |
| | UCUUCGUA | | GGCGGGCC | | |
| 3 | CUAAUGAUGAUAA | 80 | UAACAUAAUUAUCAU | 156 | 1699 |
| | UUAUGUUA | | CAUUAGGC | | |
| 4 | AAGUAUAAAAUAA | 81 | UAUAGAGCUUAUUUU | 157 | 1972 |
| | GCUCUAUA | | AUACUUUU | | |
| 5 | UCCAUUUUCCCCU | 82 | UCACAUAAAGGGGAA | 158 | 1746 |
| | UUAUGUGA | | AAUGGAAA | | |
| 6 | AUGUUAAAAAGAA | 83 | UGAGAGAUUUCUUUU | 159 | 1714 |
| | AUCUCUCA | | UAACAUAA | | |
| 7 | AUAAGACAGUACA | 84 | UUAAAUACUGUACUG | 160 | 1803 |
| | GUAUUUAA | | UCUUAUCU | | |
| 8 | AACUCUAAAGUUA | 85 | UCCAUGCUUAACUUU | 161 | 1850 |
| | AGCAUGGA | | AGAGUUAU | | |
| 9 | UUUAAUUUUAUUU | 86 | UGCUUUCAAAAUAAA | 162 | 1904 |
| | UGAAAGCA | | AUUAAACA | | |
| 10 | GUUAUAGUCAUUA | 87 | UAAAAACUUAAUGAC | 163 | 1998 |
| | AGUUUUUA | | UAUAACAU | | |
| 11 | UUUAUGUGAAUGU | 88 | UGGCAAAAACAUUCA | 164 | 1758 |
| | UUUUGCCA | | CAUAAAGG | | |
| 12 | UUAUGACAUGGUG | 89 | UCUGAAGGCACCAUG | 165 | 1474 |
| | CCUUCAGA | | UCAUAAUA | | |
| 13 | UCAAAGAAGACUG | 90 | UGGCUUUGCAGUCUU | 166 | 1680 |
| | CAAAGCCA | | CUUUGACA | | |
| 14 | UGAAAAGAUAACU | 91 | UACUUUAGAGUUAUC | 167 | 1841 |
| | CUAAAGUA | | UUUUCAUG | | |
| 15 | UUUACUUGAACCA | 92 | UGUGCUCUUGGUUCA | 168 | 2020 |
| | AGAGCACA | | AGUAAAAU | | |
| 16 | CAACUCAUCUUUU | 93 | UUUAAAUAAAAAGAU | 169 | 1931 |
| | UAUUUAAA | | GAGUUGAG | | |
| 17 | AUGAUGAUAAUUA | 94 | UUUUAACAUAAUUAU | 170 | 1702 |
| | UGUUAAAA | | CAUCAUUA | | |
| 18 | UAUAUUACAAAAG | 95 | UUUUUAUACUUUUGU | 171 | 1962 |
| | UAUAAAAA | | AAUAUAUU | | |
| 19 | GUGAUAGUACAUG | 96 | UAUGCUAACAUGUAC | 172 | 3167 |
| | UUAGCAUA | | UAUCACUU | | |
| 20 | UUGCCUUAUUAUG | 97 | UACCAUGUCAUAAUA | 173 | 1466 |
| | ACAUGGUA | | AGGCAAUU | | |
| 21 | UAGCAUAUAAUGU | 98 | UUCAAAAUACAUUAU | 174 | 3181 |
| | AUUUUGAA | | AUGCUAAC | | |
| 22 | ACUUCUGUAUUUU | 99 | UCUAUGAGAAAAUAC | 175 | 3416 |
| | CUCAUAGA | | AGAAGUAU | | |
| 23 | UUUUGUGUGAUUC | 100 | UGUUUGAAGAAUCAC | 176 | 312 |
| | UUCAAACA | | ACAAAAGA | | |
| 24 | UCUAAAGUUAAGC | 101 | UUGCCCAUGCUUAAC | 177 | 1853 |
| | AUGGGCAA | | UUUAGAGU | | |
| 25 | GACAUGGUGCCUU | 102 | UGGAUCUGAAGGCAC | 178 | 1478 |
| | CAGAUCCA | | CAUGUCAU | | |
| 26 | GCCCGCCUAACUG | 103 | UCGAAGAGCAGUUAG | 179 | 1631 |
| | CUCUUCGA | | GCGGGCCG | | |
| 27 | UCAAAGAAUAUCU | 104 | UUUCACUAAGAUAUU | 180 | 1196 |
| | UAGUGAAA | | CUUUGAUU | | |
| 28 | GUAUCCCAAACCA | 105 | UUUGCCACUGGUUUG | 181 | 1548 |
| | GUGGCAAA | | GGAUACUU | | |
| 29 | UUUUAUUAUGAUA | 106 | UCCACAAAUAUCAUA | 182 | 2513 |
| | UUUGUGGA | | AUAAAAGA | | |
| 30 | UUUUUUUAAACUG | 107 | UAGCAAUCCAGUUUA | 183 | 3277 |
| | GAUUGCUA | | AAAAAAAA | | |
| 31 | UGUCUUUUGUGUG | 108 | UGAAGAAUCACACAA | 184 | 308 |
| | AUUCUUCA | | AAGACAUA | | |
| 32 | CUCUAAAGUUAAG | 109 | UGCCCAUGCUUAACU | 185 | 1852 |
| | CAUGGGCA | | UUAGAGUU | | |
| 33 | UUUUACUUGAACC | 110 | UUGCUCUUGGUUCAA | 186 | 2019 |
| | AAGAGCAA | | GUAAAAUA | | |
| 34 | UCCUGAAAUGCUU | 111 | UUAUCAUCAAGCAUU | 187 | 1330 |
| | GAUGAUAA | | UCAGGAGC | | |
| 35 | UGAAAUGCUUGAU | 112 | UUUGUAUCAUCAAGC | 188 | 1333 |
| | GAUACAAA | | AUUUCAGG | | |
| 36 | CAAGGUAAACCUG | 113 | UGCAAUAGCAGGUUU | 189 | 1160 |
| | CUAUUGCA | | ACCUUGUG | | |
| 37 | GGGCUCCUUAUAU | 114 | UAAUAGUCAUAUAAG | 190 | 1045 |
| | GACUAUUA | | GAGCCCUG | | |
| 38 | GGCUCCUGAAAUG | 115 | UCAUCAAGCAUUUCA | 191 | 1327 |
| | CUUGAUGA | | GGAGCCAU | | |
| 39 | UUUAUUUUACUUG | 116 | UCUUGGUUCAAGUAA | 192 | 2015 |
| | AACCAAGA | | AAUAAAAA | | |
| 40 | UUGAUGAUACAAU | 117 | UCACAUUCAUUGUAU | 193 | 1341 |
| | GAAUGUGA | | CAUCAAGC | | |
| 41 | UGAUUCAAUACUG | 118 | UUAGUGUUCAGUAUU | 194 | 1264 |
| | AACACUAA | | GAAUCAUG | | |
| 42 | AUCUAUUCUGUUG | 119 | UACCAGACCAACAGA | 195 | 1391 |
| | GUCUGGUA | | AUAGAUGU | | |
| 43 | AGUUGACUUCAUC | 120 | UGAGAUUGGAUGAAG | 196 | 1875 |
| | CAAUCUCA | | UCAACUCC | | |
| 44 | CUCAUCGAGACAU | 121 | UUGAUUUUAUGUCUC | 197 | 1179 |
| | AAAAUCAA | | GAUGAGCA | | |
| 45 | UACCUCAAAGAUA | 122 | UACUGUCUUAUCUUU | 198 | 1793 |
| | AGACAGUA | | GAGGUAGA | | |
| 46 | UAUAGUCAUUAAG | 123 | UAUAAAAACUUAAUG | 199 | 2000 |
| | UUUUUAUA | | ACUAUAAC | | |
| 47 | CAAAGAUAAGACA | 124 | UACUGUACUGUCUUA | 200 | 1798 |
| | GUACAGUA | | UCUUUGAG | | |
| 48 | AAAGAAUAUCUUA | 125 | UUUUUCACUAAGAUA | 201 | 1198 |
| | GUGAAAAA | | UUCUUUGA | | |
| 49 | ACCAAUUGCCUUA | 126 | UGUCAUAAUAAGGCA | 202 | 1461 |
| | UUAUGACA | | AUUGGUAC | | |
| 50 | UUUUUGUUCUACC | 127 | UUCUUUGAGGUAGAA | 203 | 1784 |
| | UCAAAGAA | | CAAAAAAA | | |
| 51 | UUCCAUUUUCCCC | 128 | UACAUAAAGGGGAAA | 204 | 1745 |
| | UUUAUGUA | | AUGGAAAA | | |
| 52 | CUACCUCAAAGAU | 129 | UCUGUCUUAUCUUUG | 205 | 1792 |
| | AAGACAGA | | AGGUAGAA | | |
| 53 | AAUCAAAGAAUAU | 130 | UCACUAAGAUAUUCU | 206 | 1194 |
| | CUUAGUGA | | UUGAUUUU | | |
| 54 | AUGCUUGAUGAUA | 131 | UUUCAUUGUAUCAUC | 207 | 1337 |
| | CAAUGAAA | | AAGCAUUU | | |
| 55 | UAGGUGAUGCAAU | 132 | UAACAUAGAUUGCAU | 208 | 2126 |
| | CUAUGUUA | | CACCUAAA | | |
| 56 | AUAUCUCAACUUU | 133 | UUUGACACAAAGUUG | 209 | 1664 |
| | GUGUCAAA | | AGAUAUAG | | |
| 57 | AUGAAUGUGAAUA | 134 | UUCAAAGAUAUUCAC | 210 | 1352 |
| | UCUUUGAA | | AUUCAUUG | | |
| 58 | UUCAAACGAGCUG | 135 | UUAGAUGUCAGCUCG | 211 | 1376 |
| | ACAUCUAA | | UUUGAAGG | | |
| 59 | AAAAAUAAAUAUU | 136 | UUUAGAGCAAUAUUU | 212 | 2175 |
| | GCUCUAAA | | AUUUUUAA | | |
| 60 | CUCUCAUAGCUUU | 137 | UGGAAAAGAAAGCUA | 213 | 1729 |
| | CUUUUCCA | | UGAGAGAU | | |
| 61 | AUAGAGGAAAUGA | 138 | UACCUUUCUCAUUUCC | 214 | 1502 |
| | GAAAGGUA | | UCUAUCG | | |
| 62 | GUGAAAAAGUGUG | 139 | UCAAGUUUCACACUU | 215 | 1211 |
| | AAACUUGA | | UUUCACUA | | |
| 63 | UGGACUCAACUUU | 140 | UACCAGCCAAAGUUG | 216 | 1007 |
| | GGCUGGUA | | AGUCCAAG | | |
| 64 | UUAAGUUUUUAUU | 141 | UCAAGUAAAAUAAAA | 217 | 2008 |
| | UUACUUGA | | ACUUAAUG | | |
| 65 | GAAGAUGUGGCUG | 142 | UAUUUUCACAGCCAC | 218 | 869 |
| | UGAAAAUA | | AUCUUCCC | | |
| 66 | UAUGUUAAAAAGA | 143 | UAGAGAUUUCUUUUU | 219 | 1713 |
| | AAUCUCUA | | AACAUAAU | | |
| 67 | UAAUGAUGAUAAU | 144 | UUAACAUAAUUAUCA | 220 | 1700 |
| | UAUGUUAA | | UCAUUAGG | | |
| 68 | GAUAAUUAUGUUA | 145 | UUUCUUUUUAACAUA | 221 | 1707 |
| | AAAAGAAA | | AUUAUCAU | | |
| 69 | GACUGCAAAGCCU | 146 | UCAUCAUUAGGCUUU | 222 | 1688 |
| | AAUGAUGA | | GCAGUCUU | | |
| 70 | CAUUAAGUUUUUA | 147 | UAGUAAAAUAAAAAC | 223 | 2006 |
| | UUUUACUA | | UUAAUGAC | | |
| 71 | ACUGCUCUUCGUA | 148 | UUUCUUAAUACGAAG | 224 | 1640 |
| | UUAAGAAA | | AGCAGUUA | | |
| 72 | UUAUGUUAAAAAG | 149 | UGAGAUUUCUUUUUA | 225 | 1712 |
| | AAAUCUCA | | ACAUAAUU | | |
| 73 | GUUGACUUCAUCC | 150 | UAGAGAUUGGAUGAA | 226 | 1876 |
| | AAUCUCUA | | GUCAACUC | | |
| 74 | CCUGAAAUGCUUG | 151 | UGUAUCAUCAAGCAU | 227 | 1331 |
| | AUGAUACA | | UUCAGGAG | | |
| 75 | GAAAUGCUUGAUG | 152 | UAUUGUAUCAUCAAG | 228 | 1334 |
| | AUACAAUA | | CAUUUCAG | | |
| 76 | UCCUUAUAUGACU | 153 | UUUCAAAUAGUCAUA | 229 | 1049 |
| | AUUUGAAA | | UAAGGAGC | | |
| 163 | CUUGAUGAUACAA | 396 | UACAUUCAUUGUAUC | 457 | 1340 |
| | UGAAUGUA | | AUCAAGCA | | |
| 164 | UGCUUGAUGAUAC | 397 | UAUUCAUUGUAUCAU | 458 | 1338 |
| | AAUGAAUA | | CAAGCAUU | | |
| 165 | UGUUAGCAUAUAA | 398 | UAAAUACAUUAUAUG | 459 | 3178 |
| | UGUAUUUA | | CUAACAUG | | |
| 166 | UCAUUAAGUUUUU | 399 | UGUAAAAUAAAAACU | 460 | 2005 |
| | AUUUUACA | | UAAUGACU | | |
| 167 | AGACUGCAAAGCC | 400 | UAUCAUUAGGCUUUG | 461 | 1687 |
| | UAAUGAUA | | CAGUCUUC | | |
| 168 | GGAAGAUGUGGCU | 401 | UUUUUCACAGCCACA | 462 | 868 |
| | GUGAAAAA | | UCUUCCCC | | |
| 169 | UAGAGGAAAUGAG | 402 | UAACCUUUCUCAUUU | 463 | 1503 |
| | AAAGGUUA | | CCUCUAUC | | |
| 170 | GGUGAUGCAAUCU | 403 | UGAAACAUAGAUUGC | 464 | 2128 |
| | AUGUUUCA | | AUCACCUA | | |
| 171 | UUCUACCUCAAAG | 404 | UGUCUUAUCUUUGAG | 465 | 1790 |
| | AUAAGACA | | GUAGAACA | | |
| 172 | UAACUGCUCUUCG | 405 | UCUUAAUACGAAGAG | 466 | 1638 |
| | UAUUAAGA | | CAGUUAGG | | |
| 173 | AGGUAAACCUGCU | 406 | UGAGCAAUAGCAGGU | 467 | 1162 |
| | AUUGCUCA | | UUACCUUG | | |
| 174 | UUUCCAUUUUCCC | 407 | UCAUAAAGGGGAAAA | 468 | 1744 |
| | CUUUAUGA | | UGGAAAAG | | |
| 175 | GCUGGUAUCUGAA | 408 | UCAUGAUAUUCAGAU | 469 | 1021 |
| | UAUCAUGA | | ACCAGCCA | | |
| 176 | AAUAUAUUACAAA | 409 | UUUAUACUUUUGUAA | 470 | 1960 |
| | AGUAUAAA | | UAUAUUUC | | |
| 177 | AUUAUGUUAAAAA | 410 | UAGAUUUCUUUUUAA | 471 | 1711 |
| | GAAAUCUA | | CAUAAUUA | | |
| 178 | ACUAUAUCUCAAC | 411 | UACACAAAGUUGAGA | 472 | 1661 |
| | UUUGUGUA | | UAUAGUCU | | |
| 179 | GGAGUUGACUUCA | 412 | UGAUUGGAUGAAGUC | 473 | 1873 |
| | UCCAAUCA | | AACUCCUG | | |
| 180 | AAAAUAAAUAUUG | 413 | UUUUAGAGCAAUAUU | 474 | 2176 |
| | CUCUAAAA | | UAUUUUUA | | |
| 181 | CUGUCAUAGUUCC | 414 | UCAUUGUUGGAACUA | 475 | 442 |
| | AACAAUGA | | UGACAGAA | | |
| 182 | GCCUAAUGAUGAU | 415 | UCAUAAUUAUCAUCA | 476 | 1697 |
| | AAUUAUGA | | UUAGGCUU | | |
| 183 | AUAACUCUAAAGU | 416 | UAUGCUUAACUUUAG | 477 | 1848 |
| | UAAGCAUA | | AGUUAUCU | | |
| 184 | UAAUUUUAUUUUG | 417 | UUUGCUUUCAAAAUA | 478 | 1906 |
| | AAAGCAAA | | AAAUUAAA | | |
| 185 | AAGAUAAGACAGU | 418 | UAUACUGUACUGUCU | 479 | 1800 |
| | ACAGUAUA | | UAUCUUUG | | |
| 186 | AUGAUAAUUAUGU | 419 | UCUUUUUAACAUAAU | 480 | 1705 |
| | UAAAAAGA | | UAUCAUCA | | |
| 187 | UCUCAACUUUGUG | 420 | UUCUUUGACACAAAG | 481 | 1667 |
| | UCAAAGAA | | UUGAGAUA | | |
| 188 | UUUUUUGUUCUAC | 421 | UCUUUGAGGUAGAAC | 482 | 1783 |
| | CUCAAAGA | | AAAAAAAA | | |
| 189 | AUUACAAAAGUAU | 422 | UUUAUUUUAUACUUU | 483 | 1965 |
| | AAAAUAAA | | UGUAAUAU | | |
| 190 | AAAGCCUAAUGAU | 423 | UAAUUAUCAUCAUUA | 484 | 1694 |
| | GAUAAUUA | | GGCUUUGC | | |
| 191 | CUCAUAGCUUUCU | 424 | UAUGGAAAAGAAAGC | 485 | 1731 |
| | UUUCCAUA | | UAUGAGAG | | |
| 192 | GCAUAUAAUGUAU | 425 | UCUUCAAAAUACAUU | 486 | 3183 |
| | UUUGAAGA | | AUAUGCUA | | |
| 193 | CUGCUCUUCGUAU | 426 | UCUUCUUAAUACGAA | 487 | 1641 |
| | UAAGAAGA | | GAGCAGUU | | |
| 194 | GUUUUUAUUUUAC | 427 | UGGUUCAAGUAAAAU | 488 | 2012 |
| | UUGAACCA | | AAAAACUU | | |
| 195 | CCAUUUUCCCCUU | 428 | UUCACAUAAAGGGGA | 489 | 1747 |
| | UAUGUGAA | | AAAUGGAA | | |
| 196 | AUAUUACAAAAGU | 429 | UAUUUUAUACUUUUG | 490 | 1963 |
| | AUAAAAUA | | UAAUAUAU | | |
| 197 | GCUCCUGAAAUGC | 430 | UUCAUCAAGCAUUUC | 491 | 1328 |
| | UUGAUGAA | | AGGAGCCA | | |
| 198 | AUAAUUAUGUUAA | 431 | UUUUCUUUUUAACAU | 492 | 1708 |
| | AAAGAAAA | | AAUUAUCA | | |
| 199 | UGAUGAUAAUUAU | 432 | UUUUUAACAUAAUUA | 493 | 1703 |
| | GUUAAAAA | | UCAUCAUU | | |
| 200 | ACUGCAAAGCCUA | 433 | UUCAUCAUUAGGCUU | 494 | 1689 |
| | AUGAUGAA | | UGCAGUCU | | |
| 201 | ACACAAGGUAAAC | 434 | UAUAGCAGGUUUACC | 495 | 1157 |
| | CUGCUAUA | | UUGUGUAC | | |
| 202 | UGAUAAUUAUGUU | 435 | UUCUUUUUAACAUAA | 496 | 1706 |
| | AAAAAGAA | | UUAUCAUC | | |
| 203 | AAGACUGCAAAGC | 436 | UUCAUUAGGCUUUGC | 497 | 1686 |
| | CUAAUGAA | | AGUCUUCU | | |
| 204 | AAAUAUAUUACAA | 437 | UUAUACUUUUGUAAU | 498 | 1959 |
| | AAGUAUAA | | AUAUUUCU | | |
| 205 | AACUGCUCUUCGU | 438 | UUCUUAAUACGAAGA | 499 | 1639 |
| | AUUAAGAA | | GCAGUUAG | | |
| 206 | CCUCAAAGAUAAG | 439 | UGUACUGUCUUAUCU | 500 | 1795 |
| | ACAGUACA | | UUGAGGUA | | |
| 207 | UUACUUGAACCAA | 440 | UUGUGCUCUUGGUUC | 501 | 2021 |
| | GAGCACAA | | AAGUAAAA | | |
| 208 | UAUCUCAACUUUG | 441 | UUUUGACACAAAGUU | 502 | 1665 |
| | UGUCAAAA | | GAGAUAUA | | |
| 209 | UGCAAAGCCUAAU | 442 | UUAUCAUCAUUAGGC | 503 | 1691 |
| | GAUGAUAA | | UUUGCAGU | | |
| 210 | UAUUACAAAAGUA | 443 | UUAUUUUAUACUUUU | 504 | 1964 |
| | UAAAAUAA | | GUAAUAUA | | |
| 211 | UGGUAUCUGAAUA | 444 | UUUCAUGAUAUUCAG | 505 | 1023 |
| | UCAUGAAA | | AUACCAGC | | |
| 212 | AUGAAUGUGAAUA | 134 | UUCAAAGAUAUUCAC | 210 | 1352 |
| | UCUUUGAA | | AUUCAUUG | | |
| 213 | GGCUCCUGAAAUG | 115 | UCAUCAAGCAUUUCA | 191 | 1327 |
| | CUUGAUGA | | GGAGCCAU | | |
| 214 | GUCUGUCAUAGUU | 445 | AUUGUUGGAACUAUG | 506 | 440 |
| | CCAACAAU | | ACAGAC | | |
| 215 | AUGAAUGUGAAUA | 134 | UUCAAACAUAUUCAC | 507 | 1352 |
| | UCUUUGAA | | AUUCAUUG | | |
| 216 | AUGAAUGUGAAUA | 134 | UUCAAAIAUAUUCACA | 508 | 1352 |
| | UCUUUGAA | | UUCAUUG | | |
| 217 | AUGAAUGUGAAUA | 446 | UUCAAACAUAUUCAC | 507 | 1352 |
| | UGUUUGAA | | AUUCAUUG | | |
| 218 | AUGAUUGUGAAUA | 447 | UUCAAAGAUAUUCAC | 509 | 1352 |
| | UCUUUGAA | | AAUCAUUG | | |
| 219 | UUCCAUUUUCCCC | 128 | UACAUAAAGGGGAAA | 510 | 1745 |
| | UUUAUGUA | | AUGGAA | | |
| 220 | GGCUCCUGAAAUG | 448 | UCAUCAIGCAUUUCAG | 511 | 1327 |
| | CCUGAUGA | | GAGCCAU | | |
| 221 | CAUAGCUUUCUUU | 449 | UAAAUGGAAAAGAAA | 512 | 1733 |
| | UCCAUUUA | | GCUAUGAG | | |
| 222 | UUGAUGAUACAAU | 450 | UCACAUICAUUGUAUC | 513 | 1341 |
| | GCAUGUGA | | AUCAAGC | | |
| 223 | UUCAAACGAGCUG | 451 | UUAGAUCUCAGCUCG | 514 | 1376 |
| | AGAUCUAA | | UUUGAAGG | | |
| 224 | UUCAUACGAGCUG | 452 | UUAGAUGUCAGCUCG | 515 | 1376 |
| | ACAUCUAA | | UAUGAAGG | | |
| 225 | UUCAAACGAGCUG | 135 | UUAGAUIUCAGCUCGU | 516 | 1376 |
| | ACAUCUAA | | UUGAAGG | | |
| 226 | AUCUCAACUUUGU | 453 | UUUGACACAAAGUUG | 517 | 1666 |
| | GUCAAA | | AGAUAU | | |
| 227 | UAUCUCAACUUUG | 454 | UUUGACACAAAGUUG | 518 | 1665 |
| | UGUCAAA | | AGAUAUA | | |
| 228 | AUAUCUCAACUUU | 133 | UUUGACACAAAGUUG | 517 | 1664 |
| | GUGUCAAA | | AGAUAU | | |
| 229 | UAUAUCUCAACUU | 455 | UUUGACACAAAGUUG | 518 | 1663 |
| | UGUGUCAAA | | AGAUAUA | | |
| 230 | CUAUAUCUCAACU | 456 | UUUGACACAAAGUUG | 209 | 1662 |
| | UUGUGUCAAA | | AGAUAUAG | | |

The dsRNA can include modifications. The modifications can be made to one or more nucleotides of the sense and/or antisense strand or to the internucleotide linkages, which are the bonds between two nucleotides in the sense or antisense strand. For example, some 2′-modifications of ribose or deoxyribose can increase RNA or DNA stability and half-life. Such 2′-modifications can be 2′-fluoro, 2′-O-methyl (i.e., 2′-methoxy), or 2′-O-alkyl (e.g., 2′-O—C₁₆ alkyl).

In some embodiments, one or more nucleotides of the sense strand and/or the antisense strand are independently modified nucleotides, which means the sense strand and the antisense strand can have different modified nucleotides. In some embodiments, each nucleotide of the sense strand is a modified nucleotide. In some embodiments, each nucleotide of the antisense strand is a modified nucleotide. In some embodiments, the modified nucleotide is a 2′-fluoro modified nucleotide, 2′-O-methyl modified nucleotide, or 2′-O-alkyl (e.g., 2′-O—C₁₆ alkyl) modified nucleotide. In some embodiments, each nucleotide of the sense strand and the antisense strand is independently a modified nucleotide, e.g., a 2′-fluoro modified nucleotide, 2′-O-methyl modified nucleotide, or 2′-O-alkyl (e.g., 2′-O—C₁₆ alkyl) modified nucleotide.

In some embodiments, the sense strand has four 2′-fluoro modified nucleotides, e.g., at positions 7, 9, 10, 11 from the 5′ end of the sense strand. In some embodiments, the other nucleotides of the sense strand are 2′-O-methyl modified nucleotides. In some embodiments, the antisense strand has four 2′-fluoro modified nucleotides, e.g., at positions 2, 6, 14, 16 from the 5′ end of the antisense strand. In some embodiments, the other nucleotides of the antisense strand are 2′-O-methyl modified nucleotides.

In some embodiments, the sense strand has three 2′-fluoro modified nucleotides, e.g., at positions 9, 10, 11 from the 5′ end of the sense strand. In some embodiments, the other nucleotides of the sense strand are 2′-O-methyl modified nucleotides. In some embodiments, the antisense strand has five 2′-fluoro modified nucleotides, e.g., at positions 2, 5, 7, 14, 16 from the 5′ end of the antisense strand. In some embodiments, the antisense strand has five 2′-fluoro modified nucleotides, e.g., at positions 2, 5, 8, 14, 16 from the 5′ end of the antisense strand. In some embodiments, the antisense strand has five 2′-fluoro modified nucleotides, e.g., at positions 2, 3, 7, 14, 16 from the 5′ end of the antisense strand. In some embodiments, the antisense strand has three 2′-fluoro modified nucleotides, e.g., at positions 2, 14, 16 from the 5′ end of the antisense strand. In some embodiments, the other nucleotides of the antisense strand are 2′-O-methyl modified nucleotides.

In some embodiments, the 5′ end of the antisense strand has a phosphate analog, e.g., 5′-vinylphosphonate (5′-VP).

In some embodiments, the sense strand or the antisense strand comprises an abasic moiety or inverted abasic moiety, e.g., a moiety shown in Table 5.

TABLE 5
Abasic or inverted abasic (iAb) moieties

| | Structure |
|---|---|
| 1 (abasic) | 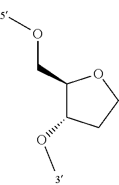 |
| 2 (iAb) | 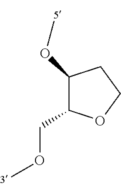 |

“5′” and “3′” indicate the 5′ to 3′ direction of the sequences.

In some embodiments, the sense strand and the antisense strand have one or more modified internucleotide linkages. In some embodiments, the modified internucleotide linkage is phosphorothioate linkage. In some embodiments, the sense strand has four or five phosphorothioate linkages. In some embodiments, the antisense strand has four or five phosphorothioate linkages. In some embodiments, the sense strand and the antisense strand each has four or five phosphorothioate linkages. In some embodiments, the sense strand has four phosphorothioate linkages and the antisense strand has five phosphorothioate linkages.

In some embodiments, the abasic moiety or inverted abasic moiety may be attached to the sense strand or the antisense strand via a phosphorothioate moiety. In one example, an inverted abasic moiety attached via a phosphorothioate internucleotide linkage has a formula as follows, where 3′ indicates the direction of the sequence::

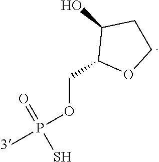

In some embodiments, the sense strand or antisense strand comprises a modified nucleotide with a lipid moiety at the 2′ position of a ribose (e.g., 2′-O—C₁₆ alkyl). In some embodiments, the modified nucleic acid is at position 6 from the 5′ end of the sense strand.

Exemplary modified sense strand and antisense strand sequences of dsRNA targeting ALK7 mRNA are provided in Table 6.

TABLE 6
Modified Nucleic Acid Sequences of dsRNA targeting human ALK7 mRNA
(ALK7 siRNA)

| | SEQ | | |
|---|---|---|---|
| dsRNA | ID | | |
| NO | NO | Strand | Modified 5′ to 3′ |
| 77 | 230 | s | mU*mU*mUmUmAmUmUmUfUfAfCmUmUmGmAmAmCmCmA*mA*mA |
| | 316 | as | mU*fU*mUmGfGmUfUmCmAmAmGmUmAfAmAfAmUmAmAmAmA*m |
| | | | A*mC |
| 78 | 231 | s | mC*mC*mCmGmCmCmUmAfAfCfUmGmCmUmCmUmUmCmG*mU*mA |
| | 317 | as | mU*fA*mCmGfAmAfGmAmGmCmAmGmUfUmAfGmGmCmGmGmG*mC |
| | | | *mC |
| 79 | 232 | s | mC*mU*mAmAmUmGmAmUfGfAfUmAmAmUmUmAmUmGmU*mU*mA |
| | 318 | as | mU*fA*mAmCfAmUfAmAmUmUmAmUmCfAmUfCmAmUmUmAmG*mG |
| | | | *mC |
| 80 | 233 | s | mA*mA*mGmUmAmUmAmAfAfAfUmAmAmGmCmUmCmUmA*mU*mA |
| | 319 | as | mU*fA*mUmAfGmAfGmCmUmUmAmUmUfUmUfAmUmAmCmUmU*mU |
| | | | *mU |
| 81 | 234 | s | mU*mC*mCmAmUmUmUmUfCfCfCmCmUmUmUmAmUmGmU*mG*mA |
| | 320 | as | mU*fC*mAmCfAmUfAmAmAmGmGmGmGfAmAfAmAmUmGmGmA*mA |
| | | | *mA |
| 82 | 235 | s | mA*mU*mGmUmUmAmAmAfAfAfGmAmAmAmUmCmUmCmU*mC*mA |
| | 321 | as | mU*fG*mAmGfAmGfAmUmUmUmCmUmUfUmUfUmAmAmCmAmU*mA |
| | | | *mA |
| 83 | 236 | s | mA*mU*mAmAmGmAmCmAfGfUfAmCmAmGmUmAmUmUmU*mA*mA |
| | 322 | as | mU*fU*mAmAfAmUfAmCmUmGmUmAmCfUmGfUmCmUmUmAmU*mC |
| | | | *mU |
| 84 | 237 | s | mA*mA*mCmUmCmUmAmAfAfGfUmUmAmAmGmCmAmUmG*mG*mA |
| | 323 | as | mU*fC*mCmAfUmGfCmUmUmAmAmCmUfUmUfAmGmAmGmUmU*mA |
| | | | *mU |
| 85 | 238 | s | mU*mU*mUmAmAmUmUmUfUfAfUmUmUmUmGmAmAmAmG*mC*mA |
| | 324 | as | mU*fG*mCmUfUmUfCmAmAmAmAmUmAfAmAfAmUmUmAmAmA*mC |
| | | | *mA |
| 86 | 239 | s | mG*mU*mUmAmUmAmGmUfCfAfUmUmAmAmGmUmUmUmU*mU*mA |
| | 325 | as | mU*fA*mAmAfAmAfCmUmUmAmAmUmGfAmCfUmAmUmAmAmC*mA |
| | | | *mU |
| 87 | 240 | s | mU*mU*mUmAmUmGmUmGfAfAfUmGmUmUmUmUmUmGmC*mC*mA |
| | 326 | as | mU*fG*mGmCfAmAfAmAmAmCmAmUmUfCmAfCmAmUmAmAmA*mG |
| | | | *mG |
| 88 | 241 | s | mU*mU*mAmUmGmAmCmAfUfGfGmUmGmCmCmUmUmCmA*mG*mA |
| | 327 | as | mU*fC*mUmGfAmAfGmGmCmAmCmCmAfUmGfUmCmAmUmAmA*mU |
| | | | *mA |
| 89 | 242 | s | mU*mC*mAmAmAmGmAmAfGfAfCmUmGmCmAmAmAmGmC*mC*mA |
| | 328 | as | mU*fG*mGmCfUmUfUmGmCmAmGmUmCfUmUfCmUmUmUmGmA*mC |
| | | | *mA |
| 90 | 243 | s | mU*mG*mAmAmAmAmGmAfUfAfAmCmUmCmUmAmAmAmG*mU*mA |
| | 329 | as | mU*fA*mCmUfUmUfAmGmAmGmUmUmAfUmCfUmUmUmUmCmA*mU |
| | | | *mG |
| 91 | 244 | s | mU*mU*mUmAmCmUmUmGfAfAfCmCmAmAmGmAmGmCmA*mC*mA |
| | 330 | as | mU*fG*mUmGfCmUfCmUmUmGmGmUmUfCmAfAmGmUmAmAmA*mA |
| | | | *mU |
| 92 | 245 | s | mC*mA*mAmCmUmCmAmUfCfUfUmUmUmUmAmUmUmUmA*mA*mA |
| | 331 | as | mU*fU*mUmAfAmAfUmAmAmAmAmAmGfAmUfGmAmGmUmUmG*m |
| | | | A*mG |
| 93 | 246 | s | mA*mUmGmAmUmGmAmUfAfAfUmUmAmUmGmUmUmAmA*mA*mA |
| | 332 | as | mU*fU*mUmUfAmAfCmAmUmAmAmUmUfAmUfCmAmUmCmAmU*mU |
| | | | *mA |
| 94 | 247 | s | mU*mA*mUmAmUmUmAmCfAfAfAmAmGmUmAmUmAmAmA*mA*mA |
| | 333 | as | mU*fU*mUmUfUmAfUmAmCmUmUmUmUfGmUfAmAmUmAmUmA*m |
| | | | U*mU |
| 95 | 248 | s | mG*mU*mGmAmUmAmGmUfAfCfAmUmGmUmUmAmGmCmA*mU*mA |
| | 334 | as | mU*fA*mUmGfCmUfAmAmCmAmUmGmUfAmCfUmAmUmCmAmC*mU |
| | | | *mU |
| 96 | 249 | s | mU*mU*mGmCmCmUmUmAfUfUfAmUmGmAmCmAmUmGmG*mU*mA |
| | 335 | as | mU*fA*mCmCfAmUfGmUmCmAmUmAmAfUmAfAmGmGmCmAmA*mU |
| | | | *mU |
| 97 | 250 | s | mU*mA*mGmCmAmUmAmUfAfAfUmGmUmAmUmUmUmUmG*mA*mA |
| | 336 | as | mU*fU*mCmAfAmAfAmUmAmCmAmUmUfAmUfAmUmGmCmUmA*mA |
| | | | *mC |
| 98 | 251 | s | mA*mC*mUmUmCmUmGmUfAfUfUmUmUmCmUmCmAmUmA*mG*mA |
| | 337 | as | mU*fC*mUmAfUmGfAmGmAmAmAmAmUfAmCfAmGmAmAmGmU*mA |
| | | | *mU |
| 99 | 252 | s | mU*mU*mUmUmGmUmGmUfGfAfUmUmCmUmUmCmAmAmA*mC*mA |
| | 338 | as | mU*fG*mUmUfUmGfAmAmGmAmAmUmCfAmCfAmCmAmAmAmA*mG |
| | | | *mA |
| 100 | 253 | s | mU*mC*mUmAmAmAmGmUfUfAfAmGmCmAmUmGmGmGmC*mA*mA |
| | 339 | as | mU*fU*mGmCfCmCfAmUmGmCmUmUmAfAmCfUmUmUmAmGmA*mG |
| | | | *mU |
| 101 | 254 | s | mG*mA*mCmAmUmGmGmUfGfCfCmUmUmCmAmGmAmUmC*mC*mA |
| | 340 | as | mU*fG*mGmAfUmCfUmGmAmAmGmGmCfAmCfCmAmUmGmUmC*mA |
| | | | *mU |
| 102 | 255 | s | mG*mC*mCmCmGmCmCmUfAfAfCmUmGmCmUmCmUmUmC*mG*mA |
| | 341 | as | mU*fC*mGmAfAmGfAmGmCmAmGmUmUfAmGfGmCmGmGmGmC*mC |
| | | | *mG |
| 103 | 256 | s | mU*mC*mAmAmAmGmAmAfUfAfUmCmUmUmAmGmUmGmA*mA*mA |
| | 342 | as | mU*fU*mUmCfAmCfUmAmAmGmAmUmAfUmUfCmUmUmUmGmA*mU |
| | | | *mU |
| 104 | 257 | s | mG*mU*mAmUmCmCmCmAfAfAfCmCmAmGmUmGmGmCmA*mA*mA |
| | 343 | as | mU*fU*mUmGfCmCfAmCmUmGmGmUmUfUmGfGmGmAmUmAmC*mU |
| | | | *mU |
| 105 | 258 | s | mU*mU*mUmUmAmUmUmAfUfGfAmUmAmUmUmUmGmUmG*mG*mA |
| | 344 | as | mU*fC*mCmAfCmAfAmAmUmAmUmCmAfUmAfAmUmAmAmAmA*mG |
| | | | *mA |
| 106 | 259 | s | mU*mU*mUmUmUmUmUmAfAfAfCmUmGmGmAmUmUmGmC*mU*mA |
| | 345 | as | mU*fA*mGmCfAmAfUmCmCmAmGmUmUfUmAfAmAmAmAmAmA*mA |
| | | | *mA |
| 107 | 260 | s | mU*mG*mUmCmUmUmUmUfGfUfGmUmGmAmUmUmCmUmU*mC*mA |
| | 346 | as | mU*fG*mAmAfGmAfAmUmCmAmCmAmCfAmAfAmAmGmAmCmA*mU |
| | | | *mA |
| 108 | 261 | s | mC*mU*mCmUmAmAmAmGfUfUfAmAmGmCmAmUmGmGmG*mC*mA |
| | 347 | as | mU*fG*mCmCfCmAfUmGmCmUmUmAmAfCmUfUmUmAmGmAmG*mU |
| | | | *mU |
| 109 | 262 | s | mU*mU*mUmUmAmCmUmUfGfAfAmCmCmAmAmGmAmGmC*mA*mA |
| | 348 | as | mU*fU*mGmCfUmCfUmUmGmGmUmUmCfAmAfGmUmAmAmAmA*mU |
| | | | *mA |
| 110 | 263 | s | mU*mC*mCmUmGmAmAmAfUfGfCmUmUmGmAmUmGmAmU*mA*mA |
| | 349 | as | mU*fU*mAmUfCmAfUmCmAmAmGmCmAfUmUfUmCmAmGmGmA*mG |
| | | | *mC |
| 111 | 264 | s | mU*mG*mAmAmAmUmGmCfUfUfGmAmUmGmAmUmAmCmA*mA*mA |
| | 350 | as | mU*fU*mUmGfUmAfUmCmAmUmCmAmAfGmCfAmUmUmUmCmA*mG |
| | | | *mG |
| 112 | 265 | s | mC*mA*mAmGmGmUmAmAfAfCfCmUmGmCmUmAmUmUmG*mC*mA |
| | 351 | as | mU*fG*mCmAfAmUfAmGmCmAmGmGmUfUmUfAmCmCmUmUmG*mU |
| | | | *mG |
| 113 | 266 | s | mG*mG*mGmCmUmCmCmUfUfAfUmAmUmGmAmCmUmAmU*mU*mA |
| | 352 | as | mU*fA*mAmUfAmGfUmCmAmUmAmUmAfAmGfGmAmGmCmCmC*mU |
| | | | *mG |
| 114 | 267 | s | mG*mG*mCmUmCmCmUmGfAfAfAmUmGmCmUmUmGmAmU*mG*mA |
| | 353 | as | mU*fC*mAmUfCmAfAmGmCmAmUmUmUfCmAfGmGmAmGmCmC*mA |
| | | | *mU |
| 115 | 268 | s | mU*mU*mUmAmUmUmUmUfAfCfUmUmGmAmAmCmCmAmA*mG*mA |
| | 354 | as | mU*fC*mUmUfGmGfUmUmCmAmAmGmUfAmAfAmAmUmAmAmA*mA |
| | | | *mA |
| 116 | 269 | s | mU*mU*mGmAmUmGmAmUfAfCfAmAmUmGmAmAmUmGmU*mG*mA |
| | 355 | as | mU*fC*mAmCfAmUfUmCmAmUmUmGmUfAmUfCmAmUmCmAmA*mG |
| | | | *mC |
| 117 | 270 | s | mU*mG*mAmUmUmCmAmAfUfAfCmUmGmAmAmCmAmCmU*mA*mA |
| | 356 | as | mU*fU*mAmGfUmGfUmUmCmAmGmUmAfUmUfGmAmAmUmCmA*mU |
| | | | *mG |
| 118 | 271 | s | mA*mU*mCmUmAmUmUmCfUfGfUmUmGmGmUmCmUmGmG*mU*mA |
| | 357 | as | mU*fA*mCmCfAmGfAmCmCmAmAmCmAfGmAfAmUmAmGmAmU*mG |
| | | | *mU |
| 119 | 272 | s | mA*mG*mUmUmGmAmCmUfUfCfAmUmCmCmAmAmUmCmU*mC*mA |
| | 358 | as | mU*fG*mAmGfAmUfUmGmGmAmUmGmAfAmGfUmCmAmAmCmU*mC |
| | | | *mC |
| 120 | 273 | s | mC*mU*mCmAmUmCmGmAfGfAfCmAmUmAmAmAmAmUmC*mA*mA |
| | 359 | as | mU*fU*mGmAfUmUfUmUmAmUmGmUmCfUmCfGmAmUmGmAmG*mC |
| | | | *mA |
| 121 | 274 | s | mU*mA*mCmCmUmCmAmAfAfGfAmUmAmAmGmAmCmAmG*mU*mA |
| | 360 | as | mU*fA*mCmUfGmUfCmUmUmAmUmCmUfUmUfGmAmGmGmUmA*mG |
| | | | *mA |
| 122 | 275 | s | mU*mA*mUmAmGmUmCmAfUfUfAmAmGmUmUmUmUmUmA*mU*mA |
| | 361 | as | mU*fA*mUmAfAmAfAmAmCmUmUmAmAfUmGfAmCmUmAmUmA*mA |
| | | | *mC |
| 123 | 276 | s | mC*mA*mAmAmGmAmUmAfAfGfAmCmAmGmUmAmCmAmG*mU*mA |
| | 362 | as | mU*fA*mCmUfGmUfAmCmUmGmUmCmUfUmAfUmCmUmUmUmG*mA |
| | | | *mG |
| 124 | 277 | s | mA*mA*mAmGmAmAmUmAfUfCfUmUmAmGmUmGmAmAmA*mA*mA |
| | 363 | as | mU*fU*mUmUfUmCfAmCmUmAmAmGmAfUmAfUmUmCmUmUmU*mG |
| | | | *mA |
| 125 | 278 | s | mA*mC*mCmAmAmUmUmGfCfCfUmUmAmUmUmAmUmGmA*mC*mA |
| | 364 | as | mU*fG*mUmCfAmUfAmAmUmAmAmGmGfCmAfAmUmUmGmGmU*mA |
| | | | *mC |
| 126 | 279 | s | mU*mU*mUmUmUmGmUmUfCfUfAmCmCmUmCmAmAmAmG*mA*mA |
| | 365 | as | mU*fU*mCmUfUmUfGmAmGmGmUmAmGfAmAfCmAmAmAmAmA*mA |
| | | | *mA |
| 127 | 280 | s | mU*mU*mCmCmAmUmUmUfUfCfCmCmCmUmUmUmAmUmG*mU*mA |
| | 366 | as | mU*fA*mCmAfUmAfAmAmGmGmGmGmAfAmAfAmUmGmGmAmA*m |
| | | | A*mA |
| 128 | 281 | s | mC*mU*mAmCmCmUmCmAfAfAfGmAmUmAmAmGmAmCmA*mG*mA |
| | 367 | as | mU*fC*mUmGfUmCfUmUmAmUmCmUmUfUmGfAmGmGmUmAmG*mA |
| | | | *mA |
| 129 | 282 | s | mA*mA*mUmCmAmAmAmGfAfAfUmAmUmCmUmUmAmGmU*mG*mA |
| | 368 | as | mU*fC*mAmCfUmAfAmGmAmUmAmUmUfCmUfUmUmGmAmUmU*mU |
| | | | *mU |
| 130 | 283 | s | mA*mU*mGmCmUmUmGmAfUfGfAmUmAmCmAmAmUmGmA*mA*mA |
| | 369 | as | mU*fU*mUmCfAmUfUmGmUmAmUmCmAfUmCfAmAmGmCmAmU*mU |
| | | | *mU |
| 131 | 284 | s | mU*mA*mGmGmUmGmAmUfGfCfAmAmUmCmUmAmUmGmU*mU*mA |
| | 370 | as | mU*fA*mAmCfAmUfAmGmAmUmUmGmCfAmUfCmAmCmCmUmA*mA |
| | | | *mA |
| 132 | 285 | s | mA*mU*mAmUmCmUmCmAfAfCfUmUmUmGmUmGmUmCmA*mA*mA |
| | 371 | as | mU*fU*mUmGfAmCfAmCmAmAmAmGmUfUmGfAmGmAmUmAmU*mA |
| | | | *mG |
| 133 | 286 | s | mA*mU*mGmAmAmUmGmUfGfAfAmUmAmUmCmUmUmUmG*mA*mA |
| | 372 | as | mU*fU*mCmAfAmAfGmAmUmAmUmUmCfAmCfAmUmUmCmAmU*mU |
| | | | *mG |
| 134 | 287 | s | mU*mU*mCmAmAmAmCmGfAfGfCmUmGmAmCmAmUmCmU*mA*mA |
| | 373 | as | mU*fU*mAmGfAmUfGmUmCmAmGmCmUfCmGfUmUmUmGmAmA*mG |
| | | | kmG |
| 135 | 288 | s | mA*mA*mAmAmAmUmAmAfAfUfAmUmUmGmCmUmCmUmA*mA*mA |
| | 374 | as | mU*fU*mUmAfGmAfGmCmAmAmUmAmUfUmUfAmUmUmUmUmU*m |
| | | | A*mA |
| 136 | 289 | s | mC*mU*mCmUmCmAmUmAfGfCfUmUmUmCmUmUmUmUmC*mC*mA |
| | 375 | as | mU*fG*mGmAfAmAfAmGmAmAmAmGmCfUmAfUmGmAmGmAmG*m |
| | | | A*mU |
| 137 | 290 | s | mA*mU*mAmGmAmGmGmAfAfAfUmGmAmGmAmAmAmGmG*mU*mA |
| | 376 | as | mU*fA*mCmCfUmUfUmCmUmCmAmUmUfUmCfCmUmCmUmAmU*mC |
| | | | *mG |
| 138 | 291 | s | mG*mU*mGmAmAmAmAmAfGfUfGmUmGmAmAmAmCmUmU*mG*mA |
| | 377 | as | mU*fC*mAmAfGmUfUmUmCmAmCmAmCfUmUfUmUmUmCmAmC*mU |
| | | | *mA |
| 139 | 292 | s | mU*mG*mGmAmCmUmCmAfAfCfUmUmUmGmGmCmUmGmG*mU*mA |
| | 378 | as | mU*fA*mCmCfAmGfCmCmAmAmAmGmUfUmGfAmGmUmCmCmA*mA |
| | | | *mG |
| 140 | 293 | s | mU*mU*mAmAmGmUmUmUfUfUfAmUmUmUmUmAmCmUmU*mG*mA |
| | 379 | as | mU*fC*mAmAfGmUfAmAmAmAmUmAmAfAmAfAmCmUmUmAmA*mU |
| | | | *mG |
| 141 | 294 | s | mG*mA*mAmGmAmUmGmUfGfGfCmUmGmUmGmAmAmAmA*mU*mA |
| | 380 | as | mU*fA*mUmUfUmUfCmAmCmAmGmCmCfAmCfAmUmCmUmUmC*mC |
| | | | *mC |
| 142 | 295 | s | mU*mA*mUmGmUmUmAmAfAfAfAmGmAmAmAmUmCmUmC*mU*mA |
| | 381 | as | mU*fA*mGmAfGmAfUmUmUmCmUmUmUfUmUfAmAmCmAmUmA*mA |
| | | | *mU |
| 143 | 296 | s | mU*mA*mAmUmGmAmUmGfAfUfAmAmUmUmAmUmGmUmU*mA*mA |
| | 382 | as | mU*fU*mAmAfCmAfUmAmAmUmUmAmUfCmAfUmCmAmUmUmA*mG |
| | | | *mG |
| 144 | 297 | s | mG*mA*mUmAmAmUmUmAfUfGfUmUmAmAmAmAmAmGmA*mA*mA |
| | 383 | as | mU*fU*mUmCfUmUfUmUmUmAmAmCmAfUmAfAmUmUmAmUmC*mA |
| | | | *mU |
| 145 | 298 | s | mG*mA*mCmUmGmCmAmAfAfGfCmCmUmAmAmUmGmAmU*mG*mA |
| | 384 | as | mU*fC*mAmUfCmAfUmUmAmGmGmCmUfUmUfGmCmAmGmUmC*mU |
| | | | *mU |
| 146 | 299 | s | mC*mA*mUmUmAmAmGmUfUfUfUmUmAmUmUmUmUmAmC*mU*mA |
| | 385 | as | mU*fA*mGmUfAmAfAmAmUmAmAmAmAfAmCfUmUmAmAmUmG*m |
| | | | A*mC |
| 147 | 300 | s | mA*mC*mUmGmCmUmCmUfUfCfGmUmAmUmUmAmAmGmA*mA*mA |
| | 386 | as | mU*fU*mUmCfUmUfAmAmUmAmCmGmAfAmGfAmGmCmAmGmU*mU |
| | | | *mA |
| 148 | 301 | s | mU*mU*mAmUmGmUmUmAfAfAfAmAmGmAmAmAmUmCmU*mC*mA |
| | 387 | as | mU*fG*mAmGfAmUfUmUmCmUmUmUmUfUmAfAmCmAmUmAmA*mU |
| | | | ′mU |
| 149 | 302 | s | mG*mU*mUmGmAmCmUmUfCfAfUmCmCmAmAmUmCmUmC*mU*mA |
| | 388 | as | mU*fA*mGmAfGmAfUmUmGmGmAmUmGfAmAfGmUmCmAmAmC*mU |
| | | | *mC |
| 150 | 303 | s | mU*mU*mUmUmG[Uhd]mGmUfGfAfUmUmCmUmUmCmAmAmA*mC*m |
| | | | A |
| | 338 | as | mU*fG*mUmUfUmGfAmAmGmAmAmUmCfAmCfAmCmAmAmAmA*mG |
| | | | *mA |
| 151 | 304 | s | mU*mC*mUmAmA[Ahd]mGmUfUfAfAmGmCmAmUmGmGmGmC*mA*m |
| | | | A |
| | 339 | as | mU*fU*mGmCfCmCfAmUmGmCmUmUmAfAmCfUmUmUmAmGmA*mG |
| | | | *mU |
| 152 | 305 | s | mG*mA*mCmAmU[Ghd]mGmUfGfCfCmUmUmCmAmGmAmUmC*mC*m |
| | | | A |
| | 340 | as | mU*fG*mGmAfUmCfUmGmAmAmGmGmCfAmCfCmAmUmGmUmC*mA |
| | | | *mU |
| 153 | 306 | s | mU*mG*mUmCmU[Uhd]mUmUfGfUfGmUmGmAmUmUmCmUmU*mC*m |
| | | | A |
| | 346 | as | mU*fG*mAmAfGmAfAmUmCmAmCmAmCfAmAfAmAmGmAmCmA*mU |
| | | | *mA |
| 154 | 307 | s | mC*mU*mCmUmA[Ahd]mAmGfUfUfAmAmGmCmAmUmGmGmG*mC*m |
| | | | A |
| | 347 | as | mU*fG*mCmCfCmAfUmGmCmUmUmAmAfCmUfUmUmAmGmAmG*mU |
| | | | *mU |
| 155 | 308 | s | mU*mU*mUmUmA[Chd]mUmUfGfAfAmCmCmAmAmGmAmGmC*mA*m |
| | | | A |
| | 348 | as | mU*fU*mGmCfUmCfUmUmGmGmUmUmCfAmAfGmUmAmAmAmA*mU |
| | | | *mA |
| 156 | 309 | s | mU*mC*mCmUmG[Ahd]mAmAfUfGfCmUmUmGmAmUmGmAmU*mA*m |
| | | | A |
| | 349 | as | mU*fU*mAmUfCmAfUmCmAmAmGmCmAfUmUfUmCmAmGmGmA*mG |
| | | | *mC |
| 157 | 310 | s | mC*mC*mUmGmAmAmAmUfGfCfUmUmGmAmUmGmAmUmA*mC*mA |
| | 389 | as | mU*fG*mUmAfUmCfAmUmCmAmAmGmCfAmUfUmUmCmAmGmG*mA |
| | | | *mG |
| 158 | 311 | s | mG*mA*mAmAmUmGmCmUfUfGfAmUmGmAmUmAmCmAmA*mU*mA |
| | 390 | as | mU*fA*mUmUfGmUfAmUmCmAmUmCmAfAmGfCmAmUmUmUmC*mA |
| | | | *mG |
| 159 | 312 | s | mU*mC*mCmUmUmAmUmAfUfGfAmCmUmAmUmUmUmGmA*mA*mA |
| | 391 | as | mU*fU*mUmCfAmAfAmUmAmGmUmCmAfUmAfUmAmAmGmGmA*mG |
| | | | *mC |
| 160 | 313 | s | mC*mC*mUmGmA[Ahd]mAmUfGfCfUmUmGmAmUmGmAmUmA*mC*m |
| | | | A |
| | 389 | as | mU*fG*mUmAfUmCfAmUmCmAmAmGmCfAmUfUmUmCmAmGmG*mA |
| | | | *mG |
| 161 | 314 | s | mG*mA*mAmAmU[Ghd]mCmUfUfGfAmUmGmAmUmAmCmAmA*mU* |
| | | | mA |
| | 390 | as | mU*fA*mUmUfGmUfAmUmCmAmUmCmAfAmGfCmAmUmUmUmC*mA |
| | | | *mG |
| 162 | 315 | s | mU*mC*mCmUmU[Ahd]mUmAfUfGfAmCmUmAmUmUmUmGmA*mA*m |
| | | | A |
| | 391 | as | mU*fU*mUmCfAmAfAmUmAmGmUmCmAfUmAfUmAmAmGmGmA*mG |
| | | | *mC |
| 231 | 519 | s | mC*mU*mUmGmAmUmGmAfUfAfCmAmAmUmGmAmAmUmG*mU*mA |
| | 599 | as | mU*fA*mCmAfUmUfCmAmUmUmGmUmAfUmCfAmUmCmAmAmG*mC |
| | | | *mA |
| 232 | 520 | s | mU*mG*mCmUmUmGmAmUfGfAfUmAmCmAmAmUmGmAmA*mU*mA |
| | 600 | as | mU*fA*mUmUfCmAfUmUmGmUmAmUmCfAmUfCmAmAmGmCmA*mU |
| | | | *mU |
| 233 | 521 | s | mU*mG*mUmUmAmGmCmAfUfAfUmAmAmUmGmUmAmUmU*mU*mA |
| | 601 | as | mU*fA*mAmAfUmAfCmAmUmUmAmUmAfUmGfCmUmAmAmCmA*mU |
| | | | *mG |
| 234 | 522 | s | mU*mC*mAmUmUmAmAmGfUfUfUmUmUmAmUmUmUmUmA*mC*mA |
| | 602 | as | mU*fG*mUmAfAmAfAmUmAmAmAmAmAfCmUfUmAmAmUmGmA*mC |
| | | | *mU |
| 235 | 523 | s | mA*mG*mAmCmUmGmCmAfAfAfGmCmCmUmAmAmUmGmA*mU*mA |
| | 603 | as | mU*fA*mUmCfAmUfUmAmGmGmCmUmUfUmGfCmAmGmUmCmU*mU |
| | | | *mC |
| 236 | 524 | s | mG*mG*mAmAmGmAmUmGfUfGfGmCmUmGmUmGmAmAmA*mA*mA |
| | 604 | as | mU*fU*mUmUfUmCfAmCmAmGmCmCmAfCmAfUmCmUmUmCmC*mC |
| | | | *mC |
| 237 | 525 | s | mU*mA*mGmAmGmGmAmAfAfUfGmAmGmAmAmAmGmGmU*mU*mA |
| | 605 | as | mU*fA*mAmCfCmUfUmUmCmUmCmAmUfUmUfCmCmUmCmUmA*mU |
| | | | *mC |
| 238 | 526 | s | mG*mG*mUmGmAmUmGmCfAfAfUmCmUmAmUmGmUmUmU*mC*mA |
| | 606 | as | mU*fG*mAmAfAmCfAmUmAmGmAmUmUfGmCfAmUmCmAmCmC*mU |
| | | | *mA |
| 239 | 527 | s | mU*mU*mCmUmAmCmCmUfCfAfAmAmGmAmUmAmAmGmA*mC*mA |
| | 607 | as | mU*fG*mUmCfUmUfAmUmCmUmUmUmGfAmGfGmUmAmGmAmA*mC |
| | | | *mA |
| 240 | 528 | s | mU*mA*mAmCmUmGmCmUfCfUfUmCmGmUmAmUmUmAmA*mG*mA |
| | 608 | as | mU*fC*mUmUfAmAfUmAmCmGmAmAmGfAmGfCmAmGmUmUmA*mG |
| | | | *mG |
| 241 | 529 | s | mA*mG*mGmUmAmAmAmCfCfUfGmCmUmAmUmUmGmCmU*mC*mA |
| | 609 | as | mU*fG*mAmGfCmAfAmUmAmGmCmAmGfGmUfUmUmAmCmCmU*mU |
| | | | *mG |
| 242 | 530 | s | mU*mU*mUmCmCmAmUmUfUfUfCmCmCmCmUmUmUmAmU*mG*mA |
| | 610 | as | mU*fC*mAmUfAmAfAmGmGmGmGmAmAfAmAfUmGmGmAmAmA*m |
| | | | A*mG |
| 243 | 531 | s | mG*mC*mUmGmGmUmAmUfCfUfGmAmAmUmAmUmCmAmU*mG*mA |
| | 611 | as | mU*fC*mAmUfGmAfUmAmUmUmCmAmGfAmUfAmCmCmAmGmC*mC |
| | | | *mA |
| 244 | 532 | s | mA*mA*mUmAmUmAmUmUfAfCfAmAmAmAmGmUmAmUmA*mA*mA |
| | 612 | as | mU*fU*mUmAfUmAfCmUmUmUmUmGmUfAmAfUmAmUmAmUmU*m |
| | | | U*mC |
| 245 | 533 | s | mA*mU*mUmAmUmGmUmUfAfAfAmAmAmGmAmAmAmUmC*mU*mA |
| | 613 | as | mU*fA*mGmAfUmUfUmCmUmUmUmUmUfAmAfCmAmUmAmAmU*mU |
| | | | *mA |
| 246 | 534 | s | mA*mC*mUmAmUmAmUmCfUfCfAmAmCmUmUmUmGmUmG*mU*mA |
| | 614 | as | mU*fA*mCmAfCmAfAmAmGmUmUmGmAfGmAfUmAmUmAmGmU*mC |
| | | | *mU |
| 247 | 535 | s | mG*mG*mAmGmUmUmGmAfCfUfUmCmAmUmCmCmAmAmU*mC*mA |
| | 615 | as | mU*fG*mAmUfUmGfGmAmUmGmAmAmGfUmCfAmAmCmUmCmC*mU |
| | | | *mG |
| 248 | 536 | s | mA*mA*mAmAmUmAmAmAfUfAfUmUmGmCmUmCmUmAmA*mA*mA |
| | 616 | as | mU*fU*mUmUfAmGfAmGmCmAmAmUmAfUmUfUmAmUmUmUmU*m |
| | | | U*mA |
| 249 | 537 | s | mC*mU*mGmUmCmAmUmAfGfUfUmCmCmAmAmCmAmAmU*mG*mA |
| | 617 | as | mU*fC*mAmUfUmGfUmUmGmGmAmAmCfUmAfUmGmAmCmAmG*mA |
| | | | *mA |
| 250 | 538 | s | mG*mC*mCmUmAmAmUmGfAfUfGmAmUmAmAmUmUmAmU*mG*mA |
| | 618 | as | mU*fC*mAmUfAmAfUmUmAmUmCmAmUfCmAfUmUmAmGmGmC*mU |
| | | | *mU |
| 251 | 539 | s | mA*mU*mAmAmCmUmCmUfAfAfAmGmUmUmAmAmGmCmA*mU*mA |
| | 619 | as | mU*fA*mUmGfCmUfUmAmAmCmUmUmUfAmGfAmGmUmUmAmU*mC |
| | | | kmU |
| 252 | 540 | s | mU*mA*mAmUmUmUmUmAfUfUfUmUmGmAmAmAmGmCmA*mA*mA |
| | 620 | as | mU*fU*mUmGfCmUfUmUmCmAmAmAmAfUmAfAmAmAmUmUmA*mA |
| | | | *mA |
| 253 | 541 | s | mA*mA*mGmAmUmAmAmGfAfCfAmGmUmAmCmAmGmUmA*mU*mA |
| | 621 | as | mU*fA*mUmAfCmUfGmUmAmCmUmGmUfCmUfUmAmUmCmUmU*mU |
| | | | *mG |
| 254 | 542 | s | mA*mU*mGmAmUmAmAmUfUfAfUmGmUmUmAmAmAmAmA*mG*mA |
| | 622 | as | mU*fC*mUmUfUmUfUmAmAmCmAmUmAfAmUfUmAmUmCmAmU*mC |
| | | | *mA |
| 255 | 543 | s | mU*mC*mUmCmAmAmCmUfUfUfGmUmGmUmCmAmAmAmG*mA*mA |
| | 623 | as | mU*fU*mCmUfUmUfGmAmCmAmCmAmAfAmGfUmUmGmAmGmA*mU |
| | | | *mA |
| 256 | 544 | s | mU*mU*mUmUmUmUmGmUfUfCfUmAmCmCmUmCmAmAmA*mG*mA |
| | 624 | as | mU*fC*mUmUfUmGfAmGmGmUmAmGmAfAmCfAmAmAmAmAmA*mA |
| | | | *mA |
| 257 | 545 | s | mA*mU*mUmAmCmAmAmAfAfGfUmAmUmAmAmAmAmUmA*mA*mA |
| | 625 | as | mU*fU*mUmAfUmUfUmUmAmUmAmCmUfUmUfUmGmUmAmAmU*m |
| | | | A*mU |
| 258 | 546 | s | mA*mA*mAmGmCmCmUmAfAfUfGmAmUmGmAmUmAmAmU*mU*mA |
| | 625 | as | mU*fU*mUmAfUmUfUmUmAmUmAmCmUfUmUfUmGmUmAmAmU*m |
| | | | A*mU |
| 259 | 547 | s | mC*mU*mCmAmUmAmGmCfUfUfUmCmUmUmUmUmCmCmA*mU*mA |
| | 627 | as | mU*fA*mUmGfGmAfAmAmAmGmAmAmAfGmCfUmAmUmGmAmG*m |
| | | | A*mG |
| 260 | 548 | s | mG*mC*mAmUmAmUmAmAfUfGfUmAmUmUmUmUmGmAmA*mG*mA |
| | 628 | as | mU*fC*mUmUfCmAfAmAmAmUmAmCmAfUmUfAmUmAmUmGmC*mU |
| | | | *mA |
| 261 | 549 | s | mC*mU*mGmCmUmCmUmUfCfGfUmAmUmUmAmAmGmAmA*mG*mA |
| | 629 | as | mU*fC*mUmUfCmUfUmAmAmUmAmCmGfAmAfGmAmGmCmAmG*mU |
| | | | *mU |
| 262 | 550 | s | mG*mU*mUmUmUmUmAmUfUfUfUmAmCmUmUmGmAmAmC*mC*mA |
| | 630 | as | mU*fG*mGmUfUmCfAmAmGmUmAmAmAfAmUfAmAmAmAmAmC*mU |
| | | | *mU |
| 263 | 551 | s | mC*mC*mAmUmUmUmUmCfCfCfCmUmUmUmAmUmGmUmG*mA*mA |
| | 631 | as | mU*fU*mCmAfCmAfUmAmAmAmGmGmGfGmAfAmAmAmUmGmG*mA |
| | | | *mA |
| 264 | 552 | s | mA*mU*mAmUmUmAmCmAfAfAfAmGmUmAmUmAmAmAmA*mU*mA |
| | 632 | as | mU*fA*mUmUfUmUfAmUmAmCmUmUmUfUmGfUmAmAmUmAmU*m |
| | | | A*mU |
| 265 | 553 | s | mG*mC*mUmCmCmUmGmAfAfAfUmGmCmUmUmGmAmUmG*mA*mA |
| | 633 | as | mU*fU*mCmAfUmCfAmAmGmCmAmUmUfUmCfAmGmGmAmGmC*mC |
| | | | *mA |
| 266 | 554 | s | mA*mU*mAmAmUmUmAmUfGfUfUmAmAmAmAmAmGmAmA*mA*mA |
| | 634 | as | mU*fU*mUmUfCmUfUmUmUmUmAmAmCfAmUfAmAmUmUmAmU*mC |
| | | | *mA |
| 267 | 555 | s | mU*mG*mAmUmGmAmUmAfAfUfUmAmUmGmUmUmAmAmA*mA*mA |
| | 635 | as | mU*fU*mUmUfUmAfAmCmAmUmAmAmUfUmAfUmCmAmUmCmA*mU |
| | | | *mU |
| 268 | 556 | s | mA*mC*mUmGmCmAmAmAfGfCfCmUmAmAmUmGmAmUmG*mA*mA |
| | 636 | as | mU*fU*mCmAfUmCfAmUmUmAmGmGmCfUmUfUmGmCmAmGmU*mC |
| | | | *mU |
| 269 | 557 | s | mA*mC*mAmCmAmAmGmGfUfAfAmAmCmCmUmGmCmUmA*mU*mA |
| | 636 | as | mU*fU*mCmAfUmCfAmUmUmAmGmGmCfUmUfUmGmCmAmGmU*mC |
| | | | *mU |
| 270 | 558 | s | mU*mG*mAmUmAmAmUmUfAfUfGmUmUmAmAmAmAmAmG*mA*mA |
| | 638 | as | mU*fU*mCmUfUmUfUmUmAmAmCmAmUfAmAfUmUmAmUmCmA*mU |
| | | | *mC |
| 271 | 559 | s | mA*mA*mGmAmCmUmGmCfAfAfAmGmCmCmUmAmAmUmG*mA*mA |
| | 639 | as | mU*fU*mCmAfUmUfAmGmGmCmUmUmUfGmCfAmGmUmCmUmU*mC |
| | | | *mU |
| 272 | 560 | s | mA*mA*mAmUmAmUmAmUfUfAfCmAmAmAmAmGmUmAmU*mA*mA |
| | 640 | as | mU*fU*mAmUfAmCfUmUmUmUmGmUmAfAmUfAmUmAmUmUmU*mC |
| | | | *mU |
| 273 | 561 | s | mA*mA*mCmUmGmCmUmCfUfUfCmGmUmAmUmUmAmAmG*mA*mA |
| | 641 | as | mU*fU*mCmUfUmAfAmUmAmCmGmAmAfGmAfGmCmAmGmUmU*mA |
| | | | *mG |
| 274 | 562 | s | mC*mC*mUmCmAmAmAmGfAfUfAmAmGmAmCmAmGmUmA*mC*mA |
| | 642 | as | mU*fG*mUmAfCmUfGmUmCmUmUmAmUfCmUfUmUmGmAmGmG*mU |
| | | | *mA |
| 275 | 563 | s | mU*mU*mAmCmUmUmGmAfAfCfCmAmAmGmAmGmCmAmC*mA*mA |
| | 643 | as | mU*fU*mGmUfGmCfUmCmUmUmGmGmUfUmCfAmAmGmUmAmA*mA |
| | | | *mA |
| 276 | 564 | s | mU*mA*mUmCmUmCmAmAfCfUfUmUmGmUmGmUmCmAmA*mA*mA |
| | 644 | as | mU*fU*mUmUfGmAfCmAmCmAmAmAmGfUmUfGmAmGmAmUmA*mU |
| | | | *mA |
| 277 | 565 | s | mUmG*mCmAmAmAmGmCfCfUfAmAmUmGmAmUmGmAmU*mA*mA |
| | 645 | as | mU*fU*mAmUfCmAfUmCmAmUmUmAmGfGmCfUmUmUmGmCmA*mG |
| | | | *mU |
| 278 | 566 | s | mU*mA*mUmUmAmCmAmAfAfAfGmUmAmUmAmAmAmAmU*mA*mA |
| | 646 | as | mU*fU*mAmUfUmUfUmAmUmAmCmUmUfUmUfGmUmAmAmUmA*m |
| | | | U*mA |
| 279 | 567 | s | mU*mG*mGmUmAmUmCmUfGfAfAmUmAmUmCmAmUmGmA*mA*mA |
| | 647 | as | mU*fU*mUmCfAmUfGmAmUmAmUmUmCfAmGfAmUmAmCmCmA*mG |
| | | | *mC |
| 280 | 568 | s | [iAb]*mU*mUmCmAmAmAmCmGfAfGfCmUmGmAmCmAmUmCmU*mA |
| | | | *mA |
| | 648 | as | mU*fU*mAmGfAmUfGmUmCmAmGmCmUfCmGfUmUmUmGmA*mA*m |
| | | | G*mG |
| 281 | 569 | s | [iAb]*mA*mUmAmUmCmUmCmAfAfCfUmUmUmGmUmGmUmCmA*mA |
| | | | *mA |
| | 649 | as | mU*fU*mUmGfAmCfAmCmAmAmAmGmUfUmGfAmGmAmUmA*mU*m |
| | | | A*mG |
| 282 | 570 | s | [iAb]*mU*mUmGmAmUmGmAmUfAfCfAmAmUmGmAmAmUmGmU*m |
| | | | G*mA |
| | 650 | as | mU*fC*mAmCfAmUfUmCmAmUmUmGmUfAmUfCmAmUmCmA*mA*m |
| | | | G*mC |
| 283 | 571 | s | [iAb]*mA*mUmGmAmAmUmGmUfGfAfAmUmAmUmCmUmUmUmG*m |
| | | | A*mA |
| | 651 | as | mU*fU*mCmAfAmAfGmAmUmAmUmUmCfAmCfAmUmUmCmA*mU*m |
| | | | U*mG |
| 284 | 572 | s | [iAb]*mG*mGmCmUmCmCmUmGfAfAfAmUmGmCmUmUmGmAmU*mG |
| | | | ′mA |
| | 652 | as | mU*fC*mAmUfCmAfAmGmCmAmUmUmUfCmAfGmGmAmGmC*mC*m |
| | | | A*mU |
| 285 | 573 | s | [iAb]*mA*mUmAmUmCmUmCmAfAfCfUmUmUmGmU[Ghd]mUmCmA* |
| | | | mA*mA |
| | 649 | as | mU*fU*mUmGfAmCfAmCmAmAmAmGmUfUmGfAmGmAmUmA*mU*m |
| | | | A*mG |
| 286 | 574 | s | [iAb]*mU*mUmGmAmUmGmAmUfAfCfAmAmUmGmA[Ahd]mUmGmU* |
| | | | mG*mA |
| | 650 | as | mU*fC*mAmCfAmUfUmCmAmUmUmGmUfAmUfCmAmUmCmA*mA*m |
| | | | G*mC |
| 287 | 575 | s | [iAb]*mG*mGmCmUmCmCmUmGfAfAfAmUmGmCmU[Uhd]mGmAmU* |
| | | | mG*mA |
| | 652 | as | mU*fC*mAmUfCmAfAmGmCmAmUmUmUfCmAfGmGmAmGmC*mC*m |
| | | | A*mU |
| 288 | 568 | s | [iAb]*mU*mUmCmAmAmAmCmGfAfGfCmUmGmAmCmAmUmCmU*mA |
| | | | *mA |
| | 648 | as | mU*fU*mAmGfAmUfGmUmCmAmGmCmUfCmGfUmUmUmGmA*mA*m |
| | | | G*mG |
| 289 | 576 | s | [iAb]*mU*mUmCmAmAmAmCmGfAfGfCmUmGmAmC[Ahd]mUmCmU* |
| | | | mA*mA |
| | 648 | as | mU*fU*mAmGfAmUfGmUmCmAmGmCmUfCmGfUmUmUmGmA*mA*m |
| | | | G*mG |
| 290 | 577 | s | [iAb]*mA*mUmGmAmAmUmGmUfGfAfAmUmAmUmC[Uhd]mUmUmG* |
| | | | mA*mA |
| | 651 | as | mU*fU*mCmAfAmAfGmAmUmAmUmUmCfAmCfAmUmUmCmA*mU*m |
| | | | U*mG |
| 291 | 578 | s | mC*mU*mAmAmUmGfAmUfGfAfUmAmAmUmU[Ada]mUmGmU*mU*m |
| | | | A |
| | 653 | as | mU*fA*mAmCmAfUmAmAmUmUmAmUmCfAmUfCmAmUmUmAmG*m |
| | | | G*mC |
| 292 | 579 | s | [iAb]*mGmUmCmUmGmUmCmAfUfAfGmUmUmCmCmAmAmCmAmAm |
| | | | U*[iAb] |
| | 654 | as | [CpmA]*fU*mUmGmUmUfGmGmAmAmCfUmAfUmGfAmCmAmGmA*m |
| | | | C |
| 293 | 580 | s | [iAb]*mCmUmAmAmUmGfAmUfGfAfUmAmAmUmUmAmUmGmUmUm |
| | | | A*[iAb] |
| | 653 | as | mU*fA*mAmCmAfUmAmAmUmUmAmUmCfAmUfCmAmUmUmAmG*m |
| | | | G*mC |
| 294 | 581 | s | [iAb]*mC*mUmAmAmUmGfAmUfGfAfUmAmAmUmUmAmUmGmU*mU |
| | | | *mA |
| | 653 | as | mU*fA*mAmCmAfUmAmAmUmUmAmUmCfAmUfCmAmUmUmAmG*m |
| | | | G*mC |
| 295 | 582 | s | [iAb]*mC*mUmAmAmUmGfAmUfGfAfUmAmAmUmU[Ahd]mUmGmU*m |
| | | | U*mA |
| | 653 | as | mU*fA*mAmCmAfUmAmAmUmUmAmUmCfAmUfCmAmUmUmAmG*m |
| | | | G*mC |
| 296 | 310 | s | mC*mC*mUmGmAmAmAmUfGfCfUmUmGmAmUmGmAmUmA*mC*mA |
| | 655 | as | mU*fG*mUmAfUmCfAmUmCmAmAmGmCfAmUfUmUmCmAmG*mG*m |
| | | | A*mG |
| 297 | 583 | s | [iAb]*mC*mAmAmAmGmAmUmAfAfGfAmCmAmGmUmAmCmAmG*mU |
| | | | *mA |
| | 656 | as | mU*fA*mCmUfGmUfAmCmUmGmUmCmUfUmAfUmCmUmUmU*mG*m |
| | | | A*mG |
| 298 | 584 | s | [iAb]*mU*mUmCmCmAmUmUmUfUfCfCmCmCmUmUmUmAmUmG*mU |
| | | | *mA |
| | 657 | as | mU*fA*mCmAfUmAfAmAmGmGmGmGmAfAmAfAmUmGmGmA*mA*m |
| | | | A*mA |
| 299 | 250 | s | mU*mA*mGmCmAmUmAmUfAfAfUmGmUmAmUmUmUmUmG*mA*mA |
| | 658 | as | mU*fU*mCmAfAmAfAmUmAmCmAmUmUfAmUfAmUmGmCmU*mA*m |
| | | | A*mC |
| 300 | 571 | s | [iAb]*mA*mUmGmAmAmUmGmUfGfAfAmUmAmUmCmUmUmUmG*m |
| | | | A*mA |
| | 659 | as | mU*fU*mCmAfAmAfCmAmUmAmUmUmCfAmCfAmUmUmCmA*mU*m |
| | | | U*mG |
| 301 | 571 | s | [iAb]*mA*mUmGmAmAmUmGmUfGfAfAmUmAmUmCmUmUmUmG*m |
| | | | A*mA |
| | 660 | as | mU*fU*mCmAfAmAfImAmUmAmUmUmCfAmCfAmUmUmCmA*mU*mU |
| | | | *mG |
| 302 | 585 | s | [iAb]*mA*mUmGmAmAmUmGmUfGfAfAmUmAmUmGmUmUmUmG*m |
| | | | A*mA |
| | 659 | as | mU*fU*mCmAfAmAfCmAmUmAmUmUmCfAmCfAmUmUmCmA*mU*m |
| | | | U*mG |
| 303 | 586 | s | [iAb]*mA*mUmGmAmUmUmGmUfGfAfAmUmAmUmCmUmUmUmG*m |
| | | | A*mA |
| | 661 | as | mU*fU*mCmAfAmAfGmAmUmAmUmUmCfAmCfAmAmUmCmA*mU*m |
| | | | U*mG |
| 304 | 584 | s | [iAb]*mU*mUmCmCmAmUmUmUfUfCfCmCmCmUmUmUmAmUmG*mU |
| | | | *mA |
| | 662 | as | mU*fA*mCmAfUmAfAmAmGmGmGmGmAfAmAfAmUmG*mG*mA*mA |
| 305 | 572 | s | [iAb]*mG*mGmCmUmCmCmUmGfAfAfAmUmGmCmUmUmGmAmU*mG |
| | | | *mA |
| | 663 | as | mU*fC*mAmUmCmAmAmGmCmAmUmUmUfCmAfGmGmAmGmC*mC* |
| | | | mA*mU |
| 306 | 572 | s | [iAb]*mG*mGmCmUmCmCmUmGfAfAfAmUmGmCmUmUmGmAmU*mG |
| | | | *mA |
| | 664 | as | mU*fC*mAmUfC[UNA- |
| | | | A]fAmGmCmAmUmUmUfCmAfGmGmAmGmC*mC*mA*mU |
| 307 | 572 | s | [iAb]*mG*mGmCmUmCmCmUmGfAfAfAmUmGmCmUmUmGmAmU*mG |
| | | | *mA |
| | 665 | as | mU*fC*mAmUfCmA[UNA- |
| | | | A]mGmCmAmUmUmUfCmAfGmGmAmGmC*mC*mA*mU |
| 308 | 587 | s | [iAb]*mG*mGmCmUmCmCmUmGfAfAfAmUmGmCmCmUmGmAmU*mG |
| | | | *mA |
| | 666 | as | mU*fC*mAmUfCmAfImGmCmAmUmUmUfCmAfGmGmAmGmC*mC*mA |
| | | | *mU |
| 309 | 588 | s | [iAb]*mU*mAmUmGmUmUmAmAfAfAfAmGmAmAmAmUmCmUmC*m |
| | | | U*mA |
| | 667 | as | mU*fA*mGmAfGmAfUmUmUmCmUmUmUfUmUfAmAmCmAmU*mA*m |
| | | | A*mU |
| 310 | 589 | s | mC*mA*mUmAmGmCmUmUfUfCfUmUmUmUmCmCmAmUmU*mU*mA |
| | 668 | as | mU*fA*mAmAfUmGfGmAmAmAmAmGmAfAmAfGmCmUmAmUmG*m |
| | | | A*mG |
| 311 | 590 | s | [iAb]*mC*mAmUmAmGmCmUmUfUfCfUmUmUmUmCmCmAmUmU*mU |
| | | | *mA |
| | 669 | as | mU*fA*mAmAfUmGfGmAmAmAmAmGmAfAmAfGmCmUmAmU*mG*m |
| | | | A*mG |
| 312 | 570 | s | [iAb]*mU*mUmGmAmUmGmAmUfAfCfAmAmUmGmAmAmUmGmU*m |
| | | | G*mA |
| | 670 | as | mU*fC*mAmCmAmUmUmCmAmUmUmGmUfAmUfCmAmUmCmA*mA* |
| | | | mG*mC |
| 313 | 570 | s | [iAb]*mU*mUmGmAmUmGmAmUfAfCfAmAmUmGmAmAmUmGmU*m |
| | | | G*mA |
| | 671 | as | mU*fC*mAmCfA[UNA- |
| | | | U]fUmCmAmUmUmGmUfAmUfCmAmUmCmA*mA*mG*mC |
| 314 | 570 | s | [iAb]*mU*mUmGmAmUmGmAmUfAfCfAmAmUmGmAmAmUmGmU*m |
| | | | G*mA |
| | 672 | as | mU*fC*mAmCfAmU[UNA- |
| | | | U]mCmAmUmUmGmUfAmUfCmAmUmCmA*mA*mG*mC |
| 315 | 591 | s | [iAb]*mU*mUmGmAmUmGmAmUfAfCfAmAmUmGmCmAmUmGmU*m |
| | | | G*mA |
| | 673 | as | mU*fC*mAmCfAmUfImCmAmUmUmGmUfAmUfCmAmUmCmA*mA*mG |
| | | | *mC |
| 316 | 592 | s | [iAb]*mU*mUmCmAmAmAmCmGfAfGfCmUmGmAmGmAmUmCmU*mA |
| | | | *mA |
| | 674 | as | mU*fU*mAmGfAmUfCmUmCmAmGmCmUfCmGfUmUmUmGmA*mA*m |
| | | | G*mG |
| 317 | 593 | s | [iAb]*mU*mUmCmAmUmAmCmGfAfGfCmUmGmAmCmAmUmCmU*mA |
| | | | *mA |
| | 675 | as | mU*fU*mAmGfAmUfGmUmCmAmGmCmUfCmGfUmAmUmGmA*mA*m |
| | | | G*mG |
| 318 | 568 | s | [iAb]*mU*mUmCmAmAmAmCmGfAfGfCmUmGmAmCmAmUmCmU*mA |
| | | | *mA |
| | 676 | as | mU*fU*mAmGfAmUfImUmCmAmGmCmUfCmGfUmUmUmGmA*mA*m |
| | | | G*mG |
| 319 | 594 | s | [iAb]*mA*mUmCmUmCmAfAfCfUmUmUmGmUmGmUmCmA*mA*mA |
| | 677 | as | mU*fU*mUmGfAmCfAmCmAmAmAmGmUfUmGfAmGmA*mU*mA*mU |
| 320 | 595 | s | [iAb]*mU*mAmUmCmUmCmAfAfCfUmUmUmGmUmGmUmCmA*mA*m |
| | | | A |
| | 678 | as | mU*fU*mUmGfAmCfAmCmAmAmAmGmUfUmGfAmGmAmU*mA*mU* |
| | | | mA |
| 321 | 569 | s | [iAb]*mA*mUmAmUmCmUmCmAfAfCfUmUmUmGmUmGmUmCmA*mA |
| | | | *mA |
| | 677 | as | mU*fU*mUmGfAmCfAmCmAmAmAmGmUfUmGfAmGmA*mU*mA*mU |
| 322 | 596 | s | [iAb]*mU*mAmUmAmUmCmUmCmAfAfCfUmUmUmGmUmGmUmCmA* |
| | | | mA*mA |
| | 678 | as | mU*fU*mUmGfAmCfAmCmAmAmAmGmUfUmGfAmGmAmU*mA*mU* |
| | | | mA |
| 323 | 597 | s | [iAb]*mC*mUmAmUmAmUmCmUmCmAfAfCfUmUmUmGmUmGmUmC |
| | | | mA*mA*mA |
| | 678 | as | mU*fU*mUmGfAmCfAmCmAmAmAmGmUfUmGfAmGmAmU*mA*mU* |
| | | | mA |
| 324 | 569 | s | [iAb]*mA*mUmAmUmCmUmCmAfAfCfUmUmUmGmUmGmUmCmA*mA |
| | | | *mA |
| | 679 | as | mU*fU*mUmGfAmCfAmCmAmAmAmGmUfUmGfAmGmAmU?mA?mU* |
| | | | mA*mG |
| 325 | 598 | s | [iAb]*mA*mAmUmCmAmAmAmGfAfAfUmAmUmCmUmUmAmGmU*m |
| | | | G*mA |
| | 680 | as | mU*fC*mAmCfUmAfAmGmAmUmAmUmUfCmUfUmUmGmAmU*mU*m |
| | | | U*mU |
| 326 | 598 | s | [iAb]*mA*mAmUmCmAmAmAmGfAfAfUmAmUmCmUmUmAmGmU*m |
| | | | G*mA |
| | 681 | as | mU*fC*mAmCfUmAfAmGmAmUmAmUmUfCmUfUmUmGmA?mU?mU* |
| | | | mU*mU |

Abbreviations-“m” indicates 2′-OMe; “f” indicated 2′-fluoro; “*” indicates phosphorothioate linkage; “Uhd” indicates 2′-O-hexadecyl uridine; “Ahd” indicates 2′-O-hexadecyl adenosine; “Chd” indicates 2′O-hexadecyl cytidine; “Ghd” indicates 2′-O-hexadecyl guanosine; “Upi” indicates 2′-O-icosanamidopropyl uridine; “iAb” indicates inverted abasic; “A” indicates a 3′ 1,3-dimethylimidazolidine-2-imine phosphoryl guanidine phosphoryl guanidine linkage; “UNA” indicates unlocked nucleotide; “I” indicates inosine: “Cp” indicates cyclopropyl phosphonate; “Ada” indicates 2′-O-docosyl adenosine; “s” means the sense strand; “as” means the antisense strand.

The sense strand and antisense strand of dsRNA can be synthesized using any nucleic acid polymerization methods known in the art, for example, solid-phase synthesis by employing phosphoramidite chemistry methodology (e.g., Current Protocols in Nucleic Acid Chemistry, Beaucage, S. L. et al. (Edrs.), John Wiley & Sons, Inc., New York, NY, USA), H-phosphonate, phosphortriester chemistry, or enzymatic synthesis. Automated commercial synthesizers can be used, for example, MerMade™ 12 from LGC Biosearch Technologies, or other synthesizers from BioAutomation or Applied Biosystems. Phosphorothioate linkages can be introduced using a sulfurizing reagent such as phenylacetyl disulfide or DDTT (((dimethylaminomethylidene) amino)-3H-1,2,4-dithiazaoline-3-thione). It is well known to use similar techniques and commercially available modified amidites and controlled-pore glass (CPG) products to synthesize modified oligonucleotides or conjugated oligonucleotides.

Purification methods can be used to exclude the unwanted impurities from the final oligonucleotide product. Commonly used purification techniques for single stranded oligonucleotides include reverse-phase ion pair high performance liquid chromatography (RP-IP-HPLC), capillary gel electrophoresis (CGE), anion exchange HPLC (AX-HPLC), and size exclusion chromatography (SEC). After purification, oligonucleotides can be analyzed by mass spectrometry and quantified by spectrophotometry at a wavelength of 260 nm. The sense strand and antisense strand can then be annealed to form a dsRNA.

### Linker

The conjugates described herein can include a linker (L).

In some embodiments, L comprises a linker core (LC) and one or more spacers. Exemplary LC structures are provided in Table 7. The spacers may be small organic groups or polymer chains or the like. In some embodiments, L comprises Spacer1-Linker Core-Spacer2. In some embodiments, Spacer1 and Spacer 2 are selected from Table 8. Exemplary linkers are provided in Table 9.

TABLE 7
Exemplary Linker Core (LC) Structures

| Linker Core | Structure |
|---|---|
| LC1 | -CO-C2-CO-DBCO/N3-CH2-CO- |
| | 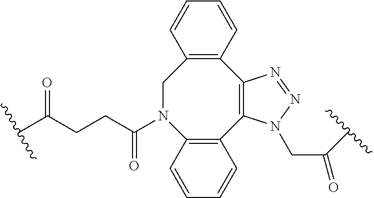 [SMILES: *C(=O)CCC(=O)N1Cc2ccccc2-c2nnn(CC(*)=O)c2-c2ccccc21] |
| LC2 | CO-SMCC-Cys-CO- |
| | 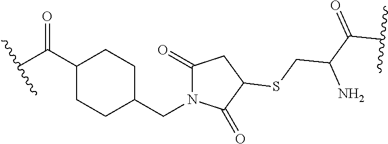 [SMILES: *C(=O)C(N)CSC1CC(=O)N(CC2CCC(C(*)=O)CC2)C1=O] |
| LC3 | -CO-SMCC/Mpa-CO- |
| | 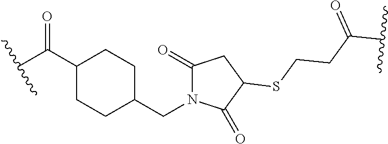 [SMILES: *C(=O)CCSC1CC(=O)N(CC2CCC(C(*)=O)CC2)C1=O] |
| LC4 | -CO-PEG-POD/Mpa-CO- |
| | 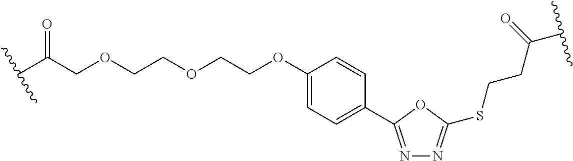 [SMILES: *C(=O)CCSc1nnc(-c2ccc(OCCOCCOCC(*)=O)cc2)o1] |
| LC5 | -CO-PEG-PT/Mpa-CO- |
| | 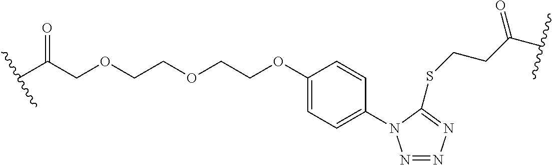 [SMILES: *C(=O)CCSc1nnnn1-c1ccc(OCCOCCOCC(*)=O)cc1] |
| LC6 | -CO-CH2-Mpa-CO- |
| | 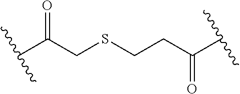 [SMILES: *C(=O)CCSCC(*)=O] |
| LC7 | -CO-Mpa/Mal(RO)-Dap- |
| | Regioisomer 1: 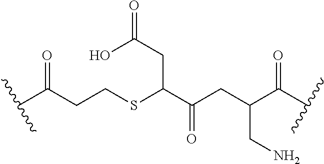 [SMILES: *C(=O)CCSC(CC(=O)O)C(=O)CC(CN)C(*)=O] |
| | Regioisomer 2: 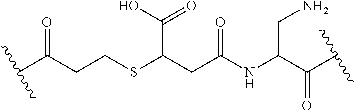 [SMILES: *C(=O)CCSC(CC(=O)NC(CN)C(*)=O)C(=O)O] |
| LC8 | -CO-SMCC(RO)/Mpa-CO- |
| | Regioisomer 1: 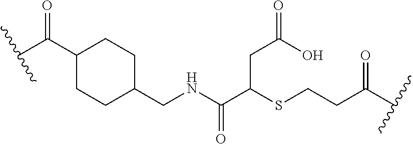 [SMILES: *C(=O)CCSC(CC(=O)O)C(=O)NCC1CCC(C(*)=O)CC1] |
| | Regioisomer 2: 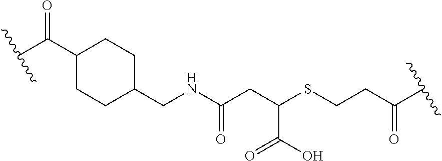 [SMILES: *C(=O)CCSC(CC(=O)NCC1CCC(C(*)=O)CC1)C(=O)O] |
| LC9 | -/Mal(RO)-Dap-CO- |
| | Regioisomer 1: 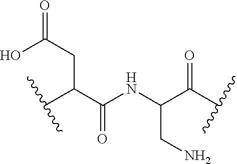 [SMILES: *C(=O)C(CN)NC(=O)C(*)CC(=O)O] |
| | Regioisomer 2: 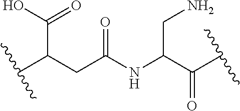 [SMILES: *C(=O)C(CN)NC(=O)CC(*)C(=O)O] |
| LC10 | -CO-Mpa/PT-PEG-CO- |
| | 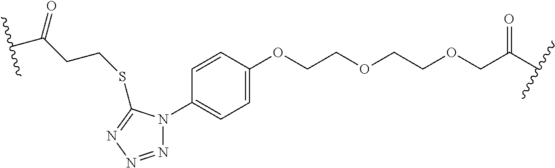 [SMILES: *C(=O)CCSc1nnnn1-c1ccc(OCCOCCOCC(*)=O)cc1] |
| LC11 | -/PT-PEG-CO- |
| | 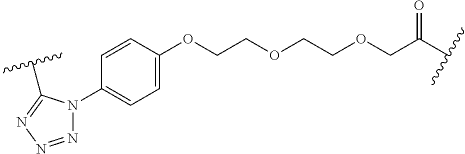 [SMILES: *C(=O)COCCOCCOc1ccc(-n2nnnc2*)cc1] |

It will be appreciated that in some instances, the linker structures above represent final structural configurations of the linkers where precursor molecules on different portions of the conjugate have been reacted to yield such structures. That is, a first precursor to the linker may be covalently attached to the oligonucleotide, and a second precursor to the linker may be covalently attached to the peptide. Then the two precursors are brought into proximity with one another under proper conditions and form a covalent linkage yielding one of the structures shown herein.

TABLE 8
Exemplary Linker Spacers (SL, SP)

| Spacer | |
|---|---|
| name | Spacer structure |
| SL1 | -P(O)SH-O-C6-NH- |
| | 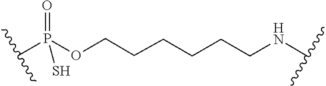 [SMILES: *NCCCCCCOP(*)(=O)S] |
| SL2 | (2′-OH) -C3-NH- |
| | 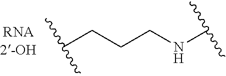 [SMILES: *CCCN*] |
| SL3 | -P(O)SH-O-C6-S- |
| | 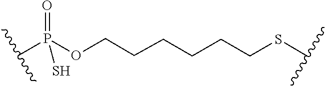 [SMILES: *SCCCCCCOP(*)(=O)S] |
| SL4 | P(O)OH-O-C6-NH- |
| | 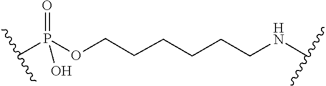 [SMILES: *NCCCCCCOP(*)(=O)O] |
| SL5 | P(O)OH-O-C6-S- |
| | 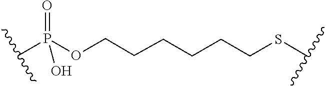 [SMILES: *SCCCCCCOP(*)(=O)O] |
| SP1 | -NH-AEEA3-CO- |
| | 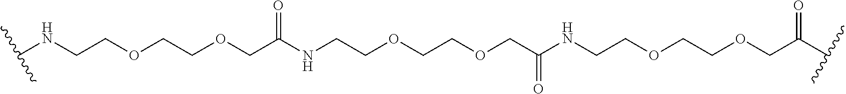 [SMILES: *NCCOCCOCC(=O)NCCOCCOCC(=O)NCCOCCOCC(*)=O] |
| SP2 | AEEA3-fSp-Cha-aGPIDRIGGGGSGGGGS |
| (SEQ | |
| ID: 58) | |
| SP3 | GGGGSGGGGSfSp-Cha-aGPIDRIGGGGSGGGGS |
| (SEQ | |
| ID: 59) | |
| SP4 | GGGGSGGGGSGGGGSGGGGS |
| (SEQ | |
| ID: 60) | |
| SP5 | AEEA3-GGGGSGGGGSGGGGSGGGGS |
| (SEQ | |
| ID: 61) | |
| SP6 | -NH-AEEA6-CO- |
| | 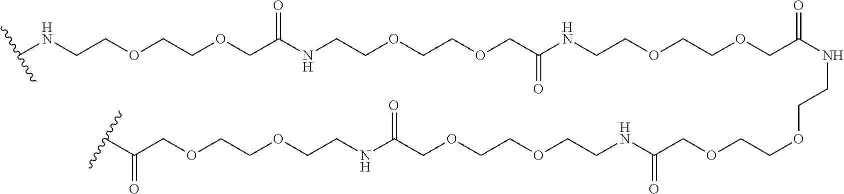 [SMILES: *NCCOCCOCC(=O)NCCOCCOCC(=O)NCCOCCOCC(=O)NCCOCCOCC(=O)NCCOCCOCC(=O)NCCOCCOCC(*)=O] |
| SP7 | -NH-AEEA8-CO- |
| | 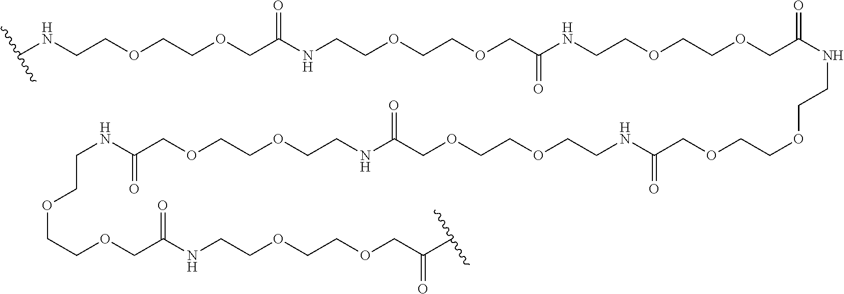 [SMILES: *NCCOCCOCC(=O)NCCOCCOCC(=O)NCCOCCOCC(=O)NCCOCCOCC(=O)NCCOCCOCC(=O)NCCOCCOCC(=O)NCCOCCOCC(=O)NCCOCCOCC(*)=O] |
| SP8 | GGGGHGGGGHGGGGHGGGGH |
| (SEQ | |
| ID: 62) | |
| SP9 | GGGG-4Pal-GGGG-4Pal-GGGG-4Pal-GGGG-4Pal |
| (SEQ | |
| ID: 63) | |
| SP10 | GGGGSHHHGGGGSHHHGGGGS |
| (SEQ | |
| ID: 64) | |
| SP11 | AEEA3-GGGGSGGGGS |
| (SEQ | |
| ID: 65) | |
| SP12 | GGGGSGGGGSGGGGSGGGGSGGGGSGGGGS |
| (SEQ | |
| ID: 66) | |
| SP13 | -NH-PEG24-CO- |
| | 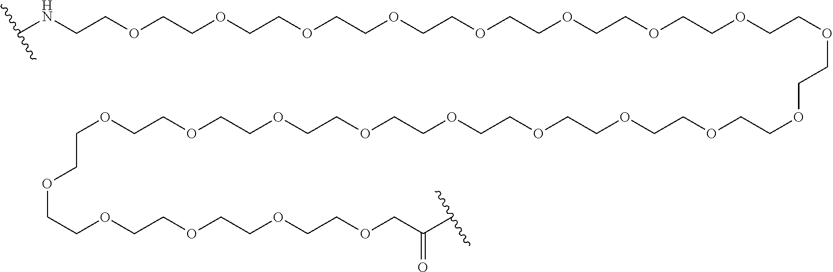 [SMILES: *NCCOCCOCCOCCOCCOCCOCCOCCOCCOCCOCCOCCOCCOCCOCCOCCOCCOCCOCCOCCOCCOCCOCCOCC(*)=O] |
| SP14 | GGGGSGGGGSGGGGSGGGGSP |
| (SEQ | |
| ID: 67) | |
| SP15 | GGGG-4Pal-GGGG-4Pal-GGGG-4Pal-GGGG-4Pal-P |
| (SEQ | |
| ID: 68) | |
| SP16 | GGGG-4Pal-GGGG-4Pal-GGGG-4Pal-GGGG-4Pal-GGGG-4Pal-GGGG-4Pal |
| (SEQ | |
| ID: 69) | |
| SP17 | GGGGEGGGGEGGGGSGGGGSGGGGSGGGGS |
| (SEQ | |
| ID: 70) | |
| SP18 | GGGGEGGGGSGGGGEGGGGSGGGGEGGGGS |
| (SEQ | |
| ID: 71) | |
| SP19 | GGGGEGGGGEGGGGEKEKEKGGGGSGGGGS |
| (SEQ | |
| ID: 72) | |

TABLE 9
Exemplary Linkers (L)

| Linker Name | Spacer1(SL)-Linker Core(LC)-Spacer2(SP) |
|---|---|
| L1 | SL1-LC1-SP1 |
| L2 | SL1-LC2-SP1 |
| L3 | SL1-LC3-SP1 |
| L4 | SL1-LC4-SP1 |
| L5 | SL1-LC5-SP1 |
| L6 | SL1-LC6-SP1 |
| L7 | SL1-LC7-SP1 |
| L8 | SL1-LC8-SP1 |
| L9 | SL-LC10-SP1 |
| L10 | SL3-LC9-SP1 |
| L11 | SL2-LC3-SP1 |
| L12 | SL1-LC3-SP2 |
| L13 | SL1-LC3-SP3 |
| L14 | SL1-LC3-SP7 |
| L15 | SL1-LC3-SP8 |
| L16 | SL1-LC3-SP9 |
| L17 | SL1-LC3-SP10 |
| L18 | SL1-LC3-SP12 |
| L19 | SL1-LC5-SP12 |
| L20 | SL1-LC10-SP12 |
| L21 | SL1-LC7-SP12 |
| L22 | SL3-LC9-SP12 |
| L23 | SL3-LC11-SP12 |
| L24 | SL3-LC11-SP1 |
| L25 | SL3-LC11-SP17 |
| L26 | SL3-LC11-SP18 |
| L27 | SL3-LC11-SP19 |

### Fatty Acids

In some embodiments, the conjugates further comprise a fatty acid (FA) or lipid. The FA can be saturated or unsaturated. In some embodiments, the FA is a C₁₂-C₂₂ fatty acid. Exemplary fatty acids are provided in Table 10.

In some embodiments, FA is attached to O. In some embodiments, FA is attached to P. In some embodiments, FA is attached to L.

In some embodiments, the conjugate comprises (FA)ₘ-O-L-P or O-L-P-(FA)ₘ, wherein m is an integer of 1 to 4. In some embodiments, m is 1 or 2.

In some embodiments, the conjugate further comprises a Spacer3. Exemplary fatty acid spacers (Spacer3) are provided in Table 11.

Exemplary FA-Spacer3 pairs are provided in Table 12.

In some embodiments, the conjugate comprises (FA-Spacer3)ₘ-O-L-P or O-L-P-(Spacer3-FA)ₘ, wherein m is an integer of 1 to 4. In some embodiments, m is 1 or 2. In some embodiments, the conjugate comprises FA-Spacer3-O-L-P or O-L-P-Spacer3-FA. In some embodiments, the conjugate comprises FA-Spacer3-O-Spacer1-LinkerCore-Spacer2-P or O-Spacer1-LinkerCore-Spacer2-P-Spacer3-FA.

In some embodiments, the conjugate comprises O-L-(FA-Spacer3)ₘ-P or 0-(Spacer3-FA)ₘ-L-P, wherein m is an integer of 1 to 4. In some embodiments, m is 1 or 2. In some embodiments, the conjugate comprises O-L-FA-Spacer3-P or O-Spacer3-FA-L-P. In some embodiments, the conjugate comprises O-Spacer1-LinkerCore-Spacer2-FA-Spacer3-P or O-Spacer3-FA-Spacer1-LinkerCore-Spacer2-P.

The fatty acids FA of Table 10 may be incorporated into conjugates of the present disclosure by several different methods. For example, these lipids may be conjugated to an RNA molecule after synthesis is complete, or they may be incorporated at an internal position of the RNA by being provided as part of an amidite, or by any other conventional method.

TABLE 10
Exemplary Fatty Acids (FA)

| Fatty Acid | Structure |
|---|---|
| FA1 | Ac-L-Cpa(/L-Cys-CO-)-K(AEEA2-yE-C20 diacid)-NH2 |
| | 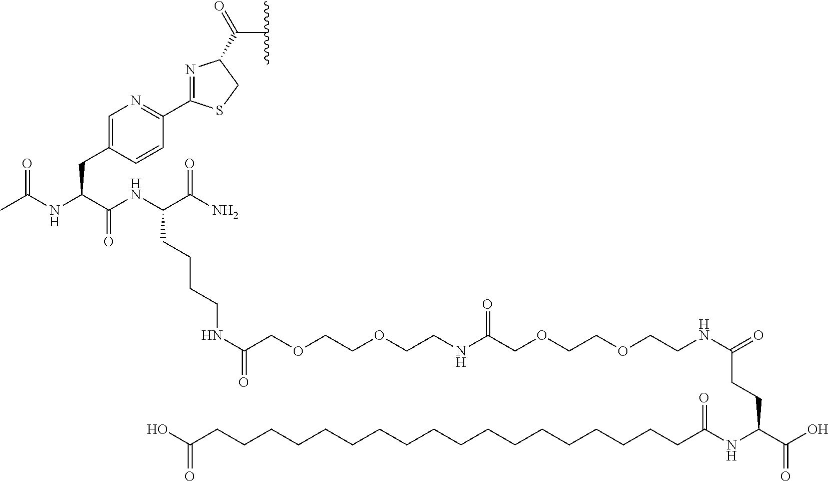 [SMILES: *C(=O)[C@@H]1CSC(c2ccc(C[C@H](NC(C)=O)C(=O)N[C@@H](CCCCNC(=O)COCCOCCNC(=O)COCCOCCNC(=O)CC[C@H](NC(=O)CCCCCCCCCCCCCCCCCCC(=O)O)C(=O)O)C(N)=O)cn2)=N1] |
| FA2 | -CO-AEEA2-yE-C20 diacid |
| | 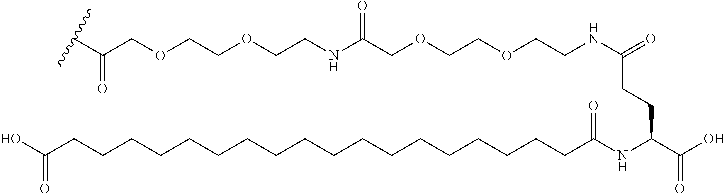 [SMILES: *C(=O)COCCOCCNC(=O)COCCOCCNC(=O)CC[C@H](NC(=O)CCCCCCCCCCCCCCCCCCC(=O)O)C(=O)O] |
| FA3 | -K(AEEA2-yE-C20 diacid)-NH2 |
| | 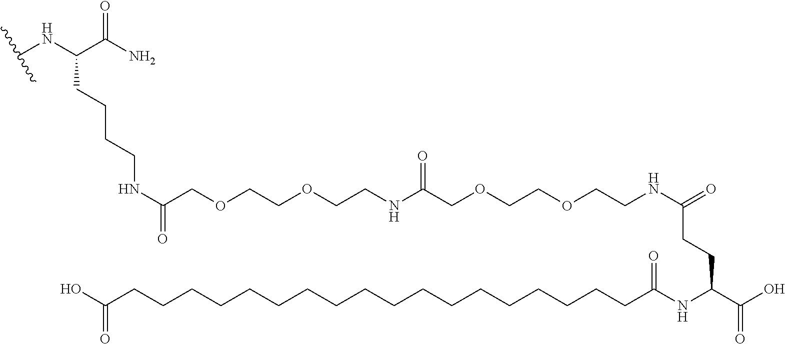 [SMILES: *N[C@@H](CCCCNC(=O)COCCOCCNC(=O)COCCOCCNC(=O)CC[C@H](NC(=O)CCCCCCCCCCCCCCCCCCC(=O)O)C(=O)O)C(N)=O] |
| FA4 | -K(AEEA2-yE-C20 diacid)- |
| | 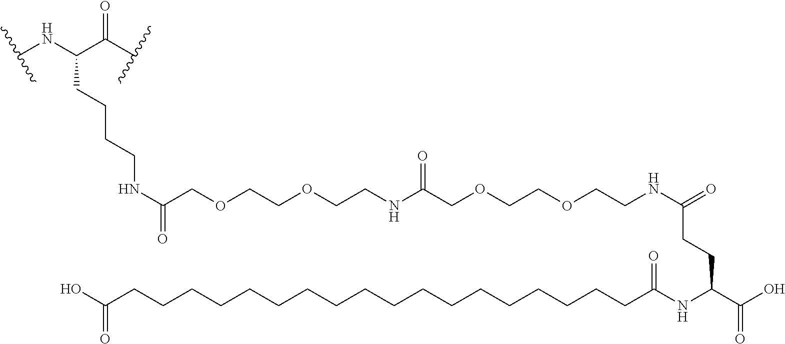 [SMILES: *N[C@@H](CCCCNC(=O)COCCOCCNC(=O)COCCOCCNC(=O)CC[C@H](NC(=O)CCCCCCCCCCCCCCCCCCC(=O)O)C(=O)O)C(*)=O] |
| FA5 | -P(O)SH-O-C16 |
| | 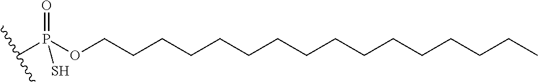 [SMILES: *P(=O)(S)OCCCCCCCCCCCCCCCC] |
| FA6 | -P(O)SH-O-C22 |
| | 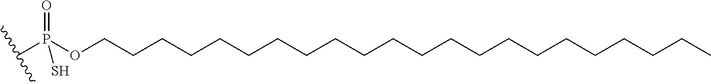 [SMILES: *P(=O)(S)OCCCCCCCCCCCCCCCCCCCCCC] |
| FA7 | -P(O)SH-O-C18 unsaturated (-oleyl) |
| | 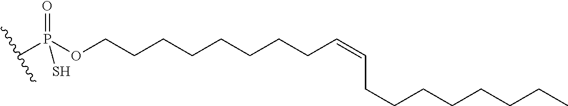 [SMILES: *P(=O)(S)OCCCCCCCC/C=C\CCCCCCCC] |
| FA8 | -P(O)SH-O-C20 branched |
| | 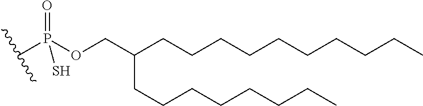 [SMILES: *P(=O)(S)OCC(CCCCCCCC)CCCCCCCCCC] |
| FA9 | -P(O)SH-O-C12 caged (-ethyl adamantyl) |
| | 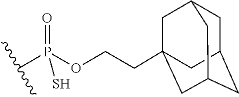 [SMILES: *P(=O)(S)OCCC12CC3CC(CC(C3)C1)C2] |
| FA10 | -C20 diacid |
| | 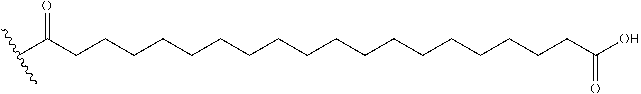 [SMILES: *C(=O)CCCCCCCCCCCCCCCCCCC(=O)O] |
| FA11 | -P(O)OH-O-C16 |
| | 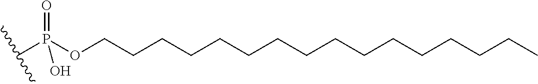 [SMILES: *P(=O)(O)OCCCCCCCCCCCCCCCC] |
| FA12 | -P(O)OH-O-C22 |
| | 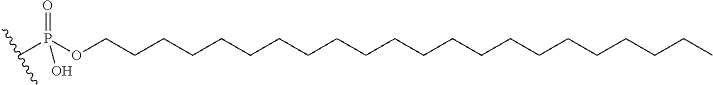 [SMILES: *P(=O)(O)OCCCCCCCCCCCCCCCCCCCCCC] |
| FA13 | -P(O)OH-O-C18 unsaturated (-oleyl) |
| | 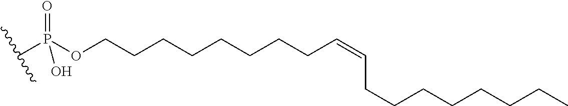 [SMILES: *P(=O)(O)OCCCCCCCC/C=C\CCCCCCCC] |
| FA14 | -P(O)OH-O-C20 branched |
| | 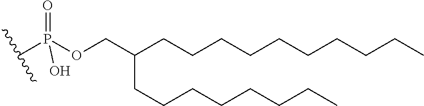 [SMILES: *P(=O)(O)OCC(CCCCCCCC)CCCCCCCCCC] |
| FA15 | -P(O)OH-O-C12 caged (-ethyl adamantyl) |
| | 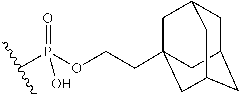 [SMILES: *P(=O)(O)OCCC12CC3CC(CC(C3)C1)C2] |
| FA16 | -P(O)SH-O-glycol-( P(O-C16)SH)2 doubler |
| | 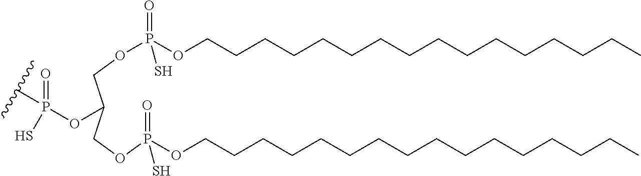 [SMILES: *P(=O)(S)OC(COP(=O)(S)OCCCCCCCCCCCCCCCC)COP(=O)(S)OCCCCCCCCCCCCCCCC] |
| FA17 | -P(O)SH-O-glycol-( P(O-C16)OH)2 doubler |
| | 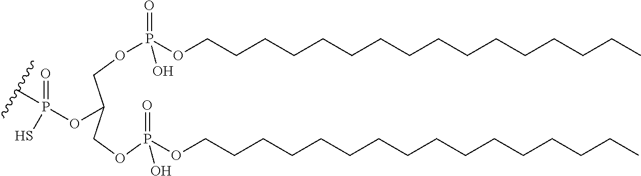 [SMILES: *P(=O)(S)OC(COP(=O)(O)OCCCCCCCCCCCCCCCC)COP(=O)(O)OCCCCCCCCCCCCCCCC] |
| FA18 | -P(O)OH-O-glycol-( P(O-C16)OH)2 doubler |
| | 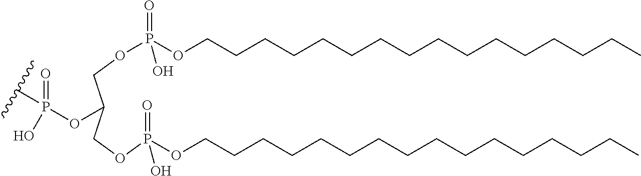 [SMILES: *P(=O)(O)OC(COP(=O)(O)OCCCCCCCCCCCCCCCC)COP(=O)(O)OCCCCCCCCCCCCCCCC] |
| FA19 | -P(O)SH-O-C16-Acid |
| | 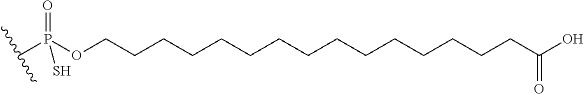 [SMILES: *P(=O)(S)OCCCCCCCCCCCCCCCC(=O)O] |
| FA20 | -P(O)SH-O-C18-Acid |
| | 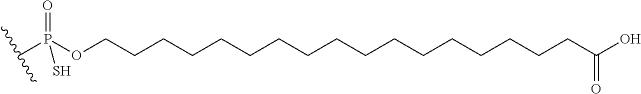 [SMILES: *P(=O)(S)OCCCCCCCCCCCCCCCCCC(=O)O] |
| FA21 | -P(O)SH-O-C20-Acid |
| | 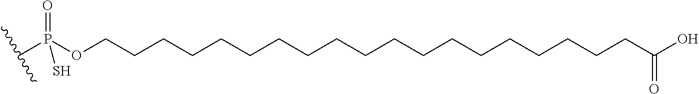 [SMILES: *P(=O)(S)OCCCCCCCCCCCCCCCCCCCC(=O)O] |
| FA22 | -P(O)OH-O-C16-Acid |
| | 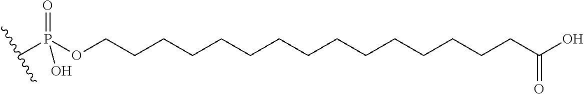 [SMILES: *P(=O)(O)OCCCCCCCCCCCCCCCC(=O)O] |
| FA23 | -P(O)OH-O-C18-Acid |
| | 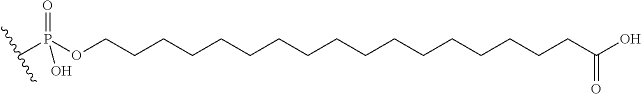 [SMILES: *P(=O)(O)OCCCCCCCCCCCCCCCCCC(=O)O] |
| FA24 | -P(O)OH-O-C20-Acid |
| | 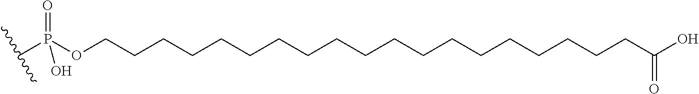 [SMILES: *P(=O)(O)OCCCCCCCCCCCCCCCCCCCC(=O)O] |
| FA25 | -yE-C16 diacid |
| | 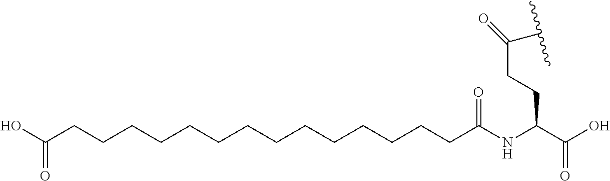 [SMILES: *C(=O)CC[C@H](NC(=O)CCCCCCCCCCCCCCC(=O)O)C(=O)O] |
| FA26 | -C16 diacid |
| | 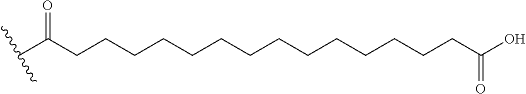 [SMILES: *C(=O)CCCCCCCCCCCCCCC(=O)O] |
| FA27 | -C20 |
| | 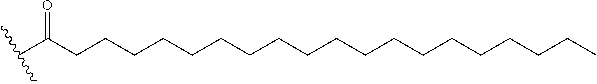 [SMILES: *C(=O)CCCCCCCCCCCCCCCCCCC] |

TABLE 11
Exemplary Spacers for Fatty Acids (Spacer3)

| | Spacers For Fatty Acids (Spacer3) |
|---|---|
| SF1* | (2′-OH)-C3-NH- |
| | 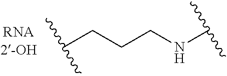 [SMILES: *CCCN*] |
| SF2 | -P(O)OH-O-cHex-pNH- |
| | 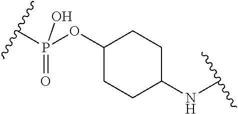 [SMILES: *NC1CCC(OP(*)(=O)O)CC1] |
| SF3 | -P(O)SH-O-C6-NH- |
| | 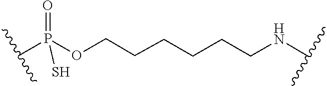 [SMILES: *NCCCCCCOP(*)(=O)S] |
| SF4 | GSPSSGAPPPS |
| (SEQ ID: | |
| 73) | |
| SF5 | -NH-AEEA3-CO- |
| | 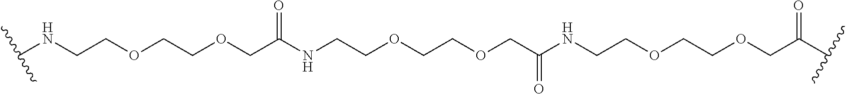 [SMILES: *NCCOCCOCC(=O)NCCOCCOCC(=O)NCCOCCOCC(*)=O] |
| SF6 | -P(O)SH-O-cHex-pNH- |
| | 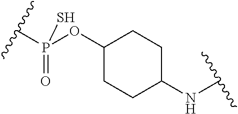 [SMILES: *NC1CCC(OP(*)(=O)S)CC1] |
| SF7 | -P(O)OH-O-C6-NH- |
| | 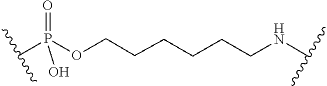 [SMILES: *NCCCCCCOP(*)(=O)O] |
| SF8 | -P(O)SH-O-C2-NH- |
| | 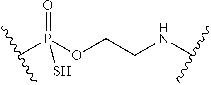 [SMILES: *NCCOP(*)(=O)S] |
| SF9 | -P(O)OH-O-C2-NH- |
| | 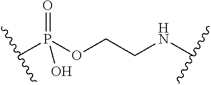 [SMILES: *NCCOP(*)(=O)O] |
| SF10 | -P(O)SH-O-PEG6-O- |
| | 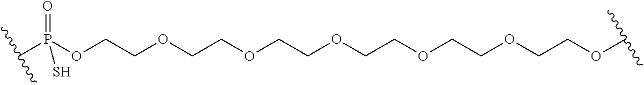 [SMILES: *OCCOCCOCCOCCOCCOCCOP(*)(=O)S] |
| SF11 | -P(O)OH-O-PEG6-O- |
| | 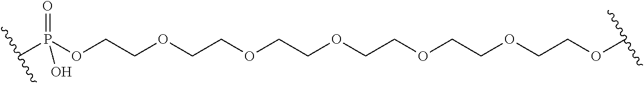 [SMILES: *OCCOCCOCCOCCOCCOCCOP(*)(=O)O] |
| SF12 | -P(O)SH-O-PEG6-O-P(O)SH-O-C2-NH- |
| | 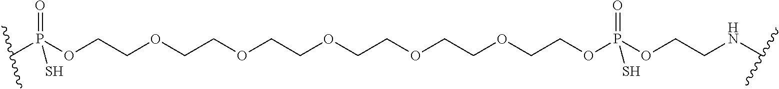 [SMILES: *NCCOP(=O)(S)OCCOCCOCCOCCOCCOCCOP(*)(=O)S] |
| SF13 | -P(O)SH-O-PEG6-O-P(O)OH-O-C2-NH- |
| | 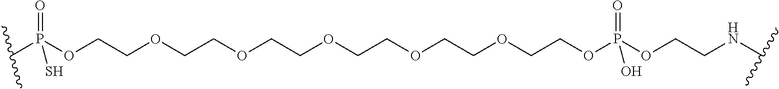 [SMILES: *NCCOP(=O)(O)OCCOCCOCCOCCOCCOCCOP(*)(=O)S] |
| SF14 | -P(O)OH-O-PEG6-O-P(O)SH-O-C2-NH- |
| | 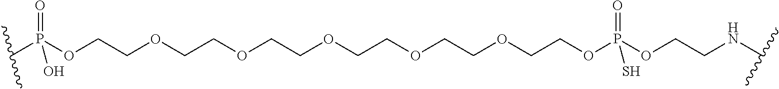 [SMILES: *NCCOP(=O)(S)OCCOCCOCCOCCOCCOCCOP(*)(=O)O] |
| SF15 | -P(O)OH-O-PEG6-O-P(O)OH-O-C2-NH- |
| | 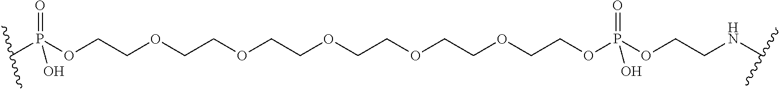 [SMILES: *NCCOP(=O)(O)OCCOCCOCCOCCOCCOCCOP(*)(=O)O] |

*For SF1, “(2′-OH)-C3-NH-” the aminopropyl moiety extends from the position where the 2′-hydroxyl hydrogen of an RNA would otherwise exist, with reference to the Upa 2′-O-propylamino uridine modified base.

TABLE 12
Exemplary FA-Spacer3 Pairs

| Pair No. | Fatty Acid-Spacer3 Pairs |
|---|---|
| 1 | FA1-SF2 |
| 2 | FA2-SF3 |
| 3 | FA3-SF4 |
| 4 | FA4-SF5 |
| 5 | FA4-SF4 |
| 6 | FA10-SF8 |
| 7 | FA5-SF10 |
| 8 | FA6-SF10 |
| 9 | FA19-SF10 |
| 10 | FA20-SF10 |
| 11 | FA21-SF10 |
| 12 | FA10-SF12 |
| 13 | FA25-SF10 |
| 14 | FA26-SF8 |

### Conjugates

Certain exemplary conjugates of the present disclosure are provided in Table 13:

TABLE 13
Exemplary conjugates comprising NPR-C binding peptide and dsRNA

| | | | | | | | Peptide | |
|---|---|---|---|---|---|---|---|---|
| | | | | | Linker | | (P) | |
| | | | dsRNA | | Core | | SEQ | |
| Conjugate | FA | SF | (R) | SL | (LC) | SP | ID | Configuration |
| NO | NO | NO | NO | NO | NO | NO | NO | (5′ end to 3′ end) |
| C1 | — | — | 99 | 1 | 3 | 1 | 20 | R-SL-LC-SP-P |
| C2 | — | — | 101 | 1 | 3 | 1 | 20 | R-SL-LC-SP-P |
| C3 | — | — | 157 | 1 | 3 | 1 | 20 | R-SL-LC-SP-P |
| C4 | — | — | 77 | 3 | 9 | 1 | 21 | R-SL-LC-SP-P |
| C5 | — | — | 85 | 3 | 9 | 1 | 21 | R-SL-LC-SP-P |
| C6 | — | — | 97 | 3 | 9 | 1 | 21 | R-SL-LC-SP-P |
| C7 | — | — | 157 | 3 | 9 | 1 | 21 | R-SL-LC-SP-P |
| C8 | — | — | 94 | 3 | 9 | 1 | 21 | R-SL-LC-SP-P |
| C9 | — | — | 91 | 3 | 9 | 1 | 21 | R-SL-LC-SP-P |
| C10 | — | — | 99 | 3 | 9 | 1 | 21 | R-SL-LC-SP-P |
| C11 | — | — | 100 | 3 | 9 | 1 | 21 | R-SL-LC-SP-P |
| C12 | — | — | 157* | 3 | 11 | 1 | 21 | R-SL-LC-SP-P |
| C13 | — | — | 160* | 3 | 11 | 1 | 21 | R-SL-LC-SP-P |
| C14 | 5 | — | 157 | 3 | 11 | 1 | 21 | FA-R-SL-LC-SP-P |
| C15 | 10 | 8 | 157 | 3 | 11 | 1 | 21 | FA-SF-R-SL-LC- |
| | | | | | | | | SP-P |
| C16 | 7 | — | 157 | 3 | 11 | 1 | 21 | FA-R-SL-LC-SP-P |
| C17 | 2 | 3 | 157 | 3 | 11 | 1 | 21 | FA-R-SL-LC-SP-P |
| C18 | — | — | 157* | 3 | 11 | 12 | 21 | R-SL-LC-SP-P |
| C19 | — | — | 157* | 3 | 11 | 17 | 21 | R-SL-LC-SP-P |
| C20 | — | — | 157* | 3 | 11 | 18 | 21 | R-SL-LC-SP-P |
| C21 | — | — | 157* | 3 | 11 | 19 | 21 | R-SL-LC-SP-P |
| C22 | 10 | 8 | 280 | 3 | 11 | 1 | 21 | FA-SF-R-SL- |
| | | | | | | | | LC-SP-P |
| C23 | — | — | 280 | 3 | 11 | 19 | 21 | R-SL-LC-SP-P |
| C24 | — | — | 281 | 3 | 11 | 19 | 21 | R-SL-LC-SP-P |
| C25 | — | — | 282 | 3 | 11 | 19 | 21 | R-SL-LC-SP-P |
| C26 | — | — | 283 | 3 | 11 | 19 | 21 | R-SL-LC-SP-P |
| C27 | — | — | 284 | 3 | 11 | 19 | 21 | R-SL-LC-SP-P |
| C28 | 10 | 8 | 281 | 3 | 11 | 1 | 21 | FA-SF-R-SL- |
| | | | | | | | | LC-SP-P |
| C29 | 10 | 8 | 282 | 3 | 11 | 1 | 21 | FA-SF-R-SL- |
| | | | | | | | | LC-SP-P |
| C30 | 10 | 8 | 283 | 3 | 11 | 1 | 21 | FA-SF-R-SL- |
| | | | | | | | | LC-SP-P |
| C31 | 10 | 8 | 284 | 3 | 11 | 1 | 21 | FA-SF-R-SL- |
| | | | | | | | | LC-SP-P |
| C32 | 26 | 8 | 281 | 3 | 11 | 19 | 21 | FA-SF-R-SL- |
| | | | | | | | | LC-SP-P |
| C33 | — | — | 285 | 3 | 11 | 19 | 21 | R-SL-LC-SP-P |
| C34 | 26 | 8 | 282 | 3 | 11 | 19 | 21 | FA-SF-R-SL- |
| | | | | | | | | LC-SP-P |
| C35 | — | — | 286 | 3 | 11 | 19 | 21 | R-SL-LC-SP-P |
| C36 | 26 | 8 | 283 | 3 | 11 | 19 | 21 | FA-SF-R-SL- |
| | | | | | | | | LC-SP-P |
| C37 | — | — | 290 | 3 | 11 | 19 | 21 | R-SL-LC-SP-P |
| C38 | 26 | 8 | 288 | 3 | 11 | 19 | 21 | FA-SF-R-SL- |
| | | | | | | | | LC-SP-P |
| C39 | — | — | 289 | 3 | 11 | 19 | 21 | R-SL-LC-SP-P |
| C40 | 26 | 8 | 284 | 3 | 11 | 19 | 21 | FA-SF-R-SL- |
| | | | | | | | | LC-SP-P |
| C41 | — | — | 287 | 3 | 11 | 19 | 21 | R-SL-LC-SP-P |
| C42 | — | — | 294 | 3 | 11 | 19 | 21 | R-SL-LC-SP-P |
| C43 | 10 | 8 | 294 | 3 | 11 | 1 | 21 | FA-SF-R-SL- |
| | | | | | | | | LC-SP-P |
| C44 | 10 | 8 | 294 | 3 | 11 | 19 | 21 | FA-SF-R-SL- |
| | | | | | | | | LC-SP-P |
| C45 | 26 | 8 | 294 | 3 | 11 | 19 | 21 | FA-SF-R-SL- |
| | | | | | | | | LC-SP-P |
| C46 | 26 | 8 | 294 | 3 | 11 | 19 | 21 | FA-SF-R-SL- |
| | | | | | | | | LC-SP-P |
| C47 | 5 | — | 294 | 3 | 11 | 19 | 21 | FA-R-SL-LC- |
| | | | | | | | | SP-P |
| C48 | 7 | — | 294 | 3 | 11 | 19 | 21 | FA-R-SL-LC- |
| | | | | | | | | SP-P |
| C49 | — | — | 295 | 3 | 11 | 19 | 21 | R-SL-LC-SP-P |

The dsRNAs of conjugates C1-C49 listed in this conjugate table were made with a 5′-vinylphosphonate modification on the antisense strand.
*These dsRNAs were made with a 5′-inverted abasic moiety attached to the sense strand by a phosphorothiolate linkage.

The conjugates described herein can be made by a variety of procedures known to one of ordinary skill in the art, some of which are illustrated in the preparations and examples below. One of ordinary skill in the art recognizes that the specific synthetic steps for each of the routes described may be combined in different ways, or in conjunction with steps from different schemes, to prepare conjugates. The product of each step can be recovered by conventional methods well known in the art, including extraction, evaporation, precipitation, chromatography, filtration, trituration, and crystallization. The reagents and starting materials are readily available to one of ordinary skill in the art.

### Pharmaceutical Composition

In another aspect, provided herein are pharmaceutical compositions comprising any of the oligonucleotides, RNAi agents, or conjugates described herein and a pharmaceutically acceptable carrier. Such pharmaceutical compositions target ALK7 in a patient in need of treatment. The pharmaceutical compositions can also comprise one or more pharmaceutically acceptable excipient, diluent, or carrier. Pharmaceutical compositions can be prepared by methods well known in the art (e.g., Remington: The Science and Practice of Pharmacy, 23rd edition (2020), A. Loyd et al., Academic Press).

### Method of Treatment and Therapeutic Use

In another aspect, provided herein are methods of treating a disease or condition of adipose tissue, e.g., obesity or obesity-related comorbidity, in a patient in need thereof, and such the method comprises administering to the patient an effective amount of an oligonucleotide, RNAi agent, conjugate, or pharmaceutical composition described herein. The conjugate or pharmaceutical composition can be administered to the patient intravenously or subcutaneously.

Also provided herein are methods of delivering an oligonucleotide to an adipose tissue, comprising administering to a subject a conjugate or pharmaceutical composition described herein.

Dosage regimens for the conjugates described herein may be adjusted to provide the optimum desired response (e.g., a therapeutic response). For example, a single bolus may be administered, several divided doses may be administered over time, or the dose may be proportionally reduced or increased as indicated by the exigencies of the therapeutic situation.

Dosage values may vary with the type and severity of the condition to be alleviated. It is further understood that for any particular subject, specific dosage regimens should be adjusted over time according to the individual need and the professional judgment of the person administering or supervising the administration of the compositions.

In another aspect, provided herein are conjugates or pharmaceutical compositions described herein for use in a therapy. In some embodiments, provided herein are conjugates or pharmaceutical compositions described herein for use in the treatment of a disease or condition involving ALK7, e.g., obesity or obesity-related comorbidity.

Also provided herein are uses of conjugates or pharmaceutical compositions described herein in the manufacture of a medicament for treating a disease or condition involving ALK7, including in or of adipose tissue, e.g., obesity or obesity-related comorbidity.

Also provided herein are methods of delivering an oligonucleotide to an adipose tissue, comprising administering to a subject a conjugate or pharmaceutical composition described herein.

As used herein, the terms “a,” “an,” “the,” and similar terms used in the context of the present disclosure (especially in the context of the claims) are to be construed to cover both the singular and plural unless otherwise indicated herein or clearly contradicted by the context.

As used herein, the term “alkyl” means saturated linear or branched-chain monovalent hydrocarbon radical, containing the indicated number of carbon atoms. For example, “C₁-C₂₂ alkyl” means a radical having 1-22 carbon atoms in a linear or branched arrangement.

As used herein, “antisense strand” means a single-stranded oligonucleotide that is complementary to a region of a target sequence. Likewise, and as used herein, “sense strand” means a single-stranded oligonucleotide that is complementary to a region of an antisense strand.

The terms “bind” and “binds” as used herein are intended to mean, unless indicated otherwise, the ability of a protein or molecule to form a chemical bond or attractive interaction with another protein or molecule, which results in proximity of the two proteins or molecules as determined by common methods known in the art.

As used herein, “complementary” means a structural relationship between two nucleotides (e.g., on two opposing nucleic acids or on opposing regions of a single nucleic acid strand, e.g., a hairpin) that permits the two nucleotides to form base pairs with one another. For example, a purine nucleotide of one nucleic acid that is complementary to a pyrimidine nucleotide of an opposing nucleic acid may base pair together by forming hydrogen bonds with one another. Complementary nucleotides can base pair in the Watson-Crick manner or in any other manner that allows for the formation of stable duplexes. Likewise, two nucleic acids may have regions of multiple nucleotides that are complementary with each other to form regions of complementarity, as described herein.

As used herein, “duplex,” in reference to nucleic acids or oligonucleotides, means a structure formed through complementary base pairing of two antiparallel sequences of nucleotides (i.e., in opposite directions), whether formed by two separate nucleic acid strands or by a single, folded strand (e.g., via a hairpin).

An “effective amount” refers to an amount necessary (for periods of time and for the means of administration) to achieve the desired therapeutic result. An effective amount of a protein or conjugate may vary according to factors such as the disease state, age, sex, and weight of the individual, and the ability of the protein or conjugate to elicit a desired response in the individual. An effective amount is also one in which any toxic or detrimental effects of the protein or conjugate are outweighed by the therapeutically beneficial effects.

The term “knockdown” or “expression knockdown” refers to reduced mRNA or protein expression of a gene after treatment of a reagent.

As used herein, “modified internucleotide linkage” means an internucleotide linkage having one or more chemical modifications when compared with a reference internucleotide linkage having a phosphodiester bond. A modified internucleotide linkage can be a non-naturally occurring linkage. In some embodiments, the modified internucleotide linkage is phosphorothioate linkage.

As used herein, “modified nucleotide” refers to a nucleotide having one or more chemical modifications when compared with a corresponding reference nucleotide selected from: adenine ribonucleotide, guanine ribonucleotide, cytosine ribonucleotide, uracil ribonucleotide, adenine deoxyribonucleotide, guanine deoxyribonucleotide, cytosine deoxyribonucleotide, and thymidine deoxyribonucleotide. A modified nucleotide can have, for example, one or more chemical modification in its sugar, nucleobase, and/or phosphate group. Additionally, or alternatively, a modified nucleotide can have one or more chemical moieties conjugated to a corresponding reference nucleotide. In some embodiments, the modified nucleotide is a 2′-fluoro modified nucleotide, 2′-O-methyl modified nucleotide, or 2′-O-alkyl (e.g., 2′-O—C₁₆ alkyl) modified nucleotide. In some embodiments, the modified nucleotide has a phosphate analog, e.g., 5′-vinylphosphonate. In some embodiments, the modified nucleotide has an abasic moiety or inverted abasic moiety, e.g., a moiety shown in Table 6.

As used herein, “nucleotide” means an organic compound having a nucleoside (a nucleobase, e.g., adenine, cytosine, guanine, thymine, or uracil, and a pentose sugar, e.g., ribose or 2′-deoxyribose) linked to a phosphate group. A “nucleotide” can serve as a monomeric unit of nucleic acid polymers such as deoxyribonucleic acid (DNA) and ribonucleic acid (RNA).

As used herein, “oligonucleotide” means a polymer of linked nucleotides, each of which can be modified or unmodified. An oligonucleotide is typically less than about 100 nucleotides in length.

The term “patient”, as used herein, refers to a human patient.

As used herein, “phosphate analog” means a chemical moiety that mimics the electrostatic and/or steric properties of a phosphate group. In some embodiments, a phosphate analog is positioned at the 5′ terminal nucleotide of an oligonucleotide in place of a 5′-phosphate, which is often susceptible to enzymatic removal. A 5′ phosphate analog can include a phosphatase-resistant linkage. Examples of phosphate analogs include 5′ methylene phosphonate (5′-MP) and 5′-(E)-vinylphosphonate (5′-VP). In some embodiments, the phosphate analog is 5′-VP.

As used herein, “polypeptide” or “peptide” means a polymer of amino acid residues comprising two (2) or more amino acids and/or amino acid derivatives which, in general, are linked via peptide bonds. The term applies to polymers comprising naturally occurring amino acids and polymers comprising one or more non-naturally occurring amino acids. Embodiments may include modifications or amino acid derivatives, including synthetic modifications, some of which may resemble post-translational modifications such as, phosphorylation, hydroxylation, sulfonation, acylation, glycosylation and disulfide formation. As used herein, “iRNA,” “iRNA agent,” “RNAi,” “RNAi agent” and “RNA interference agent” means an agent that contains RNA and mediates the targeted cleavage of a RNA transcript via RNA interference, e.g., through a RNA-induced silencing complex (RISC) pathway. In some embodiments, the RNAi agent has a sense strand and an antisense strand, and the sense strand and the antisense strand form a duplex. In some embodiments, the sense and antisense strands of RNAi agent are 21-23 nucleotides in length. In other embodiments, the sense and antisense strands can be longer, for example 25-30 nucleotides in length, in which case the longer RNAi sequences are first processed by the Dicer enzyme. The iRNA attenuates, inhibits, modulates, or reduces ACVR1C expression in a cell. The RNAi agent as disclosed herein may include portions or moieties other than nucleic acids, including but not limited to a peptide portion (such as an NPR-C binding peptide); a fatty acid; and linkers and spaces, among others.

In some embodiments, the peptides described herein include a fatty acid conjugated, for example, by way of a direct bond or linker to a natural or non-natural amino acid with a functional group available for conjugation. Such a conjugation is sometimes referred to as acylation. In certain instances, the amino acid with a functional group available for conjugation can be K, C, E, and D. In particular instances, the amino acid with a functional group available for conjugation is K, where the conjugation is to an F-amino group of a K side-chain. In some embodiments, the peptides described herein are amidated. In some embodiments, the peptides described herein have a modification of the C-terminal group, wherein the modification is NH₂. In some embodiments, the peptides described herein have a modification of the C-terminal group where the modification is absent.

Amino acids which may be incorporated into the peptides of the present disclosure include the twenty standard or canonical amino acids, and a number of other nonstandard amino acids. Some of the nonstandard amino acids are shown below in Table 14.

TABLE 14
Selected nonstandard amino acids

| NAME | ABBREVIATION(S) | STRUCTURE |
|---|---|---|
| D-Alanine | a, D-Ala | 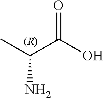 |
| D-Phenylalanine | f, D-Phe | 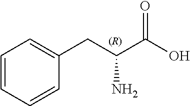 |
| D-Proline | p, D-Pro | 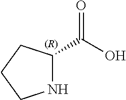 |
| D-Serine | s, D-Ser | 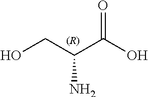 |
| D-Tyrosine | y, D-Tyr | 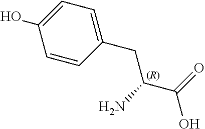 |
| 2-Aminoisobutryic Acid | Aib | 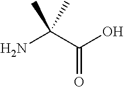 |
| β-Cyclohexyl-L- Alanine | Cha | 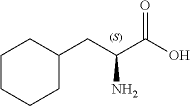 |
| L-2,3- Diaminopropionic Acid | Dap | 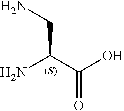 |
| L-Hydroxyproline | Hyp | 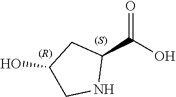 |
| D-Hydroxyproline | D-Hyp | 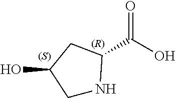 |
| L-Ornithine | Orn | 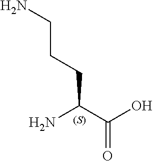 |
| Alpha-Methyl-2- Fluoro-L- Phenylalanie | αMePhe(2F) | 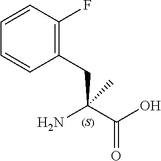 |
| 3- Mercaptopropanoic Acid | Mpa | 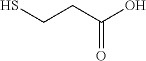 |
| N^{w}-Methyl-L- Arginine | Arg(Me) | 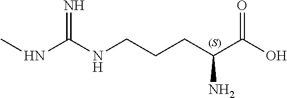 |
| 4-Pyridyl-L-Alanine | 4-Pal | 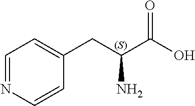 |
| 3-(2-Naphthyl)-L- Alanine | 2Nal | 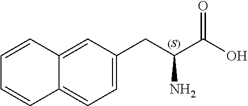 |
| L- Homophenylalanine | hPhe | 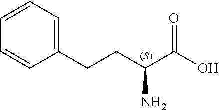 |
| D-Homoproline | D-Pip | 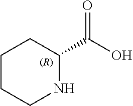 |
| L-Homoglutamic Acid | Aad | 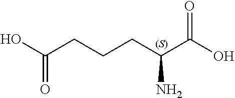 |
| N-Methyl-L- Arginine | NMe-Arg | 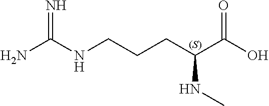 |
| Gamma-L-Glutamic Acid | γE | 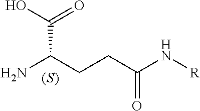 |
| 2-[2-(2-amino- ethoxy)-ethoxy]- acetic acid | AEEA | 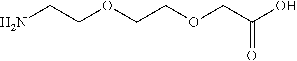 [SMILES: NCCOCCOCC(=O)O] |

As used herein, where the full name of an amino acid is spelled out (“arginine,” etc.), all stereoisomers of that amino acid is encompassed (e.g., L-arginine and D-arginine.) Where a single-letter abbreviation is used, an uppercase letter denotes the L isomer (that is, the isomer incorporated into polypeptides in nature), and a lowercase letter denotes the D isomer. Noncanonical amino acids are generally denoted by a three-letter abbreviation rather than a single letter, or as specified in the table above.

The peptides described herein may react with any number of inorganic and organic acids/bases to form pharmaceutically acceptable acid/base addition salts. Pharmaceutically acceptable salts and common techniques for preparing them are well known in the art (see, e.g, Stahl et. al, Handbook of Pharmaceutical Salts: Properties, Selection, and Use, 2^{nd} Revised Edition (Wiley-VCH, 2011)). Pharmaceutically acceptable salts for use herein include sodium, potassium, trifluoroacetate, hydrochloride and/or acetate salts. The disclosure also provides and therefore encompasses novel intermediates and methods of synthesizing the polypeptides described herein, or pharmaceutically acceptable salts thereof. The intermediates and polypeptides described herein can be prepared by a variety of techniques known in the art. For example, a method using chemical synthesis is illustrated in the Examples below.

The specific synthetic steps for each of the routes described may be combined in different ways to prepare the polypeptides described herein. The reagents and starting materials are readily available to one of skill in the art.

As used herein, “strand” refers to a single, contiguous sequence of nucleotides linked together through internucleotide linkages (e.g., phosphodiester linkages or phosphorothioate linkages). A strand can have two free ends (e.g., a 5′ end and a 3′ end).

As used herein, “treatment” or “treating” refers to all processes wherein there may be a slowing, controlling, delaying, or stopping of the progression of the disorders or disease disclosed herein, or ameliorating disorder or disease symptoms, but does not necessarily indicate a total elimination of all disorder or disease symptoms. Treatment includes administration of a protein or nucleic acid or vector or composition for treatment of a disease or condition in a patient, particularly in a human.

In one aspect, provided herein are ALK7 RNAi agents comprising a sense strand and an antisense strand, wherein the sense strand and the antisense strand form a duplex, and wherein the antisense strand is complementary to a region of ALK7 mRNA. In some embodiments, the sense strand and the antisense strand are each 15-30 nucleotides in length, e.g., 20-25 nucleotides in length. In some embodiments, provided herein are ALK7 RNAi agents comprising a sense strand of 21 nucleotides and an antisense strand of 23 nucleotides. In some embodiments, the sense strand and antisense strand of the ALK7 RNAi agent may have overhangs at either the 5′ end or the 3′ end (i.e., 5′ overhang or 3′ overhang). For example, the sense strand and the antisense strand may have 5′ or 3′ overhangs of 1 to 5 nucleotides.

In some embodiments, provided herein are ALK7 RNAi agent comprising a sense strand and an antisense strand, wherein the sense strand and the antisense strand form a duplex, wherein the sense strand comprises a nucleic acid sequence selected from any one of SEQ ID NOs: 78-153 and 396-456, and the antisense strand comprises a nucleic acid sequence selected from any one of SEQ ID NOs: 154-229 and 457-518, wherein optionally one or more nucleotides of the sense strand and the antisense strand are independently modified nucleotides, and wherein optionally one or more internucleotide linkages of the sense strand and the antisense strand are modified internucleotide linkages.

In some embodiments, the sense strand is linked to a GalNAc (N-acetylgalactosamine) delivery moiety. The delivery moiety can facilitate the entry of RNAi agent into the cells. In some embodiments, the delivery moiety is linked to the 3′ end of the sense strand, optionally via a linker. In some embodiments, the delivery moiety comprises the following formula (Ac means acetyl):

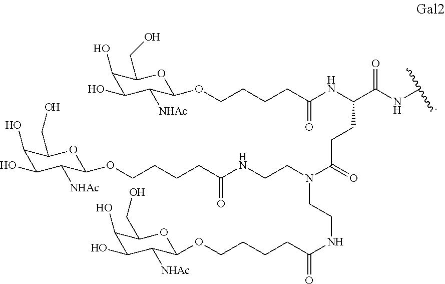 [SMILES: *NC(=O)[C@H](CCC(=O)N(CCNC(=O)CCCCOC1OC(CO)C(O)C(O)C1NC(C)=O)CCNC(=O)CCCCOC1OC(CO)C(O)C(O)C1NC(C)=O)NC(=O)CCCCOC1OC(CO)C(O)C(O)C1NC(C)=O]

In another aspect, provided herein are ALK7 RNAi agents comprising Formula R-L_{G}-D, wherein R is a double stranded RNA (dsRNA) comprising a sense stand and an antisense strand, wherein the antisense strand is complementary to ALK7/ACVR1C mRNA, wherein L_{G} is a linker, or optionally absent, and wherein D is a delivery moiety comprising Formula:

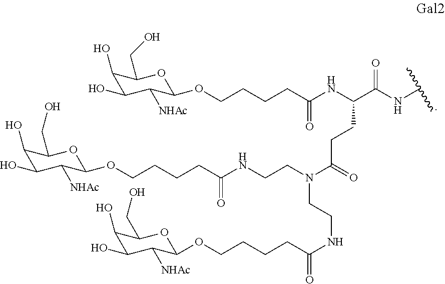 [SMILES: *NC(=O)[C@H](CCC(=O)N(CCNC(=O)CCCCOC1OC(CO)C(O)C(O)C1NC(C)=O)CCNC(=O)CCCCOC1OC(CO)C(O)C(O)C1NC(C)=O)NC(=O)CCCCOC1OC(CO)C(O)C(O)C1NC(C)=O]

In some embodiments, the GalNAc delivery moiety is conjugated to the 3′ end of the sense stand via a linker (LG). Suitable linkers are known in the art. In some embodiments, linker (L_{G}) comprises Formula (III) or Formula (IV):

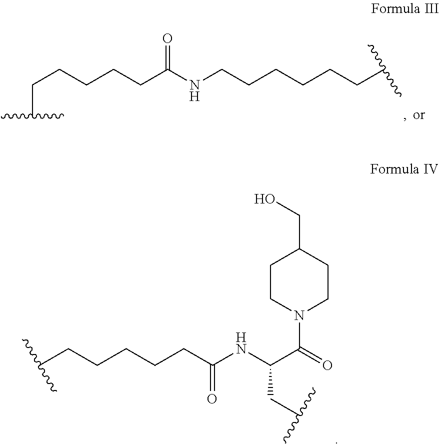 [SMILES: *CCCCCCNC(=O)CCCCC* ; *CCCCCC(=O)N[C@@H](C*)C(=O)N1CCC(CO)CC1]

In some embodiments, the sense strand comprises a first nucleic acid sequence having at least 90% sequence identity to SEQ ID NO: 78-153 or 396-456, and the antisense strand comprises a second nucleic acid sequence having at least 90% sequence identity to SEQ ID NO: 154-229 or 457-518, wherein optionally one or more nucleotides of the sense strand and the antisense strand are independently modified nucleotides, and wherein optionally one or more internucleotide linkages of the sense strand and the antisense strand are modified internucleotide linkages.

In some embodiments, the sense strand comprises a first nucleic acid sequence having at least 95% sequence identity to SEQ ID NO: 78-153 or 396-456, and the antisense strand comprises a second nucleic acid sequence having at least 95% sequence identity to SEQ ID NO: 154-229 or 457-518, wherein optionally one or more nucleotides of the sense strand and the antisense strand are independently modified nucleotides, and wherein optionally one or more internucleotide linkages of the sense strand and the antisense strand are modified internucleotide linkages.

In some embodiments, the sense strand comprises a first nucleic acid sequence having at least 18 contiguous nucleotides of one of SEQ ID NO: 78-153 or 396-456, and the antisense strand comprises a second nucleic acid sequence having at least 18 contiguous nucleotides of one of SEQ ID NO: 154-229 or 457-518, wherein optionally one or more nucleotides of the sense strand and the antisense strand are independently modified nucleotides, and wherein optionally one or more internucleotide linkages of the sense strand and the antisense strand are modified internucleotide linkages.

In some embodiments, the first nucleic acid sequence (of the sense strand) has at least 19 contiguous nucleotides of one of SEQ ID NO: 78-153 or 396-456, or at least 20 contiguous nucleotides of one of SEQ ID NO: 78-153 or 396-456.

In some embodiments, the second nucleic acid sequence (of the antisense strand) has at least 19 contiguous nucleotides of one of SEQ ID NO: 154-229 or 457-518, or at least 20 contiguous nucleotides of one of SEQ ID NO: 154-229 or 457-518, or at least 21 contiguous nucleotides of one of SEQ ID NO: 154-229 or 457-518, or at least 22 contiguous nucleotides of one of SEQ ID NO: 154-229 or 457-518.

In some embodiments, the sense strand and the antisense strand comprise a pair of nucleic acid sequences selected from the group consisting of: the sense strand comprises a first nucleic acid sequence of SEQ ID NO: 115, and the antisense strand comprises a second nucleic acid sequence of SEQ ID NO: 191; or the sense strand comprises a first nucleic acid sequence of SEQ ID NO: 117, and the antisense strand comprises a second nucleic acid sequence of SEQ ID NO: 193; or the sense strand comprises a first nucleic acid sequence of SEQ ID NO: 133, and the antisense strand comprises a second nucleic acid sequence of SEQ ID NO: 209; or the sense strand comprises a first nucleic acid sequence of SEQ ID NO: 134, and the antisense strand comprises a second nucleic acid sequence of SEQ ID NO: 210; or the sense strand comprises a first nucleic acid sequence of SEQ ID NO: 135, and the antisense strand comprises a second nucleic acid sequence of SEQ ID NO: 211, wherein optionally one or more nucleotides of the sense strand and the antisense strand are independently modified nucleotides, and wherein optionally one or more internucleotide linkages of the sense strand and the antisense strand are modified internucleotide linkages.

In some embodiments, the sense strand and the antisense strand comprise a pair of nucleic acid sequences wherein the sense strand comprises a first nucleic acid sequence including 18 consecutive nucleotides of SEQ ID NO: 78-153 or SEQ ID NO: 396-456, and the antisense strand comprises a second nucleic acid sequence including 18 consecutive nucleotides of SEQ ID NO: 154-229 or SEQ ID NO: 457-518; wherein optionally one or more nucleotides of the sense strand and the antisense strand are independently modified nucleotides, and wherein optionally one or more internucleotide linkages of the sense strand and the antisense strand are modified internucleotide linkages.

As used herein, “NPR-C” refers to natriuretic receptor C protein or polypeptide. NPR-C is also known as NPR3 (natriuretic peptide receptor 3). NPR-C and NPR3 are used interchangeably throughout this disclosure. Several human NPR-C isoforms exist.

The amino acid sequence of the longest human NPR-C isoform (isoform 1) can be found at NP_001191304.1:

| (SEQ ID NO: 74) | |
|---|---|
| 1 | MPSLLVLTFS PCVLLGWALL AGGTGGGGVG GGGGGAGIGG GRQEREALPP QKIEVLVLLP |
| 61 | QDDSYLFSLT RVRPAIEYAL RSVEGNGTGR RLLPPGTRFQ VAYEDSDCGN RALFSLVDRV |
| 121 | AAARGAKPDL ILGPVCEYAA APVARLASHW DLPMLSAGAL AAGFQHKDSE YSHLTRVAPA |
| 181 | YAKMGEMMLA LFRHHHWSRA ALVYSDDKLE RNCYFTLEGV HEVFQEEGLH TSIYSFDETK |
| 241 | DLDLEDIVRN IQASERVVIM CASSDTIRSI MLVAHRHGMT SGDYAFFNIE LFNSSSYGDG |
| 301 | SWKRGDKHDF EAKQAYSSLQ TVTLLRTVKP EFEKFSMEVK SSVEKQGLNM EDYVNMFVEG |
| 361 | FHDAILLYVL ALHEVLRAGY SKKDGGKIIQ QTWNRIFEGI AGQVSIDANG DRYGDFSVIA |
| 421 | MTDVEAGTQE VIGDYFGKEG RFEMRPNVKY PWGPLKLRID ENRIVEHTNS SPCKSSGGLE |
| 481 | ESAVTGIVVG ALLGAGLLMA FYFFRKKYRI TIERRTQQEE SNLGKHRELR EDSIRSHFSV |
| 541 | A. |

The amino acid sequence of NPR-C isoform 2 can be found at NP_000899.1:

| (SEQ ID NO: 75) | |
|---|---|
| 1 | MPSLLVLTFS PCVLLGWALL AGGTGGGGVG GGGGGAGIGG GRQEREALPP QKIEVLVLLP |
| 61 | QDDSYLFSLT RVRPAIEYAL RSVEGNGTGR RLLPPGTRFQ VAYEDSDCGN RALFSLVDRV |
| 121 | AAARGAKPDL ILGPVCEYAA APVARLASHW DLPMLSAGAL AAGFQHKDSE YSHLTRVAPA |
| 181 | YAKMGEMMLA LFRHHHWSRA ALVYSDDKLE RNCYFTLEGV HEVFQEEGLH TSIYSEDETK |
| 241 | DLDLEDIVRN IQASERVVIM CASSDTIRSI MLVAHRHGMT SGDYAFFNIE LFNSSSYGDG |
| 301 | SWKRGDKHDF EAKQAYSSLQ TVTLLRTVKP EFEKFSMEVK SSVEKQGLNM EDYVNMFVEG |
| 361 | FHDAILLYVL ALHEVLRAGY SKKDGGKIIQ QTWNRTFEGI AGQVSIDANG DRYGDFSVIA |
| 421 | MTDVEAGTQE VIGDYFGKEG RFEMRPNVKY PWGPLKLRID ENRIVEHTNS SPCKSCGLEE |
| 481 | SAVTGIVVGA LLGAGLLMAF YFFRKKYRIT IERRTQQEES NLGKHRELRE DSIRSHFSVA |

Other NPR-C isoforms include isoform 3 (NP_001191305.1), isoform 4 (NP_001350581.1), isoform 5 (NP_001351387.1), isoform 6 (NP_001351389.1).

As used herein, “ALK7” refers to the activin A receptor, and is also known as ACVR1C, for activin A receptor type 1C. “ALK7” is used equivalently and interchangeably with “ACVR1C” herein throughout. The nucleic acid sequence of human ALK7 mRNA can be found at NM_145259.3 (SEQ ID NO: 76). The amino acid sequence of human ALK7 protein can be found at NP_660302.2 (SEQ ID NO: 77). The nucleic acid sequence of mouse ALK7 mRNA can be found at NM_001033369.3 (SEQ ID NO: 393). The amino acid sequence of mouse ALK7 protein can be found at NP_001028541.1 (SEQ ID NO: 394).

As used herein, “ALK7 associated metabolic disorder” means a metabolic disorder associated with abnormal ALK7 expression, activity, or function.

The following examples are offered to illustrate, but not to limit, the claimed inventions.

### EXAMPLES

### Example 1: General Strategy for Synthesis of dsRNA-Peptide Conjugates

As depicted in Route 1, NPR-C binding peptides (Route 1A), RNA sense strands (Route 1B) and RNA antisense strands (Route 1C) were all synthesized using standard solid phase peptide or oligonucleotide synthesis techniques. Further functionalization steps to incorporate optional spacers (SL, SP, SF), fatty acids (FA), and linker fragments activated for subsequent conjugation (L_{F}), were performed either directly on solid support, or in solution following cleavage from the solid support, depending on the chemistry involved.

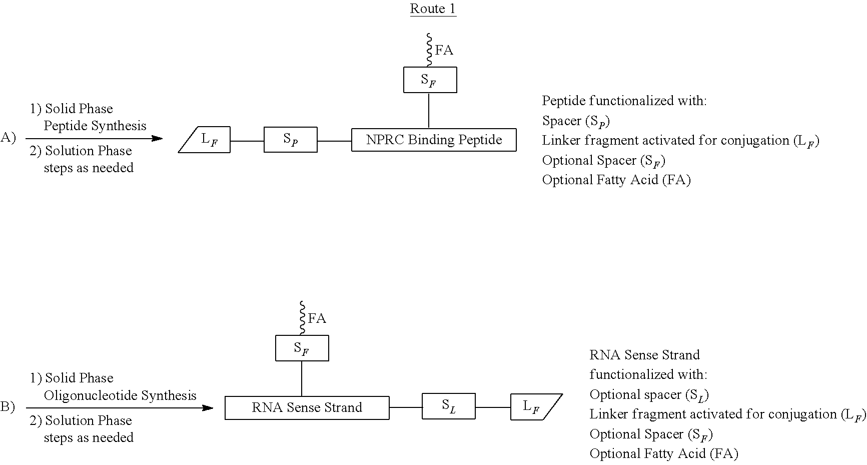

As depicted in Route 2, functionalized RNA sense strand intermediates were conjugated in solution to functionalized NPR-C binding peptides using appropriate conditions. The resulting RNA sense strand-peptide conjugate intermediates were then annealed to the corresponding RNA antisense strand, to provide the dsRNA-peptide conjugate, with appropriate purification steps.

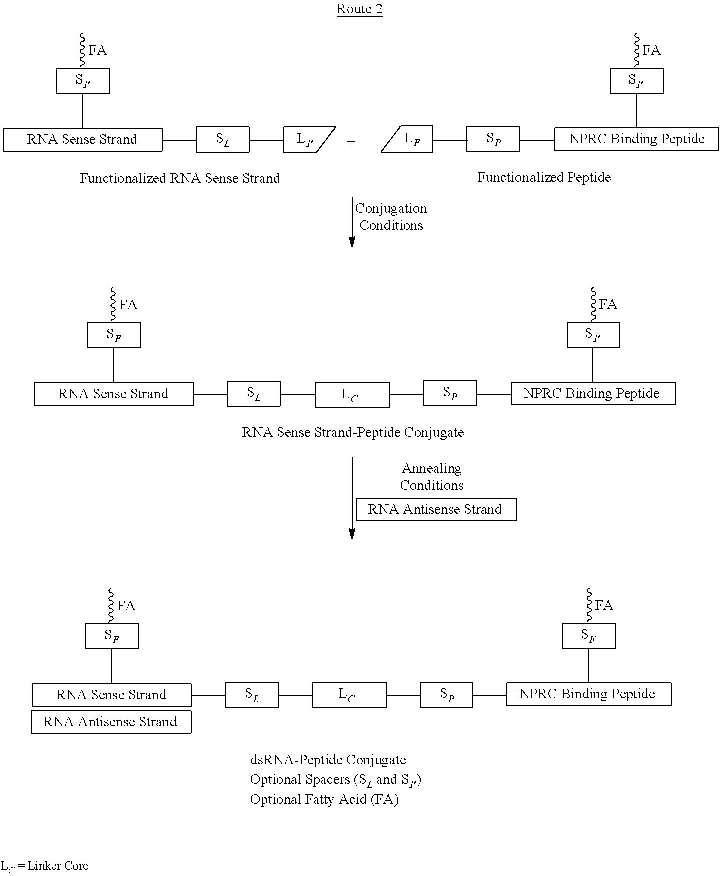

As depicted in Route 3, an alternative synthetic approach entails annealing the functionalized RNA sense strand to the corresponding RNA antisense strand, prior to conjugation. Using appropriate conditions and purification steps, the functionalized NPR-C binding peptide can then be conjugated to the functionalized dsRNA in solution, to provide the dsRNA-peptide conjugate.

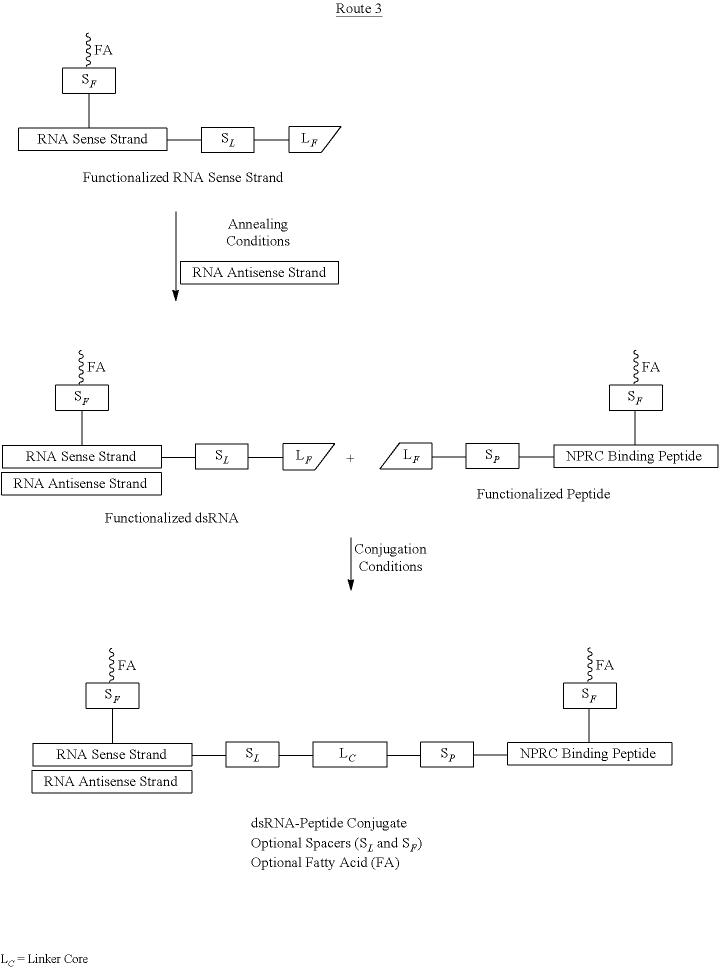

### Example 2: Preparation of Functionalized Peptide Intermediates for Use in Synthesis of dsRNA-NPR-C-Binding Peptide Conjugates

### Examples 2A-2E

### Example 2A: General Procedure for Solid Phase Synthesis of NPR-C Binding Peptides

Peptide synthesis was carried out using standard 9-fluorenyl-methyloxycarbonyl (Fmoc) tert-Butyl (t-Bu) solid phase peptide chemistry protocols on either a Symphony 12-channel multiplex peptide synthesizer or a Symphony-X 24-channel multiplex peptide synthesizer (Gyros Protein Technologies, Inc.), at a 0.1 mmol scale.

The solid support used was pre-loaded H-Cys(Trt)-2-CTC resin (S-trityl-L-cysteine-2-Chlorotrityl Resin, Peptides International), (100-200 mesh) with a 1% DVB cross-linked polystyrene core and a substitution of ~0.50 meq/g for the generation of peptide acids, or Low Loading 4-(2′,4′-Dimethoxyphenyl-Fmoc-aminomethyl)-phenoxyacetamido-norleucyl-4-Methylbenzhydrylamine resin (Fmoc-Rink-MBHA Low Loading resin, EMD Millipore), (100-200 mesh) with a 1% DVB cross-linked polystyrene core and a substitution range of 0.3-0.4 meq/g for peptide amides. Standard sidechain protecting groups were used for all Fmoc-L-Amino Acids utilized. Non-standard amino acids used in the syntheses herein can be found in Table 15 or 16.

Fmoc deprotection prior to each coupling step was accomplished by treatments with 20% piperidine (PIP; Sigma Aldrich) in DMF (Fisher Chemicals), 2×7 minutes with nitrogen mixing, followed by 8×DMF wash cycles. For the synthesis of peptide acids on 2-chlorotrityl resin, typical unhindered couplings were performed for 1 hour using the Fmoc amino acid (0.3 M in DMF), N,N,N′,N′-Tetramethyl-O-(1H-benzotriazol-1-yl)uronium hexafluorophosphate (HBTU, Ambeed Inc.; 0.9 M in DMF) and N,N-diisopropylethylamine (DIPEA, Sigma Aldrich; 1.2 M in DMF), at a 9-fold molar excess of AA/HBTU and a 12-fold molar excess of DIPEA over the reported resin loading level.

For the coupling of hindered building blocks such as Fmoc-Arg(Pbf)-OH, Fmoc-N-Methyl-AAs or Fmoc-αMeAAs, a double coupling protocol was used, and the coupling time was extended to 2 hours each. For the synthesis of peptide amides on Fmoc-Rink-MBHA resin, typical unhindered couplings were performed for 1 hour using the Fmoc amino acid (0.3 M, Advanced ChemTech, in DMF), N,N′-diisopropylcarbodiimide (DIC, ChemImpex, 1.2 M in DCM) and ethyl cyanohydroxyiminoacetate (Oxyma Pure, ChemImpex; 0.9 M in DMF), at a 9-fold molar excess of AA/Oxyma and a 12-fold molar excess of DIC over the reported resin loading level. For hindered couplings such as Fmoc-Arg(Pbf)-OH, Fmoc-N-Methyl-AAs or Fmoc-αMeAA, the coupling time was extended to 6 hours.

For the solid phase coupling of active esters, such as MSPT-PEG2-NHS, a manual addition of a 3x excess of the ester along with a 10× excess of DIPEA was used after the final Fmoc was removed from the peptidyl resin. After the primary sequence of the desired peptide was completed, the peptidyl resin was transferred as a DCM slurry to disposable fritted plastic syringe fitted with Teflon stopcock. To prepare the resin for cleavage, further washes with DCM were done, and the resin was thoroughly dried in vacuo.

TFA cleavage: The dry peptidyl resins were treated with 10 mL of cleavage cocktail consisting of trifluoroacetic acid (TFA, Acros Organics), Water, Thioanisole (Sigma-Aldrich) and Triisopropylsilane (TIPS; Acros Organics), (TFA:Water:Thioanisole:TIPS; 90.5:2.5:2.5 v/v) or TFA, water, 3,6-dioxa-1,8-octanedithiol (DODT; Sigma Aldrich), triisopropylsilane, (TFA:Water:DODT:TIPS; 90:5:2.5:2.5 v/v) for 2 hours at room temperature. After the 2 hour cleavage incubation, the resin was filtered off, washed twice with 2 mL of neat TFA, and the combined filtrates/washes were collected in a 50 ml conical disposable tube. The cleavage solution was then treated with 35 mL of cold diethyl ether (Fisher Chemicals) (−20° C.) to precipitate the crude peptide. The peptide/ether suspension was then centrifuged at 4000 rpm for 2 min to form a solid pellet, the supernatant was decanted, and the solid pellet was triturated with fresh ether and the process was repeated two additional times, finally drying the peptide pellet in vacuo.

TABLE 15
Nonstandard amino acids and other peptide components

| | | AA/Residue |
|---|---|---|
| Building Block | AA/Residue | Abbreviation |
| 2-Azidoacetic Acid | 2-Azidoacetic Acid | |
| Fmoc-L-Hyp(tBu)-OH | L-Hydroxyproline | Hyp |
| Fmoc-D-Hyp(tBu)-OH | D-Hydroxyproline | D-Hyp |
| Fmoc-2-Aminoisobutyric Acid | 2-Aminoisobutyric Acid | Aib |
| Fmoc-L-Orn(Boc)-OH | L-Ornithine | Orn |
| Fmoc-L-Dap(Boc)-OH | L-2,3-Diaminopropionic Acid | Dap |
| Fmoc-αMe-L-Phe(2F)-OH | alpha-methyl-2-fluoro-L-phenylalanine | αMePhe(2F) |
| Trtityl-3-Mercaptopropionic Acid | 3-Mercaptopropionic Acid | Mpa |
| Fmoc-L-Cha-OH | β-cyclohexyl-L-alanine | Cha |
| Fmoc-AEEA-OH | 2-[2-(2-amino-ethoxy)-ethoxy]-acetyl | AEEA |
| Fmoc-L-Arg(Me, Pbf)-OH | N^{ω}-methyl-L-arginine | Arg(Me) |
| Fmoc-L-4-Pal-OH | 4-Pyridyl-L-Alanine | 4-Pal |
| Fmoc-L-2-Nal-OH | 3-(2-naphthyl)-L-alanine | 2Nal |
| Fmoc-L-hPhe-OH | L-Homophenylalanine | hPhe |
| Fmoc-D-Pip-OH | D-Pipecolic Acid (D-Homoproline) | D-Pip |
| Fmoc-L-Glu-OtBu | gamma-L-Glutamic acid | γE |
| Fmoc-Aad(OtBu)-OH | L-Homoglutamic Acid (Aminoadipic Acid) | Aad |
| Fmoc-N-Methyl-L-Arg(Pbf)-OH | N-Methyl-L-arginine | NMe-Arg |
| Mal-Dap(Boc)-OH | Maleimido-L-2,3-Diaminopropionic Acid | Mal-Dap |
| MSPT-PEG2-NHS Ester | 2-(2-(2-(4-(5-(methylsulfonyl)-1H-tetrazol-1- | PT-PEG2 |
| | yl)phenoxy)ethoxy)ethoxy)acetic acid | |
| MSPOD-PEG2-NHS Ester | 2-(2-(2-(4-(5-(methylsulfonyl)-1,3,4-oxadiazol-2- | POD-PEG2 |
| | yl)phenoxy)ethoxy)ethoxy)acetic acid | |
| Fmoc-Cpa-OH | Fmoc-3-(2-cyano-4-pyridyl)alanine | Cpa |
| HO-C20-OtBu | Eicosanedioic acid | HO-C20- |
| C15-COOH | Palmitic Acid | C16- |

### Example 2B: General Procedure for Disulfide Bridge Formation in NPR-C Binding Peptides

Crude peptides were solubilized and diluted, in a suitable glass vessel, with 25% aqueous acetic acid to relatively low concentration (0.2-0.5 mg/ml crude peptide). The solution was then placed on magnetic stirrer with a spin vane, mixed vigorously and titrated with a few drops a saturated Iodine in methanol solution until a faint yellow endpoint was achieved. After reaching the yellow endpoint, the reaction was incubated at RT for 15 min, at which point the excess Iodine was quenched by the addition of a few drops of 0.1 M aqueous ascorbic acid (Sigma Aldrich).

### Example 2C: General Procedure for Thioacetal Bridge Formation in NPR-C Binding Peptides

Peptides were solubilized in 3 mL of water; the solution was then diluted with water and AcCN to achieve about a 30/70 mixture of Water/AcCN (~400 mL total volume) with a low concentration of peptide (~0.2-0.5 mg/ml). The solution was then adjusted to pH 8 with triethylamine (TEA, Acros Organics) ~10 equivalents, reducing conditions to prevent disulfide bridge formation were achieved by the addition of 2-4 equivalents of Tris(2-carboxyethyl) phosphine hydrochloride (TCEP-HCl, Sigma Aldrich) reducing agent. The thioacetal bridge was formed by the addition of 7-10 equivalents of Diiodomethane (CH₂12, Alfa Aesar). The thioacetal formation reaction was carried out by incubating the solution for 18 hours at RT with magnetic stirring. Progress of the reaction was monitored using analytical LC-MS and by observing the change in mass of +12 Daltons from the starting reduced peptide molecular weight and an accompanying shift in retention of the starting material HPLC peak.

### Example 2D: General Procedure for Solution-Phase Acylation of NPR-C Peptide N-Terminus with Linker Precursors: Mal-Dap(Boc)-OPfp, MSPT-PEG2-NHS, Mpa-NHS, and Fmoc-Cys(Trt)-Opfp

For cyclic peptides containing a Thioacetal bridge, a solution phase acylation step was necessary to introduce the requisite reactive handle for conjugation. In a 15 mL falcon tube equipped with a spin vane, the purified, lyophilized precursor peptide was dissolved in 1000 μL of DMSO (Acros Organics) to it were added 75-100 μL of DIPEA (TCI America) to adjust the pH to ~8-9. The reaction was magnetically stirred at RT. The active ester of the desired residue/moiety was dissolved in DMSO (-200-300 uL), 50 μL aliquots were added one at time while monitoring the progression of the reaction by LC-MS. Once the starting material was consumed, the reaction was stopped by the addition of −10 mL cold diethyl ether (Fisher Chemicals) (−20° C.). The tube was mixed vigorously and centrifuged to force the peptide into a pellet or oil phase. Another fresh volume of cold diethyl ether was added, and the process was repeated, this time a white, tacky precipitate was formed as the final crude product.

### Additional Steps Following Solution Phase Coupling of Fmoc-Cys(Trt)-OPfp:

After the acylation and ether precipitation step, the pellet was further treated with 1 ml of 20% PIP in DMF (for −15 min) to remove the Fmoc protecting group. Another round of precipitation was carried out using cold diethyl ether, washed and additional time with the cold ether. The pellet was finally treated with TFA (with 2% TIPS) to remove the Trityl protecting group from the Cys residue. Cold diethyl ether precipitation was once again used to isolate the final crude product.

### Additional Steps Following Solution Phase Coupling of Mal-Dap(Boc)-OPfp:

After the acylation and ether precipitation step, the pellet was further treated with neat TFA to remove the Boc protecting group from the Dap residue. Cold diethyl ether precipitation was once again used to isolate the final crude product.

### Example 2E: General Procedure for Reverse-Phase HPLC Purification of Peptides

Preparative HPLC was carried out using either a Waters 2545 Binary HPLC Systems or a Shimadzu LC-8A Binary HPLC Systems, both equipped with a column heater; using a Luna Phenyl-Hexyl RP-HPLC column (Phenomenex Inc.; 5 μm, 100 Å; 250×21.2 mm). The running buffers used were either Formic acid; A: 0.1% Formic Acid/H2O and B: 0.1% Formic/Acetonitrile (AcCN, Fisher Chemicals) or TFA; A: 0.1% TFA/H2O and B: 0.1% TFA/Acetonitrile (AcCN, Fisher Chemicals). The initial loadings of the peptides were typically done either at 0%, 5% B, or 10% depending on the hydrophobicity of the peptide, with a 10 min isocratic step after loading at the respective starting conditions for column equilibration. Linear gradients of 0-60% B, or 5-65% B or 10-70% B over 60 min, at a flow of 15, 20 or 25 mL/min, with column heating set at 60° C. were used. UV monitoring was done at either 214 or 220 nm. Fractions that were determined to contain the desired product, as confirmed by LC-MS analysis (Agilent LC/MSD XT), were pooled together. Typically, the solutions were then frozen and lyophilized to give a white amorphous solid product, as the TFA salts of the peptides (TFA was added to the pooled fraction isolated with Formic acid buffers). The purity was assessed by RP-HPLC (Agilent Infinity II), and MW was confirmed by LC-MS analysis (Agilent LC/MSD XT).

### Example 3: Representative Examples of NPRC-Binding Peptide Synthesis and Functionalization

### Examples 3 Å-3G

### Example 3 Å: Synthesis of Azidoacetyl-(SP1)-(SEQ ID: 11)

Below is a depiction of the structure of the title compound using the standard single letter code for L-Amino Acids except for the 2-(2-(2-Aminoethoxy)-ethoxy) acetic acid spacer (AEEA), L-Cysteine (Cys) residues at positions 7 and 23, and Azidoacetic Acid at the N-Terminus, where the structures of the residues have been expanded.

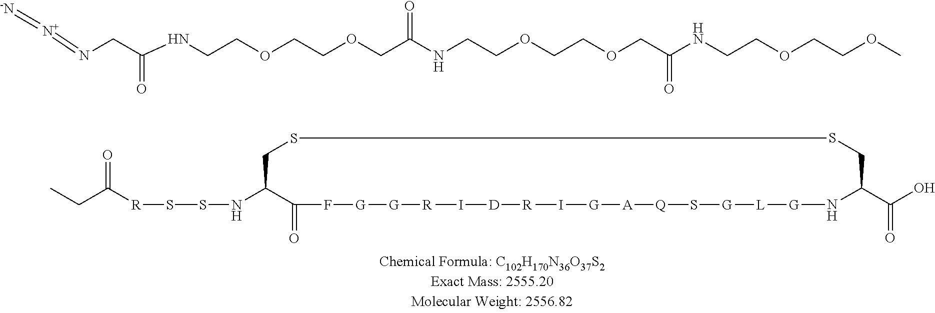

The primary peptide sequence of the title compound was synthesized using standard 9-Fluorenyl-methyloxycarbonyl (Fmoc) tert-Butyl (t-Bu) solid phase peptide chemistry protocols on a Symphony, 12-channel multiplex peptide synthesizer (Gyros Protein Technologies, Inc.), at a 0.1 mmol scale. The solid support used consisted of pre-loaded H-Cys(Trt)-2-CTC resin (S-trityl-L-cysteine-2-Chlorotrityl Resin, Peptides International), (100-200 mesh) with a 1% DVB cross-linked polystyrene core and a substitution of ~0.50 meq/g. Standard sidechain protecting groups were used for all Fmoc-L-Amino Acids utilized. The non-standard amino acid used in the synthesis of the title compound was 2-[2-[2-(Fmoc-amino) ethoxy]ethoxy]acetic acid (Fmoc-AEEA-OH, AappTec Peptides). Azidoacetic Acid was used to cap the N-terminus of the sequence; the residue provides a reactive handle for conjugation chemistries. Fmoc deprotection prior to each coupling step was accomplished by treatments with 20% Piperidine (PIP; Sigma Aldrich) in Dimethylformamide (DMF; Fisher Chemicals), 2×7 minutes with nitrogen mixing, followed by 8 x DMF wash cycles. All amino acid couplings were performed for 1 hour using the Fmoc Amino Acid (0.3 M in DMF), N, N, N′,N′-Tetramethyl-O-(1H-benzotriazol-1-yl)uronium hexafluorophosphate (HBTU, Ambeed Inc.; 0.9 M in DMF) and N,N-Diisopropylethylamine (DIPEA, Sigma Aldrich; 1.2 M in DMF), at a 9-fold molar excess of AA/HBTU and a 12-fold molar excess of DIPEA over the reported resin loading level. After the primary sequence of the peptide was synthesized up the third Fmoc-AEEA-OH residue, the final N-Terminal Fmoc was removed with PIP, and the DMF washes were carried out, the peptidyl-resin was capped with a 9-fold excess of Azidoacetic Acid. Coupling was done using DIC/Oxyma (1.2M/0.9M); the coupling time was extended to 5 hrs. After coupling, 3x DMF washes were done, then the peptidyl resin was transferred as a DCM slurry to disposable fritted plastic syringe fitted with Teflon stopcock. Further washes with DCM were done, and finally, the resin was thoroughly dried in vacuo. The dry resin was then treated with 10 mL of cleavage cocktail consisting of trifluoroacetic acid (TFA, Acros Organics), water, and Triisopropylsilane (TIPS; Acros Organics), (TFA:Water:TIPS; 92.5:5:2.5 v/v) for 2 hours at room temperature. After the 2 hr cleavage incubation, the resin was filtered off, washed twice with 2 mL of neat TFA, and the combined filtrates/washes were collected in a 50 ml conical disposable tube. The cleavage solution was then treated with 35 mL of cold diethyl ether (Fisher Chemicals) (−20° C.) to precipitate the crude peptide. The peptide/ether suspension was then centrifuged at 4000 rpm for 2 min to form a solid pellet, the supernatant was decanted, and the solid pellet was triturated with fresh ether and the process was repeated two additional times, finally drying the peptide pellet in vacuo.

### Disulfide Bridge Formation

The crude peptide was solubilized, in a suitable glass vessel, with 25% aqueous Acetic Acid to a relatively low concentration (0.2-0.5 mg/ml crude peptide). The solution was then placed on magnetic stirrer with the necessary spin vane, mixed vigorously and titrated with a few drops a saturated Iodine in methanol solution until a faint yellow endpoint was achieved. After reaching the yellow endpoint, the reaction was incubated at RT for 15 min, at which point the excess Iodine was quenched by the addition of a few drops of 0.1 M aqueous ascorbic acid (Sigma Aldrich).

### HPLC Purification

The reaction solution containing the crude oxidized peptide was then loaded, via injection valve, onto a preparative HPLC system (Shimadzu LC-8A Binary Preparative HPLC Systems) using a Luna Phenyl-Hexyl RP-HPLC column (Phenomenex Inc.; 5 μm, 100 Å; 250 x 21.2 mm). The running buffers used were A: 0.1% TFA/H₂O and B: 0.1% TFA/Acetonitrile (ACCN, Fisher Chemicals). The initial loading was done at 0% B, with 10 min isocratic equilibration after loading. The sample was eluted using a linear 0-60% B gradient over 60 min, at a flow of 25 mL/min, with column heating set at 60° C. Fractions containing the desired product (analysis by Agilent LC-MS) were pooled, frozen and lyophilized to give a white amorphous solid product, as the TFA salt of the title compound. The purity assessed by RP-HPLC 1 (Agilent HPLC System) was found to be >95%, with the observed LC-MS molecular weight of 2556.20 Dalton; matching the theoretical calculated molecular weight of 2556.82 Dalton.

### Example 3B: Synthesis of Cys-(SP1)-(SEQ ID: 20)

Below is a depiction of the structure of the title compound using the standard single letter code for L-Amino Acids except for the 2-(2-(2-Aminoethoxy)-ethoxy) acetic acid spacer (AEEA) and L-Cysteine (Cys) residues at the N-Terminus and at positions 7 and 23 where the structures of the residues have been expanded.

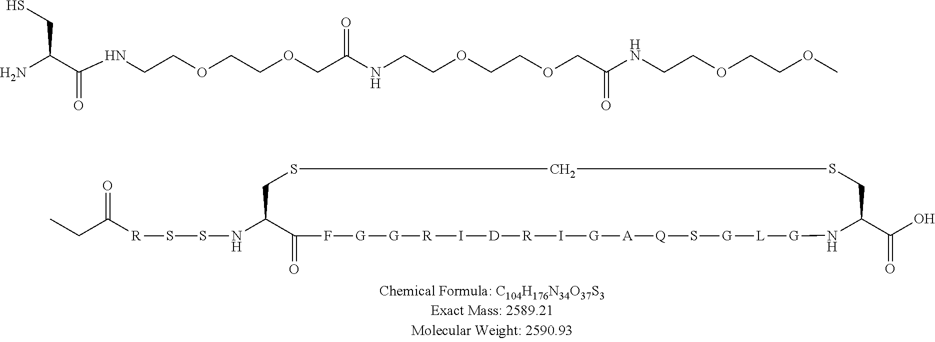

The primary peptide sequence of the title compound was synthesized using standard 9-Fluorenyl-methyloxycarbonyl (Fmoc) tert-Butyl (t-Bu) solid phase peptide chemistry protocols on a Symphony-X, 24-channel multiplex peptide synthesizer (Gyros Protein Technologies, Inc.), at a 0.1 mmol scale. The N-terminal Cysteine residue is omitted from the solid phase peptide synthesis protocol and was added in solution after the isolation of the thioacetal bridged pre-cursor peptide: H-AEEA-AEEA-AEEA-RSS[CFGGRIDRIGAQSGLGC]-OH (Thioacetal Bridge Cys7-Cys23). The solid support used consisted of pre-loaded Fmoc-Cys(Trt)-2-CTC resin (Fmoc-S-trityl-L-cysteine-2-Chlorotrityl Resin, ChemImpex), (200-400 mesh) with a 1% DVB cross-linked polystyrene core and a substitution range of 0.3-0.8 meq/g. Standard sidechain protecting groups were used for all Fmoc-L-Amino Acids used. The non-standard amino acid used in the synthesis of the title compound was 2-[2-[2-(Fmoc-amino) ethoxy]ethoxy]acetic acid (Fmoc-AEEA-OH, AappTec Peptides). Fmoc deprotection prior to each coupling step was accomplished by treatments with 20% Piperidine (PIP; Sigma Aldrich) in Dimethylformamide (DMF; Fisher Chemicals), 2×7 minutes with nitrogen mixing, followed by 8 x DMF washing cycles. All amino acid couplings were performed for 1 hour using the Fmoc Amino Acid (0.3 M in DMF), N, N, N′,N′-Tetramethyl-O-(1H-benzotriazol-1-yl)uronium hexafluorophosphate (HBTU, Ambeed Inc.; 0.9 M in DMF) and N,N-Diisopropylethylamine (DIPEA, Sigma Aldrich; 1.2 M in DMF), at a 9-fold molar excess of AA/HBTU and a 12-fold molar excess of DIPEA over the reported resin loading level. After the primary sequence of the peptide was synthesized up the third Fmoc-AEEA-OH residue, the final N-Terminal Fmoc was removed, the required DMF washes were completed, the peptidyl resin was transferred as a DCM slurry to disposable fritted plastic syringe fitted with Teflon stopcock. Further washes with DCM were done, and finally, the resin was thoroughly dried in vacuo. The dry resin was then treated with 10 mL of cleavage cocktail consisting of trifluoroacetic acid (TFA, Acros Organics), water, 3,6-dioxa-1,8-octanedithiol (DODT; Sigma Aldrich), triisopropylsilane (TIPS; Acros Organics), (TFA:Water:DODT:TIPS; 90:5:2.5:2.5 v/v) for 2 hours at room temperature. After the 2 hr cleavage incubation, the resin was filtered off, washed twice with 2 mL of neat TFA, and the combined filtrates/washes were collected in a 50 ml conical disposable tube, the solution was then treated with 35 mL of cold diethyl ether (Fisher Chemicals) (−20° C.) to precipitate the crude peptide. The peptide/ether suspension was then centrifuged at 4000 rpm for 2 min to form a solid pellet, the supernatant was decanted, and the solid pellet was triturated with fresh ether and the process was repeated two additional times, finally drying the peptide pellet in vacuo.

### Disulfide Bridge Formation

The crude peptide was solubilized, in a suitable glass vessel, with 25% aqueous Acetic Acid to relatively low concentration (0.2-0.5 mg/ml crude peptide). The solution was then placed on magnetic stirrer with the requisite spin vane, mixed vigorously and titrated with a few drops a saturated Iodine in methanol solution until a faint yellow endpoint was achieved. After reaching the yellow endpoint, the reaction was incubated at RT for 15 min, at which point the excess iodine was quenched by the addition of a few drops of 0.1 M aqueous ascorbic acid (Sigma Aldrich).

### HPLC Purification

The oxidation solution containing the crude peptide was loaded directly onto a preparative HPLC system (Waters 2545 Binary Systems) equipped with a column heater and using a Luna Phenyl-Hexyl RP-HPLC column (Phenomenex Inc.; 5 μm, 100 Å; 250×21.2 mm). The running buffers used were A: 0.1% TFA/H₂O and B: 0.1% TFA/Acetonitrile (AcCN, Fisher Chemicals). The initial loading was done at 5% B, with 10 min isocratic wash after loading for column equilibration. The sample was eluted using a linear 5-65% B gradient over 60 min, at a flow of 20 mL/min, with column heating set at 60° C. Fractions that were determined to contain the desired product (analysis by LC-MS) were pooled, frozen and lyophilized to give a white amorphous solid product, as the TFA salt of the title compound. The purity assessed by RP-HPLC was found to be >95%, with the observed LC-MS molecular weight of 2473.73 Dalton, matching the theoretical calculated molecular weight of 2473.77 Dalton.

### Thioacetal Bridge Formation

After the purification of the disulfide bridged peptide, the lyophilized disulfide material was solubilized in 3 mL of water, the solution was then diluted with water and AcCN to achieve about a 30/70 mixture of Water/AcCN (-200-400 mL total volume) with a low concentration of peptide (-0.2-0.5 mg/ml). The solution was then adjusted to pH 8 with Triethylamine (TEA, Acros Organics) ~10 equivalents, and the peptide's disulfide bridge was reduced with the addition of 2-4 equivalents of Tris(2-carboxyethyl) phosphine hydrochloride (TCEP-HCl, Sigma Aldrich) reducing agent. After the disulfide bridge reduction, the thioacetal bridge was formed by the addition of 7-10 equivalents of Diiodomethane (CH₂12, Alfa Aesar). The Thioacetal formation reaction was carried out by incubating the solution for 18 hours at RT with magnetic stirring. Progress of the reaction was monitored using analytical LC-MS and by observing the change in mass of +12 Daltons from the starting reduced peptide molecular weight and an accompanying shift in retention of the starting material HPLC peak.

### HPLC Purification

The thioacetal reaction solution was loaded directly onto a preparative HPLC system (Waters 2545 Binary Systems) equipped with a column heater and using a Luna Phenyl-Hexyl RP-HPLC column (Phenomenex Inc.; 5 μm, 100 Å; 250×21.2 mm). The running buffers used were A: 0.1% Formic Acid/H₂O and B: 0.1% Formic/Acetonitrile (AcCN, Fisher Chemicals). The initial loading was done at 5% B, with 10 min isocratic wash after loading equilibration. The sample was eluted using a linear 5-65% B gradient over 60 min, at a flow of 15 mL/min, with column heating set at 60° C. Fractions that were determined to contain the desired product (analysis by LC-MS) were pooled, 0.05 mL of TFA were added to convert the final product to a TFA salt, the solution was then frozen and lyophilized to give a white amorphous solid product, as the TFA salt of the title compound. The purity assessed by RP-HPLC was found to be >95%, with the observed LC-MS molecular weight of 2487.77 Dalton, matching the theoretical calculated molecular weight of 2487.79 Dalton.

Below is a depiction of the structure of intermediate product H-AEEA-AEEA-AEEA-RSS[CFGGRIDRIGAQSGLGC]-OH (Thioacetal Bridge Cys7-Cys23), using the standard single letter code for L-Amino Acids except for the 2-(2-(2-Aminoethoxy)-ethoxy) acetic acid spacer (AEEA) where the structures of the residues have been expanded.

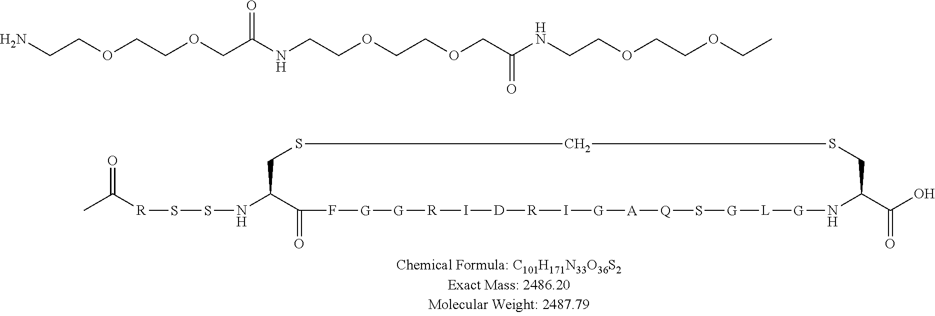

Acylation of Intermediate Thioacetal Peptide with Fmoc-Cys(Trt)-Opfp

In a 15 mL falcon tube equipped with a spin vane, the purified, lyophilized precursor peptide was dissolved in 1000 μL of DMSO (Acros Organics) to it were added 75 μL of DIPEA (TCI America) to adjust the pH to ~8-9, the reaction was magnetically stirred. Fmoc-Cys(Trt)-Opfp (-200 mg, ChemImpex) was dissolved in DMSO, 50 μL aliquots were added one at time while monitoring the progression of the reaction by LC-MS. Once the starting material was consumed, the reaction was stopped by the addition of ~10 mL cold diethyl ether (Fisher Chemicals) (−20° C.). The tube was mixed vigorously and centrifuged to force the peptide into a pellet (oil phase). Another fresh volume of cold diethyl ether was added, and the process was repeated, this time a white tacky precipitate was formed. The pellet was treated with 1 ml of 20% PIP in DMF (for ~15 min). Another round of precipitation was carried out using cold diethyl ether, washed and additional time with the cold ether. The pellet was finally treated with TFA (2% TIPS) to remove the Trityl protecting group from the Cys residue. The cold diethyl ether precipitation was once again used to isolate the final crude product.

### HPLC Purification

The crude peptide was dissolved in 15 mL of water and then loaded, via injection valve onto a preparative HPLC system (Shimadzu LC-8A Binary Systems) using a Luna Phenyl-Hexyl RP-HPLC column (Phenomenex Inc.; 5 μm, 100 Å; 250×21.2 mm). The running buffers used were A: 0.1% TFA/H₂O and B: 0.1% TFA/Acetonitrile (AcCN, Fisher Chemicals). The loading was done at 0% B, with 5 min isocratic wash at 0% B for column equilibration. The sample was eluted using a linear 0-60% B gradient over 60 min, at a flow of 25 mL/min, with column heating set at 60° C. Fractions that were determined to contain the desired product (analysis by LC-MS) were pooled, frozen and lyophilized to give a white amorphous solid product, as the TFA salt of the title compound. The purity assessed by RP-HPLC 1 was found to be >95%, with the observed molecular weight of 2590.40 Dalton; matching the theoretical calculated molecular weight of 2590.93 Dalton.

### Example 3C: Synthesis of Cys-(SP1)-(SEQ ID: 17)

Below is a depiction of the structure of the title compound using the standard single letter code for L-Amino Acids except for the 2-(2-(2-Aminoethoxy)-ethoxy) acetic acid spacer (AEEA) and L-Cysteine (Cys) at the N-Terminus where the structures of the residues have been expanded.

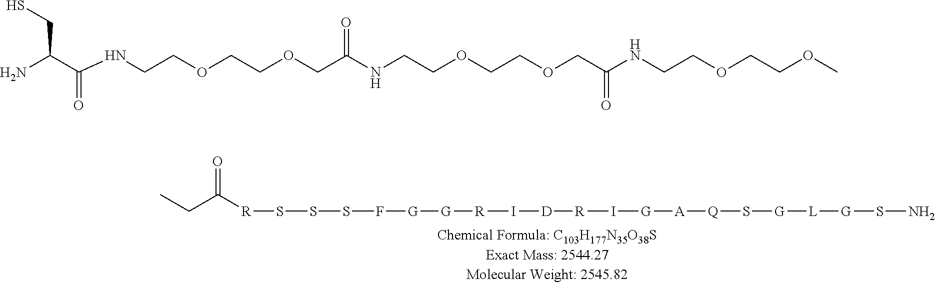

The primary peptide sequence of the title compound is essentially similar to Example 3B. For this iteration, the disulfide bridge was removed by replacing Cys7 and Cys23 with Ser7 and Ser23 residues resulting in a linear analog without a connecting disulfide or thioacetal bridge. The synthesis of the title compound was carried out in a similar manner as Example 3B, with the noted Cys->Ser replacements, and was made as a C-Terminal Amide instead of a C-Terminal acid. The solid support resin used consists of low loading 4-(2′,4′-Dimethoxyphenyl-Fmoc-aminomethyl)-phenoxyacetamido-norleucyl-4-Methylbenzhydrylamine resin (Fmoc-Rink-MBHA Low Loading resin, EMD Millipore), (100-200 mesh) with a 1% DVB cross-linked polystyrene core and a substitution range of 0.3-0.4 meq/g. Amide couplings were performed for 1 hour using the Fmoc Amino Acid (0.3 M, Advanced ChemTech, in DMF), N,N′-Diisopropylcarbodiimide (DIC, ChemImpex, 1.2 M in DCM) and Ethyl Cyanohydroxyiminoacetate (Oxyma Pure, ChemImpex; 0.9 M in DMF), at a 9-fold molar excess of AA/Oxyma and a 12-fold molar excess of DIC over the reported resin loading level. After the SPPS was complete, the peptidyl resin was transferred as a DCM slurry to disposable fritted plastic syringe fitted with Teflon stopcock. Further washes with DCM were done, and finally, the resin was thoroughly dried in vacuo. The dry resin was then treated with 10 mL of cleavage cocktail consisting of trifluoroacetic acid (TFA, Acros Organics), water, 3,6-dioxa-1,8-octanedithiol (DODT; Sigma Aldrich), triisopropylsilane (TIPS; Acros Organics), (TFA:Water:DODT:TIPS; 90:5:2.5:2.5 v/v) for 2 hours at room temperature. After the 2 hr cleavage incubation, the resin was filtered off, washed twice with 2 mL of neat TFA, and the combined filtrates/washes were collected in a 50 ml conical disposable tube, the solution was then treated with 35 mL of cold diethyl ether (Fisher Chemicals) (−20° C.) to precipitate the crude peptide. The peptide/ether suspension was then centrifuged at 4000 rpm for 2 min to form a solid pellet, the supernatant was decanted, and the solid pellet was triturated with fresh ether and the process was repeated two additional times, finally drying the peptide pellet in vacuo. HPLC Purification

The crude peptide was dissolved in 15 mL of water and then loaded, via injection valve onto a preparative HPLC system (Shimadzu LC-8A Binary Systems) using a Luna Phenyl-Hexyl RP-HPLC column (Phenomenex Inc.; 5 μm, 100 Å; 250×21.2 mm). The running buffers used were A: 0.1% TFA/H₂O and B: 0.1% TFA/Acetonitrile (AcCN, Fisher Chemicals). The loading was done at 0% B, with 5 min isocratic wash at 0% B for equilibration. The sample was eluted using a linear 0-60% B gradient over 60 min, at a flow of 25 mL/min, with column heating set at 60° C. Fractions that were determined to contain the desired product (analysis by LC-MS) were pooled, frozen and lyophilized to give a white amorphous solid product, as the TFA salt of the title compound. The purity assessed by RP-HPLC 1 was found to be >95%, with the observed molecular weight of 2545.60 Dalton; matching the theoretical calculated molecular weight of 2545.82 Dalton.

### Example 3D: Synthesis of Mpa-(SP1)-(SEQ ID: 22)

Below is a depiction of the structure of the title compound with all residues expanded.

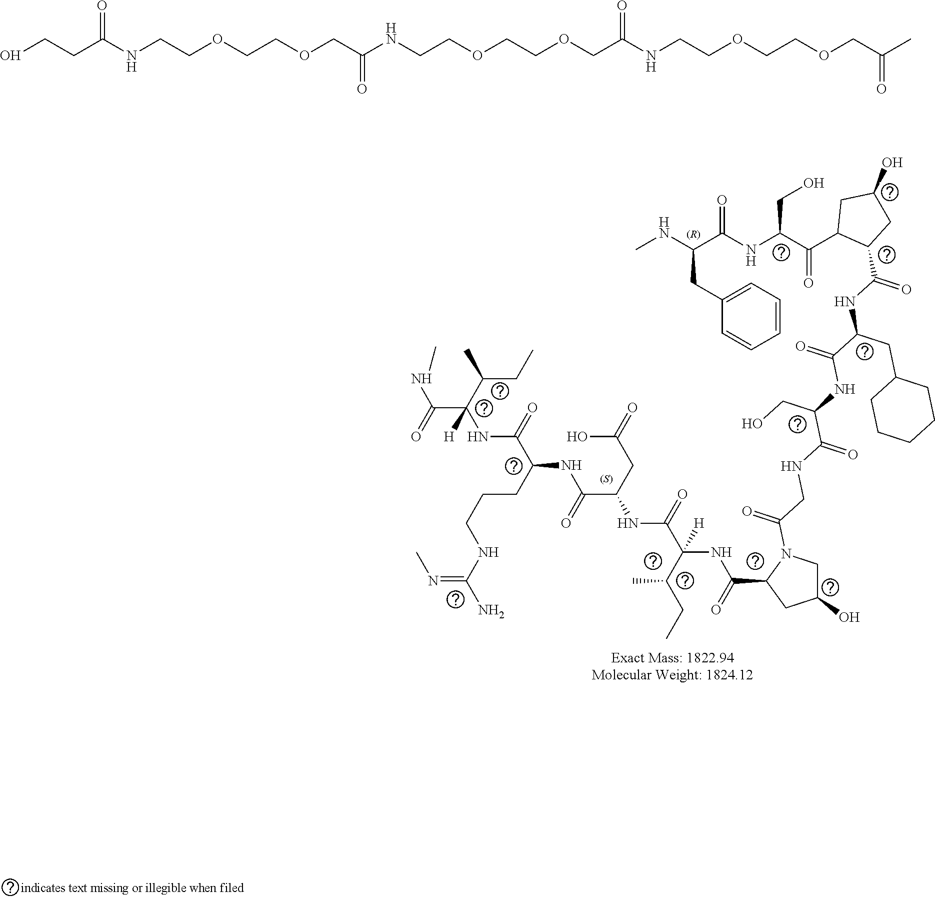

The protocol used for the synthesis of the title compound is essentially similar as that used in Example 3C. In this iteration, the starting resin used was 2 [3-((Methyl-Fmoc-amino)-methyl)indol-1-yl]acetyl AM resin (Methyl Indole AM Resin), (100-200 mesh) with a 1% DVB cross-linked polystyrene core with a substitution range of 0.3-0.4 meq/g. This solid support generates an N-methyl substituted carboxamide containing peptide (—NH—CH₃). The previously outlined cleavage and purification protocols were used for the workup of the material.

### Example 3E: Synthesis of MalDap-(SP12)-(SEQ ID: 21)

Below is a depiction of the structure of the title compound using the standard single letter code for L-Amino Acids, except for the 2-(2-(2-Aminoethoxy)-ethoxy) acetic acid spacer (AEEA), D-Phe, D-Pro, D-Ala, β-cyclohexyl-L-alanine (Cha), and Mal-Dap-at the N-Terminus where the structures of the residues have been expanded.

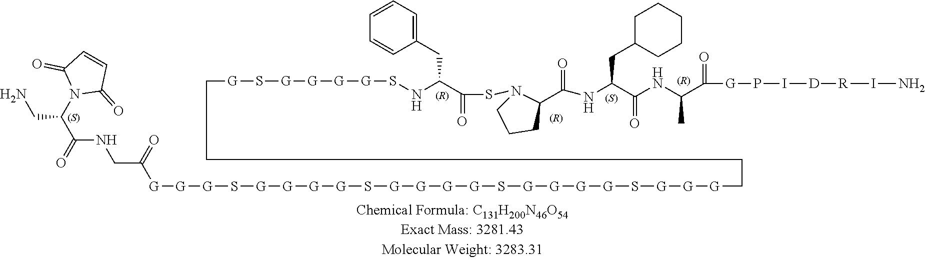

The synthesis of the title compound was carried out in a similar manner as previous preparations. The solid support resin used Fmoc-Rink-MBHA Low Loading resin(EMD Millipore), (100-200 mesh) with a 1% DVB cross-linked polystyrene core and a substitution range of 0.3-0.4 meq/g. Standard 1 hour couplings were performed using Fmoc Amino Acid (0.3 M, Advanced ChemTech, in DMF), N,N′-Diisopropylcarbodiimide (DIC, ChemImpex, 1.2 M in DCM) and Ethyl Cyanohydroxyiminoacetate (Oxyma Pure, ChemImpex; 0.9 M in DMF), at a 9-fold molar excess of AA/Oxyma and a 12-fold molar excess of DIC over the reported resin loading level. Of note, Fmoc-Gly-Gly-OH dipeptide (ChemImpex) was used to build the (GGGGS)6 (SEQ ID: 79) repeat, coupling time was extended to 2 hrs for the dipeptide. Mal-Dap(Boc)-OH (ChemImpex) was used to cap the N-terminus of the peptide using standard coupling conditions. The previously outlined cleavage and purification 90rotocolls were used for the workup of the material.

### Example 3F: Synthesis of Mpa-(SP1)-(SEQ ID: 21)-(SF4)-(FA4)

Below is a depiction of the structure of the title compound using the standard single letter code for L-Amino Acids, except for the 2-(2-(2-Aminoethoxy)-ethoxy) acetic acid spacer (AEEA), γ-Glutamic Acid, D-Phe, D-Pro, D-Ala, β-cyclohexyl-L-alanine (Cha), C-Terminal Lys, and 3-Mercaptopropionic Acid at the N-Terminus where the structures of the residues have been expanded.

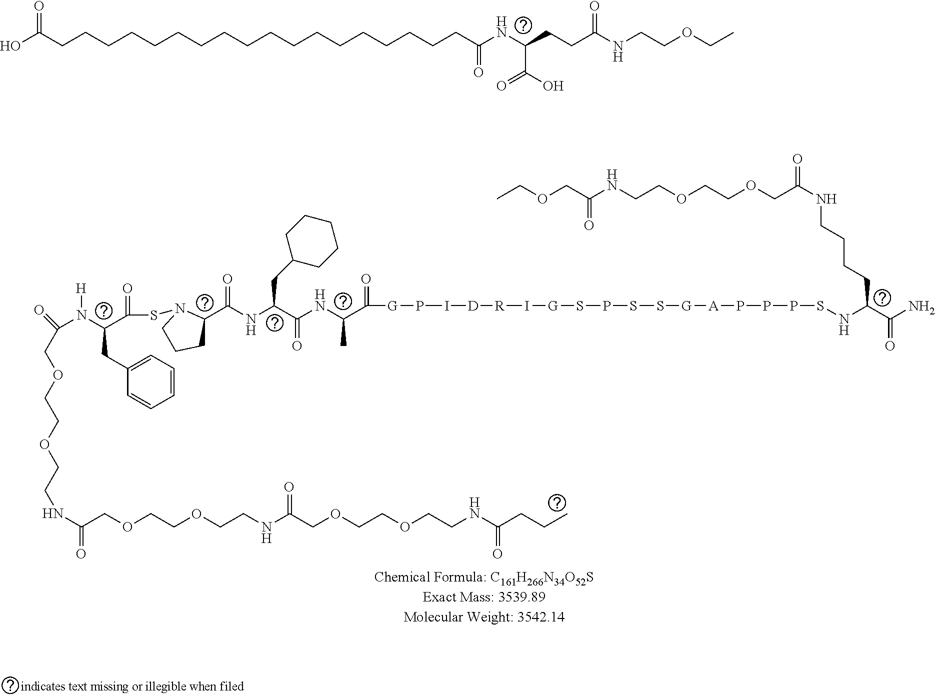

The synthesis of the title compound was carried out in a similar manner as described previously. The solid support resin used Fmoc-Rink-MBHA Low Loading resin (EMD Millipore), (100-200 mesh) with a 1% DVB cross-linked polystyrene core and a substitution range of 0.3-0.4 meq/g. Standard 1 hour couplings were performed using Fmoc Amino Acid (0.3 M, Advanced ChemTech, in DMF), N,N′-Diisopropylcarbodiimide (DIC, ChemImpex, 1.2 M in DCM) and Ethyl Cyanohydroxyiminoacetate (Oxyma Pure, ChemImpex; 0.9 M in DMF), at a 9-fold molar excess of AA/Oxyma and a 12-fold molar excess of DIC over the reported resin loading level. The first amino acid coupled to the resin was Fmoc-Lys(Mtt)-OH (Advanced ChemTech) and was done using standard coupling conditions. The use of orthogonal Mtt protection on Lysine residues allows for selective on-resin side chain deprotection (s-amino group) and directed lipidation of the peptides. For the Mtt deprotection, the resin is thoroughly washed with DCM after the last coupling step of Trt-3-Mpa-OH and then treated 4 times with a sufficient volume of 30% 1,1,1,3,3,3-Hexafluoroisopropanol (HFIP, ChemIpex) in DCM. Each HFIP treatment is done for 30 minutes with nitrogen mixing. After Mtt removal the resin was thoroughly washed with DCM (8x), and subsequently DMF (8×). Acylation with OtBu-C20-(γGlu-OtBu)-AEEA-AEEA-OH afforded the side chain linker fatty diacid, coupling was done using DIC/Oxyma at 3× excess for ~18 hrs. The previously outlined cleavage and purification protocols were used for the workup of the material.

### Example 3G: Synthesis of FA1

Below is a depiction of the structure of the title compound:

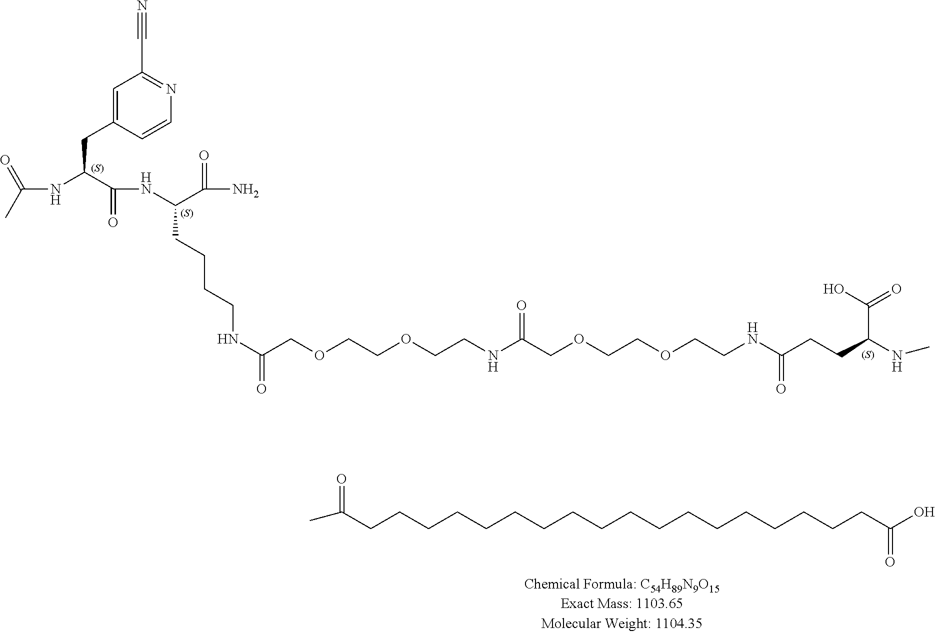

The title compound was synthesized manually in a fritted glass reaction vessel (50 mL) using standard 9-Fluorenyl-methyloxycarbonyl (Fmoc) tert-Butyl (t-Bu) solid phase peptide chemistry protocols at a 1.0 mmol scale. The solid support used consisted of 4-(2′,4′-Dimethoxyphenyl-Fmoc-aminomethyl)-phenoxy resin (Rink-Resin LS, CreoSalus), (100-200 mesh) with a 1% DVB cross-linked polystyrene core and a substitution of ~0.29 meq/g. The building blocks used for the synthesis were: Fmoc-L-Lys(Mtt)-OH (AappTec Peptides), Fmoc-3-(2-cyano-4-pyridyl)alanine (Fmoc-Cpa-OH, WuXi), Fmoc-Glu-OtBu (Advanced ChemTech), 1-Acetylimidazole (Acros Organics) Fmoc-AEEA-OH, and 20-(tert-Butoxy)-20-oxoicosanoic acid (HO-C20-OtBu, WuXi). Fmoc deprotection prior to each coupling step was accomplished by treatments with 20% Piperidine (PIP; Sigma Aldrich) in Dimethylformamide (DMF; Fisher Chemicals), 2×10 minutes with nitrogen mixing, followed by 8 × DMF wash cycles. Fmoc-Lys(Mtt)-OH and Fmoc-Cpa-OH were performed for 3 hours using a 5× excess of the Fmoc Amino Acids, DIC and Oxyma. Capping or acetylation of the N-terminus was done using Acetyl-imidazole in DCM (6× excess) reaction time 2 hrs. After the capping step, the resin was washed with DCM 8×. The Mtt side chain deprotection of Lysine was done with using 20% HFIP in DCM (3×20 min), the resin washed 3× DCM, 4× DMF. Coupling of Fmoc-AEEA-OH residues and Fmoc-Glu-OtBu were performed for 5 hours using a 5× excess of the Fmoc Amino Acids, DIC and Oxyma. Coupling of OtBu-C20-OH was done using PyBop/DIPEA 3× excess (overnight). After the coupling of the fatty diacid, the resin washed 3× DMF, 3× DCM, 2 Ether and dried in vacuo. The dry resin was then treated with 100 mL of cleavage cocktail consisting of trifluoroacetic acid (TFA, Acros Organics), water, triisopropylsilane (TIPS; Acros Organics), (TFA:Water:TIPS; 90:5:5 v/v) for 2 hours at room temperature. After the 2 hr cleavage incubation, the resin was filtered off, washed 00 mL of DCM TFA, and the combined filtrates/washes were collected in six 50 ml conical disposable tubes, the solutions were then treated with 35 mL of cold diethyl ether (Fisher Chemicals) (−20° C.) each to precipitate the crude product. The ether suspensions were then centrifuged at 4000 rpm for 2 min to form solid pellets, the supernatant was decanted, and the solid pellets were triturated with fresh ether and the process was repeated two additional times, finally drying the pellets in vacuo. The crude material was used without further purification.

### Example 4: Synthesis of Oligonucleotides for Targeting ALK7 (e.g., siRNA) and Reagents for Oligonucleotide Functionalization

### Examples 4 Å-4VV

Single strands (sense and antisense) were synthesized on solid support via a MerMade 12 (LGC Biosearch Technologies), K&A H-8 SE (K&A Labs GmbH) oligonucleotide synthesizer. The sequences of the sense and antisense strands were shown in Tables 4 and 6. In addition to the strand sequence, appropriate amidites and CPGs were utilized to append appropriate linkers to enable the conjugates in Table 13.

Strands that were conjugated to peptides were synthesized using an appropriate conjugatable CPG such as “3′-Amino Modifier C-3 lcaa CPG 500 Å” (part number N-8271-05, Chemgenes) to provide a reactive amino on the 3′-end, or “3′ Thiol Modifier C6 SS” (part number ON-526-UC, Hongene-C6SSC6-CPG) to provide a reactive thiol on the 3′-end. Antisense strands used standard 2′-O-Methyl supports (LGC Biosearch Technologies). Strands that were conjugated to GalNAc moieties were synthesized using an appropriate pre-functionalized CPG such as commercially available GalNAc (TEG)-CPG (MRS1279038APT, Amerigo Scientific) for the dsRNAs herein bearing the GalNAc referred to as Gal-1, or custom CPG bearing the GalNAc moiety referred to below as Gal-2, which was synthesized according to procedures found in WO2022271806A1.

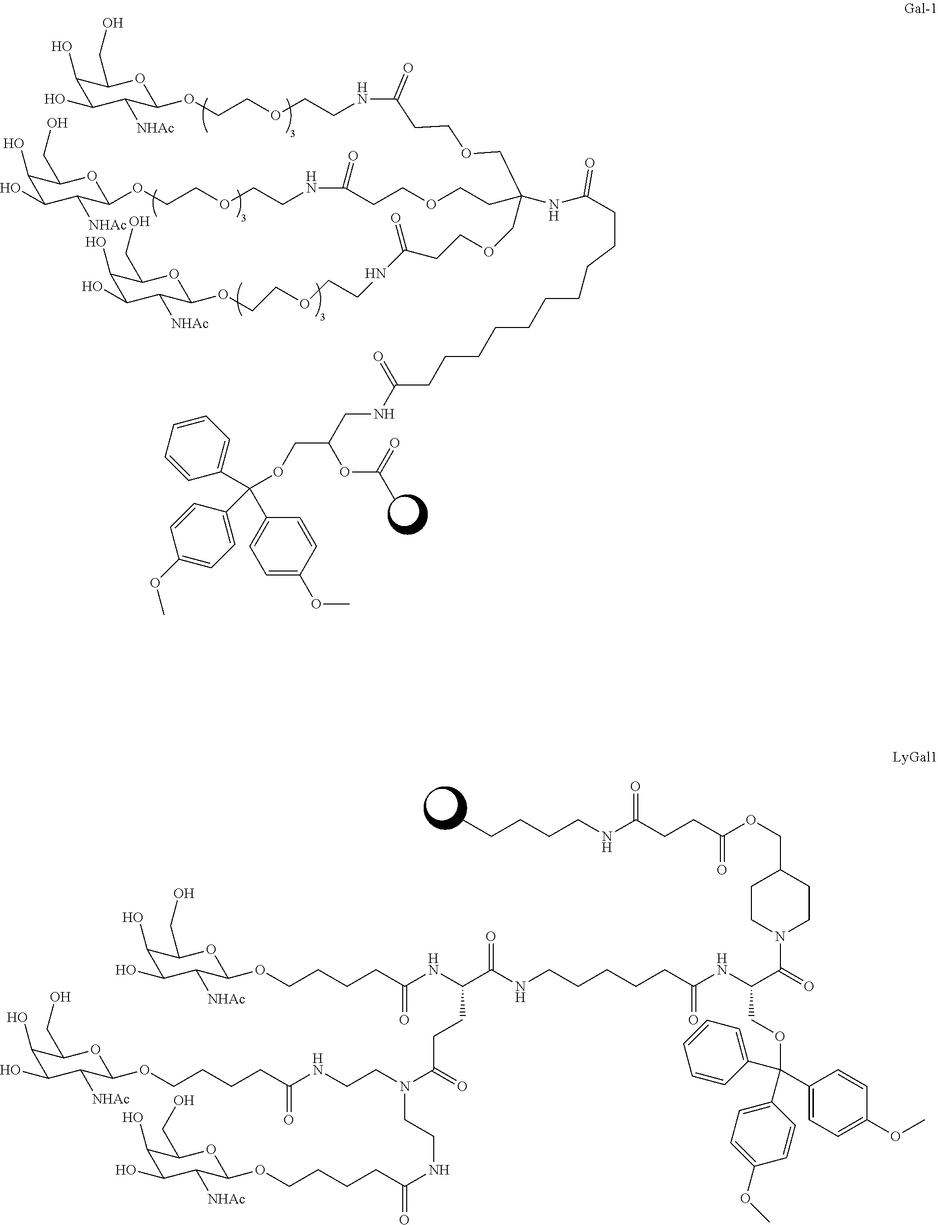

The oligonucleotides were synthesized via phosphoramidite chemistry at an appropriate scale for in-vitro or in-vivo experimentation. Standard reagents were used in the oligo synthesis (Table 16), where 0.1M xanthane hydride in pyridine was used as the sulfurization reagent and 20% DEA in ACN was used as an auxiliary wash post synthesis. For antisense strands, the 20% DEA wash was typically omitted and ACN wash was used instead. All monomers (Table 17, phosphoramidites) were made at 0.1M in ACN and contained a molecular sieves trap bag. When necessary, inclusion of 10% volume equivalents of DMF or 50% volume equivalents of DCM were utilized to maintain amidite solubility (i.e. mU amidite). For lipid-containing amidites such as those which were used to synthesize sense strands bearing 5′-terminal FA5 through FA9, FA11-FA18 and internal Upa-conjugated FA27, the appropriate monomers were typically dissolved at 0.1 or 0.2M in 50% volume equivalents of DCM:ACN or 100% DCM and used under standard phosphoramidite coupling conditions. For lipid-containing amidites bearing carboxylic acid esters such as those which were used to synthesize sense strands bearing 5′-terminal FA10-SF8, FA26-SF8, or FA19-FA25, the appropriate monomers were typically dissolved at 0.1 or 0.2M in 50% volume equivalents of DCM:ACN or 100% DCM.

The sense strands made with 3′-phthalimido C6 amino CPG were typically cleaved and deprotected from the CPG using 50% (methylamine/ammonia hydroxide 28-30%) at ambient temperature (about 25° C.) for 2-3 hrs. For lipid-containing sense strands bearing carboxylic acid such as those bearing FA10-SF8, FA26-SF8, or any of FA19-FA25, cleavage and deprotection (C/D) typically was achieved using 0.4 M NaOH in 4:1 MeOH/H2O at ambient temperature (about 25° C.) for 24 hours and GalNAc sense strands were typically cleaved and deprotected (C/D) at 38 to 45° C. for 17 hours using a solution of 3% DEA in ammonia hydroxide (28-30%, cold).C/D was determined complete by IP-RP LCMS when the resulting mass data confirmed the identity of sequence. Dependent on scale, the CPG was filtered via 0.45 um PVDF syringeless filter, 0.22 μm PVDF Steriflip® vacuum filtration or 0.22 μm PVDF Stericup® Quick release.

The CPG was typically back washed/rinsed with RNAse free water or 30% EtOH/RNAse free water, then filtered through the same filtering device and combined with the first filtrate. This was repeated twice. The material was then divided evenly into conical centrifuge tubes to remove organics via Genevac™. After concentration, the crude oligonucleotides were diluted back to synthesized scale with RNAse free water and filtered either by 0.45 μm PVDF syringeless filter, 0.22 μm PVDF Steriflip® vacuum filtration or 0.22 μm PVDF Stericup® Quick release.

Sense strands containing C6SSC6 protected thiol moiety installed on solid phase either originating from a 3′-modification via C6SSC6-CPG or 5′-modification via C6SSC6-PA were typically treated with an excess (tris(2-carboxyethyl)phosphine)-HCl to reduce the dithio bond after removal of volatiles and prior to purification, unless functionalization of an amino moiety is necessary prior to functionalization of the liberated thiol moiety. In this case, purification as below may take place and disulfide cleavage may be similarly employed following amino functionalization.

The crude oligonucleotides were purified via AKTA™ Pure purification system using anion-exchange (AEX) or reverse-phase (RP) chromatography utilizing a gradient of Mobile Phase A (MPA) to Mobile Phase B as appropriate for the product. For AEX, an ES Industry Source™ 15Q column with MPA: 20 mM NaH₂PO₄, 15% ACN, pH 7.4 and MPB: 20 mM NaH₂PO₄, 1M NaBr, 15% ACN, pH 7.4. For RP, an ES Industry Source™ 15RPC with MPA: 10 mM NaOAc 2% Acetonitrile and MPB: 80% Acetonitrile in water. Fractions were analyzed by IP-RP LCMS and those which contained a mass purity greater than 85% without impurities >5% were combined.

The purified oligonucleotides were typically desalted using 15 mL 3K MWCO centrifugal spin tubes at 3500×g for −30 min. The oligonucleotides were rinsed with RNAse free water until the eluent conductivity reached <100 usemi/cm. After desalting was complete, 2-3 mL of RNAse free water was added then aspirated 10×, the retainment was transferred to an appropriately sized conical tube, this was repeated until complete transfer of oligo by measuring concentration of compound on filter via nanodrop. The final oligonucleotide was then typically nano filtered via 15 mL 100K MWCO centrifugal spin tubes at 3500×g for 2 min. The final desalted oligonucleotides were analyzed for concentration (nanodrop at A260). The molecular weight of the product was confirmed by LC-MS analysis using a Linear Ion Trap Mass Spectrometer (LTQ XL, Thermo Fisher), and purity was confirmed by UPLC analysis.

Oligonucleotide UPLC analysis was typically conducted under Ion-Pairing Reversed-Phase Ultra Performance Liquid Chromatography (IP-RP UPLC) conditions using a Waters™ ACQUITY™ UPLC Oligonucleotide BEH C18 Column 2.1×50 mm, 1.7 μm column. The gradient used Mobile Phase A (MPA) of 7 mM triethylamine with 100 mM hexafluoroisopropanol, and a Mobile Phase B (MPB) of 7:3 Methanol to Acetonitrile. Oligonucleotides with low lipophilicity were typically analyzed over a gradient of 0 to 25% MPB over 10 minutes while oligonucleotides with high lipophilicity were analyzed over a gradient of 5 to 90% MPB over 10 minutes.

Lipid bearing constructs characterized by mass spectrometry in Table 20b were constructed according to the following.

The construct containing dsRNA NO 291 was synthesized with 5′-vinylphosphonate antisense strand and Ada-bearing sense strand. This construct was synthesized according to methods substantially similar to those described in WO2025064660—ACTIVIN A RECEPTOR TYPE 1C (ACVR1C) IRNA COMPOSITIONS AND METHODS OF USE THEREOF for 2′-OC22 containing oligonucleotides, in particular the construct described as AD-2640060. The sense strand was synthesized using the amidite DMT-2′-O-C22-A(Bz)-CE-Phosphoramidite in Table 17.

The construct with dsRNA NO 292 was synthesized with a 5′-cyclopropylphophonate bearing antisense strand substantially similar to antisense strands described in WO2025137390—RNAI AGENTS FOR INHIBITING EXPRESSION OF ACTIVIN RECEPTOR-LIKE KINASE 7 (ALK7), COMPOSITIONS THEREOF, AND METHODS OF USE, according to methods described therein. It's sense strand was constructed in a substantially similar fashion to the sense strand of AC006188 described therein.

The construct with dsRNA NO 293 was synthesized with a 5′-vinylphosphonate bearing antisense strand according to methods described herein, and a sense strand utilizing substantially similar methods and lipids as constructed in WO2025137390 for the sense strand of AC006188 described therein, except with the amidite coupling sequence necessary to implement SEQ ID 580 in conjunction with those lipid chemistries. Post-synthesis functionalization methods and materials were substantially similar to those described therein.

TABLE 16
Oligonucleotide Synthesis Reagents
Reagents

Activator Solution (0.5M ETT in ACN)
Cap A (Acetic Anhydride, Pyridine in THF, 1:1:8)
Cap B (1-Methylimidazole in THF, 16:84)
Oxidation Solution (0.02M Iodine in THF/Pyridine/Water,
70:20:10)
Deblock Solution, 3% TCA in DCM (w/v)
Acetonitrile (Anhydrosolv, Water max. 10 ppm)
Xanthane Hydride (0.1M in Pyridine)
Diethylamine (20% in Acetonitrile)
PN Solution 2-Azido-1,3-dimethylimidazolinium
Hexafluorophosphate (0.5M in Acetonitrile)

TABLE 17
Phosphoramidites

| | | | Catalog | |
|---|---|---|---|---|
| Phosphoramidite | Abbreviation | Supplier | # | CAS |
| DMT-2′-F-A(Bz)-CE | fA | Hongene | PD1-001 | 136834-22-5 |
| Phosphoroamidite | | | | |
| DMT-2′-F-C(Ac)-CE | fC | Hongene | PD3-001 | 159414-99-0 |
| Phosphoroamidite | | | | |
| DMT-2′-F-G(iBu)-CE | fG | Hongene | PD2-002 | 144089-97-4 |
| Phosphoroamidite | | | | |
| DMT-2′-F-U-CE | fU | Hongene | PD5-001 | 146954-75-8 |
| Phosphoroamidite | | | | |
| DMT-2′-O-Me-A(Bz)-CE | mA | Hongene | PR1-001 | 110782-31-5 |
| Phosphoroamidite | | | | |
| DMT-2′-O-Me-C(Ac)-CE | mC | Hongene | PR3-001 | 199593-09-4 |
| Phosphoroamidite | | | | |
| DMT-2′-O-Me-G(iBu)-CE | mG | Hongene | PR2-002 | 150780-67-9 |
| Phosphoroamidite | | | | |
| DMT-2′-O-Me-U-CE | mU | Hongene | PR5-001 | 110764-79-9 |
| Phosphoroamidite | | | | |
| 5′bis(POM) vinyl phosphate- | POM-VPmU | Hongene | PR5-032 | BVPMUP23B2A1 |
| 2′-Ome-U3′CE | | | | |
| phosphoroamidite | | | | |
| Reverse Abasic | iAb | Chemgenes | ANP- | 401813-16-9 |
| phosphoroamidite | | | 1422 | |
| Abasic phosphoroamidite | Aba | Chemgenes | ANP- | 129821-76-7 |
| | | | 7058 | |
| 2′-O-Trifluoroacetamido propyl | Upa | Chemgenes | ANP- | 165381-49-7 |
| Uridine CED phosphoramidite | | | 7115 | |
| 5′-Thio-modifier C6 S-S | C6SSC6-PA | Hongene | OP-043 | 148254-21-1 |
| phosphoramidite | | | | |
| 5′-Amino-Modifier C6-TFA | 5′-C6Am-PA | Hongene | OP-007 | 133975-85-6 |
| phosphoramidite | | | | |
| Hexadecyl A | Ahd | Lilly | N/A | 2382942-35-8 |
| phosphoroamidite | | | | |
| Hexadecyl G | Ghd | Lilly | N/A | 2382942-32-5 |
| phosphoroamidite | | | | |
| Hexadecyl C | Chd | Lilly | N/A | 2382942-38-1 |
| phosphoroamidite | | | | |
| Hexadecyl U | Uhd | Lilly | N/A | 2382942-83-6 |
| phosphoroamidite | | | | |
| S-mercaptotertbutyl-L-cystine- | tBuCys | Lilly | N/A | 294172-35-3 |
| transcyclohexylamido | | | | |
| phosphoramidite | | | | |
| C20-Diacid-CE phosphoramidite | C20DA | Lilly | N/A | N/A |
| C20-Acid-Ethanolamide | C20AEA | Lilly | N/A | N/A |
| phosphoramidite | | | | |
| Oleyl phosphoramidite | Ole | Lilly | N/A | 847595-74-8 |
| Cetyl phosphoramidite | Cet | Lilly | N/A | 141186-19-8 |
| C22-Docosyl phosphoramidite | Doco | Lilly | N/A | 2093462-10-1 |
| Adamantaneethanol | Adam | Lilly | N/A | 143723-72-2 |
| phosphoramidite | | | | |
| Glycol bis(DMT)doubler | Doubler | Hongene | OP-189 | 125922-95-4 |
| phosphoramidite | | | | |
| DMT-2′-O-MOE-G(iBu)-CE- | MOE-G | Hongene | PR2-006 | 251647-55-9 |
| Phosphoramidite | | | | |
| DMT-2′-O-MOE-T-CE- | MOE-T | Hongene | PR4-002 | 163878-63-5 |
| Phosphoramidite | | | | |
| UNA-U-CE Phosphoramidite | UNA-U | PR5-007 | Hongene | 1120329-48-7 |
| UNA-G(iBu)-CE | UNA-G | PR2-014 | Hongene | 1120329-61-4 |
| Phosphoramidite | | | | |
| UNA-A(Bz)-CE | UNA-A | PR1-012 | Hongene | 1120329-52-3 |
| Phosphoramidite | | | | |
| UNA-C(Ac)-CE | UNA-C | PR3-018 | Hongene | 1120329-56-7 |
| Phosphoramidite | | | | |
| T-LA-CE Phosphoramidite | LNA-T | 10-2030 | Glen | 206055-75-6 |
| | | | Research | |
| Bz-5-Me-C-LA-CE | LNA-C | 10-2011 | Glen | 206055-82-5 |
| Phosphoramidite | | | Research | |
| dmf-G-LA-CE | LNA-G | 10-2029 | Glen | 709641-79-2 |
| Phosphoramidite | | | Research | |
| Bz-A-LA-CE | LNA-A | 10-2000 | Glen | 206055-79-0 |
| Phosphoramidite | | | Research | |
| O-C20-Acid phosphoramidite | OC20A | Lilly | N/A | N/A |
| O-C18-Acid phosphoramidite | OC18A | Lilly | N/A | N/A |
| O-C16-Acid phosphoramidite | OC16A | Lilly | N/A | N/A |
| C16-Acid Ethanolamide | C16AEA | Lilly | N/A | N/A |
| phosphoramidite | | | | |
| γ-Glu-C16-Acid | γEC16AEA | Lilly | N/A | N/A |
| Ethanolamide | | | | |
| phosphoramidite | | | | |
| 2′-O-icosanamidopropyl Uridine | Upi | Lilly | N/A | N/A |
| CED phosphoramidite | | | | |
| DMT-2′-O-C22-A(Bz)-CE- | Ada | Hongene | N/A | 2923115-67-5 |
| Phosphoramidite | | | | |

N/A denotes not applicable.

### Example 4 Å: Hexadecyl A phosphoroamidite

### N-[9-[(2R,3R,4R,5R)-5-[[Bis(4-methoxyphenyl)-phenyl-methoxy]methyl]-4-[2-cyanoethoxy-(diisopropylamino)phosphanyl]oxy-3-hexadecoxy-tetrahydrofuran-2-yl]purin-6-yl]benzamide

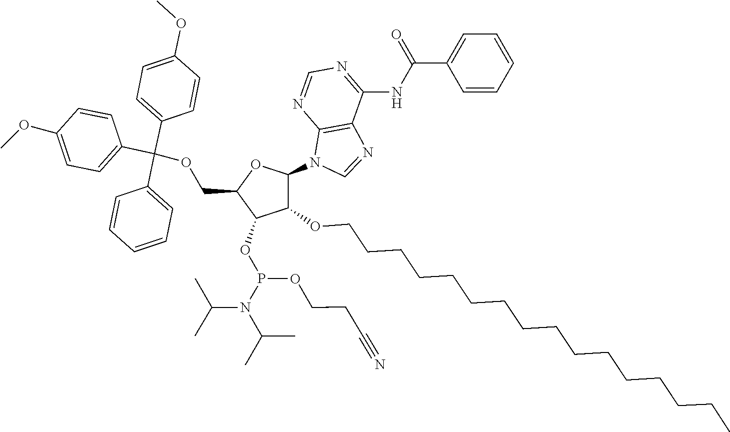 [SMILES: CCCCCCCCCCCCCCCCO[C@@H]1[C@H](OP(OCCC#N)N(C(C)C)C(C)C)[C@@H](COC(c2ccccc2)(c2ccc(OC)cc2)c2ccc(OC)cc2)O[C@H]1n1cnc2c(NC(=O)c3ccccc3)ncnc21]

Prepared the title compound, referred to herein as Hexadecyl A phosphoroamidite, according to the protocols described in WO2019217459. ¹H-NMR (CD₃CN) δ 9.37 (s, 1H), 8.57 (d, J=9.4 Hz, 1H), 8.27 (d, J=10.3 Hz, 1H), 7.99 (d, J=7.6 Hz, 2H), 7.61 (d, J=7.4 Hz, 1H), 7.52 (t, J=7.6 Hz, 2H), 7.42 (t, J=7.3 Hz, 2H), 7.34-7.16 (m, 7H), 6.85-6.77 (m, 4H), 6.11 (dd, J=5.0, 2.5 Hz, 1H), 4.80 (m, 1H), 4.69 (m, 1H), 4.32 (m, 1H), 3.97-3.78 (m, 1H), 3.74 (d, J=3.1 Hz, 7H), 3.64 (m, 4H), 3.56-3.40 (m, 2H), 3.33 (m, 1H), 2.73-2.59 (m, 1H), 2.50 (t, J=6.0 Hz, 1H), 1.52-1.45 (m, 2H), 1.33-1.12 (m, 37H), 1.09 (d, J=6.8 Hz, 3H), 0.87 (t, J=6.8 Hz, 3H). ³¹P NMR (CD₃CN) δ 151.19, 150.78.

### Example 4B: Hexadecyl U Phosphoroamidite

### 3-[[(2R,3R,4R,5R)-2-[[Bis(4-methoxyphenyl)-phenyl-methoxy]methyl]-4-hexadecoxy-5-(2-hydroxy-4-oxo-pyrimidin-1-yl)THF-3-yl]oxy-(diisopropylamino)phosphanyl]oxypropanenitrile

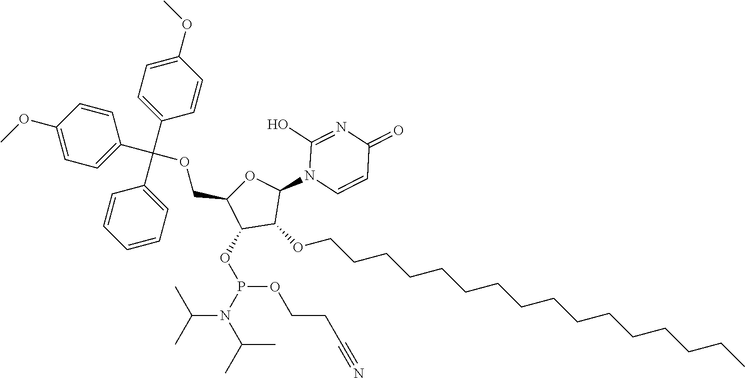 [SMILES: CCCCCCCCCCCCCCCCO[C@@H]1[C@H](OP(OCCC#N)N(C(C)C)C(C)C)[C@@H](COC(c2ccccc2)(c2ccc(OC)cc2)c2ccc(OC)cc2)O[C@H]1n1ccc(=O)nc1O]

Prepared the title compound, referred to herein as Hexadecyl U phosphoramidite, according to the protocols described in WO2019217459. 1H NMR (CD3CN): 7.86-7.73 (m, 1H), 7.51-7.43 (m, 2H), 7.40-7.23 (m, 7H), 6.95-6.87 (m, 4H), 5.90-5.84 (m, 1H), 5.29-5.21 (m, 1H), 4.54-4.40 (m, 1H), 4.21-4.13 (m, 1H), 4.10-3.56 (m, 13H), 3.50-3.34 (m, 2H), 2.75-2.62 (m, 1H), 2.55 (t, J=6.0 Hz, 1H), 1.66-1.51 (m, 2H), 1.40-1.14 (m, 35H), 1.08 (d, J=6.8 Hz, 3H), 0.91 (t, J=6.8 Hz, 3H). 31P NMR (CD3CN): 149.6, 149.2.

### Example 4C: Hexadecyl C Phosphoroamidite

### N-[1-[(2R,3R,4R,5R)-5-[[Bis(4-methoxyphenyl)-phenyl-methoxy]methyl]-4-[2-cyanoethoxy-(diisopropylamino)phosphanyl]oxy-3-hexadecoxy-tetrahydrofuran-2-yl]-2-oxo-pyrimidin-4-yl]acetamide

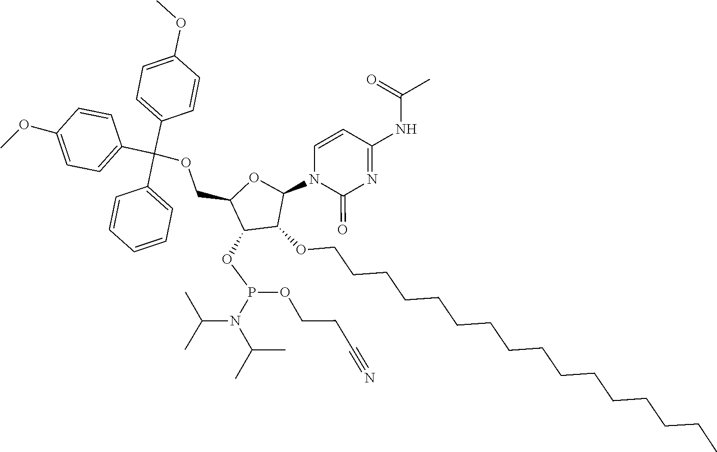 [SMILES: CCCCCCCCCCCCCCCCO[C@@H]1[C@H](OP(OCCC#N)N(C(C)C)C(C)C)[C@@H](COC(c2ccccc2)(c2ccc(OC)cc2)c2ccc(OC)cc2)O[C@H]1n1ccc(NC(C)=O)nc1=O]

The title compound, referred to herein as Hexadecyl C phosphoramidite, was prepared according to the protocols described in WO2019217459. 1H-NMR (CD3CN) 9.15 (s, 1H), 8.46 (dd, J=7.5 Hz, 1H), 7.95 (d, J=7.6 Hz, 2H), 7.63 (t, J=7.5 Hz, 1H), 7.57-7.41 (m, 5H), 7.41-7.31 (m, 6H), 7.28 (m, 1H), 7.04 (d, J=15.8 Hz, 1H), 6.90 (t, J=7.9 Hz, 4H), 5.90 (d, J=7.8 Hz, 1H), 4.51 (m, 1H), 4.20 (dd, J=10.6, 8.1 Hz, 1H), 4.04 (dd, J=31.3, 4.6 Hz, 1H), 3.91-3.81 (m, 2H), 3.79 (d, J=3.1 Hz, 6H), 3.74 (m, 2H), 3.69-3.41 (m, 6H), 2.67-2.59 (m, 1H), 2.54-2.48 (m, 1H), 1.58 (m, 2H), 1.36 (m, 2H), 1.25 (d, J=4.7 Hz, 26H), 1.21-1.09 (m, 10H), 1.04 (d, J=6.8 Hz, 3H), 0.87 (t, J=6.8 Hz, 3H). 31P NMR (CD3CN) δ 151.10, 150.19.

### Example 4D: Hexadecyl G Phosphoroamidite

### N-[9-[(2R,3R,4R,5R)-5-[[Bis(4-methoxyphenyl)-phenyl-methoxy]methyl]-4-[2-cyanoethoxy-(diisopropylamino)phosphanyl]oxy-3-hexadecoxy-tetrahydrofuran-2-yl]-6-oxo-1H-purin-2-yl]-2-methyl-propanamide

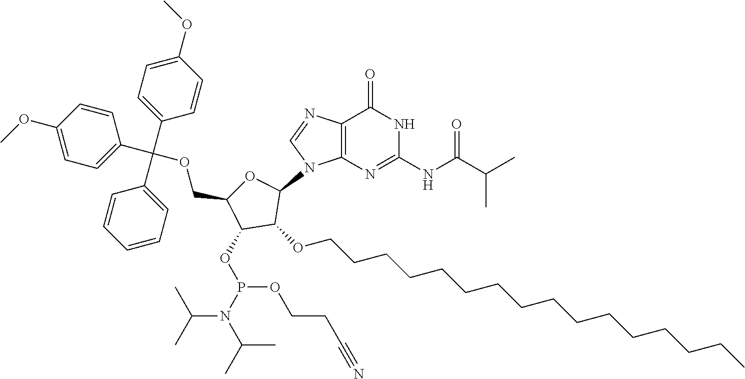 [SMILES: CCCCCCCCCCCCCCCCO[C@@H]1[C@H](OP(OCCC#N)N(C(C)C)C(C)C)[C@@H](COC(c2ccccc2)(c2ccc(OC)cc2)c2ccc(OC)cc2)O[C@H]1n1cnc2c(=O)[nH]c(NC(=O)C(C)C)nc21]

Prepared the title compound according to the protocols described in WO2019217459. 1H-NMR (CDCl3) δ 12.01-11.96 (m, 1H), 7.82-7.78 (m, 1H), 7.59-7.53 (m, 1H), 7.47-7.42 (m, 1H), 7.41-7.37 (m, 2H), 7.34-7.29 (m, 2H), 7.27-7.22 (m, 3H), 6.85-6.80 (m, 4H), 5.99-5.82 (m, 1H), 4.40-4.36 (m, 1H), 4.17-4.11 (m, 1H), 3.80-3.77 (m, 6H), 3.76-3.68 (m, 6H), 3.22-3.17 (m, 1H), 2.84-2.79 (m, 1H), 1.60-1.54 (m, 4H), 1.35-1.30 (m, 6H), 1.27 (s, 19H), 1.24-1.15 (m, 13H), 1.06-1.03 (m, 5H), 0.93-0.88 (m, 6H), 0.74-0.70 (m, 1H). 31P NMR (CDCl3) δ 150.20, 149.92.

### Example 4E: S-mercaptotertbutyl-L-cystine-transcyclohexylamideo phosphoramidite

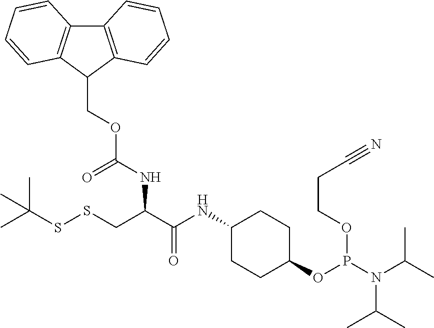 [SMILES: CC(C)N(C(C)C)P(OCCC#N)O[C@H]1CC[C@H](NC(=O)[C@@H](CSSC(C)(C)C)NC(=O)OCC2c3ccccc3-c3ccccc32)CC1]

(9H-fluoren-9-yl)methyl ((2S)-3-(tert-butyldisulfaneyl)-1-(((trans)-4-(((2-cyanoethoxy)(diisopropylamino)phosphaneyl)oxy)cyclohexyl)amino)-1-oxopropan-2-yl)carbamate, referred to herein as S-mercaptotertbutyl-L-cystine-transcyclohexylamido phosphoramidite, was prepared according to the protocol described in Nucleosides, Nucleotides & Nucleic Acids (2000), 19(10-12), 1751-1764.

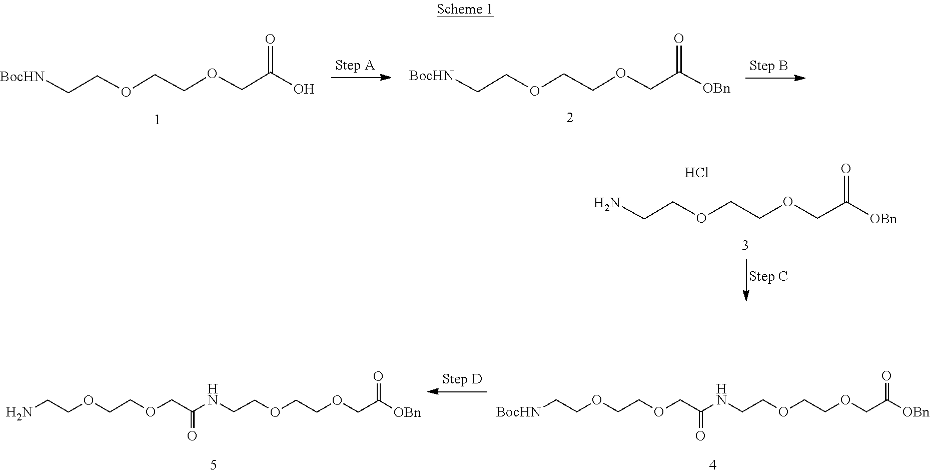 [SMILES: CC(C)(C)OC(=O)NCCOCCOCC(=O)O ; CC(C)(C)OC(=O)NCCOCCOCC(=O)OCc1ccccc1 ; NCCOCCOCC(=O)OCc1ccccc1 ; Cl ; NCCOCCOCC(=O)NCCOCCOCC(=O)OCc1ccccc1 ; CC(C)(C)OC(=O)NCCOCCOCC(=O)NCCOCCOCC(=O)OCc1ccccc1]

Scheme 1, step A shows the alkylative esterification of commercially available 2,2-dimethyl-4-oxo-3,8,11-trioxa-5-azatridecan-13-oic acid (1, CAS number 108466-89-3) which took place utilizing benzyl bromide in acetone in presence of the base potassium carbonate to give compound (2). Step B shows the acidic deprotection of compound (2) which took place by treating with hydrochloric acid in the solvent ethyl acetate to give compound (3). Step C shows the amide coupling of (3) with (1) which took place using the amide coupling reagent EDCI in presence of HOBt and the base DIEA in the solvent DCM to give compound (4). Step D shows the acidic deprotection of compound (4) which took place with hydrochloric acid in the solvent ethyl acetate to give compound (5). One skilled in the art will recognize that a variety of coupling reagents, bases, and solvents can be used to perform an amide coupling, and a variety of acids can be used to perform a BOC deprotection.

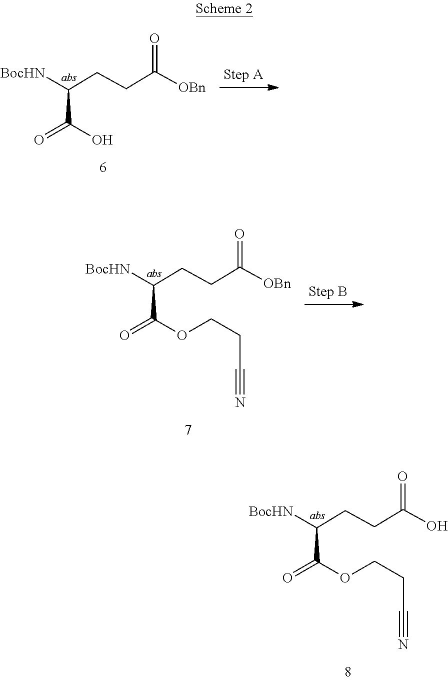 [SMILES: CC(C)(C)OC(=O)N[C@@H](CCC(=O)OCc1ccccc1)C(=O)O ; CC(C)(C)OC(=O)N[C@@H](CCC(=O)OCc1ccccc1)C(=O)OCCC#N ; CC(C)(C)OC(=O)N[C@@H](CCC(=O)O)C(=O)OCCC#N]

Scheme 2, step A shows the coupling of commercially available N-Boc-L-glutamic acid 5-benzyl ester (6, CAS number 13574-13-5) with 3-hydroxypropionitrile which took place under coupling conditions utilizing DCC in presence of the base DMAP in the solvent DCM to give compound (7). Step B shows the hydrogenative debenzylation of compound (7) which took place in presence of hydrogen gas and the catalyst palladium on carbon in the solvent THF to give compound (8). One skilled in the art will recognize that a variety coupling reagents, bases and solvents can be used to perform an ester coupling, and a variety of catalysts and hydrogen sources can be used to perform a benzyl ester removal.

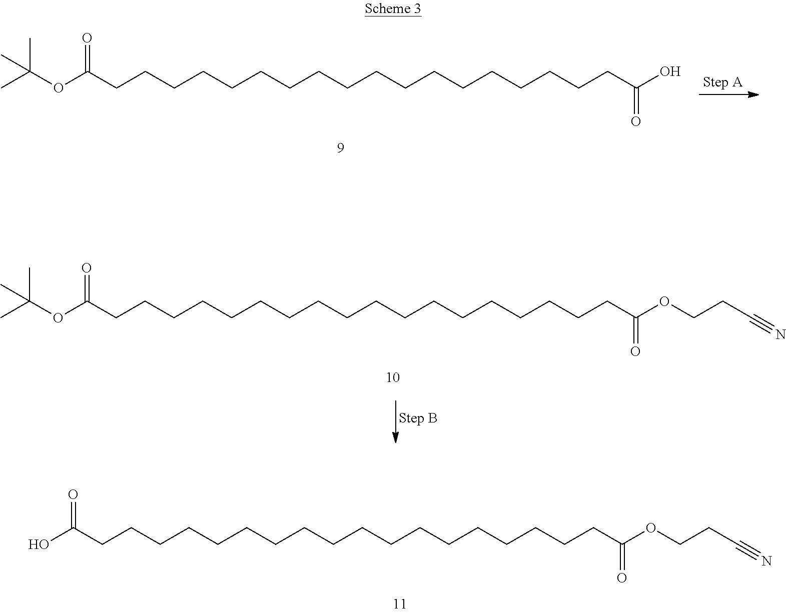 [SMILES: CC(C)(C)OC(=O)CCCCCCCCCCCCCCCCCCC(=O)O ; CC(C)(C)OC(=O)CCCCCCCCCCCCCCCCCCC(=O)OCCC#N ; N#CCCOC(=O)CCCCCCCCCCCCCCCCCCC(=O)O]

Scheme 3, step A shows the coupling of commercially available 20-(tert-Butoxy)-20-oxoicosanoic acid (9, CAS number 683239-16-9) with 3-hydroxypropionitrile which took place under coupling conditions utilizing DCC in presence of the base DMAP in the solvent DCM to give compound (10). Step B shows the acidic deprotection of compound (10) which took place with the acid TFA in the solvent DCM to give compound (11). One skilled in the art will recognize that a variety coupling reagents, bases and solvents can be used to perform an ester coupling, and a variety of acids can be used to perform a BOC deprotection.

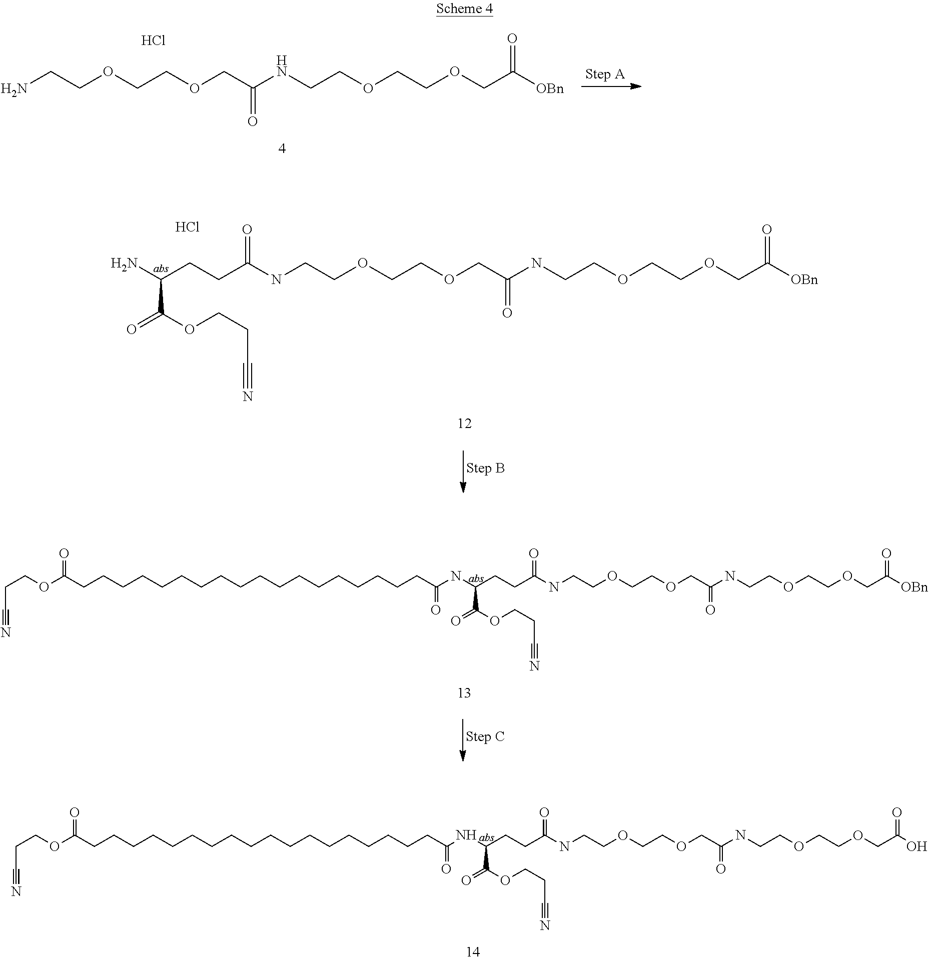

Scheme 4, step A shows the amide coupling of (4) with compound (8) which took place using the amide coupling reagent EDCI in presence of HOBt and the base DIEA in the solvent DCM followed by acidic deprotection with hydrochloric acid in the solvent ethyl acetate to give compound (12). Step B shows the amide coupling of (12) with compound (11) using the amide coupling reagent EDCI in presence of HOBt and the base DIEA in the solvent DCM to give compound (13). Step C shows the hydrogenative debenzylation of compound (13) which took place in presence of hydrogen gas and the catalyst palladium on carbon in the solvent ethyl acetate to give compound (14). One skilled in the art will recognize that a variety coupling reagents, bases and solvents can be used to perform an ester coupling, and a variety of acids can be used to perform a BOC deprotection. One skilled in the art will also recognize a variety of catalysts and hydrogen sources can be used to perform a benzyl ester removal.

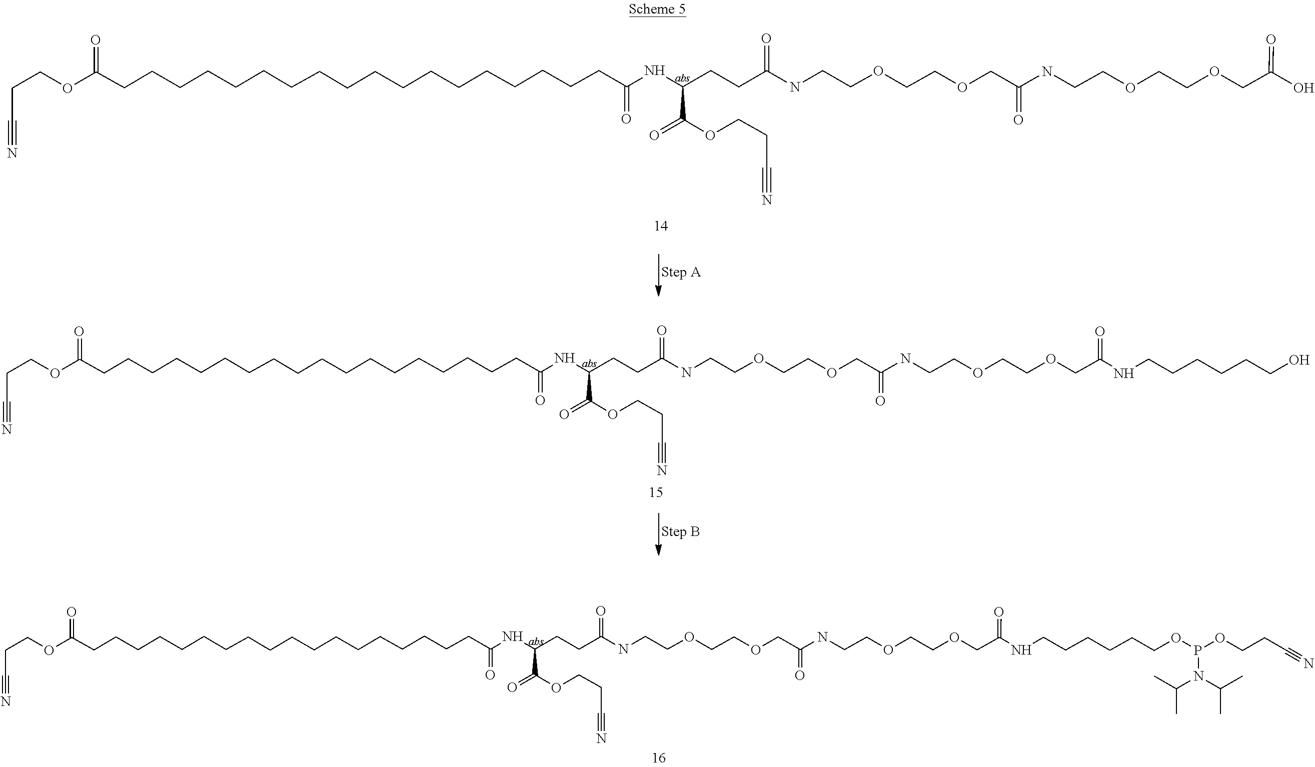

Scheme 5, step A shows the amide coupling of (14) with 6-amino-1-hexanol which took place in presence of the amide coupling reagent EDC in the solvent DCM to give compound (15). Step B shows the conversion of alcohol (15) to a phosphoramidite which took place by treatment with the phosphoramidite precursor 3-((bis(diisopropylamino)phosphaneyl)oxy)propanenitrile in presence of the activator 5-(ethylthio)-1H-tetrazole in the solvent DCM to give compound (16) “C20-Diacid-CE phosphoramidite”. One skilled in the art will recognize a variety of amide coupling reagents and solvents can be used to perform an amide coupling, and a variety of phosphoramidite precursor reagents and activators can be used to form a phosphoramidite.

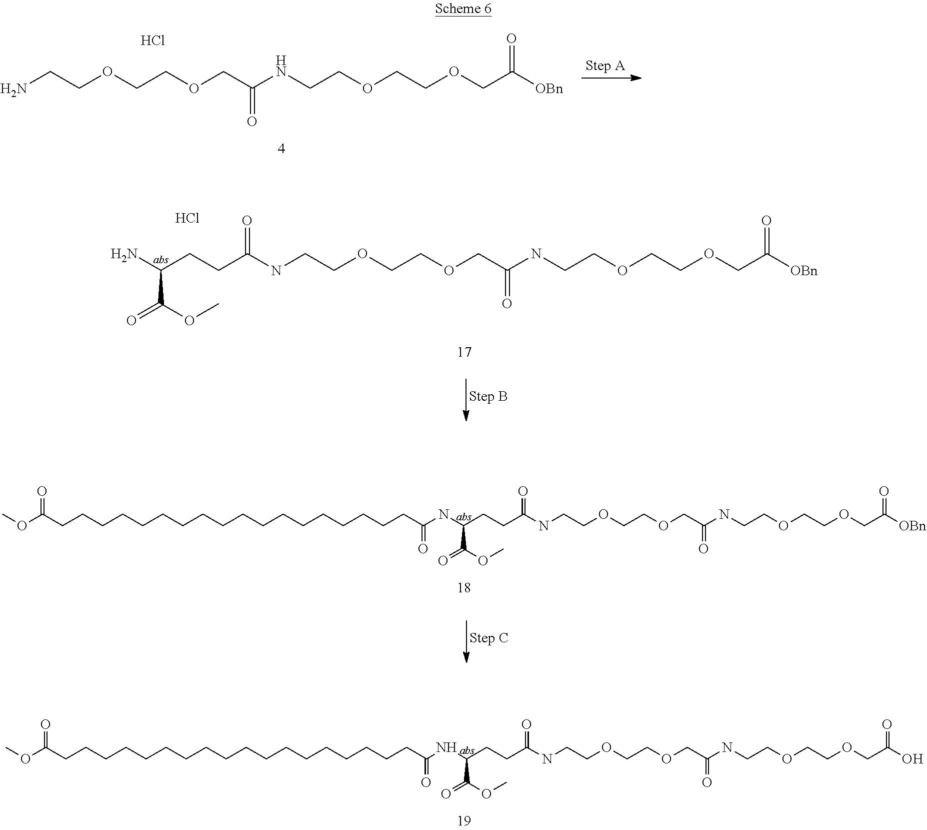

Scheme 6, step A shows the amide coupling of (4) with commercially available N-Boc-L-glutamic acid 5-methyl ester (CAS number 45214-91-3) which took place using the amide coupling reagent EDCI in presence of HOBt and the base DIEA in the solvent DCM followed by acidic deprotection with hydrochloric acid in the solvent ethyl acetate to give compound (17). Step B shows the amide coupling of (17) with commercially available 20-Methoxy-20-oxoicosanoic acid (CAS number 1767-98-2) which took place using the amide coupling reagent EDCI in presence of HOBt and the base DIEA in the solvent DCM to give compound (13). Step C shows the hydrogenative debenzylation of compound (13) which took place in presence of hydrogen gas and the catalyst palladium on carbon in the solvent ethyl acetate to give compound (19). One skilled in the art will recognize that a variety coupling reagents, bases and solvents can be used to perform an ester coupling, and a variety of acids can be used to perform a BOC deprotection. One skilled in the art will also recognize a variety of catalysts and hydrogen sources can be used to perform a benzyl ester removal.

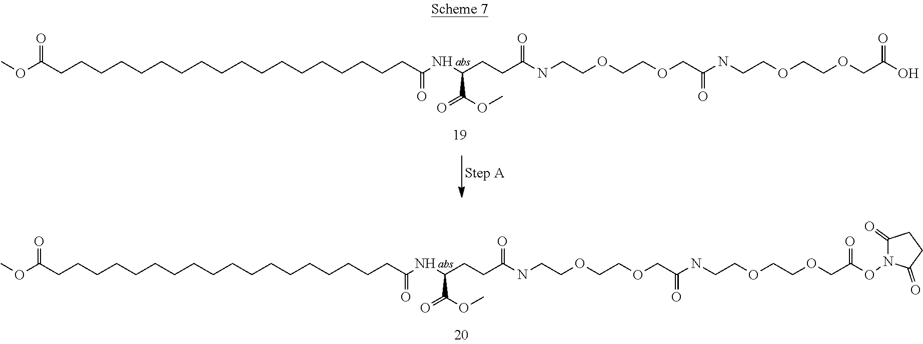

Scheme 7, step A shows the formation of an activated N-hydroxysuccinimide ester by treatment of compound (19) with 1-hydroxypyrrolidine-2,5-dione in presence of a coupling reagent such as EDC in a solvent such as DCM to give compound (20) referred herein as “C20-Diacid-ME NHS Ester.” One skilled in the art will recognize that a variety coupling reagents and solvents can be used to perform an activated ester formation coupling.

### Example 4F: Benzyl 2,2-dimethyl-4-oxo-3,8,11-trioxa-5-azatridecan-13-oate

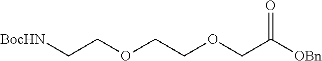 [SMILES: CC(C)(C)OC(=O)NCCOCCOCC(=O)OCc1ccccc1]

To a mixture of 2,2-dimethyl-4-oxo-3,8,11-trioxa-5-azatridecan-13-oic acid (50.0 g, 99% Wt, 1 Eq, 188 mmol) in Acetone (500 mL) were added Benzyl bromide (36.1 g, 25.1 mL, 98% Wt, 1.1 Eq, 207 mmol) and Potassium carbonate (65.6 g, 99% Wt, 2.5 Eq, 470 mmol) at 0° C., then the reaction mixture was stirred at 65° C. for 90 min under N2. This procedure was repeated twice and the reaction mixtures of were combined to work-up and purify. The reaction mixture was filtered and the filter cake was washed with EtOAc (100 mL*3). The filtrate was concentrated under reduced pressure to give a residue. To the residue was added H2O (500 mL) and extracted with EtOAc (500 mL*2). The combined organic layers were washed with brine (1000 mL*2), dried over anhydrous Na2SO4, filtered and concentrated under reduced pressure to give benzyl 2,2-dimethyl-4-oxo-3,8,11-trioxa-5-azatridecan-13-oate (128 g, 0.35 mol, 95%, 97% Purity) as a yellow oil. LCMS m/z=354.1 (M+1). ¹H NMR (400 MHz, DMSO-d₆) δ ppm 7.20-7.47 (m, 5H) 6.73 (br t, J=5.20 Hz, 1H) 5.14 (s, 2H) 4.18 (s, 2H) 3.56-3.64 (m, 2H) 3.47-3.54 (m, 2H) 3.34-3.37 (m, 2H) 3.05 (q, J=6.00 Hz, 2H) 1.36 (s, 9H).

### Example 4G: Benzyl 2-(2-(2-aminoethoxy)ethoxy)acetate hydrochloride

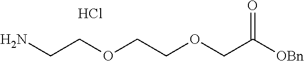 [SMILES: NCCOCCOCC(=O)OCc1ccccc1 ; Cl]

A mixture of benzyl 2,2-dimethyl-4-oxo-3,8,11-trioxa-5-azatridecan-13-oate (60 g, 93% Wt, 1 Eq, 0.16 mol) in 2.0 M HCl in EtOAc (43.8 g, 600 mL, 2 molar, 7.6 Eq, 1.20 mol) was stirred at 26° C. for 60 min. The reaction mixture was concentrated under reduced pressure to give benzyl 2-(2-(2-aminoethoxy)ethoxy)acetate hydrochloride (50 g, 0.16 mol, 99%, 91% Purity) as a pink oil. LCMS m/z=254.1 (M+1).

### Example 4H: benzyl 2,2-dimethyl-4,13-dioxo-3,8,11,17,20-pentaoxa-5,14-diazadocosan-22-oate

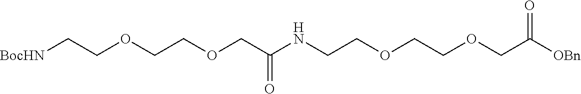 [SMILES: CC(C)(C)OC(=O)NCCOCCOCC(=O)NCCOCCOCC(=O)OCc1ccccc1]

To a mixture of benzyl 2-(2-(2-aminoethoxy)ethoxy)acetate hydrochloride (54 g, 90% Wt, 1 Eq, 0.17 mol) in DCM (500 mL) were added boc-8-amino-3,6-dioxaoctanoic acid (46 g, 40 mL, 95% Wt, 1 Eq, 0.17 mol), 1-(3-Dimethylaminopropyl)-3-ethylcarbodiimideHydrochloride(EDCI) (49 g, 99% Wt, 1.5 Eq, 0.25 mol), 1-Hydroxy-1H-benzotriazole (34 g, 99% Wt, 1.5 Eq, 0.25 mol) and Diisopropylethylamine (0.11 kg, 0.15 L, 99% Wt, 5 Eq, 0.84 mol) at 0° C., then the reaction mixture was stirred at 27° C. for 16 hour. The reaction mixture was concentrated under reduced pressure to give the crude product. The crude product was purified by flash silica gel chromatography (Biotage®; Agela®Flash Column Silica-CS (330×2 g), Eluent of 0-50% EtOAc/petroleum ether gradient @100 mL/min) to give benzyl 2,2-dimethyl-4,13-dioxo-3,8,11,17,20-pentaoxa-5,14-diazadocosan-22-oate (85 g, 0.16 mol, 97%, 95% Purity) as a colorless gel. LCMS m/z=499.3 (M+1).

### Example 41: benzyl 17-amino-10-oxo-3,6,12,15-tetraoxa-9-azaheptadecanoate hydrochloride

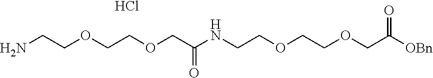 [SMILES: Cl ; NCCOCCOCC(=O)NCCOCCOCC(=O)OCc1ccccc1]

A mixture of benzyl 2,2-dimethyl-4,13-dioxo-3,8,11,17,20-pentaoxa-5,14-diazadocosan-22-oate (60 g, 80% Wt, 1 Eq, 96 mmol) in 2.0 M HCl in EtOAc (43.8 g, 600 mL, 2 molar, 12 Eq, 1.20 mol) was stirred at 26° C. for 60 min. The reaction mixture was concentrated under reduced pressure to give benzyl 17-amino-10-oxo-3,6,12,15-tetraoxa-9-azaheptadecanoate hydrochloride (50 g, 92 mmol, 96%, 80% Purity) as pink gel. LCMS m/z=399.2. ¹H NMR (400 MHz, DMSO-d₆) δ ppm 8.02-8.29 (m, 3H) 7.75 (br t, J=5.20 Hz, 1H) 7.23-7.47 (m, 5H) 5.14 (s, 2H) 4.19 (s, 2H) 3.89 (s, 2H) 3.58-3.65 (m, 8H) 3.50-3.54 (m, 2H) 3.43 (t, J=6.00 Hz, 2H) 3.26 (q, J=6.00 Hz, 2H) 2.90-2.99 (m, 2H).

### Example 4J: 5-benzyl 1-(2-cyanoethyl) (tert-butoxycarbonyl)-L-glutamate

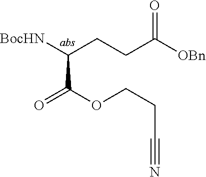 [SMILES: CC(C)(C)OC(=O)N[C@@H](CCC(=O)OCc1ccccc1)C(=O)OCCC#N]

To a solution of (S)-5-(benzyloxy)-2-((tert-butoxycarbonyl)amino)-5-oxopentanoic acid (50.0 g, 1 Eq, 148 mmol) in DCM (500 mL) were added 4-Dimethylaminopyridine (3.62 g, 0.2 Eq, 29.6 mmol) and Dicyclohexylcarbodiimide (61.2 g, 2 Eq, 296 mmol) and Ethylene cyanohydrin (12.6 g, 12.1 mL, 1.2 Eq, 178 mmol) at 0° C. The solution was stirred at 26° C. for 16 hour. The reaction mixture was filtered and the filter cake was washed with DCM (100 mL*3). The filtrate was concentrated under reduced pressure to give the crude product. The crude product was purified by flash silica gel chromatography (Biotage®; Agela®Flash Column Silica-CS (330 g), Eluent of 0~30% EtOAc/hexanes gradient @100 mL/min) to give 5-benzyl 1-(2-cyanoethyl) (tert-butoxycarbonyl)-L-glutamate (50 g, 0.10 mol, 69%, 80% Purity) as a white solid. LCMS m/z=291.1 (M+1-100). ¹H NMR (400 MHz, DMSO-d₆) δ ppm 7.30-7.39 (m, 5H) 5.09 (s, 2H) 4.14-4.34 (m, 2H) 3.98-4.11 (m, 1H) 2.86 (t, J=6.00 Hz, 2H) 2.44-2.52 (m, 2H) 1.95-2.10 (m, 1H) 1.74-1.92 (m, 1H) 1.28-1.46 (m, 9H).

### Example 4K: (S)-4-((tert-butoxycarbonyl)amino)-5-(2-cyanoethoxy)-5-oxopentanoic acid

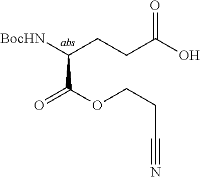 [SMILES: CC(C)(C)OC(=O)N[C@@H](CCC(=O)O)C(=O)OCCC#N]

A solution of 5-benzyl 1-(2-cyanoethyl) (tert-butoxycarbonyl)-L-glutamate (50 g, 80% Wt, 1 Eq, 0.10 mol) in THF (500 mL) was degassed with Argon, and Pd/C(dry basis) (25 g, 10% Wt, 0.23 Eq, 23 mmol) was added. The reaction mixture was evacuated and backfilled three times with hydrogen. The mixture was stirred at 25° C. for 2 hour under an atmosphere of hydrogen 15 psi. Upon completion, the reaction mixture was filtered through a pad of celite and the filtrate was concentrated under reduced pressure to afford the crude product. Pd/C is recycled into an activated metal recycling bucket. The crude product was purified by flash silica gel chromatography (Biotage®; Agela®Flash Column Silica-CS (220 g), Eluent of 0-50% EtOAc/hexanes gradient @100 mL/min) to give (S)-4-((tert-butoxycarbonyl)amino)-5-(2-cyanoethoxy)-5-oxopentanoic acid (25 g, 75 mmol, 73%, 90% Purity) as colorless oil. ¹H NMR (400 MHz, METHANOL-d₄) δ ppm 4.26-4.38 (m, 2H) 4.19 (dd, J=9.20, 5.20 Hz, 1H) 2.85 (t, J=6.00 Hz, 2H) 2.42 (t, J=7.60 Hz, 2H) 2.15 (m, 1H) 1.80-1.98 (m, 1H) 1.44 (s, 9H).

### Example 4L: 1-(tert-butyl) 20-(2-cyanoethyl) icosanedioate

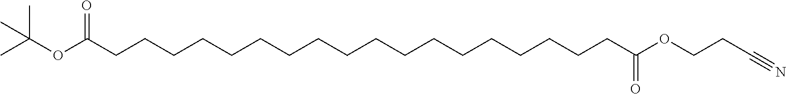 [SMILES: CC(C)(C)OC(=O)CCCCCCCCCCCCCCCCCCC(=O)OCCC#N]

To a mixture of 20-(tert-butoxy)-20-oxoicosanoic acid (25.0 g, 1 Eq, 62.7 mmol) in DCM (300 mL) were added Ethylene cyanohydrin (8.92 g, 8.56 mL, 2 Eq, 125 mmol), DCC (25.9 g, 2 Eq, 125 mmol) and DMAP (1.53 g, 0.2 Eq, 12.5 mmol) at 0° C., then the reaction mixture was stirred at 27° C. for 16 hour under N2. The reaction mixture was filtered and the filter cake was washed with DCM (100 mL*3). The filtrate was concentrated under reduced pressure to give the crude product. The crude product was purified by flash silica gel chromatography (Biotage®; Agela®Flash Column Silica-CS (220 g), Eluent of 0-10% EtOAc/hexanes gradient @100 mL/min) to give 1-(tert-butyl) 20-(2-cyanoethyl) icosanedioate (20 g, 40 mmol, 64%, 90% Purity) as a white solid. ¹H NMR (400 MHz, CHLOROFORM-d) δ ppm 4.27 (t, J=6.00 Hz, 2H) 2.70 (t, J=6.40 Hz, 2H) 2.34 (t, J=7.20 Hz, 2H) 2.18 (t, J=7.60 Hz, 2H) 1.60-1.67 (m, 2H) 1.52-1.59 (m, 2H) 1.43 (s, 9H) 1.22-1.31 (m, 28H).

### Example 4M: 20-(2-cyanoethoxy)-20-oxoicosanoic acid

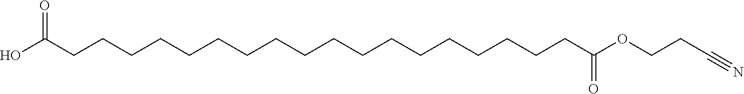 [SMILES: N#CCCOC(=O)CCCCCCCCCCCCCCCCCCC(=O)O]

To a solution of 1-(tert-butyl) 20-(2-cyanoethyl) icosanedioate (17 g, 90% Wt, 1 Eq, 34 mmol) in DCM (150 mL) was added TFA (3.9 g, 90 mL, 1 Eq, 34 mmol). The reaction mixture was stirred at 26° C. for 60 min. The reaction mixture was concentrated under reduced pressure to give 20-(2-cyanoethoxy)-20-oxoicosanoic acid (15 g, 34 mmol, 100%, 90% Purity) as a pink solid. ¹H NMR (400 MHz, DMSO-d₆) δ ppm 4.14-4.21 (m, 2H) 2.86 (t, J=6.00 Hz, 1H) 2.32 (t, J=7.20 Hz, 2H) 2.17 (br t, J=7.20 Hz, 2H) 1.50 (td, J=13.60, 7.20 Hz, 4H) 1.23 (s, 28 H).

### Example 4N: 11-benzyl 23-(2-cyanoethyl) (S)-22-((tert-butoxycarbonyl)amino)-10,19-dioxo-3,6,12,15-tetraoxa-9,18-diazatricosanedioate

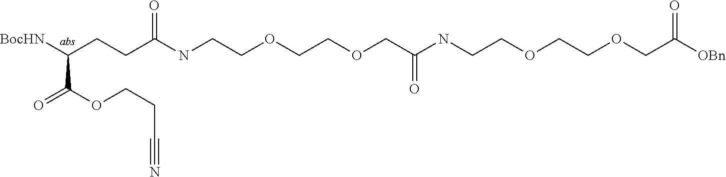 [SMILES: CC(C)(C)OC(=O)N[C@@H](CCC(=O)NCCOCCOCC(=O)NCCOCCOCC(=O)OCc1ccccc1)C(=O)OCCC#N]

To a mixture of benzyl 17-amino-10-oxo-3,6,12,15-tetraoxa-9-azaheptadecanoate hydrochloride (45 g, 80% Wt, 1 Eq, 83 mmol) in DCM (500 mL) were added DIEA (32 g, 43 mL, 3 Eq, 0.25 mol), (S)-4-((tert-butoxycarbonyl)amino)-5-(2-cyanoethoxy)-5-oxopentanoic acid (25 g, 90% Wt, 0.91 Eq, 75 mmol), 1-(3-Dimethylaminopropyl)-3-ethylcarbodiimide hydrochloride(EDCI) (24 g, 1.5 Eq, 0.12 mol) and 1-hydroxy-1H-benzotriazole (17 g, 1.5 Eq, 0.12 mol) at 0° C., then the reaction mixture was stirred at 26° C. for 16 hour. The reaction mixture was concentrated under reduced pressure to give the residue. The residue was added to H2O (500 mL) and extracted with EtOAc (400 mL*2). The combined organic layers were washed with brine (400 mL*2), dried over anhydrous Na2SO4, filtered and concentrated under reduced pressure to give a crude product. The crude product was purified by flash silica gel chromatography (Biotage®; Agela®Flash Column Silica-CS (330 g), Eluent of 0-100% EtOAc/hexanes gradient @100 mL/min) to give 1-benzyl 23-(2-cyanoethyl) (S)-22-((tert-butoxycarbonyl)amino)-10,19-dioxo-3,6,12,15-tetraoxa-9,18-diazatricosanedioate (40 g, 52 mmol, 62%, 88% Purity) as a yellow oil. LCMS m/z=681.3 (M+1). ¹H NMR (400 MHz, DMSO-d₆) δ ppm 7.85 (br t, J=5.20 Hz, 1H) 7.63 (br t, J=5.60 Hz, 1H) 7.25-7.41 (m, 5H) 5.15 (s, 2H) 4.15-4.30 (m, 4H) 3.91-4.01 (m, 1H) 3.87 (s, 2H) 3.50-3.63 (m, 8H) 3.42 (dt, J=11.60, 6.00 Hz, 4H) 3.32 (s, 2H) 3.26 (q, J=6.00 Hz, 2H) 3.20 (q, J=5.60 Hz, 2H) 2.87 (t, J=5.60 Hz, 2H) 2.18 (br t, J=7.60 Hz, 2H) 1.87-1.98 (m, 1H) 1.76 (m, 1H) 1.38 (s, 9H).

### Example 40: 1-benzyl 23-(2-cyanoethyl) (S)-22-amino-10,19-dioxo-3,6,12,15-tetraoxa-9,18-diazatricosanedioate hydrochloride

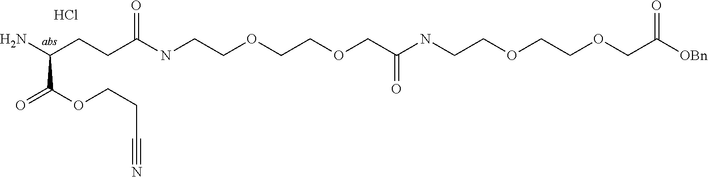 [SMILES: N#CCCOC(=O)[C@@H](N)CCC(=O)NCCOCCOCC(=O)NCCOCCOCC(=O)OCc1ccccc1 ; Cl]

A mixture of 1-benzyl 23-(2-cyanoethyl) (S)-22-((tert-butoxycarbonyl)amino)-10,19-dioxo-3,6,12,15-tetraoxa-9,18-diazatricosanedioate (38 g, 88% Wt, 1 Eq, 49 mmol) in 2.0 M HCl in EtOAc (29.2 g, 400 mL, 2 molar, 16 Eq, 800 mmol) was stirred at 27° C. for 60 min. The reaction mixture was concentrated under reduced pressure to give 1-benzyl 23-(2-cyanoethyl) (S)-22-amino-10,19-dioxo-3,6,12,15-tetraoxa-9,18-diazatricosanedioate hydrochloride (34 g, 45 mmol, 92%, 82% Purity) as a red oil. LCMS m/z=581.4 (M+1). ¹H NMR (400 MHz, DMSO-d₆) δ ppm 8.59-8.85 (m, 3H) 8.11 (br t, J=5.60 Hz, 1H) 7.69 (br t, J=5.60 Hz, 1H) 7.17-7.46 (m, 5H) 5.14 (s, 2H) 4.25-4.40 (m, 2H) 4.19 (s, 2H) 3.87 (s, 2H) 3.49-3.64 (m, 9H) 3.39-3.45 (m, 4H) 3.17-3.29 (m, 4H) 2.96 (t, J=6.00 Hz, 2H) 2.23-2.41 (m, 2H) 1.99-2.06 (m, 2H).

### Example 4P: 1-benzyl 21,41-bis(2-cyanoethyl) (S)-9,18,23-trioxo-2,5,11,14-tetraoxa-8,17,22-triazahentetracontane-1,21,41-tricarboxylate

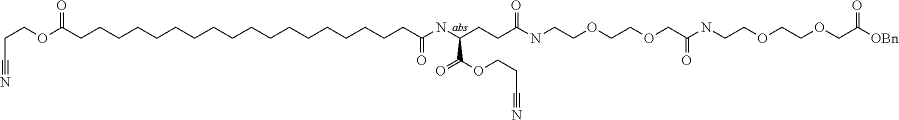

To a mixture of 1-benzyl 23-(2-cyanoethyl) (S)-22-amino-10,19-dioxo-3,6,12,15-tetraoxa-9,18-diazatricosanedioate hydrochloride (20.5 g, 82% Wt, 1 Eq, 27.2 mmol) in DCM (300 mL) were added N-ethyl-N-isopropylpropan-2-amine (14.1 g, 4 Eq, 109 mmol), 20-(2-cyanoethoxy)-20-oxoicosanoic acid (12.0 g, 90% Wt, 1 Eq, 27.2 mmol), 1H-benzo[d][1,2,3]triazol-1-ol (5.52 g, 1.5 Eq, 40.9 mmol) and 3-(((ethylimino)methylene)amino)-N,N-dimethylpropan-1-amine hydrochloride (7.83 g, 1.5 Eq, 40.9 mmol) at 26° C., then the reaction mixture was stirred at 26° C. for 16 hour. The reaction mixture was concentrated under reduced pressure to give the residue. The residue was added to H2O (500 mL) and extracted with EtOAc (400 mL*2). The combined organic layers were washed with brine (400 mL*2), dried over anhydrous Na2SO4, filtered and concentrated under reduced pressure to give a crude product. The crude product was purified by flash silica gel chromatography (Biotage®; Agela®Flash Column Silica-CS (220 g), Eluent of 0-10% MeOH/DCM gradient @100 mL/min) to give 1-benzyl 21,41-bis(2-cyanoethyl) (S)-9,18,23-trioxo-2,5,11,14-tetraoxa-8,17,22-triazahentetracontane-1,21,41-tricarboxylate (24 g, 18 mmol, 66%, 72% Purity) as a white solid. LCMS m/z=958.5 (M+1). ¹H NMR (400 MHz, DMSO-d₆) δ ppm 8.22 (d, J=7.20 Hz, 1H) 7.86 (br t, J=5.20 Hz, 1H) 7.64 (br t, J=5.60 Hz, 1H) 7.22-7.44 (m, 5H) 5.14 (s, 2H) 4.17-4.21 (m, 5H) 3.86 (s, 2H) 3.48-3.64 (m, 9H) 3.40-3.47 (m, 4H) 3.13-3.30 (m, 5H) 2.86 (td, J=6.00, 2.80 Hz, 4H) 2.32 (t, J=7.60 Hz, 2H) 2.14-2.20 (m, 2H) 2.10 (t, J=7.60 Hz, 2H) 1.90-1.99 (m, 1H) 1.74-1.85 (m, 1H) 1.43-1.56 (m, 4H) 1.22 (s, 28H).

### Example 4Q: (S)-46-cyano-22-((2-cyanoethoxy)carbonyl)-10,19,24,43-tetraoxo-3,6,12,15,44-pentaoxa-9,18,23-triazahexatetracontanoic acid

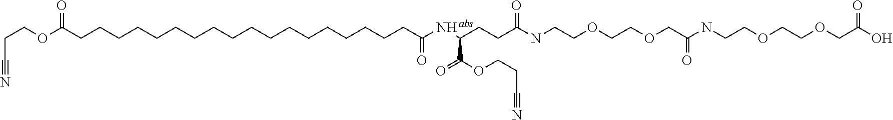

A solution of 1-benzyl 21,41-bis(2-cyanoethyl) (S)-9,18,23-trioxo-2,5,11,14-tetraoxa-8,17,22-triazahentetracontane-1,21,41-tricarboxylate (12.0 g, 72% Wt, 1 Eq, 9.02 mmol) in THF (120 mL) was degassed with Argon and Pd/C(wet basis) (3.0 g, 10% Wt, 0.31 Eq, 2.8 mmol) was added. The reaction mixture was evacuated and backfilled three times with hydrogen. The mixture was stirred at 26° C. for 30 min under an atmosphere of hydrogen 15 psi. Upon completion, the reaction mixture was filtered through a pad of celite and the filtrate was concentrated under reduced pressure to afford the crude product. Pd/C is recycled into an activated metal recycling bucket. The crude product was purified by flash silica gel chromatography (Biotage®; Agela®Flash Column Silica-CS (220 g), Eluent of 0-10% MeOH/DCM gradient @60 mL/min) to give (S)-46-cyano-22-((2-cyanoethoxy)carbonyl)-10,19,24,43-tetraoxo-3,6,12,15,44-pentaoxa-9,18,23-triazahexatetracontanoic acid (11694.41 mg, 13.3 mmol, 147%, 98.4% Purity) as a white solid. LCMS m/z=869.0 (M+1). ¹H NMR (400 MHz, METHANOL-d₄) δ ppm 4.24-4.42 (m, 5H) 4.12 (s, 2H) 4.01 (s, 2H) 3.63-3.72 (m, 8H) 3.58 (dt, J=10.40, 4.80 Hz, 4H) 3.35-3.48 (m, 4H) 2.80-2.88 (m, 3H) 2.22-2.42 (m, 6H) 1.92-2.21 (m, 2H) 1.55-1.68 (m, 4H) 1.18-1.41 (m, 29H).

### Example 4R: 2-cyanoethyl (S)-29-((2-cyanoethoxy)carbonyl)-1-hydroxy-8,17,26,31-tetraoxo-10,13,19,22-tetraoxa-7,16,25,30-tetraazapentacontan-50-oate

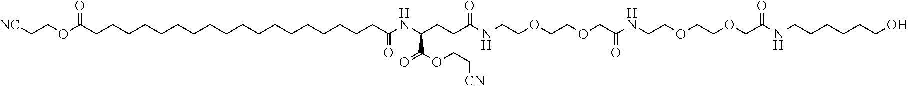 [SMILES: N#CCCOC(=O)CCCCCCCCCCCCCCCCCCC(=O)N[C@@H](CCC(=O)NCCOCCOCC(=O)NCCOCCOCC(=O)NCCCCCCO)C(=O)OCCC#N]

A solution of (S)-46-cyano-22-((2-cyanoethoxy)carbonyl)-10,19,24,43-tetraoxo-3,6,12,15,44-pentaoxa-9,18,23-triazahexatetracontanoic acid (3.95 g, 4.55 mmol), EDC (1.05 g, 5.46 mmol), 6-amino-1-hexanol (0.59 g, 5.01 mmol), and DCM (23 mL) was stirred at ambient temperature for 18 h. The crude reaction was concentrated, the residue was dissolved in 3:1 CHCl₃/i-PrOH (150 mL) and washed with brine (acidified to pH 3 with 1 N HCl). The organic layer was isolated, dried (MgSO₄), and concentrated to a light yellow, waxy solid (3.20 g, 73%), 2-cyanoethyl (29S)-1-(((2-cyanoethoxy)(diisopropylamino)phosphaneyl)oxy)-29-((2-cyanoethoxy)carbonyl)-8,17,26,31-tetraoxo-10,13,19,22-tetraoxa-7,16,25,30-tetraazapentacontan-50-oate. This was used in the next preparation without further purification or characterization.

### Example 4S: C20-Diacid-CE phosphoramidite 2-cyanoethyl (29S)-1-(((2-cyanoethoxy)(diisopropylamino)phosphaneyl)oxy)-29-((2-cyanoethoxy)carbonyl)-8,17,26,31-tetraoxo-10,13,19,22-tetraoxa-7,16,25,30-tetraazapentacontan-50-oate

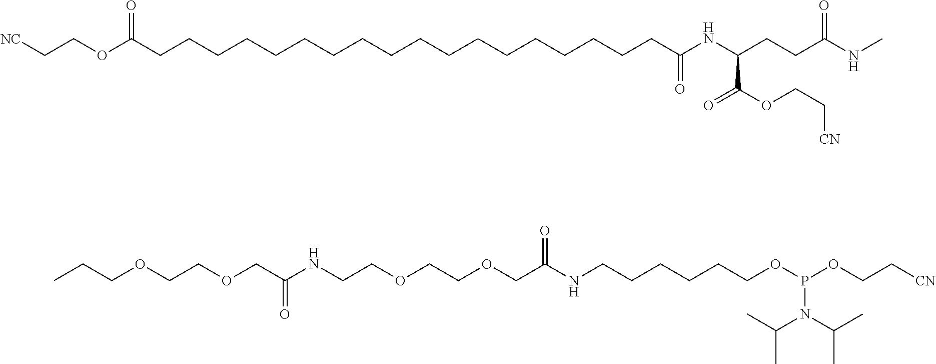

A solution of 2-cyanoethyl (S)-29-((2-cyanoethoxy)carbonyl)-1-hydroxy-8,17,26,31-tetraoxo-10,13,19,22-tetraoxa-7,16,25,30-tetraazapentacontan-50-oate (3.20 g, 3.31 mmol), 5-(ethylthio)-1H-tetrazole (0.25 M in MeCN; 6.6 mL, 1.65 mmol), 3-((bis(diisopropylamino)phosphaneyl)oxy)propanenitrile (1.26 mL, 3.97 mmol), and DCM (20 mL) was stirred at ambient temperature. After 1.5 hours, the crude reaction was poured into a slurry of silica gel (15 g) in DCM (30 mL), concentrated in vacuo to a dry powder, and purified via silica gel flash chromatography eluting with 5-30% MeOH/EtOAc to give 2-cyanoethyl (29S)-1-(((2-cyanoethoxy)(diisopropylamino)phosphaneyl)oxy)-29-((2-cyanoethoxy)carbonyl)-8,17,26,31-tetraoxo-10,13,19,22-tetraoxa-7,16,25,30-tetraazapentacontan-50-oate as a sticky, white foam (1.40 g, 36%). ¹H NMR (d6-DMSO) d 8.22 (d, 1H), 7.88 (t, 1H), 7.70-7.60 (m, 2 H), 4.29-4.12 (m, 5H), 3.88 (s, 2H), 3.85 (s, 2H), 3.82-3.49 (m, 14H), 3.49-3.37 (m, 4H), 3.32-3.25 m, 2H), 3.24-3.16 (m, 2H), 3.13-3.04 (m, 2H), 2.91-2.84 (m, 4H), 2.76 (t, 2H), 2.33 (t, 2 H), 2.18 (t, 2H), 2.11 (t, 2H), 1.99-1.90 (m, 1H), 1.86-1.74 (m, 1H), 1.59-1.04 (m, 52H). ³¹P NMR (d6-DMSO) d 146.3.

### Example 4T: 1-benzyl 23-methyl (S)-22-((tert-butoxycarbonyl)amino)-10,19-dioxo-3,6,12,15-tetraoxa-9,18-diazatricosanedioate

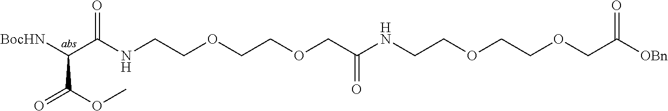 [SMILES: COC(=O)[C@@H](NC(=O)OC(C)(C)C)C(=O)NCCOCCOCC(=O)NCCOCCOCC(=O)OCc1ccccc1]

To a mixture of benzyl 17-amino-10-oxo-3,6,12,15-tetraoxa-9-azaheptadecanoate hydrochloride (35 g, 73% Wt, 1 Eq, 59 mmol) in DCM (350 mL) was added (S)-4-((tert-butoxycarbonyl)amino)-5-methoxy-5-oxopentanoic acid (16 g, 99% Wt, 1 Eq, 59 mmol, CAS number 45214-91-3), 1-(3-Dimethylaminopropyl)-3-ethylcarbodiimideHydrochloride(EDCI) (17 g, 99% Wt, 1.5 Eq, 88 mmol), 1-Hydroxy-1H-benzotriazole (12 g, 99% Wt, 1.5 Eq, 88 mmol) and Diisopropylethylamine (31 g, 41 mL, 99% Wt, 4 Eq, 0.23 mol) at 0° C., then the reaction mixture was stirred at 26° C. for 16 hour. The reaction mixture was concentrated under reduced pressure to give the residue. To the residue was added H2O (500 mL) and extracted with EtOAc (300 mL*2). The combined organic layers were washed with brine (500 mL*2), dried over anhydrous Na2SO4, filtered and concentrated under reduced pressure to give a crude product. The crude product was purified by flash silica gel chromatography (Biotage®; Agela®Flash Column Silica-CS (220 g), Eluent of 0-100% EtOAc/petroleum ether gradient @100 mL/min) to give 1-benzyl 23-methyl (S)-22-((tert-butoxycarbonyl)amino)-10,19-dioxo-3,6,12,15-tetraoxa-9,18-diazatricosanedioate (25.5 g, 38 mmol, 65%, 96% Purity) as colorless gel. LCMS m/z=642.3 (M+1)¹H NMR (400 MHz, METHANOL-d₄) δ ppm 7.24-7.44 (m, 5H) 5.19 (s, 2H) 4.21 (s, 2H) 3.98 (s, 2H) 3.69-3.73 (m, 5H) 3.60-3.68 (m, 7H) 3.55 (dt, J=10.80, 5.20 Hz, 4H) 3.40-3.46 (m, 2H) 3.34-3.39 (m, 2H) 2.30 (br t, J=7.20 Hz, 2H) 2.05-2.18 (m, 1H) 1.79-1.94 (m, 1H) 1.43 (s, 9H).

### Example 4U: 1-benzyl 23-methyl (S)-22-amino-10,19-dioxo-3,6,12,15-tetraoxa-9,18-diazatricosanedioate hydrochloride

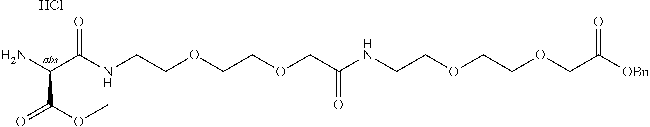 [SMILES: Cl ; COC(=O)[C@@H](N)C(=O)NCCOCCOCC(=O)NCCOCCOCC(=O)OCc1ccccc1]

To a mixture of 1-benzyl 23-methyl (S)-22-((tert-butoxycarbonyl)amino)-10,19-dioxo-3,6,12,15-tetraoxa-9,18-diazatricosanedioate (25.5 g, 96% Wt, 1 Eq, 38.1 mmol) in EtOAc (50 mL) was added to 2M Hydrogen chloride in ethyl acetate (13.9 g, 191 mL, 2 molar, 10 Eq, 381 mmol) at 0° C. Then the reaction mixture was stirred at 26° C. for 60 min. The reaction mixture was concentrated under reduced pressure to give 1-benzyl 23-methyl (S)-22-amino-10,19-dioxo-3,6,12,15-tetraoxa-9,18-diazatricosanedioate hydrochloride (25 g, 38 mmol, 100%, 89% Purity) as a pink gel. LCMS m/z=542.3(M+1). ¹H NMR (400 MHz, METHANOL-d₄) δ ppm 7.30-7.39 (m, 5H) 4.60 (s, 2H) 4.17 (s, 2H) 4.05 (s, 2H) 3.84 (s, 3H) 3.69-3.71 (m, 4H) 3.64-3.67 (m, 4H) 3.56-3.61 (m, 6H) 3.44-3.48 (m, 2H) 3.40 (t, J=5.60 Hz, 2H) 2.49-2.54 (m, 2H) 2.15-2.26 (m, 2H).

### Example 4V: 1-benzyl 21,41-dimethyl (S)-9,18,23-trioxo-2,5,11,14-tetraoxa-8,17,22-triazahentetracontane-1,21,41-tricarboxylate

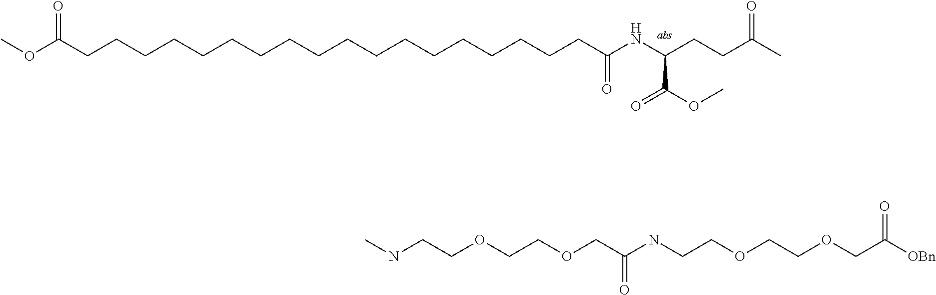

To a mixture of 1-benzyl 23-methyl (S)-22-amino-10,19-dioxo-3,6,12,15-tetraoxa-9,18-diazatricosanedioate hydrochloride (25.0 g, 89% Wt, 1 Eq, 38.5 mmol) in DCM (250 mL) were added 20-methoxy-20-oxoicosanoic acid (13.9 g, 99% Wt, 1 Eq, 38.5 mmol, CAS 1767-98-2), 1-(3-Dimethylaminopropyl)-3-ethylcarbodiimideHydrochloride(EDCI) (11.2 g, 99% Wt, 1.5 Eq, 57.7 mmol), 1-Hydroxy-1H-benzotriazole (7.88 g, 99% Wt, 1.5 Eq, 57.7 mmol) and Diisopropylethylamine (25.1 g, 33.5 mL, 99% Wt, 5 Eq, 192 mmol) at 0° C., then the reaction mixture was stirred at 26° C. for 16 hour. The reaction mixture was concentrated under reduced pressure to give the residue. To the residue was added H2O (300 mL) and extracted with EtOAc (300 mL*2). The combined organic layers were washed with brine (300 mL*2), dried over anhydrous Na2SO4, filtered and concentrated under reduced pressure to give a crude product. The crude product was purified by flash silica gel chromatography (Biotage®; Agela®Flash Column Silica-CS (120 g), Eluent of 0-7% MeOH/DCM gradient @80 mL/min) to give 1-benzyl 21,41-dimethyl (S)-9,18,23-trioxo-2,5,11,14-tetraoxa-8,17,22-triazahentetracontane-1,21,41-tricarboxylate (35 g, 39 mmol, 100%, 97% Purity) as a white solid. LCMS m/z=880.6 (M+1). ¹H NMR (400 MHz, DMSO-d₆) δ ppm 8.17 (d, J=7.20 Hz, 1H) 7.88 (br t, J=5.20 Hz, 1H) 7.64 (br t, J=5.60 Hz, 1H) 7.22-7.49 (m, 5H) 5.14 (s, 2H) 4.14-4.22 (m, 3H) 3.86 (s, 2H) 3.50-3.59 (m, 19H) 3.16-3.29 (m, 5H) 2.27 (t, J=7.60 Hz, 2H) 2.11 (m, 4H) 1.43-1.54 (m, 4H) 1.22 (s, 28H).

### Example 4W: (S)-24-(methoxycarbonyl)-3,22,27,36-tetraoxo-2,31,34,40,43-pentaoxa-23,28,37-triazapentatetracontan-45-oic acid

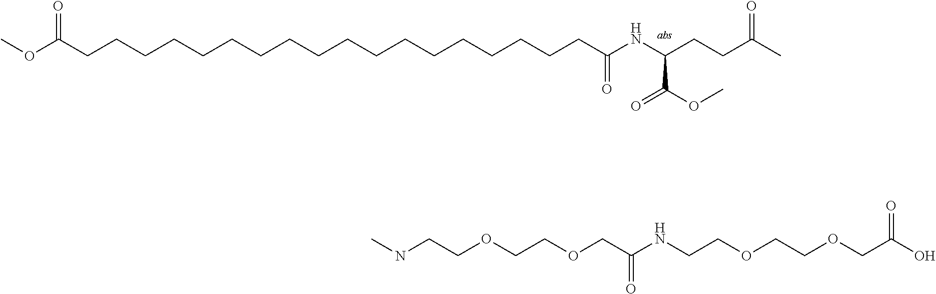

A solution of 1-benzyl 21,41-dimethyl (S)-9,18,23-trioxo-2,5,11,14-tetraoxa-8,17,22-triazahentetracontane-1,21,41-tricarboxylate (30.0 g, 97% Wt, 1 Eq, 33.1 mmol) in THF (300 mL) was degassed with Argon, and Pd/C, wet basis (14.1 g, 10% Wt, 0.4 Eq, 13.2 mmol) was added. The reaction mixture was evacuated and backfilled three times with hydrogen. The mixture was stirred at 25° C. for 120 min under an atmosphere of hydrogen 15 psi. The reaction mixture was filtered through a pad of celite and the filtrate was concentrated under reduced pressure to afford the crude product. Pd/C is recycled into a special recycling bucket. The crude product was purified by flash silica gel chromatography (Biotage®; Agela®Flash Column Silica-CS (120 g), Eluent of 0-15% MeOH/DCM gradient @80 mL/min) to give (S)-24-(methoxycarbonyl)-3,22,27,36-tetraoxo-2,31,34,40,43-pentaoxa-23,28,37-triazapentatetracontan-45-oic acid (17493.85 mg, 21.4 mmol, 64.8%, 96.8% Purity) as a white solid. LCMS m/z=791.0 (M+1). ¹H NMR (400 MHz, METHANOL-d₄) δ ppm 4.39 (dd, J=8.80, 5.20 Hz, 1H) 4.11 (s, 2H) 4.01 (s, 2H) 3.62-3.74 (m, 14H) 3.57 (dt, J=10.80, 5.20 Hz, 4H) 3.42-3.48 (m, 2H) 3.35-3.40 (m, 2H) 2.31 (td, J=7.20, 2.80 Hz, 4H) 2.24 (br t, J=7.60 Hz, 2H) 2.13 (m, 1H) 1.90-2.01 (m, 1H) 1.54-1.68 (m, 4H) 1.29 (br s, 28H).

### Example 4X: C20-Diacid-ME NHS Ester 1-(2,5-dioxopyrrolidin-1-yl) 21,41-dimethyl (S)-9,18,23-trioxo-2,5,11,14-tetraoxa-8,17,22-triazahentetracontane-1,21,41-tricarboxylate

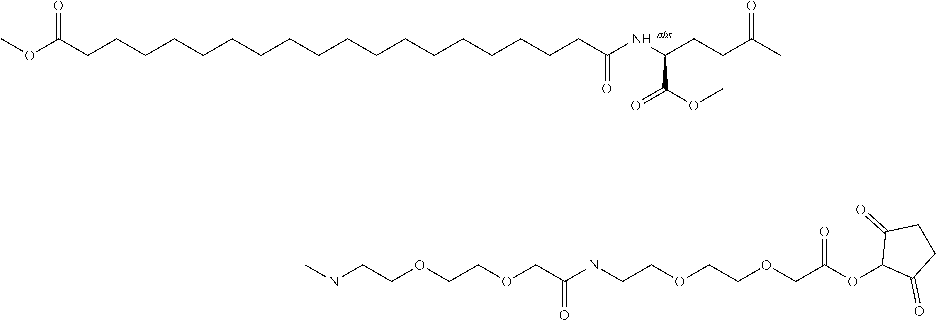

A solution of (S)-24-(methoxycarbonyl)-3,22,27,36-tetraoxo-2,31,34,40,43-pentaoxa-23,28,37-triazapentatetracontan-45-oic acid (500 mg, 1 Eq, 633 mol) lOmL of DCM, 1-hydroxypyrrolidine-2,5-dione (109 mg, 1.5 Eq, 949 mol) and 3-(((ethylimino)methylene)amino)-N,N-dimethylpropan-1-amine hydrochloride (121 mg, 1 Eq, 633 mol), was stirred at room temperature for 18 hours. The crude reaction was concentrated, and loaded onto 12g silica cartridge, purified via silica gel flash chromatography eluting with 0-30% MeOH/EtOAc to give 1-(2,5-dioxopyrrolidin-1-yl) 21,41-dimethyl (S)-9,18,23-trioxo-2,5,11,14-tetraoxa-8,17,22-triazahentetracontane-1,21,41-tricarboxylate as a sticky, white foam (320 mg, 54%). ¹H NMR (d6-DMSO) δ 8.15 (d, 1H), 7.86 (t, 1H), 7.65 (t, 1H), 4.61 (s, 2H), 4.16-4.22 (m, 2H), 4.01 (m, 1H), 3.61 (s, 3H), 3.58 (s, 3H), 3.52-3.59 (m, 12H), 3.38-3.48 (m, 4H), 2.29 (t, 2H), 2.07-2.18 (m, 6H), 1.43-1.56 (m, 4H), 1.24 (s, 28H).

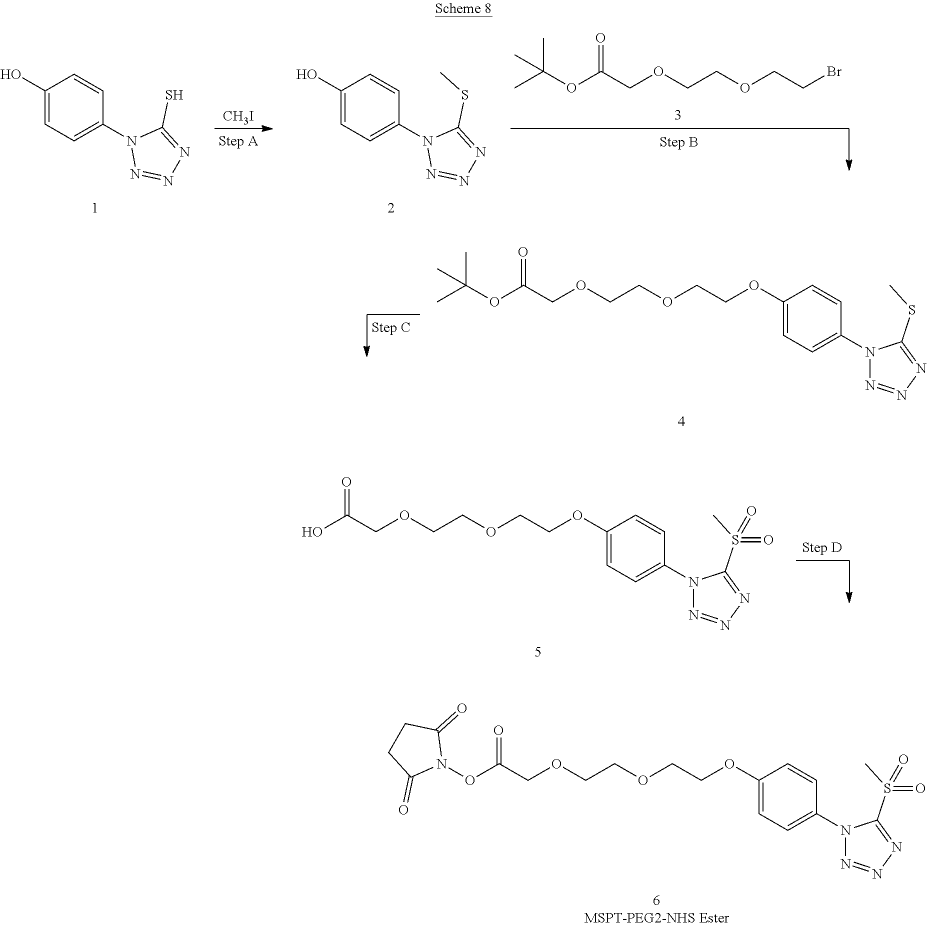

The synthesis of MSPT linker is shown in Scheme 8. The synthesis began from commercially available intermediate 4-(5-mercapto-1H-tetrazol-1-yl)phenol (CAS #52431-78-4). Alkylation of the free sulfide with methyliodide took place under the influence of DIPEA in the solvent THF to yield the methyl sulfide intermediate (2), Step A. As captured in Step B, Williamson ether reaction effected the coupling between the phenol and a bromo-PEG2 reactant that produced ether (4). Step C shows oxidation of the methyl sulfide to the methyl sulfone which used aqueous hydrogen peroxide and catalytic Molybdenum, and the crude sulfone intermediate was treated with trifluoroacetic acid in DCM to yield acid (5). The terminal carboxylic acid was transformed into the NHS-ester with EDCI and NHS-OH in Step D, which provided the activated ester (6) that is reactive to primary amines.

### Example 4Y: 4-(5-(methylthio)-1H-tetrazol-1-yl)phenol

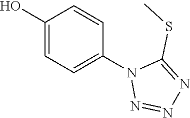

4-(5-mercapto-1H-tetrazol-1-yl)phenol (4.00 g, 20.6 mmol) was dissolved in tetrahydrofuran (50 mL), and the mixture cooled in an ice-bath, prior to the addition of N,N-diisopropylethylamine (4.31 g, 33.3 mmol). A suspension formed after stirring for 10 minutes. Iodomethane (1.54 mL, 24.7 mmol) was added dropwise via syringe over 1 min. The reaction mixture was stirred for 20 minutes with cooling in an ice-bath, then at room temperature for 12 hours. The mixture was diluted with EtOAc (100 mL), and washed with saturated aqueous NH₄Cl (2×50 mL). The organic layer was separated, then dried over sodium sulfate, filtered and concentrated in vacuo to provide 4-(5-(methylthio)-1H-tetrazol-1-yl)phenol (4.2 g, 93% yield) that can be used directly in the next step without further purification. LCMS-mz=209 (M+1).

### Example 4Z: tert-butyl 2-(2-(2-(4-(5-(methylthio)-1H-tetrazol-1-yl)phenoxy)ethoxy)ethoxy)acetate

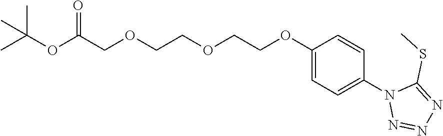 [SMILES: CSc1nnnn1-c1ccc(OCCOCCOCC(=O)OC(C)(C)C)cc1]

A 200 mL pressure vessel was charged with 4-(5-(methylthio)-1H-tetrazol-1-yl)phenol (2.50 g, 11.4 mmol), tert-butyl 2-(2-(2-bromoethoxy)ethoxy)acetate (4.33 g, 14.8 mmol) and acetone (60 mL). Potassium carbonate was added (3.15 g, 22.8 mmol), and the vessel sealed and heated at 80° C. for 8 hours with vigorous stirring. The reaction mixture was cooled to room temperature and filtered to remove potassium carbonate, then washed with acetone/DCM/EtOAc (30 mL each). The filtrate was concentrated in vacuo to provide crude material, which was purified by flash chromatography (80 g, 100% DCM for 5 minutes, then gradient to 100% EtOAc over 20 minutes). The product tert-butyl 2-(2-(2-(4-(5-(methylthio)-1H-tetrazol-1-yl)phenoxy)ethoxy)ethoxy)acetate (3.98 g, 85% yield) was isolated as a white powder. LCMS-mz=411 (M+1).

### Example 4AA: 2-(2-(2-(4-(5-(methylsulfonyl)-1H-tetrazol-1-yl)phenoxy)ethoxy)ethoxy)acetic acid

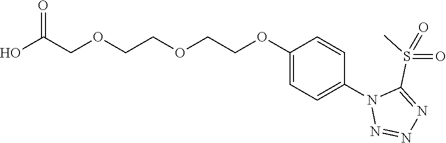 [SMILES: CS(=O)(=O)c1nnnn1-c1ccc(OCCOCCOCC(=O)O)cc1]

Tert-butyl 2-(2-(2-(4-(5-(methylthio)-1H-tetrazol-1-yl)phenoxy)ethoxy)ethoxy)acetate (3.98 g, 9.21 mmol) was dissolved in ethanol (100 mL) and cooled to 5-10° C. in an ice-water bath, prior to the addition of 30% aqueous hydrogen peroxide (19.0 mL, 184 mmol), followed by ammonium molybdate (VI) tetrahydrate (1.14 g, 0.921 mmol). The reaction mixture was stirred at room temperature for 4 hours in an ice bath, then at room temperature for 12 hours. The mixture was diluted with DCM (150 mL) and then washed with brine. The organic phase was separated, dried over sodium sulfate and concentrated in vacuo to dryness. Purification by flash column chromatography (80g silica, 100% DCM for 5 minutes, then gradient to 100% EtOAc over 20 minutes) provided 2-(2-(2-(4-(5-(methylsulfonyl)-1H-tetrazol-1-yl)phenoxy)ethoxy)ethoxy)acetic acid (3.00 g, 80% yield) as a thick oil. LCMS-mz=385 (M-1).

### Example 4BB: MSPT-PEG2-NHS Ester

### 2,5-dioxopyrrolidin-1-yl 2-(2-(2-(4-(5-(methylsulfonyl)-1H-tetrazol-1-yl)phenoxy)ethoxy)ethoxy)acetate

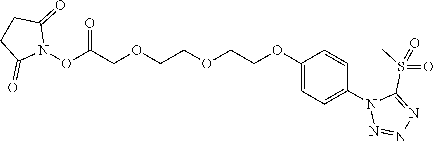 [SMILES: CS(=O)(=O)c1nnnn1-c1ccc(OCCOCCOCC(=O)ON2C(=O)CCC2=O)cc1]

1-Hydroxypyrrolidine-2,5-dione (1.33 g, 1.6 Eq, 11.6 mmol) was added to a solution of 2-(2-(2-(4-(5-(methylsulfonyl)-1H-tetrazol-1-yl)phenoxy)ethoxy)ethoxy)acetic acid (2.80 g, 7.25 mmol) in DCM (50 mL) and THF (70 mL). EDCI (1.60 g, 10.3 mmol) was added in one portion, upon which the solution became a cloudy mixture. Additional DCM (20 mL) was added to bring the mixture into solution again, followed by stirring at room temperature for 12 hours. The solvent was removed in vacuo to provide crude material as a white foam. Purification by flash column chromatography (80 g, 100% DCM for 5 minutes, then gradient to 100% EtOAc over 20 minutes) afforded 2,5-dioxopyrrolidin-1-yl 2-(2-(2-(4-(5-(methylsulfonyl)-1H-tetrazol-1-yl)phenoxy)ethoxy)ethoxy)acetate (2.61 g, 65% yield) as low melting solid. LCMS-mz=484 (M+1).

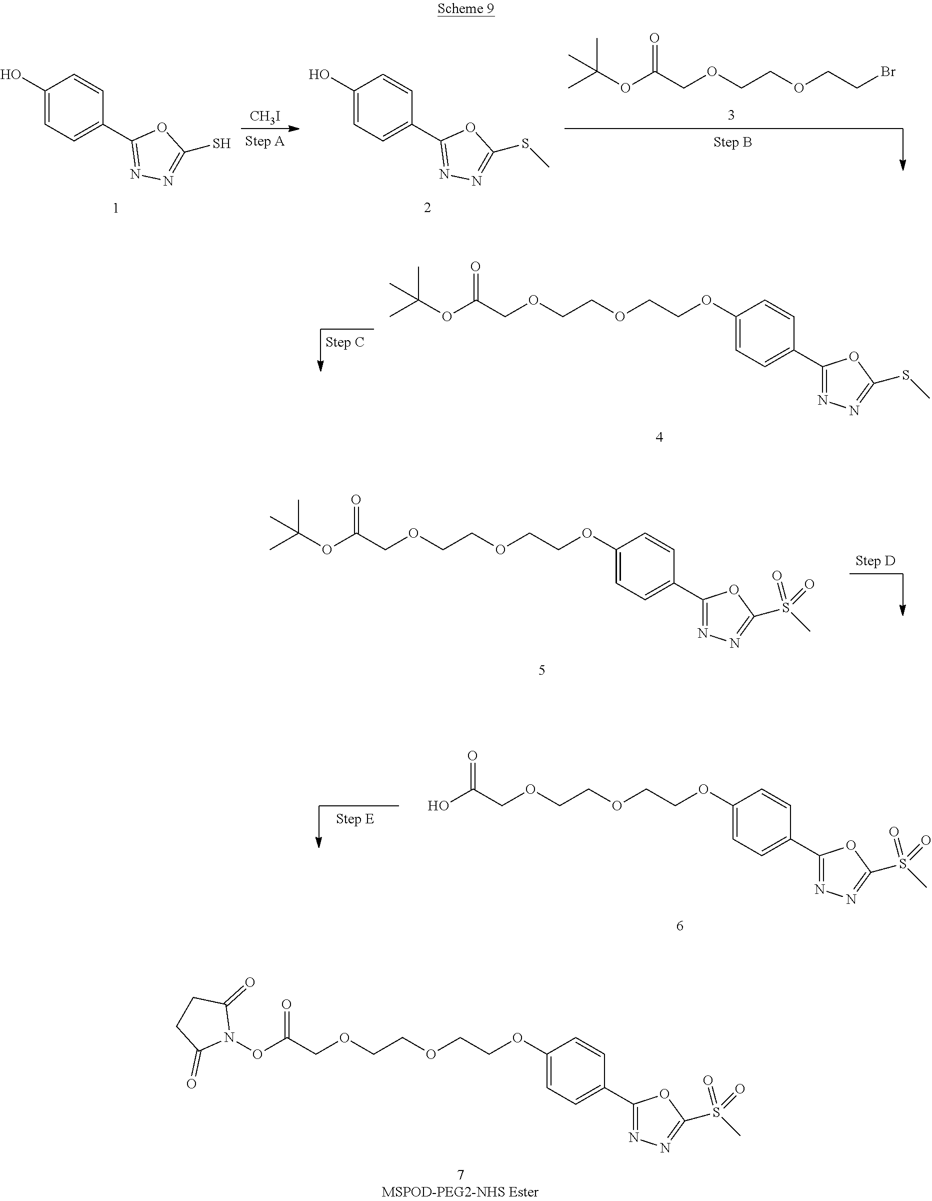

The synthesis of MSPOD linker is shown in Scheme 9. The synthesis began from commercially available intermediate 4-(5-mercapto-1,3,4-oxadiazol-2-yl)phenol (CAS #69829-90-9), and followed steps A and B as described is Scheme 8 for MPST. Oxidation with catalytic Molybdenum and aqueous hydrogen peroxide furnished the sulfone (5), Step C. Step D shows the acidic deprotection of (5) with the strong acid TFA in DCM. The terminal carboxylic acid was transformed into the NHS-ester with EDCI and NHS-OH in Step E, which provided an activated ester (7) that is reactive to primary amines.

### Example 4CC: 4-(5-(methylthio)-1,3,4-oxadiazol-2-yl)phenol

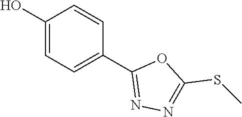

4-(5-Mercapto-1,3,4-oxadiazol-2-yl)phenol (3.00 g, 15.4 mmol) was dissolved in THF (50 mL), and cooled to 0° C. in an ice water bath. N,N-Diisopropylethylamine (3.46 mL, 2.60 g, 20.1 mmol) was added, resulting in a cloudy solution. The mixture was stirred for 5 minutes in the ice bath, then iodomethane (2.85 g, 20.1 mmol) was added drop-wise via syringe over a period of 1 minute. Upon addition, the mixture turned clear after 5 minutes. The cooling bath was removed and the mixture stirred at room temperature for 2 hours, after which it was diluted with dichloromethane (100 mL) and washed with saturated aqueous NH4Cl (pH was adjusted to ~5 by adding citric acid solution, 2×50 mL). The organic layer was separated, dried over sodium sulfate, and concentrated in vacuo to dryness to afford 4-(5-methylsulfanyl-1,3,4-oxadiazol-2-yl)phenol (520 mg, 97% yield) as a pale-yellow solid that was used in the next step without further purification. LC-MS-mz=209 (M+1).

### Example 4DD: Synthesis of tert-butyl 2-(2-(2-(4-(5-(methylthio)-1,3,4-oxadiazol-2-yl)phenoxy)ethoxy)ethoxy)acetate

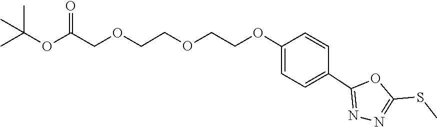 [SMILES: CSc1nnc(-c2ccc(OCCOCCOCC(=O)OC(C)(C)C)cc2)o1]

4-(5-(Methylthio)-1,3,4-oxadiazol-2-yl)phenol (3.3 g, 1 Eq, 15 mmol) and acetone (60 mL) were added to a 200 mL pressure vessel. To this solution was added tert-butyl 2-(2-(2-bromoethoxy)ethoxy)acetate (5.5 g, 20 mmol) and potassium carbonate (4.2 g, 30 mmol). The pressure vessel was sealed and heated at 70° C. for 5 hours with vigorous stirring. After cooling to room temperature, the mixture was filtered to remove solid potassium carbonate, washing with EtOAc/DCM. The filtrate was concentrated in vacuo to dryness and purified by normal phase flash column chromatography (80 g silica gold, 100% DCM for 5 minutes, then gradient to 100% EtOAc over 20 minutes). Product-containing fractions were concentrated in vacuo to afford tert-butyl 2-(2-(2-(4-(5-(methylthio)-1,3,4-oxadiazol-2-yl)phenoxy)ethoxy)ethoxy)acetate (4.8 g, 74% yield) as a white solid. LC-MS-mz=411 (M+1).

### Example 4EE: tert-butyl 2-(2-(2-(4-(5-(methylsulfonyl)-1,3,4-oxadiazol-2-yl)phenoxy)ethoxy)ethoxy)acetate

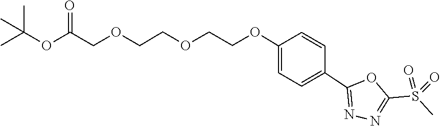 [SMILES: CC(C)(C)OC(=O)COCCOCCOc1ccc(-c2nnc(S(C)(=O)=O)o2)cc1]

Tert-butyl 2-(2-(2-(4-(5-(methylthio)-1,3,4-oxadiazol-2-yl)phenoxy)ethoxy)ethoxy)acetate (5.20 g, 12.7 mmol) was dissolved in 100 mL of ethanol, and cooled to 5-10° C. in an ice-water bath, prior to the addition of 30% hydrogen peroxide (10 mL, 97 mmol), followed by ammonium molybdate (VI) tetrahydrate (501 mg, 0.405 mmol). After two hours of vigorous stirring, an additional 15 mL of 30% hydrogen peroxide and 1 g of Ammonium molybdate (VI) tetrahydrate were added. The reaction mixture was stirred for another 6 hours, then diluted with 150 mL of DCM, and washed with brine. The organic phase was separated, dried over sodium sulfate and concentrated in vacuo to dryness. The residue was triturated with methanol to provide an initial portion of the product. The solvent was then removed from the mother liquid under reduced pressure. Purification by flash column chromatography (40 g, 100% DCM for 3 minutes, then gradient to 100% EtOAc over 20 minutes) provided additional product as a white solid. Combination of both product portions yielded tert-butyl 2-(2-(2-(4-(5-(methylsulfonyl)-1,3,4-oxadiazol-2-yl)phenoxy)ethoxy)ethoxy)acetate (5.3 g, 90% yield) as a white solid. LC-MS-mz=387 (M-tBu).

### Example 4FF: 2-(2-(2-(4-(5-(methylsulfonyl)-1,3,4-oxadiazol-2-yl)phenoxy)ethoxy)ethoxy)acetic acid

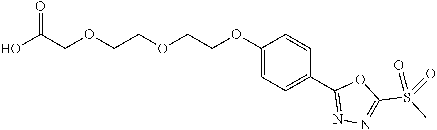 [SMILES: CS(=O)(=O)c1nnc(-c2ccc(OCCOCCOCC(=O)O)cc2)o1]

To a solution of tert-butyl 2-(2-(2-(4-(5-(methylsulfonyl)-1,3,4-oxadiazol-2-yl)phenoxy)ethoxy)ethoxy)acetate (5.60 g, 12.0 mmol) in DCM (60 mL) was added 2,2,2-trifluoroacetic acid (20 mL, 12.0 mmol). The reaction mixture was stirred at room temperature for 2 hours then concentrated under vacuo to afford a thick residue, which was purified by normal phase flash column chromatography (80g silica gold column, 100% DCM for 3 minutes, then gradient to 100% EtOAc over 20 minutes). Combination of product-containing fractions and concentration in vacuo yielded 2-(2-(2-(4-(5-(methylsulfonyl)-1,3,4-oxadiazol-2-yl)phenoxy)ethoxy)ethoxy)acetic acid (4.12 g, 82% yield). LCMS-mz=387 (M+1).

### Example 4GG: 2,5-dioxopyrrolidin-1-yl-2-(2-(2-(4-(5-(methylsulfonyl)-1,3,4-oxadiazol-2-yl)phenoxy)ethoxy)ethoxy)acetate

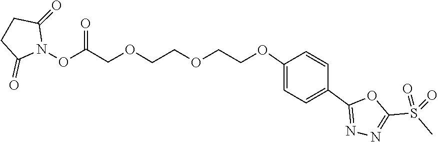 [SMILES: CS(=O)(=O)c1nnc(-c2ccc(OCCOCCOCC(=O)ON3C(=O)CCC3=O)cc2)o1]

2-(2-(2-(4-(5-(Methylsulfonyl)-1,3,4-oxadiazol-2-yl)phenoxy)ethoxy)ethoxy)acetic acid (3.00 g, 7.38 mmol) and 1-hydroxypyrrolidine-2,5-dione (1.19 g, 10.3 mmol) were dissolved in DCM (50 mL) and THF (70 mL). To this solution was added 3-(((ethylimino)methylene)amino)-N,N-dimethylpropan-1-amine (EDCI, 1.60 g, 10.3 mmol). Upon addition, the solution turned cloudy and additional DCM (20 mL) was added to bring the mixture into solution again, followed by stirring at room temperature for 12 hours. The reaction mixture was concentrated under reduced pressure, and the resulting residue dissolved in DCM purified by normal phase flash column chromatography (80 g silica gold 100% DCM for 5 minutes, then gradient to 100% EtOAc over 20 minutes). Concentration of product-containing fractions afforded 2,5-dioxopyrrolidin-1-yl-2-(2-(2-(4-(5-(methylsulfonyl)-1,3,4-oxadiazol-2-yl)phenoxy)ethoxy)ethoxy)acetate (2.61 g, 65% yield). LCMS-mz=484 (M+1).

### Example 4HH: methyl 20-((2-hydroxyethyl)amino)-20-oxoicosanoate

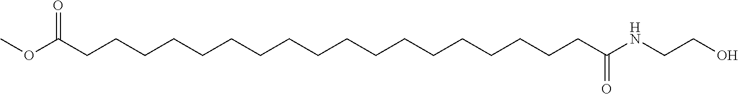 [SMILES: COC(=O)CCCCCCCCCCCCCCCCCCC(=O)NCCO]

20-methoxy-20-oxoicosanoic acid (3.00 g, 8.41 mmol), EDC (1.77 g, 9.23 mmol), HOBT (1.42 g, 9.23 mmol), DIPEA (1.62 mL, 9.23 mmol), and DCM (42 mL) were stirred at ambient temperature until all solid material dissolved (-5 min). Ethanolamine (0.56 mL. 9.23 mmol) was added at which time the reaction turned to a milky white suspension. Stirring at ambient temperature continued for 18 h. The crude reaction was concentrated, the residue was suspended in water (100 mL), acidified with 5 N HCl (2 mL), and then cooled to 0° C. for 15 min. The white solid was isolated via suction filtration, washed with water, and further dried under vacuum (3.27 g, 97%). ¹H NMR (CDCl₃) δ 6.45 (br s, 1H), 3.76 (t, 2H), 3.69 (s, 3H), 3.50-3.42 (m, 2H), 2.91 (br s, 1H), 2.37-2.24 (m, 4H), 1.72-1.58 (m, 4H), 1.39-1.20 (m, 28H).

### Example 4II: methyl 20-((2-(((2-cyanoethoxy)(diisopropylamino)phosphaneyl)oxy)ethyl)amino)-20-oxoicosanoate (C20-Acid-Ethanolamide phosphoramidite)

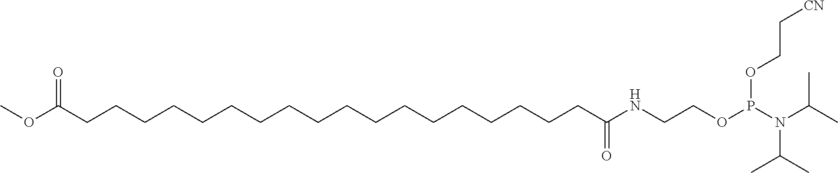 [SMILES: COC(=O)CCCCCCCCCCCCCCCCCCC(=O)NCCOP(OCCC#N)N(C(C)C)C(C)C]

A solution of methyl 20-((2-hydroxyethyl)amino)-20-oxoicosanoate (3.25 g, 8.13 mmol), 5-(ethylthio)-1H-tetrazole (0.25 M in MeCN; 16.3 mL, 4.07 mmol), 3-((bis(diisopropylamino)phosphaneyl)oxy)propanenitrile (3.36 mL, 10.6 mmol), and DCM (40 mL) was stirred at ambient temperature. After 3 hours, to crude reaction was added Celite (25 g) and the suspension was concentrated to a dry solid then purified via basic alumina flash chromatography eluting with 20-60% EtOAc/hexane to give the title compound as a white solid (4.15 g, 85%). ¹H NMR (d6-DMSO) δ 7.83 (t, 1H), 3.81-3.67 (m, 2H), 3.64-3.45 (m, 7H), 3.27-3.17 (m, 2H), 2.76 (t, 2H), 2.29 (t, 2H), 2.05 (t, 2H), 1.57-1.42 (m, 4H), 1.31-1.18 (m, 28H), 1.14 (t, 12H). ³¹P NMR (d6-DMSO) δ 146.8.

### Example 4JJ: 2-cyanoethyl (2-octyldodecyl) diisopropylphosphoramidite (C20-Octyldodecyl phosphoramidite)

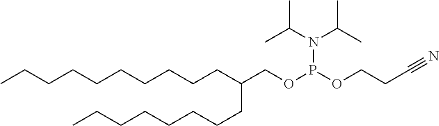 [SMILES: CCCCCCCCCCC(CCCCCCCC)COP(OCCC#N)N(C(C)C)C(C)C]

A solution of octyldodecanol (1.00 g, 3.35 mmol), dichloromethane (50 mL), 5-(ethylthio) tetrazole solution (7 mL, 0.25 M in MeCN, 2 mmol), and 3-((bis(diisopropylamino)phosphanyl)oxy)propanenitrile (1.49 mL, 4.69 mmol) was stirred at ambient temperature for 2 hours. Added Celite (12 g) and the suspension was concentrated to a dry solid then purified via 160 g neutral alumina flash chromatography eluting with 0-10% EtOAc/hexane to give the title compound as a thin oil (1.25 g, 75%). 1H NMR (CDCl3) δ 3.84 (m, 2H) 3.61 (m, 4H) 3.49 (m, 1H) 2.66 (t, 2H) 1.28 (m, 32H) 1.14 (t, 12H) 0.90 (t, 6 H). 31P NMR (CDCl₃) δ 147.6.

### Example 4KK: (Z)-2-cyanoethyl octadec-9-en-1-yl diisopropylphosphoramidite (Oleyl phosphoramidite)

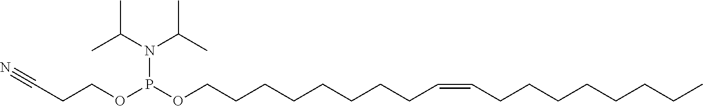 [SMILES: CCCCCCCC/C=C\CCCCCCCCOP(OCCC#N)N(C(C)C)C(C)C]

A solution of oleyl alcohol (1.00 g, 3.72 mmol), dichloromethane (50 mL), 5-(ethylthio) tetrazole solution (8 mL, 0.25 M in MeCN, 2 mmol), and 3-((bis(diisopropylamino)phosphanyl)oxy)propanenitrile (1.68 g, 5.59 mmol) was stirred at ambient temperature for 2 hours. Added Celite (12 g) and the suspension was concentrated to a dry solid then purified via 160 g neutral alumina flash chromatography eluting with 0-7% EtOAc/hexane to give the title compound as a thin oil (1.4 g, 80%). 1H NMR (d6-DMSO) δ 5.33 (t, 2H) 3.71 (m, 2H) 3.57 (m, 4H) 2.76 (t, 2H) 1.98 (m, 4H) 1.53 (q, 2H) 1.25 (m, 22H) 1.14 (t, 12H) 0.86 (t, 3 H). 31P NMR (d6-DMSO) δ 146.49

### Example 4LL: 2-cyanoethyl hexadecyl diisopropylphosphoramidite (Cetyl phosphoramidite)

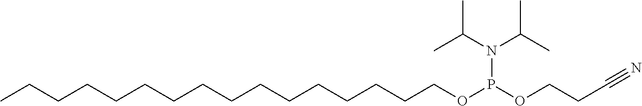 [SMILES: CCCCCCCCCCCCCCCCOP(OCCC#N)N(C(C)C)C(C)C]

A solution of cetyl alcohol (2 g, 8 mmol), dichloromethane (35 mL), 2H-tetrazole (0.45 M in MeCN, 18 mL, 7 mmol), and 3-((bis(diisopropylamino)phosphanyl)oxy)propanenitrile (3 g, 10 mmol) was stirred at ambient temperature for 3 hours. Evaporated the solvents and redissolved in hexanes. Purified via 40 g basic alumina flash chromatography eluting with 0-7% EtOAc/hexane to give the title compound as a thin oil (2.7 g, 70%). 1H NMR (CDCl3) δ 3.84 (m, 2H) 3.61 (m, 4H) 2.66 (t, 2H) 1.62 (m, 2H) 1.28 (m, 24H) 1.2 (t, 12H) 0.90 (t, 3H). 31P NMR (CDCl3) δ 147.3.

### Example 4MM: 2-cyanoethyl docosyl diisopropylphosphoramidite (C22-Docosyl phosphoramidite)

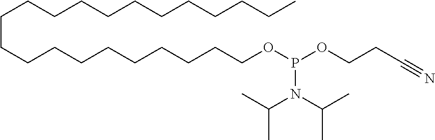 [SMILES: CCCCCCCCCCCCCCCCCCCCCCOP(OCCC#N)N(C(C)C)C(C)C]

A solution of n-docosanol (0.615 g, 1.88 mmol), dichloromethane (53 mL), 5-(ethylthio) tetrazole solution (0.25 M in MeCN, 4 mL, 1 mmol), and 3-((bis(diisopropylamino)phosphanyl)oxy)propanenitrile (851 mg, 2.82 mmol) was stirred at ambient temperature for 2 hours. Added Celite (12 g) and the suspension was concentrated to a dry solid then purified via 48 g neutral alumina flash chromatography eluting with 0-8% EtOAc/hexane to give the title compound as a white waxy solid (0.540 g, 54%). 1H NMR (CDCl3) δ 3.84 (m, 2H) 3.61 (m, 4H) 2.66 (t, 2H) 1.62 (m, 2H) 1.28 (m, 38H) 1.2 (t, 12H) 0.90 (t, 3H). 31P NMR (CDCl3) δ 147.1.

### Example 4NN: 2-((3r,5r,7r)-adamantan-1-yl)ethyl (2-cyanoethyl) diisopropylphosphoramidite (Adamantaneethanol phosphoramidite)

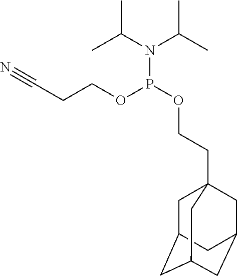 [SMILES: CC(C)N(C(C)C)P(OCCC#N)OCCC12CC3CC(CC(C3)C1)C2]

A solution of 1-admantaneethanol (2.00 g, 11.1 mmol), dichloromethane (50 mL), 5-(ethylthio) tetrazole solution (0.25 M in MeCN, 22 mL, 5.55 mmol), and 3-((bis(diisopropylamino)phosphanyl)oxy)propanenitrile (5.02 g, 16.6 mmol) was stirred at ambient temperature for 4 hours. Added Celite (12 g) and the suspension was concentrated to a dry solid then purified via 160 g neutral alumina flash chromatography eluting with 0-8% EtOAc/hexane to give the title compound as a clear thin oil (0.540 g, 54%). 1H NMR (CDCl3) δ 3.71 (m, 2H) 3.57 (m, 4H) 2.76 (t, 2H) 1.9 (s, 6H) 1.63 (q, 6H) 1.5 (m, 6H) 1.35 (t, 2H) 1.14 (t, 12H) 31P NMR (d6-DMSO) δ 146.0

### Example 4OO: methyl 20-(((2-cyanoethoxy)(diisopropylamino)phosphaneyl)oxy)icosanoate (O-C20-Acid phosphoramidite)

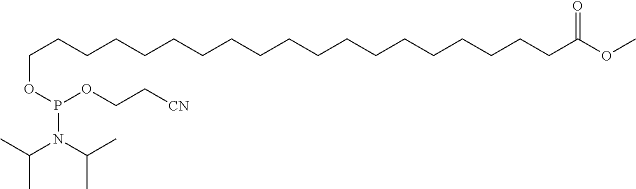 [SMILES: COC(=O)CCCCCCCCCCCCCCCCCCCOP(OCCC#N)N(C(C)C)C(C)C]

A solution of methyl 20-hydroxyicosanoate (3.10 g, 9.05 mmol), dichloromethane (45 mL), 5-(ethylthio) tetrazole solution (0.25 M in MeCN, 18 mL, 4.5 mmol), and 3-((bis(diisopropylamino)phosphanyl)oxy)propanenitrile (3.55 g, 11.8 mmol) was stirred at ambient temperature for 1 hours. Added Celite (25 g) and the suspension was concentrated to a dry solid then purified via 160 g basic alumina flash chromatography eluting with 0-10% EtOAc/hexane to give the title compound as a thick oil (4.1 g, 84%). 1H NMR (d6-DMSO) δ 3.79-3.50 (m, 9H), 2.76 (t, 2H), 2.28 (t, 2H), 1.58-1.46 (m, 4H), 1.36-1.19 (m, 30H), 1.17-1.10 (m, 12H). 31P NMR (d6-DMSO) δ 146.3.

### Example 4PP: methyl 18-(((2-cyanoethoxy)(diisopropylamino)phosphaneyl)oxy)octadecenoate (O-C18-Acid phosphoramidite)

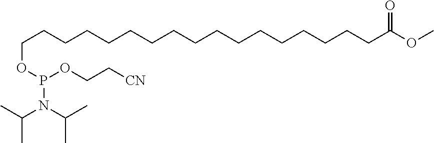 [SMILES: COC(=O)CCCCCCCCCCCCCCCCCOP(OCCC#N)N(C(C)C)C(C)C]

A solution of methyl 18-hydroxyoctadecanoate (3.15 g, 10.0 mmol), dichloromethane (50 mL), 5-(ethylthio) tetrazole solution (0.25 M in MeCN, 20 mL, 5 mmol), and 3-((bis(diisopropylamino)phosphanyl)oxy)propanenitrile (3.93 g, 13.0 mmol) was stirred at ambient temperature for 1 hours. Added Celite (25 g) and the suspension was concentrated to a dry solid then purified via 160 g basic alumina flash chromatography eluting with 0-10% EtOAc/hexane to give the title compound as a thick oil (4.0 g, 78%). 1H NMR (d6-DMSO) δ 3.79-3.50 (m, 9H), 2.76 (t, 2H), 2.28 (t, 2H), 1.58-1.46 (m, 4H), 1.36-1.19 (m, 26H), 1.17-1.10 (m, 12H). 31P NMR (d6-DMSO) δ 146.3.

### Example 4QQ: methyl 16-(((2-cyanoethoxy)(diisopropylamino)phosphaneyl)oxy)hexadecanoate (O-C16-Acid phosphoramidite)

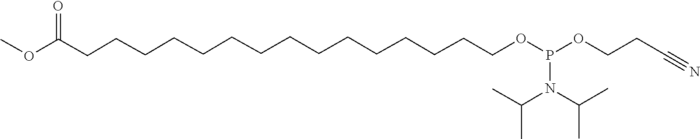 [SMILES: COC(=O)CCCCCCCCCCCCCCCOP(OCCC#N)N(C(C)C)C(C)C]

A solution of methyl 16-hydroxyhexadecanoate (3.15 g, 11.0 mmol), 5-(ethylthio)-1H-tetrazole (0.25 M in MeCN; 22.0 mL, 5.50 mmol), 3-((bis(diisopropylamino)phosphaneyl)oxy)propanenitrile (4.54 mL, 14.3 mmol), and DCM (55.0 mL) was stirred at ambient temperature. After 2 hours, to crude reaction was added Celite (25 g) and the suspension was concentrated to a dry solid then purified via basic alumina flash chromatography eluting with 100% hexane to give the title compound as a clear oil (5.03 g, 93% yield). 1H NMR (Acetonitrile-d3) δ 3.82-3.69 (m, 2H), 3.66-3.54 (m, 4H), 3.59 (s, 3H), 2.62 (t, 2H), 2.27 (t, 2H), 1.60-1.52 (m, 4H), 1.36-1.23 (m, 22H), 1.20-1.11 (m, 12H). 31P NMR (Acetonitrile-d3) δ 146.8.

### Example 4RR: methyl 16-((2-(((2-cyanoethoxy)(diisopropylamino)phosphaneyl)oxy)ethyl)amino)-16-oxohexadecanoatev (C16-Acid Ethanolamide phosphoramidite)

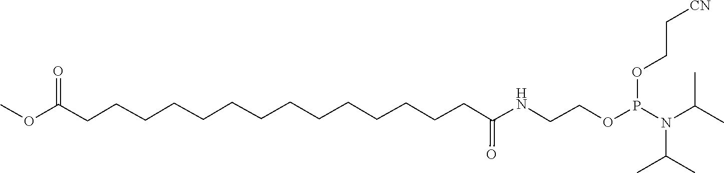 [SMILES: COC(=O)CCCCCCCCCCCCCCC(=O)NCCOP(OCCC#N)N(C(C)C)C(C)C]

A solution of 16-methoxy-16-oxohexadecanoic acid (3.05 g, 10.2 mmol), DCM (50 mL), EDC (2.14 g, 11.2 mmol), HOBt (1.71 g, 11.2 mmol), and DIPEA (1.44 g, 11.2 mmol) was stirred at ambient temperature. 2-aminoethan-1-ol (682 mg, 11.2 mmol) was added resulting in a milky white suspension and stirring continued. After 18 hours, the reaction was concentrated to remove DCM, the white solid was suspended in water (100 mL) and acidified with 5 N HCl (2 mL). The resultant aqueous suspension was cooled to 0° C. for 15 min (stirred rapidly for 5 min). White solid isolated via suction filtration, washed with 0.2 N HCl, water, then dried under vacuum to yield methyl 16-((2-hydroxyethyl)amino)-16-oxohexadecanoate (3.37 g, 97% yield).

A suspension of methyl 16-((2-hydroxyethyl)amino)-16-oxohexadecanoate (3.37 g, 9.81 mmol), 3-((bis(diisopropylamino)phosphaneyl)oxy)propanenitrile (3.84 g, 12.8 mmol), and DCM (50 mL) was stirred at ambient temperature and 5-(ethylthio)-1H-tetrazole (0.25 M in MeCN) (19.6 mL, 4.91 mmol) was added in one portion and stirring continued. The reaction turned to a clear, colorless solution after 1 hour. After 2 h, to the reaction was added Celite (-25 g), concentrated to a dry powder, and then purified via 160 g basic alumina flash chromatography eluting with 10-60% EtOAc/hexane to give the title compound as a white solid (5.0 g, 94%). 1H NMR (d6-DMSO) δ 7.83 (t, 1H), 3.81-3.67 (m, 2H), 3.81-3.67 (m, 2H), 2.76 (t, 2H), 2.28 (t, 2 H), 1.58-1.46 (m, 4H), 1.36-1.19 (m, 30H), 1.17-1.10 (m, 12H). 31P NMR (d6-DMSO) δ 146.8.

### Example 4SS: methyl 16-((2-(((2-cyanoethoxy)(diisopropylamino)phosphaneyl)oxy)ethyl)amino)-16-oxohexadecanoate (γ-Glu-C16-Acid Ethanolamide phosphoramidite)

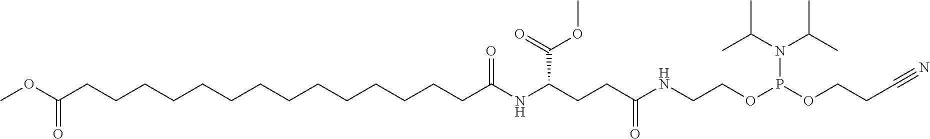 [SMILES: COC(=O)CCCCCCCCCCCCCCC(=O)N[C@@H](CCC(=O)NCCOP(OCCC#N)N(C(C)C)C(C)C)C(=O)OC]

Step 1: To a round bottomed flask was added: 16-methoxy-16-oxohexadecanoic acid (2.00 g, 1 Eq, 6.66 mmol, CAS 18451-85-9, CombiBlocks), DCM (33.3 mL), EDC (1.40 g, 1.1 Eq, 7.32 mmol), HOBt (1.12 g, 1.1 Eq, 7.32 mmol), and DIPEA (946 mg, 1.28 mL, 1.1 Eq, 7.32 mmol). The reaction mixture was stirred at room temperature for 30 minutes over the course of which solids slowly dissolved. Then to the reaction mixture was added 5-(tert-butyl) 1-methyl L-glutamate hydrochloride (1.86 g, 1.1 Eq, 7.32 mmol, CAS 6234-01-1, Alfa Aesar) and stirring at room temperature was continued. The reaction mixture was observed to be cloudy white after this addition. The reaction was allowed to proceed overnight. The next morning, reaction was still cloudy with a small amount of oily, white solid present. The reaction was diluted with DCM (50 mL) then washed with water (50 mL), again with water (50 mL) acidified with 0.5 mL 5 N HCl, and then brine. The organic layer was dried (MgSO4) and concentrated to a white solid. LCMS confirmed mass of the desired product: 5-(tert-butyl) 1-methyl (16-methoxy-16-oxohexadecanoyl)-L-glutamate. LCMS m/z=500.2 (M+1)

The product was used in subsequent reaction step without further purification.

Step 2: A round bottomed flask was charged with 5-(tert-butyl) 1-methyl (16-methoxy-16-oxohexadecanoyl)-L-glutamate (3.32 g, 1 Eq, 6.64 mmol), DCM (16.6 mL), and HCl (2.42 g, 16.6 mL, 4 molar, 10 Eq, 66.4 mmol). Stirred at RT. The reaction mixture turned light orange (from colorless) after addition of HCl. The mixture was allowed to stir for 4 hours then placed in fridge (4° C.) overnight. In the morning, the reaction mixture was removed from fridge and warmed to room temperature. The mixture was concentrated down under vacuum and then loaded onto a silica column and purified using silica gel chromatography on a gradient of 0-100% ethyl acetate in hexane. Product was observed to elute at ~100% ethyl acetate. The fractions were concentrated down, rinsed 2× with DCM and dried to afford a white solid. LCMS confirmed mass of the desired product: (S)-5-methoxy-4-(16-methoxy-16-oxohexadecanamido)-5-oxopentanoic acid. LCMS m/z=444.2 (M+1)

Step 3: A round bottomed flask was charged with (S)-5-methoxy-4-(16-methoxy-16-oxohexadecanamido)-5-oxopentanoic acid (1.3 g, 1 Eq, 2.9 mmol), DCM (15 mL), EDC (0.73 g, 1.3 Eq, 3.8 mmol), and DIPEA (0.45 g, 0.61 mL, 1.2 Eq, 3.5 mmol). The reaction mixture was stirred at room temperature for ~5 minutes as all solid material dissolved. To the reaction mixture was then added ethanolamine (0.21 g, 0.21 mL, 1.2 Eq, 3.5 mmol) in one portion and reaction immediately turned milky white. Stirring was continued at room temperature for 24 hours. The next day, the reaction mixture was concentrated en vacuo. The white solid residue was partitioned between 3:1 CHCl3/IPA (75 mL) and water (acidified with 5 N HCl). The organic layer was then washed with brine, dried (MgSO4), and concentrated and rinsed 2× with DCM and dried to afford a white solid.

LCMS confirmed mass of the desired product: methyl (S)-16-((5-((2-hydroxyethyl)amino)-1-methoxy-1,5-dioxopentan-2-yl)amino)-16-oxohexadecanoate. LCMS m/z=487.5 (M+1)

The product was used in subsequent reaction step without further purification.

Step 4: A round bottomed flask was charged with methyl (S)-16-((5-((2-hydroxyethyl)amino)-1-methoxy-1,5-dioxopentan-2-yl)amino)-16-oxohexadecanoate (1.3 g, 1 Eq, 2.7 mmol), Chloroform (13 mL), 3-((bis(diisopropylamino)phosphaneyl)oxy)propanenitrile (1.6 g, 1.7 mL, 2.0 Eq, 5.3 mmol), and 5-(ethylthio)-1H-tetrazole (0.17 g, 5.3 mL, 0.25 molar, 0.5 Eq, 1.3 mmol). The reaction mixture was stirred at room temperature. Thin layer chromatography after 1 hr indicated full consumption of starting material. The reaction was concentrated down and loaded via liquid phase for flash chromatography using a Basic Alumina column (160 g column) with 0-100% ethyl acetate/hexane gradient. A strong signal began to elute at ~90%. Fractions concentrated to a white solid.

LCMS confirmed the mass of the title product: methyl 16-(((2S)-5-((2-(((2-cyanoethoxy)(diisopropylamino)phosphaneyl)oxy)ethyl)amino)-1-methoxy-1,5-dioxopentan-2-yl)amino)-16-oxohexadecanoate. LCMS m/z=586.4 (M-Diisopropylamine fragmentation)

### Example 4TT: methyl 16-((2-(((2-cyanoethoxy)(diisopropylamino)phosphaneyl)oxy)ethyl)amino)-16-oxohexadecanoate

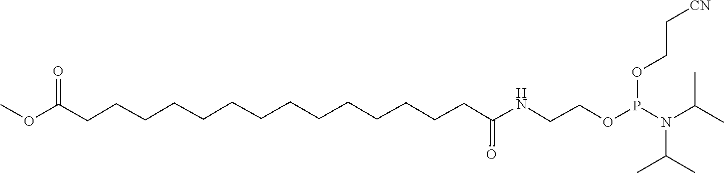 [SMILES: COC(=O)CCCCCCCCCCCCCCC(=O)NCCOP(OCCC#N)N(C(C)C)C(C)C]

A solution of 16-methoxy-16-oxohexadecanoic acid (3.05 g, 10.2 mmol), DCM (50 mL), EDC (2.14 g, 11.2 mmol), HOBt (1.71 g, 11.2 mmol), and DIPEA (1.44 g, 11.2 mmol) was stirred at ambient temperature. 2-aminoethan-1-ol (682 mg, 11.2 mmol) was added resulting in a milky white suspension and stirring continued. After 18 hours, the reaction was concentrated to remove DCM, the white solid was suspended in water (100 mL) and acidified with 5 N HCl (2 mL). The resultant aqueous suspension was cooled to 0° C. for 15 min (stirred rapidly for 5 min). White solid isolated via suction filtration, washed with 0.2 N HCl, water, then dried under vacuum to yield methyl 16-((2-hydroxyethyl)amino)-16-oxohexadecanoate (3.37 g, 97% yield).

A suspension of methyl 16-((2-hydroxyethyl)amino)-16-oxohexadecanoate (3.37 g, 9.81 mmol), 3-((bis(diisopropylamino)phosphaneyl)oxy)propanenitrile (3.84 g, 12.8 mmol), and DCM (50 mL) was stirred at ambient temperature and 5-(ethylthio)-1H-tetrazole (0.25 M in MeCN) (19.6 mL, 4.91 mmol) was added in one portion and stirring continued. The reaction turned to a clear, colorless solution after 1 hour. After 2 h, to the reaction was added Celite (-25 g), concentrated to a dry powder, and flashed (160 g basic alumina column) with 10-60% EA/H to give the title compound as a white solid (5.0 g, 94%). ¹H NMR (d6-DMSO) 7.83 (t, 1H), 3.81-3.67 (m, 2H), 3.63-3.45 (m, 7H), 3.27-3.17 (m, 2H), 2.76 (t, 2H), 2.29 (t, 2H), 2.05 (t, 2H), 1.57-1.42 (m, 4H), 1.31-1.19 (m, 20H), 1.14 (t, 12H). ³¹P NMR (d6-DMSO) d 146.8.

### Example 4UU: N-(3-(((2R,3R,4R,5R)-5-((bis(4-methoxyphenyl)(phenyl)methoxy)methyl)-2-(2,4-dioxo-3,4-dihydropyrimidin-1(2H)-yl)-4-hydroxytetrahydrofuran-3-yl)oxy)propyl)icosanamide

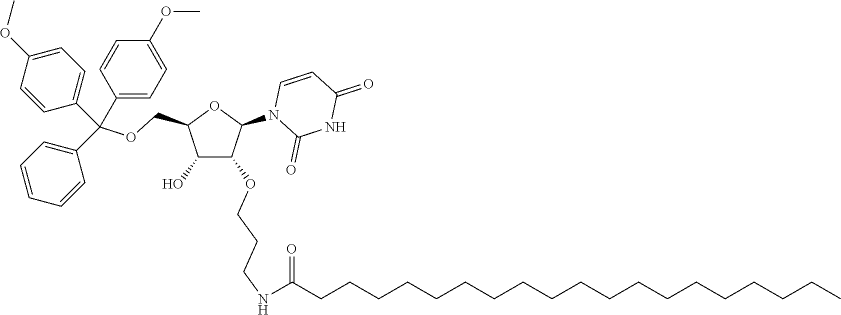 [SMILES: CCCCCCCCCCCCCCCCCCCC(=O)NCCCO[C@@H]1[C@H](O)[C@@H](COC(c2ccccc2)(c2ccc(OC)cc2)c2ccc(OC)cc2)O[C@H]1n1ccc(=O)[nH]c1=O]

A solution of 1-((2R,3R,4R,5R)-3-(3-aminopropoxy)-5-((bis(4-methoxyphenyl)(phenyl)methoxy)methyl)-4-hydroxytetrahydrofuran-2-yl)pyrimidine-2,4(1H,3H)-dione (1.00 g, 1.66 mmol), DCM (10 mL), EDC (0.35 g, 1.83 mmol), HOBt (0.28 g, 1.83 mmol), and DIPEA (0.32 mL, 1.83 mmol) was stirred at ambient temperature. icosanoic acid (0.52 g, 1.66 mmol) was added and stirring continued. After 18 hours, the reaction was diluted with DCM (30 mL) and washed with water (30 mL) acidified to pH 4 with acetic acid (95 uL). The organic layer was then washed with saturated NaHCO₃, dried (MgSO4), filtered, and concentrated to a white foam to yield methyl the title compound (1.42 g, 95% yield). ¹H NMR (d6-DMSO) 11.4 (br s, 1H), 7.76-7.70 (m, 2H), 7.42-7.21 (m, 9H), 6.91 (d, 4H), 5.79 (d, 1H), 5.28 (dd, 1H), 5.21 (d, 1H), 4.23-4.16 (m, 1H), 4.00-3.94 (m, 1H), 3.93-3.88 (m, 1H), 3.75 (s, 6H), 3.64-3.54 (m, 2H), 3.34-3.21 (m, 2H), 3.20-3.03 (m, 2H), 2.03 (t, 2H), 1.69-1.60 (m, 2H), 1.52-1.41 (m, 2H), 1.32-1.16 (m, 32H), 0.85 (t, 3H).

### Example 4VV: (2R,3R,4R,5R)-2-((bis(4-methoxyphenyl)(phenyl)methoxy)methyl)-5-(2,4-dioxo-3,4-dihydropyrimidin-1(2H)-yl)-4-(3-icosanamidopropoxy)tetrahydrofuran-3-yl (2-cyanoethyl) diisopropylphosphoramidite (2′-O-icosanamidopropyl Uridine CED phosphoramidite)

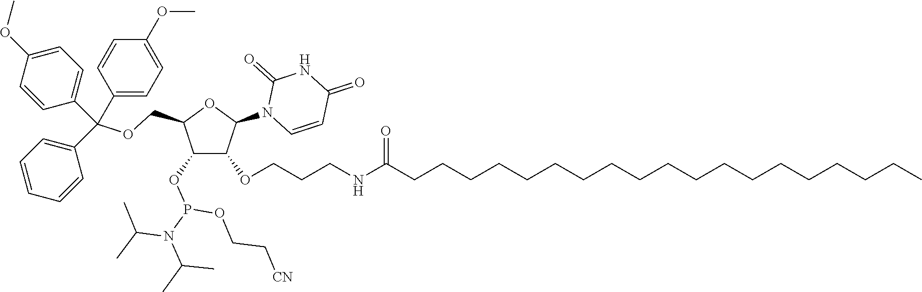 [SMILES: CCCCCCCCCCCCCCCCCCCC(=O)NCCCO[C@@H]1[C@H](OP(OCCC#N)N(C(C)C)C(C)C)[C@@H](COC(c2ccccc2)(c2ccc(OC)cc2)c2ccc(OC)cc2)O[C@H]1n1ccc(=O)[nH]c1=O]

A solution of N-(3-(((2R,3R,4R,5R)-5-((bis(4-methoxyphenyl)(phenyl)methoxy)methyl)-2-(2,4-dioxo-3,4-dihydropyrimidin-1(2H)-yl)-4-hydroxytetrahydrofuran-3-yl)oxy)propyl)icosanamide (1.42 g, 1.58 mmol), 3-((bis(diisopropylamino)phosphaneyl)oxy)propanenitrile (0.62 g, 2.06 mmol), and DCM (10 mL) was stirred at ambient temperature and 5-(ethylthio)-1H-tetrazole (0.25 M in MeCN) (3.15 mL, 0.79 mmol) was added in one portion and stirring continued. After 2 h, to the reaction was added Celite (-10 g), concentrated to a dry solid, and flashed (48 g basic alumina column) with 20-80% EA/H to give the title compound as a thick, colorless oil (1.58 g, 91%). ¹H NMR (d6-DMSO) 11.4 (br s, 1H), 7.84-7.76 (m, 1H), 7.71-7.65 (m, 1H), 7.42-7.21 (m, 9H), 6.94-6.86 (m, 4H), 5.83-5.78 (m, 1H), 5.30-5.21 (m, 1H), 4.46-4.31 (m, 1H), 4.15-3.96 (m, 4H), 3.84-3.25 (m, 12H), 3.16-3.02 (m, 2H), 2.89 (t, 2H), 2.05-1.98 (m, 2H), 1.70-1.58 (m, 2H), 1.50-1.41 (m, 2H), 1.32-1.06 (m, 44H), 0.85 (t, 3H). ³¹P NMR (d6-DMSO) d 149.1, 148.6.

### Example 5: General Procedure for Cpa/Cys Conjugation of FA1 to RNA Sense Strand

In a suitable reaction vessel, 5′ StBu-Cys protected sense strand siRNA material was dissolved in 1 mL of lx PBS and to it was added a 6-molar excess of Ac-Cpa-Lys(AEEA2-yGlu-C20-OH)-NH2 dissolved in 50/50 acetonitrile/water (-200-300 uL). The pH of the solution was adjusted to ~7.5 with 1 M Tris HCl, pH 8 (-100 uL). About 10 eq of 1,4-Dithiothreitol (DTT, Sigma Aldrich) were added to the solution and the mixture was incubated at 40′C for ~18 hrs. The reaction was monitored by analytical HPLC to observe the disappearance of StBu-Cy-siRNA and the appearance of the derivatized product sense strand. After the reaction was complete, the solution was diluted to 15 ml with water and purified by RP-HPLC. The molecular weight of the product was confirmed by LC-MS analysis using a Linear Ion Trap Mass Spectrometer (LTQ XL, Thermo Fisher Scientific) equipped with a Vanquish HPLC system (Thermo Fisher Scientific).

### Example 6: General procedure for acylation of RNA Sense Strand 3′ or 5′ reactive amino terminus or interior reactive amino prior to peptide conjugation, with: DBCO-NHS, SPDP-NHS, MSPT-PEG2-NHS, MSPOD-PEG2-NHS

In a suitable reaction vessel, the required reactive amino siRNA strand (typically 30-40 mg) was dissolved in 200-400 uL of PBS Buffer [pH 7]and 600-800 uL of DMSO along with 50-80 uL of DIPEA. The desired active ester (pre-dissolved in a minimum amount of DMSO) was then added in small increments, and the reaction was monitored accordingly by analytical HPLC. After the reaction was complete, the solution was diluted to 15 ml with water and purified by RP-HPLC. The molecular weight of the product was confirmed by LC-MS analysis using a Linear Ion Trap Mass Spectrometer (LTQ XL, Thermo Fisher Scientific) equipped with a Vanquish HPLC system (Thermo Fisher Scientific).

Alternate Procedure for Acylation of RNA Sense Strand 3′ or 5′ Reactive Amino Terminus or Interior Nucleotide Reactive Amino Prior to Peptide Conjugation, with: SMCC-NHS, Chloroacetoxy NHS or C20-Diacid-ME NHS Ester

A stock solution of NHS Ester was prepared in either a minimal concentration of acetonitrile or DMSO depending on solubility. A stock solution of sodium bicarbonate was dissolved in water at 20 mg/mL. In a suitable reaction vessel, the required reactive amino oligonucleotide strand (typically 30-40 mg) was solubilized with 15-30 molar equivalents of the stock solution of sodium bicarbonate. Then, 10-15 equivalents of reactive NHS ester solution was introduced to the reaction vessel with shaking at room temperature. Optionally, extra organic component of either DMSO or acetonitrile may be added to improve NHS ester solubility, typically between a 1:2 to 2:1 ratio of aqueous to organic. The reaction was monitored by LTQ-MS until the starting oligonucleotide was fully consumed. For reaction with chloroacetoxy NHS Ester, the reaction directly was diluted with water to >10% organics and subjected to 3KMWCO spin filtration with multiple washes to remove excess reagent and side products and no further purification was employed.

For reaction with SMCC-NHS Ester, the reaction was quenched to pH 5 with iN HCl and diluted to >10% organics. The reaction mixture was filtered through a 0.2 micron filter to remove insoluble SMCC-NHS ester byproducts, then subjected to 3KMWCO spin filtration with multiple washes to remove excess reagent and side products and no further purification was employed. The molecular weight of the product was confirmed by LC-MS analysis using a Linear Ion Trap Mass Spectrometer (LTQ XL, Thermo Fisher), and purity was typically confirmed by IP-RP UPLC. The product was quickly frozen at −20° C. or below to prevent maleimide hydrolysis and lyophilized.

For reaction with C20-Diacid-ME NHS Ester, the methyl esters were hydrolyzed with the addition of 100 equivalents of LiOH (2M LiOH solution) and shaken at room temperature for 30 minutes prior to dilution to >5% organics and spin filtration (3KMWCO). The crude functionalized oligonucleotide product was loaded onto an ES Industry Source™ 15RPC with MPA: 10 mM NaOAc 2% Acetonitrile and MPB: 80% Acetonitrile in water. Fractions were analyzed by IP-RP LCMS and those which contained a mass purity greater than 85% without impurities >5% were combined. The molecular weight of the product was confirmed by LC-MS analysis using a Linear Ion Trap Mass Spectrometer (LTQ XL, Thermo Fisher), and purity was confirmed by IP-RP UPLC analysis.

### Example 7: General Procedure for Conjugation of Functionalized RNA Sense Strands to Functionalized Peptides

For reactive dithiol-containing siRNA strands such as those derived from SPDP-NHS or C6S-SC6 moieties, any remaining dithio bonds were reduced with a reagent such as excess (tris(2-carboxyethyl)phosphine)-HCl (TCEP) prior to conjugation. The siRNA then purified from excess reducing agent using ultrafiltration (3KMWCO filter) or optionally chromatographic purification using similar methods as in Examples 4 and 10.

The conjugation of the reactive peptides and sense strands were typically done with 20-25 mg of the sense strand siRNA and a 1.5 to 2× excess of the peptide. The siRNA was dissolved in ~1.5 mL of Ultra-pure DNAse/RNAse free water, the peptide was then added as a solid. The pH of the solution was raised to ~7.5 with 1 M Tris HCl, pH 8 (~20-30 uL) The solution was incubated at 30° C. for ~1.5 hrs, additional peptide was added as needed. Reaction was monitored by analytical HPLC to observe the disappearance of siRNA and the appearance of the conjugate. After the reaction was complete, the solution was diluted to 15 ml with water and purified by RP-HPLC. The molecular weight of the product was confirmed by LC-MS analysis using a Linear Ion Trap Mass Spectrometer (LTQ XL, Thermo Fisher).

### Example 8: General Procedure for Maleimide Ring-Opening of SMCC- and MalDap-Linked Peptide-RNA Sense Strand Conjugates

For SMCC-linked conjugates: Following completion of the SMCC-thiol conjugation reaction, the pH of the reaction mixture was raised to ~9 with 1M Sodium Bicarbonate buffer and incubated at RT. The ring opening reaction was monitored by LC-MS to observe the addition of 18 Daltons. Incubation times vary from a few hours to overnight, to reach complete ring opening.

For MalDap-linked conjugates: Ring-opening can occur immediately in solution following coupling with no additional synthetic manipulation. To confirm that the ring opening is complete prior to purification, the pH of the solution was raised to ~8 with 1M Tris HCl and incubated at RT for 10-20 minutes.

### Example 9: General Procedure for HPLC Analysis of Peptide-RNA Sense Strand Conjugates

Analytical HPLC was used to monitor the conjugation reaction of the peptide and the sense strand siRNA and to check the final purity of the purified Peptide-siRNA conjugates. Analysis was done on an analytical HPLC system (Agilent 1100) using an XBridge Protein BEH C4 analytical RP-HPLC column (Waters; 3.5 μm, 300 Å; 4.6×100 mm). The running buffers used were A: 8.6 mM TEA, with 100 mM HFIP pH 8.3 and B: MeOH/Acetonitrile (50/50 v/v, Fisher Chemicals). The gradient used was a linear 0-85% B gradient over 15 min, at a flow of 1.5 mL/min, with column heating set at 60° C. UV monitoring was done at 254 nm. The molecular weight of the product was confirmed by LC-MS analysis using a Linear Ion Trap Mass Spectrometer (LTQ XL, Thermo Fisher Scientific) equipped with a Vanquish HPLC system (Thermo Fisher Scientific).

### Example 10: General Procedure for Purification of Peptide-RNA Sense Strand Conjugates by RP-HPLC

Solutions containing the peptide-siRNA conjugates were purified using a preparative HPLC system (Shimadzu LC-8A Binary Preparative HPLC Systems) using an XBridge Protein BEH C4 semi-preparative RP-HPLC column (Waters; 5 μm, 300 Å; 10×250 mm). The running buffers used were A: 8.6 mM TEA, with 100 mM HFIP pH 8.3 and B: MeOH/Acetonitrile (50/50 v/v, Fisher Chemicals). The gradient used was a linear 0-90% B gradient over 25 min, at a flow of 25 mL/min, with column heating set at 60° C. UV monitoring was done at 254 and 220 nm. Fractions containing the desired product (HPLC analysis by Agilent HPLC) were pooled, frozen, and lyophilized to give a white amorphous solid product. The molecular weight of the product was confirmed by LC-MS analysis using a Linear Ion Trap Mass Spectrometer (LTQ XL, Thermo Fisher Scientific) equipped with a Vanquish HPLC system (Thermo Fisher Scientific).

### Example 11: General Procedure for Annealing Peptide-RNA Sense Strand Conjugates to Form Duplexed dsRNA-Peptide Conjugates

In a suitable vessel, the lyophilized ssRNA-Peptide was dissolved in 2-3 mL of RNase free water and mixed thoroughly by vortexing. The concentration of the ssRNA peptide conjugate was then determined by using a NanoPhotometer NP80 (Implen) measuring optical density at 260 nm (OD260). Example calculation shown below:

$$\frac{( OD 260 )}{ε ( {mM}^{- 1} {cm}^{- 1} )} = mM$$

Where ε=extinction coefficient of the ssRNA (Peptide contribution is negligible)
mM=millimolar concentration (mmol/L)

$$( \frac{( Conc . ) mmol}{L} ) * Vol mL = mmoles$$

The concentration of a corresponding solution of anti-sense (asRNA), was also measured by OD260 using the same method. To anneal the strands, a molar excess of 1.5% asRNA was used with the annealing calculations shown below:

mmoles ssRNA-Peptide*1.015×Equivalence of asRNA=mmoles asRNA required

The volume of the asRNA solution required was then calculated. The required volume of the asRNA solution was combined with the ssRNA-Peptide solution. To anneal the strands, the mixture was incubated at 65° C. for 10 min. using a thermomixer (Eppendorf thermomixer C), and then slowly cooled to 22° C. over 40 min.

The duplexed product was then transferred to a 100,000MWL centrifugation filter tube (Millipore) and spun at 7400 rpm for 15 min. to remove any potential endotoxins. To desalt the PRC, the filtrate was then transferred into an AmiconUltra 3,000MWL centrifugation filter tube (Millipore) at 7400 rpm for 7.5 min., washed twice with 2 mL water each, and then twice with 2 mL of 1×PBS. Following endotoxin removal, water washes, and buffer exchange; the concentrated duplex solution was then transferred to a cryogenic storage vial (Coming) of the appropriate volume. The final concentration was then determined using the NanoPhotometer with OD260 measurements. The molecular weight of the product was confirmed by LC-MS analysis using a Linear Ion Trap Mass Spectrometer (LTQ XL, Thermo Fisher Scientific) equipped with a Vanquish HPLC system (Thermo Fisher Scientific). The results of this characterization are listed in Table 18.

TABLE 18
Characterization of synthesized NPR-C-binding peptide-dsRNA
conjugates with 5′-VP antisense by mass spectrometry

| | | (Calc'd) | Measured | (Calc'd) | Measured |
|---|---|---|---|---|---|
| Conju- | (Calc'd) | Sense | Sense | Antisense | Antisense |
| gate | Conjugate | Strand | Strand | Strand | Strand |
| NO | MW/Da | MW/Da | MW/Da | MW/Da | MW/Da |
| C1 | 17698.12 | 9908.95 | 9908.7 | 7789.17 | 7789.8 |
| C2 | 17734.15 | 9984.07 | 9983.8 | 7750.08 | 7750.5 |
| C3 | 17728.15 | 9993.08 | 9993 | 7735.07 | 7735.4 |
| C4 | 16695.04 | 8943.92 | 8943.7 | 7751.12 | 7751.4 |
| C5 | 16680.03 | 8984.93 | 8984.6 | 7695.09 | 7694.8 |
| C6 | 16695.04 | 9047.01 | 9046.8 | 7648.03 | 7647.7 |
| C7 | 16795.12 | 9060.06 | 9059.8 | 7735.07 | 7735.7 |
| C8 | 16642.96 | 9084.13 | 9083.8 | 7558.82 | 7558.6 |
| C9 | 16756.08 | 9043.07 | 9042.7 | 7713.01 | 7712.9 |
| C10 | 16765.09 | 8975.92 | 8975.8 | 7789.17 | 7789.3 |
| C11 | 16772.08 | 9123.12 | 9122.9 | 7648.96 | 7648.8 |
| C12 | 17079.32 | 9344.26 | 9344.0 | 7735.07 | 7734.9 |
| C13 | 17289.7258 | 9554.659 | 9553.8 | 7735.0668 | 7734.2 |
| C14 | 17219.70 | 9484.64 | 9484.3 | 7735.07 | 7734.83 |
| C15 | 17362.84 | 9627.78 | 9627.2 | 7735.07 | 7734.83 |
| C16 | 17245.74 | 9510.67 | 9510.0 | 7735.07 | 7734.83 |
| C17 | 17838.37 | 10103.30 | 10102.5 | 7735.07 | 7734.83 |
| C18 | 18535.55 | 10800.48 | 10800.2 | 7735.07 | 7735.3 |
| C19 | 18619.62 | 10884.56 | 10884.6 | 7735.07 | 7735.1 |
| C20 | 18661.66 | 10926.59 | 10926.7 | 7735.07 | 7735.3 |
| C21 | 18989.12 | 11254.06 | 11254 | 7735.07 | 7735.2 |
| C22 | 17575.003 | 9790.887 | 9790.0 | 7784.116 | 7783.6 |
| C23 | 19021.1874 | 11237.0714 | 11237.1 | 7784.116 | 7799.7 |
| C24 | 18975.1588 | 11175.977 | 11176.3 | 7799.1818 | 7799.7 |
| C25 | 18951.1341 | 11319.0892 | 11319.1 | 7632.0449 | 7632.3 |
| C26 | 18937.1043 | 11280.0499 | 11279.9 | 7657.0544 | 7657.2 |
| C27 | 18996.1779 | 11286.0544 | 11286.0 | 7710.1235 | 7710.5 |
| C28 | 17528.9744 | 9729.7926 | 9730.0 | 7799.1818 | 7800.3 |
| C29 | 17504.9497 | 9872.9048 | 9873.5 | 7632.0449 | 7632.8 |
| C30 | 17490.9199 | 9833.8655 | 9833.6 | 7657.0544 | 7657.3 |
| C31 | 17549.9935 | 9839.87 | 9839.9 | 7710.1235 | 7710.7 |
| C32 | 19382.6625 | 11583.4807 | 11583.6 | 7799.1818 | 7798.941 |
| C33 | 19185.5563 | 11386.3745 | 11387.5 | 7799.1818 | 7800.7 |
| C34 | 19358.6378 | 11726.5929 | 11727.4 | 7632.0449 | 7633.1 |
| C35 | 19161.5316 | 11529.4867 | 11530.9 | 7632.0449 | 7633.8 |
| C36 | 19344.608 | 11687.5536 | 11687.7 | 7657.0544 | 7656.808 |
| C41 | 19206.5754 | 11496.4519 | 11497.4 | 7710.1235 | 7710.9 |
| C38 | 19428.6911 | 11644.5751 | 11645.1 | 7784.116 | 7784.9 |
| C39 | 19231.5849 | 11447.4689 | 11448.4 | 7784.116 | 7784.9 |
| C40 | 19403.6816 | 11693.5581 | 11693.3 | 7710.1235 | 7710.8 |
| C37 | 19147.5018 | 11490.4474 | 11491.1 | 7657.0544 | 7657.5 |
| C42 | 18905.0393 | 11228.9752 | 11227.683 | 7676.0641 | 7675.463 |
| C43 | 17458.8549 | 9782.7908 | 9781.526 | 7676.0641 | 7675.464 |
| C44 | 19368.649 | 11692.5849 | 11691.131 | 7676.0641 | 7675.465 |
| C45 | 17402.7489 | 9726.6848 | 9725.442 | 7676.0641 | 7675.465 |
| C46 | 19312.543 | 11636.4789 | 11636.041 | 7676.0641 | 7675.465 |
| C47 | 19225.5088 | 11549.4447 | 11548.015 | 7676.0641 | 7675.463 |
| C48 | 19251.546 | 11575.4819 | 11575.041 | 7676.0641 | 7675.463 |
| C49 | 19115.4368 | 11439.3727 | 11438.006 | 7676.0641 | 7675.462 |

dsRNAs with a 3′-sense strand conjugation as indicated (unconjugated “-” or “Gal-2” conjugated via 3′-phosphonate linkage) and 5′-phosphate or 5′-vinylphophonate on the antisense strand were annealed and characterized in a substantially similar manner. The results of this characterization are listed in Table 19B.

TABLE 19B
Characterization of synthesized unconjugated ALK7-
Targeting dsRNAs with 5′-phosphate (P) antisense

| | | (Calc'd) | Measured | (Calc'd) | Measured |
|---|---|---|---|---|---|
| | (Calc'd) | Sense | Sense | Antisense | Antisense |
| dsRNA | Conjugate | Strand | Strand | Strand | Strand |
| Number | MW/Da | MW/Da | MW/Da | MW/Da | MW/Da |
| 130 | 14650.670 | 7051.772 | 7051.4 | 7598.898 | 7598.5 |
| 131 | 14711.764 | 7021.692 | 7021.3 | 7690.073 | 7689.7 |
| 132 | 14712.749 | 6925.644 | 6925.2 | 7787.105 | 7786.7 |
| 133 | 14674.695 | 7029.717 | 7029.9 | 7644.978 | 7645.0 |
| 134 | 14758.778 | 6986.739 | 6986.8 | 7772.039 | 7772.2 |
| 135 | 14651.706 | 7003.774 | 7003.9 | 7647.932 | 7647.8 |
| 136 | 14703.739 | 6791.491 | 6791.5 | 7912.248 | 7912.3 |
| 137 | 14703.739 | 7216.940 | 7217.3 | 7486.800 | 7486.9 |
| 138 | 14688.725 | 7130.836 | 7130.9 | 7557.889 | 7558.1 |
| 139 | 14772.808 | 7012.682 | 7012.3 | 7760.126 | 7759.8 |
| 140 | 14644.666 | 6882.519 | 6882.2 | 7762.147 | 7762.2 |
| 141 | 14678.730 | 7146.835 | 7146.4 | 7531.895 | 7531.5 |
| 142 | 14643.681 | 6996.734 | 6996.9 | 7646.947 | 7646.8 |
| 143 | 14683.705 | 7014.703 | 7014.5 | 7669.002 | 7668.9 |
| 144 | 14628.666 | 7083.822 | 7083.7 | 7544.844 | 7544.7 |
| 145 | 14695.714 | 7065.802 | 7065.9 | 7629.912 | 7629.8 |
| 146 | 14627.681 | 6865.535 | 6865.6 | 7762.147 | 7762.3 |
| 147 | 14688.725 | 6964.684 | 6964.3 | 7724.041 | 7723.7 |
| 148 | 14620.641 | 6996.734 | 6997.0 | 7623.907 | 7623.7 |
| 149 | 14679.714 | 6877.595 | 6877.2 | 7802.120 | 7801.8 |
| 163 | 14672.725 | 7028.732 | 7028.3 | 7643.993 | 7643.6 |
| 164 | 14650.670 | 7028.732 | 7028.3 | 7621.938 | 7621.6 |
| 165 | 14659.680 | 6967.638 | 6967.2 | 7692.042 | 7691.7 |
| 166 | 14604.641 | 6865.535 | 6865.1 | 7739.107 | 7738.7 |
| 167 | 14679.714 | 7049.803 | 7049.4 | 7629.912 | 7629.6 |
| 168 | 14693.744 | 7185.874 | 7185.5 | 7507.870 | 7507.5 |
| 169 | 14664.700 | 7193.900 | 7193.5 | 7470.800 | 7470.4 |
| 170 | 14703.739 | 6997.667 | 6997.3 | 7706.072 | 7705.7 |
| 171 | 14687.740 | 6970.739 | 6970.3 | 7717.000 | 7716.6 |
| 172 | 14743.763 | 6941.644 | 6941.3 | 7802.120 | 7801.8 |
| 173 | 14734.753 | 6979.698 | 6979.3 | 7755.055 | 7754.7 |
| 174 | 14727.764 | 6792.476 | 6792.1 | 7935.288 | 7934.9 |
| 175 | 14702.754 | 7020.707 | 7020.3 | 7682.047 | 7681.7 |
| 176 | 14589.627 | 7027.799 | 7027.4 | 7561.828 | 7561.5 |
| 177 | 14628.666 | 7020.758 | 7020.4 | 7607.908 | 7607.5 |
| 178 | 14649.685 | 6902.604 | 6902.2 | 7747.081 | 7746.7 |
| 179 | 14734.753 | 6979.698 | 6979.3 | 7755.055 | 7754.7 |
| 180 | 14628.666 | 7003.774 | 7003.4 | 7624.892 | 7624.5 |
| 181 | 14726.779 | 6963.699 | 6963.3 | 7763.080 | 7762.7 |
| 182 | 14650.670 | 7028.732 | 7028.3 | 7621.938 | 7621.6 |
| 183 | 14634.671 | 6995.749 | 6995.3 | 7638.922 | 7638.6 |
| 184 | 14651.706 | 6974.679 | 6974.3 | 7677.028 | 7676.7 |
| 185 | 14689.709 | 7097.852 | 7097.4 | 7591.857 | 7591.5 |
| 186 | 14627.681 | 7060.782 | 7060.4 | 7566.899 | 7566.5 |
| 158 | 14712.749 | 7051.772 | 7051.4 | 7660.977 | 7660.6 |
| 187 | 14688.725 | 6964.684 | 6964.3 | 7724.041 | 7723.7 |
| 188 | 14696.750 | 6879.565 | 6879.2 | 7817.185 | 7816.8 |
| 189 | 14613.652 | 7050.839 | 7050.4 | 7562.813 | 7562.5 |
| 190 | 14673.710 | 7035.773 | 7035.4 | 7637.937 | 7637.6 |
| 191 | 14727.764 | 6815.516 | 6815.1 | 7912.248 | 7911.9 |
| 192 | 14658.695 | 7029.717 | 7029.3 | 7628.978 | 7628.6 |
| 193 | 14680.699 | 6980.683 | 6980.3 | 7700.016 | 7699.7 |
| 194 | 14635.655 | 6880.549 | 6880.2 | 7755.106 | 7754.7 |
| 195 | 14726.779 | 6854.555 | 6854.2 | 7872.224 | 7871.9 |
| 196 | 14613.652 | 7027.799 | 7027.4 | 7585.853 | 7585.5 |
| 197 | 14717.769 | 7019.722 | 7019.3 | 7698.047 | 7697.7 |
| 198 | 14612.667 | 7067.823 | 7067.4 | 7544.844 | 7544.5 |
| 199 | 14605.626 | 7037.743 | 7037.3 | 7567.884 | 7567.5 |
| 200 | 14679.714 | 7049.803 | 7049.4 | 7629.912 | 7629.6 |
| 201 | 14702.754 | 7009.779 | 7009.4 | 7692.976 | 7692.6 |
| 202 | 14604.641 | 7060.782 | 7060.4 | 7543.859 | 7543.5 |
| 203 | 14679.714 | 7072.843 | 7072.4 | 7606.872 | 7606.5 |
| 204 | 14589.627 | 7027.799 | 7027.4 | 7561.828 | 7561.5 |
| 205 | 14727.764 | 6964.684 | 6964.3 | 7763.080 | 7762.7 |
| 206 | 14703.739 | 7032.819 | 7032.4 | 7670.921 | 7670.6 |
| 207 | 14726.779 | 7009.779 | 7009.4 | 7717.000 | 7716.6 |
| 208 | 14673.710 | 6925.644 | 6925.2 | 7748.066 | 7747.7 |
| 209 | 14704.724 | 7050.788 | 7050.4 | 7653.936 | 7653.6 |
| 210 | 14613.652 | 7027.799 | 7027.4 | 7585.853 | 7585.5 |
| 211 | 14688.725 | 7028.732 | 7028.3 | 7659.992 | 7659.6 |

dsRNA-GalNAc conjugates bearing a sense strand Gal-2 moiety attached to the 3′ by phosphate linkage and antisense bearing a 5′-vinylphophonate were annealed and characterized in a substantially similar manner. The results of this characterization are listed in Table 19C.

TABLE 19C
Characterization of synthesized ALK7-Targeting dsRNA-GalNAc
(Gal-2) conjugates with 5′-vinylphosphonate (VP) antisense

| | | (Calc'd) | Measured | (Calc'd) | Measured |
|---|---|---|---|---|---|
| | (Calc'd) | Sense | Sense | Antisense | Antisense |
| dsRNA | Conjugate | Strand | Strand | Strand | Strand |
| Number | MW/Da | MW/Da | MW/Da | MW/Da | MW/Da |
| 77 | 16140.27 | 8389.15 | 8390.0 | 7751.12 | 7751.9 |
| 78 | 16236.36 | 8369.16 | 8370.5 | 7867.20 | 7867.8 |
| 79 | 16156.27 | 8492.24 | 8493.3 | 7664.03 | 7664.8 |
| 80 | 16118.21 | 8521.33 | 8522.7 | 7596.88 | 7597.7 |
| 81 | 16224.35 | 8333.07 | 8333.9 | 7891.28 | 7892.1 |
| 82 | 16179.31 | 8497.31 | 8498.6 | 7682.00 | 7682.9 |
| 83 | 16132.24 | 8537.33 | 8538.4 | 7594.91 | 7595.7 |
| 84 | 16186.30 | 8552.35 | 8553.4 | 7633.95 | 7634.7 |
| 85 | 16125.25 | 8430.16 | 8431.1 | 7695.09 | 7695.8 |
| 86 | 16141.25 | 8446.16 | 8447.2 | 7695.09 | 7695.7 |
| 87 | 16226.32 | 8438.13 | 8439.4 | 7788.19 | 7788.6 |
| 88 | 16216.32 | 8498.24 | 8499.3 | 7718.08 | 7718.7 |
| 89 | 16215.34 | 8573.42 | 8574.4 | 7641.92 | 7642.7 |
| 90 | 16172.26 | 8560.37 | 8561.0 | 7611.89 | 7612.2 |
| 91 | 16201.31 | 8488.30 | 8487.9 | 7713.01 | 7713.5 |
| 92 | 16180.29 | 8349.12 | 8350.1 | 7831.17 | 7831.7 |
| 93 | 16126.23 | 8539.30 | 8540.5 | 7586.93 | 7587.6 |
| 94 | 16088.18 | 8529.36 | 8530.3 | 7558.82 | 7559.3 |
| 95 | 16163.25 | 8546.29 | 8546.9 | 7616.96 | 7617.7 |
| 96 | 16163.26 | 8460.19 | 8461.1 | 7703.07 | 7703.3 |
| 97 | 16140.27 | 8492.24 | 8493.1 | 7648.03 | 7648.3 |
| 98 | 16171.28 | 8381.12 | 8382.0 | 7790.16 | 7790.8 |
| 99 | 16210.32 | 8421.15 | 8422.1 | 7789.17 | 7789.8 |
| 100 | 16217.31 | 8568.35 | 8568.9 | 7648.96 | 7649.8 |
| 111 | 16226.32 | 8553.33 | 8553.7 | 7672.99 | 7673.5 |
| 112 | 16232.33 | 8504.30 | 8504.5 | 7728.03 | 7728.1 |
| 113 | 16217.31 | 8459.20 | 8459.2 | 7758.11 | 7758.4 |
| 114 | 16231.34 | 8537.28 | 8537.7 | 7694.06 | 7694.4 |
| 115 | 16179.31 | 8428.19 | 8429.1 | 7751.12 | 7751.7 |
| 116 | 16186.30 | 8570.32 | 8570.8 | 7615.98 | 7616.1 |
| 117 | 16187.28 | 8473.28 | 8473.6 | 7714.00 | 7714.4 |
| 118 | 16217.31 | 8453.15 | 8453.8 | 7764.16 | 7764.4 |
| 119 | 16176.30 | 8402.19 | 8402.4 | 7774.11 | 7774.4 |
| 120 | 16185.31 | 8495.34 | 8496.0 | 7689.97 | 7690.5 |
| 121 | 16225.33 | 8535.36 | 8535.7 | 7689.97 | 7690.5 |
| 122 | 16125.25 | 8430.16 | 8430.8 | 7695.09 | 7695.2 |
| 123 | 16225.34 | 8598.43 | 8598.7 | 7626.91 | 7627.2 |
| 124 | 16180.29 | 8584.40 | 8584.8 | 7595.89 | 7596.1 |
| 125 | 16185.31 | 8426.22 | 8426.7 | 7759.09 | 7759.3 |
| 126 | 16194.32 | 8404.16 | 8405.1 | 7790.16 | 7790.5 |
| 127 | 16209.33 | 8294.03 | 8294.5 | 7915.30 | 7915.6 |
| 128 | 16224.35 | 8534.38 | 8535.1 | 7689.97 | 7690.5 |
| 129 | 16133.22 | 8537.33 | 8538.1 | 7595.89 | 7596.1 |
| 130 | 16148.24 | 8553.33 | 8553.7 | 7594.91 | 7595.2 |
| 131 | 16209.33 | 8523.25 | 8523.9 | 7686.08 | 7686.4 |
| 132 | 16210.32 | 8427.20 | 8428.0 | 7783.12 | 7783.3 |
| 133 | 16172.27 | 8531.28 | 8532.0 | 7640.99 | 7641.7 |
| 134 | 16256.35 | 8488.30 | 8488.9 | 7768.05 | 7768.6 |
| 135 | 16273.38 | 8505.33 | 8506.0 | 7768.05 | 7768.6 |
| 136 | 16201.31 | 8293.05 | 8293.6 | 7908.26 | 7908.7 |
| 137 | 16201.31 | 8718.50 | 8719.1 | 7482.81 | 7483.0 |
| 138 | 16186.29 | 8632.39 | 8632.8 | 7553.90 | 7554.4 |
| 139 | 16270.38 | 8514.24 | 8514.5 | 7756.14 | 7756.1 |
| 140 | 16142.24 | 8384.08 | 8384.1 | 7758.16 | 7758.2 |
| 141 | 16176.30 | 8648.39 | 8648.8 | 7527.91 | 7527.9 |
| 142 | 16141.25 | 8498.29 | 8498.5 | 7642.96 | 7642.9 |
| 143 | 16181.27 | 8516.26 | 8516.5 | 7665.01 | 7665.1 |
| 144 | 16126.24 | 8585.38 | 8585.7 | 7540.86 | 7541.2 |
| 145 | 16193.28 | 8567.36 | 8567.4 | 7625.92 | 7626.1 |
| 146 | 16125.25 | 8367.09 | 8367.4 | 7758.16 | 7758.3 |
| 147 | 16186.29 | 8466.24 | 8466.8 | 7720.05 | 7720.1 |
| 148 | 16118.21 | 8498.29 | 8498.4 | 7619.92 | 7619.9 |
| 149 | 16177.3 | 8379.15 | 8379.4 | 7798.13 | 7798.5 |
| 157 | 16240.35 | 8505.28 | 8505.9 | 7735.07 | 7735.5 |
| 159 | 16171.28 | 8428.19 | 8428.2 | 7743.09 | 7743.4 |
| 296 | 16256.41 | 8505.28 | 8505.1 | 7751.13 | 7750.8 |
| 282 | 16382.46 | 8750.41 | 8750.1 | 7632.04 | 7632.1 |
| 283 | 16368.43 | 8711.37 | 8710.9 | 7657.05 | 7656.7 |
| 297 | 16421.49 | 8778.52 | 8777.9 | 7642.97 | 7642.8 |
| 298 | 16405.50 | 8474.13 | 8474.1 | 7931.37 | 7930.9 |
| 299 | 16156.33 | 8492.24 | 8492.1 | 7664.09 | 7663.5 |
| 281 | 16406.48 | 8607.30 | 8606.8 | 7799.18 | 7798.6 |
| 280 | 16452.51 | 8668.39 | 8668.0 | 7784.12 | 7783.4 |
| 300 | 16328.40 | 8711.37 | 8711.2 | 7617.03 | 7297.2 |
| 301 | 16353.41 | 8711.37 | 8711.8 | 7642.04 | 7642.3 |
| 302 | 16368.43 | 8751.40 | 8752.3 | 7617.03 | 7617.4 |
| 303 | 16368.43 | 8688.33 | 8688.9 | 7680.09 | 7680.7 |
| 304 | 15719.03 | 8474.13 | 8474.8 | 7244.90 | 7245.5 |
| 284 | 16427.50 | 8717.38 | 8717.5 | 7710.12 | 7709.9 |
| 305 | 16451.57 | 8717.38 | 8717.6 | 7734.19 | 7734.8 |
| 306 | 16415.49 | 8717.38 | 8717.6 | 7698.11 | 7699.0 |
| 307 | 16427.52 | 8717.38 | 8717.8 | 7710.15 | 7710.5 |
| 308 | 16427.50 | 8716.39 | 8717.0 | 7711.11 | 7711.8 |
| 309 | 16337.41 | 8678.39 | 8678.6 | 7659.02 | 7659.4 |
| 310 | 16210.32 | 8318.06 | 8317.9 | 7892.26 | 7892.3 |
| 311 | 16406.48 | 8498.15 | 8498.7 | 7908.33 | 7908.5 |
| 312 | 16406.53 | 8750.41 | 8750.8 | 7656.12 | 7657.0 |
| 313 | 16370.44 | 8750.41 | 8750.8 | 7620.03 | 7620.6 |
| 314 | 16382.48 | 8750.41 | 8750.8 | 7632.07 | 7632.5 |
| 315 | 16382.46 | 8726.39 | 8726.9 | 7656.07 | 7656.3 |
| 316 | 16452.51 | 8708.42 | 8708.7 | 7744.09 | 7744.7 |
| 317 | 16452.51 | 8645.35 | 8646.9 | 7807.16 | 7807.2 |
| 318 | 16437.49 | 8668.39 | 8690.4 | 7769.10 | 7769.3 |
| 319 | 15040.59 | 7943.87 | 7944.6 | 7096.72 | 7097.0 |
| 320 | 15704.02 | 8264.07 | 8263.8 | 7439.95 | 7440.5 |
| 321 | 15704.02 | 8607.30 | 8607.7 | 7096.72 | 7097.4 |
| 322 | 16367.44 | 8927.49 | 8928.0 | 7439.95 | 7440.2 |
| 323 | 17045.88 | 9246.70 | 9247.4 | 7799.18 | 7799.6 |
| 324 | 16580.71 | 8607.30 | 8607.7 | 7973.41 | 7973.6 |
| 325 | 16329.39 | 8717.43 | 8717.8 | 7611.96 | 7612.6 |
| 326 | 16503.61 | 8717.43 | 8717.8 | 7786.18 | 7787.4 |

dsRNAs with a conjugated 2′-XHD on the sense strand and a 5′-vinylphophonate on the antisense strand were annealed and characterized in a substantially similar manner. The results of this characterization are listed in Table 20A.

TABLE 20A
Exemplary LC/MS data for ALK7 siRNAs with 2′-XHD modifications
and 5′-vinylphophonate (VP) antisense modification

| | | | MW | MW |
|---|---|---|---|---|
| SEQ | dsRNA | | Cal. | Obs. |
| ID NO | NO. | Strand | (g/mol) | (g/mol) |
| 404 | 161 | S | 7262.17 | 7262.10 |
| 480 | | AS | 7656.99 | 7656.80 |
| 306 | 153 | S | 7099.91 | 7099.60 |
| 436 | | AS | 7795.23 | 7795.00 |
| 303 | 150 | S | 7129.99 | 7130.30 |
| 428 | | AS | 7789.17 | 7789.00 |
| 304 | 151 | S | 7277.18 | 7277.50 |
| 429 | | AS | 7648.96 | 7648.80 |
| 305 | 152 | S | 7205.11 | 7205.00 |
| 430 | | AS | 7750.08 | 7750.00 |
| 307 | 154 | S | 7253.16 | 7253.10 |
| 437 | | AS | 7648.96 | 7648.70 |
| 405 | 162 | S | 7137.03 | 7137.30 |
| 481 | | AS | 7743.09 | 7743.20 |
| 308 | 155 | S | 7198.12 | 7198.10 |
| 438 | | AS | 7697.01 | 7697.10 |
| 403 | 160 | S | 7735.07 | 7734.80 |
| 479 | | AS | 7214.12 | 7213.90 |
| 309 | 156 | S | 7215.11 | 7215.00 |
| 439 | | AS | 7695.04 | 7694.90 |

dsRNAs with various lipid builds and antisense phosphate mimics are characterized in Table 20B. Constructs containing dsRNAs NO. 291-293 were synthesized containing a 5′-vinylphosphonate antisense strand and Ada-bearing sense strand, and were synthesized in part with amidites and chemistries described in International Patent Publication Nos. WO2025064660 and WO2025137390.-Physicochemical properties of these conjugates are found in Table 20B.

TABLE 20B
Exemplary LC/MS data for ALK7 siRNAs

| SEQ | | | | MW |
|---|---|---|---|---|
| ID | dsRNA | | MW Cal. | Obs. |
| NO | NO. | Strand | (g/mol) | (g/mol) |
| 578 | 291 | S | 7273.1990 | 7272.515 |
| 653 | | as | 7676.0641 | 7675.464 |
| 579 | 292 | S | 8945.7872 | 8944.899 |
| 654 | | as | 7093.6283 | 7093.147 |
| 580 | 293 | S | 8983.7708 | 8982.886 |
| 653 | | as | 7676.0641 | 7675.464 |

### Example 12. In Vitro Knockdown of Human ALK7 in HuH7 Cells with ALK7 siRNA by Transfection

For HuH7 (JCRB Cell Bank, Part #: JCRB0403) cells, transfection was carried out by adding 24.8 μL of Opti-MEM™ (Gibco, Part #: 31985062) plus 0.2 μL of Lipofectamine™ RNAiMAX (Life Technologies, Part #: 13778-150) per well to 25 μL of each siRNA duplex with a 5′-phosphate group on the antisense strand to an individual well in a 96-well collagen I-coated plate (Coming, Part #: 354649). The mixture was then incubated at room temperature for 20 minutes. Fifty microliters of complete growth media without antibiotic containing HuH7 at 300,000 cells/mL were then added to the siRNA mixture. For single point (SP) screening, 100 nM, 0.125 nM, or 0.1 nM concentration of dsRNA agent was used, as specified in Table 21 Å. dsRNAs were bearing Gal-1 appended via phosphate linkage, as specified in Table 21 Å, except for entries with “-” which were unconjugated. Dose response experiments were done at 1000, 300, 90, 27, 8.1, 2.43, 0.73, 0.22, 0.07, 0.02, and 0.006 nM final siRNA duplex concentration. Cells were incubated for 24-48 hours prior to RNA isolation.

For two-point screening results described in Table 21B, 1 nM or 0.1 nM concentration of dsRNA agent was used, as specified in Table 21b. dsRNAs in Table 21B were unconjugated. For two-point screening results described in Table 21c, 0.5 nM or 0.05 nM concentration of dsRNA agent was used, as specified in Table 21C. dsRNAs used in Table 21C were bearing a 5′-vinyl phosphonate (VP) on their antisense and a Gal-2 conjugated via phosphate ester to the 3′-end of the sense strand as characterized in Table 19C.

Treated cells were lysed directly into the 96 well cell plate and RNA was isolated using the Quick-RNA 96 Kit (Zymo Research, Part #: R1052). The eluted RNA was used immediately or stored frozen. cDNA was synthesized using Fast Advanced RT Master Mix (Invitrogen, Part #: A39110) and using the following steps in a thermocycler: 37° C. for 30 minutes, 95° C. for 5 minutes, and 4° C. hold. Polymerase Chain Reaction (PCR) was performed via TaqMan® RT PCR (Life Technologies, Part #: 4326708) using the following cycles temperatures and times: 50° C. for 2 minutes, 95° C. for 10 minutes, 40 cycles of 95° C. for 15 seconds and 60° C. for 1 minute.

The human ALK7 levels were normalized to human RPLP0 (Life Technologies) and represented the relative knockdown (percent reduction) of human ALK7 mRNA expression as compared to vehicle-treated control cells. IC50 values were calculated using a 4-parameter fit model using XLFit.

TABLE 21A
Single-point in-vitro potency screen for ALK7 siRNAs with
5′ antisense phosphate targeting ALK7 in HuH7 cells.

| | 3′ Sense | | | | | % |
|---|---|---|---|---|---|---|
| dsRNA | Modifi- | | SP | | | Maximum |
| NO | cation | SP % KD | Concentration | | IC₅₀ (nM) | KD |
| 77 | Gal-1 | 77.98 | 100 | nM | 0.007 | 86.1 |
| 78 | Gal-1 | 90.38 | 100 | nM | 0.085 | 95.2 |
| 79 | Gal-1 | 86.64 | 100 | nM | 0.021 | 91.6 |
| 80 | Gal-1 | 79.57 | 100 | nM | 0.01 | 77.3 |
| 81 | Gal-1 | 85.01 | 100 | nM | 0.033 | 87.7 |
| 82 | Gal-1 | 86.89 | 100 | nM | 0.022 | 88.9 |
| 83 | Gal-1 | 79.20 | 100 | nM | 0.01 | 80 |
| 84 | Gal-1 | 75.33 | 100 | nM | 0.052 | 83.8 |
| 85 | Gal-1 | 79.35 | 100 | nM | 0.047 | 82.3 |
| 86 | Gal-1 | 80.38 | 100 | nM | 0.03 | 81.5 |
| 87 | Gal-1 | 83.24 | 100 | nM | 0.015 | 86.9 |
| 88 | Gal-1 | 77.92 | 100 | nM | 0.064 | 78.5 |
| 89 | Gal-1 | 82.42 | 100 | nM | 0.055 | 85.7 |
| 90 | Gal-1 | 74.50 | 100 | nM | 0.024 | 85.5 |
| 91 | Gal-1 | 76.00 | 100 | nM | 0.023 | 78.3 |
| 92 | Gal-1 | 76.15 | 100 | nM | 0.029 | 75.9 |
| 93 | Gal-1 | 87.31 | 100 | nM | 0.038 | 93.1 |
| 94 | Gal-1 | 68.98 | 100 | nM | 0.036 | 76.8 |
| 95 | Gal-1 | 72.19 | 100 | nM | 0.029 | 73.7 |
| 96 | Gal-1 | 83.50 | 100 | nM | 0.098 | 83.8 |
| 97 | Gal-1 | 67.82 | 100 | nM | 0.037 | 77.8 |
| 98 | Gal-1 | 72.81 | 100 | nM | 0.059 | 71.2 |
| 99 | Gal-1 | 25.81 | 100 | nM | — | — |
| 100 | Gal-1 | 15.05 | 100 | nM | — | — |
| 101 | Gal-1 | 59.83 | 100 | nM | — | — |
| 102 | Gal-1 | 88.93 | 100 | nM | 0.054 | 90.5 |
| 103 | Gal-1 | 83.16 | 100 | nM | 0.155 | 86.2 |
| 104 | Gal-1 | 83.00 | 100 | nM | 0.11 | 90.8 |
| 105 | Gal-1 | 67.35 | 100 | nM | 0.104 | 68.7 |
| 106 | Gal-1 | 65.52 | 100 | nM | 0.079 | 69.1 |
| 107 | Gal-1 | 7.74 | 100 | nM | — | — |
| 108 | Gal-1 | 72.33 | 100 | nM | — | — |
| 109 | Gal-1 | 65.79 | 100 | nM | — | — |
| 110 | Gal-1 | 81.50 | 100 | nM | 0.122 | 83.45 |
| 111 | Gal-1 | 78.14 | 100 | nM | 0.049 | 79.6 |
| 112 | Gal-1 | 76.36 | 100 | nM | 0.018 | 88.9 |
| 113 | Gal-1 | 75.49 | 100 | nM | 0.039 | 85.5 |
| 114 | Gal-1 | 78.91 | 100 | nM | 0.048 | 84.7 |
| 115 | Gal-1 | 80.24 | 100 | nM | 0.050 | 89.6 |
| 116 | Gal-1 | 84.03 | 100 | nM | 0.021 | 87.9 |
| 117 | Gal-1 | 78.64 | 100 | nM | 0.021 | 82.3 |
| 118 | Gal-1 | 77.82 | 100 | nM | 0.039 | 79.2 |
| 119 | Gal-1 | 78.96 | 100 | nM | 0.009 | 81.4 |
| 120 | Gal-1 | 78.86 | 100 | nM | 0.027 | 81.7 |
| 121 | Gal-1 | 74.43 | 100 | nM | 0.022 | 75.7 |
| 122 | Gal-1 | 78.24 | 100 | nM | 0.030 | 76.2 |
| 123 | Gal-1 | 76.62 | 100 | nM | 0.010 | 75.0 |
| 124 | Gal-1 | 75.82 | 100 | nM | — | — |
| 125 | Gal-1 | 79.79 | 100 | nM | 0.063 | 87.1 |
| 126 | Gal-1 | 75.06 | 100 | nM | 0.025 | 72.2 |
| 127 | Gal-1 | 78.59 | 100 | nM | 0.021 | 78.6 |
| 128 | Gal-1 | 75.94 | 100 | nM | 0.037 | 79.0 |
| 129 | Gal-1 | 77.09 | 100 | nM | — | — |
| 157 | Gal-1 | 87.35 | 100 | nM | 0.08 | 85.7 |
| 158 | Gal-1 | 87.37 | 100 | nM | 0.1093 | 85.57 |
| 159 | Gal-1 | 79.99 | 100 | nM | 0.080 | 83.0 |
| 130 | — | 69.56 | 0.125 | nM | 0.018 | 87.4 |
| 131 | — | 75.31 | 0.125 | nM | 0.007 | 84.8 |
| 132 | — | 82.78 | 0.125 | nM | 0.007 | 90.3 |
| 133 | — | 79.87 | 0.125 | nM | 0.008 | 88.2 |
| 134 | — | 76.39 | 0.125 | nM | 0.007 | 88.1 |
| 135 | — | 66.72 | 0.125 | nM | 0.007 | 76.0 |
| 136 | — | 81.95 | 0.125 | nM | 0.003 | 90.6 |
| 137 | — | 68.5 | 0.125 | nM | 0.016 | 81.7 |
| 138 | — | 53.31 | 0.125 | nM | — | — |
| 139 | — | 77.18 | 0.125 | nM | 0.0189 | 83.70 |
| 140 | — | 71.45 | 0.125 | nM | 0.0179 | 77.75 |
| 141 | — | 62.06 | 0.125 | nM | 0.0152 | 82.11 |
| 142 | — | 79.31 | 0.1 | nM | — | — |
| 143 | — | 78.01 | 0.1 | nM | — | — |
| 144 | — | 79.80 | 0.1 | nM | — | — |
| 145 | — | 82.08 | 0.1 | nM | — | — |
| 146 | — | 75.32 | 0.1 | nM | — | — |
| 147 | — | 76.26 | 0.1 | nM | — | — |
| 148 | — | 81.70 | 0.1 | nM | — | — |
| 149 | — | 79.54 | 0.1 | nM | — | — |

TABLE 21B
Two-point in-vitro potency screen for unconjugated ALK7 siRNAs
with 5′ antisense phosphate targeting ALK7 in HuH7 cells

| dsRNA | % KD, 1 nM | % KD, 0.1 nM |
|---|---|---|
| NO | Concentration | Concentration |
| 163 | 82.68 | 74.90 |
| 164 | 86.37 | 74.08 |
| 165 | 69.22 | 72.21 |
| 166 | 85.42 | 79.92 |
| 167 | 90.34 | 86.66 |
| 168 | 81.33 | 62.34 |
| 169 | 83.53 | 65.44 |
| 170 | 78.69 | 78.48 |
| 171 | 83.87 | 75.64 |
| 172 | 88.54 | 74.43 |
| 173 | 86.76 | 73.78 |
| 174 | 85.49 | 75.55 |
| 175 | 82.81 | 62.13 |
| 176 | 82.12 | 73.90 |
| 177 | 93.74 | 82.57 |
| 178 | 88.75 | 81.21 |
| 179 | 86.69 | 72.87 |
| 180 | 80.01 | 74.10 |
| 182 | 88.48 | 76.48 |
| 183 | 82.89 | 66.76 |
| 184 | 82.78 | 78.21 |
| 185 | 84.80 | 79.92 |
| 186 | 87.29 | 82.78 |
| 158 | 87.85 | 85.31 |
| 187 | 88.29 | 89.18 |
| 188 | 88.17 | 78.03 |
| 189 | 84.71 | 69.90 |
| 190 | 92.69 | 90.39 |
| 191 | 88.52 | 84.31 |
| 192 | 70.56 | 65.98 |
| 193 | 87.60 | 71.31 |
| 194 | 84.02 | 75.79 |
| 195 | 89.14 | 76.41 |
| 196 | 79.98 | 77.08 |
| 197 | 77.81 | 58.81 |
| 198 | 87.95 | 87.48 |
| 199 | 83.76 | 67.76 |
| 200 | 91.29 | 87.70 |
| 201 | 76.83 | 75.90 |
| 202 | 89.15 | 87.97 |
| 203 | 89.74 | 79.53 |
| 204 | 79.49 | 74.37 |
| 205 | 90.08 | 71.64 |
| 206 | 80.69 | 79.67 |
| 207 | 77.71 | 75.48 |
| 208 | 88.45 | 88.06 |
| 209 | 89.06 | 78.65 |
| 210 | 76.46 | 67.74 |
| 211 | 82.94 | 72.71 |

TABLE 21C
Two-point in-vitro potency screen for ALK7 siRNAs with pattern
modifications, 3′-Gal-2 conjugated sense strand and 5′-vinyl
phosphonate-antisense strand targeting ALK7 in HuH7 cells

| dsRNA | % KD, 0.5 nM | | % KD, 0.05 nM | |
|---|---|---|---|---|
| NO | Concentration | SEM | Concentration | SEM |
| 283 | 65.5 | 0.005 | 55 | 0.02 |
| 300 | 48.0 | 0.08 | 32.5 | 0.025 |
| 301 | 70.0 | 0.03 | 64.5 | 0.105 |
| 302 | 63.0 | 0.1 | 53 | 0 |
| 303 | 64.0 | 0.1 | 54.5 | 0.095 |
| 298 | 67.0 | 0.05 | 53 | 0.03 |
| 304 | 70.5 | 0.055 | 57.5 | 0.005 |
| 284 | 20.5 | 0.095 | 18.5 | 0.205 |
| 305 | 40.5 | 0.105 | 32 | 0.09 |
| 306 | 34.5 | 0.035 | 18.5 | 0.055 |
| 307 | 54.5 | 0.005 | 43 | 0.11 |
| 308 | 36.0 | 0.07 | 35 | 0.04 |
| 142 | 48.0 | 0.11 | 56 | 0.04 |
| 309 | 66.0 | 0.03 | 56 | 0.13 |
| 310 | 63.0 | 0.05 | 44.5 | 0.065 |
| 311 | 61.0 | 0.02 | 52.5 | 0.075 |
| 282 | 61.5 | 0.015 | 63.5 | 0.025 |
| 312 | 61.5 | 0.045 | 60.5 | 0.035 |
| 313 | 71.5 | 0.015 | 67 | 0.02 |
| 314 | 77.5 | 0.025 | 66.5 | 0.075 |
| 315 | 79.5 | 0.005 | 75 | 0.05 |
| 280 | 80.0 | 0.2 | 77.5 | 0.225 |
| 316 | 75.0 | 0.25 | 75.5 | 0.245 |
| 317 | 81.0 | 0.19 | 74 | 0.26 |
| 318 | 80.0 | 0.2 | 81 | 0.19 |
| 281 | 77.5 | 0.005 | 66 | 0.08 |
| 319 | 75.0 | 0.02 | 62.5 | 0.025 |
| 320 | 74.0 | 0.01 | 64 | 0.01 |
| 321 | 71.0 | 0.01 | 63 | 0.05 |
| 322 | 72.5 | 0.035 | 73.5 | 0.085 |
| 323 | 81.0 | 0.02 | 67 | 0.06 |
| 324 | 79.5 | 0.015 | 67.5 | 0.025 |

### Example 13: In Vivo Knockdown of ALK7 with XHD-ALK7 siRNA Conjugates

To assess the efficacy of the ALK7 siRNA sequences, ALK7 siRNA molecules modified with a 5′-vinylphophonate on the antisense strand were evaluated for in vivo efficacy in female C57BL/6 mice. Animals (n=5 per group) received a single subcutaneous injection of vehicle (PBS) or the ALK7 siRNA conjugated to a lipid (XHD=2′-hexadecyl X, X=A, U, C, G) conjugated on 2′-nucleotide 6 position of sense strand counting from 5′-end) at a dose of 100 nmol/kg. Adipose tissue samples were collected 2 weeks following administration of the siRNA administration. Expression of mouse ALK7 mRNA was determined using qPCR, with RPLP0 used as the reference gene. ALK7 mRNA expression for each compound was normalized relative to the vehicle control group (PBS, Table 22—set to 1). Knockdown in Table 22 was expressed in the form of decimal fraction remaining ALK7 mRNA relative to control.

TABLE 22
In Vivo Knockdown (KD) of ALK7 mRNA Expression in Adipose Tissue Collected
from Lean Mice 2 Weeks After Receiving a Single Subcutaneous Dose of ALK7
siRNAs with 2′-XHD conjugates (at nucleotide 6 counting from 5′-end)

| | | | e WAT | | iWAT | | iBAT | |
|---|---|---|---|---|---|---|---|---|
| | | | Mean | | Mean | | Mean | |
| | | | KD | | KD | | KD | |
| | | Dose | relative | | relative | | relative | |
| dsRNA | 5′ Antisense | (nmol/ | to | | to | | to | |
| NO | Modification | kg) | control | SEM | control | SEM | control | SEM |
| Control 1 | — | 0 | 1 | 0.16 | 1 | 0.2 | 1 | 0.04 |
| 161 | VP | 100 | 0.58 | 0.1 | 0.5 | 0.06 | 0.26 | 0.02 |
| 153 | VP | 100 | 0.7 | 0.1 | 0.47 | 0.06 | 0.26 | 0.04 |
| 150 | VP | 100 | 0.35 | 0.07 | 0.33 | 0.06 | 0.26 | 0.01 |
| 151 | VP | 100 | 0.31 | 0.05 | 0.43 | 0.12 | 0.39 | 0.04 |
| 152 | VP | 100 | 0.36 | 0.04 | 0.41 | 0.08 | 0.3 | 0.02 |
| Control 2 | — | 0 | 1 | 0.12 | 1 | 0.09 | 1 | 0.12 |
| 154 | VP | 100 | 0.54 | 0.09 | 0.58 | 0.05 | 0.31 | 0.1 |
| 162 | VP | 100 | 1.15 | 0.16 | 1.36 | 0.1 | 0.8 | 0.08 |
| 155 | VP | 100 | 0.54 | 0.06 | 0.68 | 0.1 | 0.39 | 0.03 |
| 160 | VP | 100 | 0.37 | 0.07 | 0.5 | 0.11 | 0.32 | 0.08 |
| 156 | VP | 100 | 0.69 | 0.17 | 0.88 | 0.22 | 0.42 | 0.04 |

n = 5 per group.
epidydimal WAT (eWAT), inguinal WAT (iWAT) and interscapular BAT (iBAT)

### Example 14: Efficacy of ALK7 siRNAs with 2′-XHD Conjugates (at Nucleotide 6 Counting from 5′-End) in Obese Mice

Four (n=4) of the ALK7-siRNA sequences with 5′-vinylphophonate on the antisense strand and with homology to the mouse ALK7 gene transcript (SEQ ID: 393) were conjugated to XHD and were evaluated for weight loss efficacy in C57BL/6 diet-induced obese (DIO) male mice fed a 60% high fat diet housed at thermoneutrality (27° C.). Mice were approximately 20 weeks of age at the start of the study. Animals received three subcutaneous doses (volume 5 mL/kg) of vehicle (PBS) or the XHD-ALK7 siRNA conjugates (Six (n=6) animals per group). Body weight was monitored daily, and body composition was analyzed prior to dosing, after two siRNA doses and at the end of study. XHD-ALK7 siRNA conjugates reduced weight gain and fat mass when compared to the vehicle treated control animals. Body weight and body composition change are presented in Table 23.

TABLE 23
In Vivo Efficacy in Obese Mice Treated with ALK7 siRNAs
with 2′-XHD conjugates (at nucleotide 6 counting from 5′-end)

| | | Body Weight | | Fat Mass | | Fat-Free Mass | |
|---|---|---|---|---|---|---|---|
| | | Change from Baseline | | Change from Baseline | | Change from Baseline | |
| dsRNA | 5′ Antisense | (%) | | (%) | | (%) | |
| NO | Modification | Mean | SEM | Mean | SEM | Mean | SEM |
| Vehicle | — | 10.52 | 1.2 | 22.59 | 1.85 | 3.25 | 0.52 |
| 150 | VP | 3.05 | 0.82 | 7.48 | 1.77 | 2.68 | 0.72 |
| 151 | VP | 2.11 | 1.12 | 8.25 | 3.08 | 1.4 | 0.62 |
| 152 | VP | 5.01 | 1.28 | 9.28 | 4.4 | 7.13 | 0.99 |
| 160 | VP | 2.38 | 1.06 | 3.99 | 2 | 5.13 | 0.6 |

n = 6 per group

### Example 15: Efficacy of ALK7 siRNA with 3′-NPRC-Binding Peptide Conjugate in Obese Mice

Three (n=3) ALK7-siRNA sequences with homology to the mouse ALK7 gene transcript were conjugated to a peptide (SEQ ID NO: 20) with affinity for the Natriuretic Peptide Clearance Receptor C (NPRC) to evaluate the effect of ALK7 knockdown on weight loss efficacy in C57BL/6 diet-induced obese (DIO) male mice fed a 60% high fat diet housed at thermoneutrality (27° C.). Mice were approximately 20-weeks of age at the start of the study. Mice received two subcutaneous doses (5 mL/kg) of either PBS, or the ANP-conjugated ALK7 siRNA (250 nmol/kg) prior to treatment with the incretin semaglutide (SEQ ID NO: 392), a GLP-1R agonist therapeutic. Following 18 days of treatment, animals received a final dose of the ALK7 siRNA (250 nmol/kg), and daily dosing with the GLP-1RA (3 nmol/kg) was initiated (n=6 per group). Body weight was monitored daily, body composition was analyzed prior to incretin dosing and at the end of study. The NPRC-binding-peptide-ALK7 siRNA conjugates promoted weight loss dosed alone and augmented weight loss induced by the semaglutide (Table 24). The ANP-ALK7 siRNA conjugates stimulated fat mass, and prevented fat-free mass loss dosed alone and in combination with the GLP-1RA (Table 24). On day 15 post start of GLP-1RA dosing, animals were euthanized.

TABLE 24
In Vivo Efficacy in Obese Mice Treated with ALK7 siRNA
with 3′-NPRC binding peptide conjugate

| | | Body Weight | | Fat Mass | | Fat-Free Mass | |
|---|---|---|---|---|---|---|---|
| | Conjugate | Change from Baseline | | Change from Baseline | | Change from Baseline | |
| dsRNA | NO +/− | (%) | | (%) | | (%) | |
| NO/ | sema | Mean | SEM | Mean | SEM | Mean | SEM |
| Vehicle | Vehicle | 1.51 | 0.5 | 10.48 | 1.6 | 0.73 | 0.8 |
| 99 | C1 | −1.12 | 1.95 | −1.95 | 0.83 | 1.29 | 1.13 |
| 101 | C2 | −5.71 | 1.97 | −10.18 | 4.2 | 1.63 | 0.67 |
| 157 | C3 | −4.32 | 0.69 | −16.3 | 1.35 | 2.39 | 0.96 |
| GLP-1RA | sema | −11.36 | 1.86 | −11.61 | 3.2 | −6.35 | 0.67 |
| 99 + GLP- | C1 + sema | −12.74 | 2.22 | −21.23 | 3.02 | −5.28 | 0.37 |
| 1RA | | | | | | | |
| 101 + | C2 + sema | −16.89 | 3.4 | −29.81 | 5.79 | −2.69 | 1.04 |
| GLP-1RA | | | | | | | |
| 157 + | C3 + sema | −17.89 | 1.91 | −39.51 | 4.33 | −2.56 | 0.55 |
| GLP-1RA | | | | | | | |

### Example 16a: Efficacy of ALK7 siRNA with 3′-GalNAc Conjugates in Lean ALK7 AAV Mice

To assess the efficacy of the human active ALK7 siRNA sequences, the ALK7 siRNA molecules were evaluated for in vivo efficacy in an AAV human ALK7 mouse model. Female C57BL/6 mice greater than 10-weeks of age were intravenously injected with 1×10¹¹ genome copies per animal of adeno-associated virus (AAV) designed to express human ALK7 gene. At least 3-weeks after AAV injection, mice (n=5 per group) received a single subcutaneous injection of vehicle (PBS) or the ALK7 siRNA molecule conjugated to Gal-2 at a dose of 3 mg/kg. Liver was collected 2-weeks following siRNA administration. Hepatic mRNA expression of human ALK7 was determined using qPCR, with RPLP0 used as the reference gene. Average ALK7 expression for each compound was normalized relative to the vehicle control group (PBS, Table 25, set to 1). Knockdown in Table 25 was expressed in the form of decimal fraction remaining ALK7 mRNA relative to control. dsRNAs used in Table 25 were bearing a 5′-vinyl phosphonate (VP) on their antisense and a Gal-2 conjugated via phosphate ester to the 3′-end of the sense strand as characterized in Table 19C.

TABLE 25
Knockdown of ALK7 mRNA Expression in Liver
Samples Collected from ALK7 AAV Mice Dosed
with ALK7 siRNA with 3′GalNAc conjugates

| | 5′ Antisense | Dose | | |
|---|---|---|---|---|
| dsRNA NO | Modification | (mg/kg) | Mean | SEM |
| Control | | 0 | 1 | 0.16 |
| 77 | VP | 3 | 0.31 | 0.05 |
| 78 | VP | 3 | 0.6 | 0.18 |
| 79 | VP | 3 | 0.6 | 0.13 |
| 80 | VF | 3 | 0.73 | 0.08 |
| 81 | VP | 3 | 0.79 | 0.2 |
| 82 | VP | 3 | 0.83 | 0.11 |
| 83 | VP | 3 | 0.93 | 0.36 |
| 84 | VP | 3 | 0.56 | 0.11 |
| 85 | VP | 3 | 0.34 | 0.07 |
| 86 | VP | 3 | 0.68 | 0.07 |
| 87 | VF | 3 | 0.83 | 0.14 |
| 88 | VP | 3 | 0.81 | 0.16 |
| 89 | VP | 3 | 0.65 | 0.08 |
| 90 | VP | 3 | 0.57 | 0.07 |
| 91 | VP | 3 | 0.51 | 0.07 |
| 92 | VP | 3 | 0.58 | 0.09 |
| 93 | VP | 3 | 0.6 | 0.08 |
| Control | | 0 | 1 | 0.14 |
| 94 | VP | 3 | 0.47 | 0.08 |
| 157 | VP | 3 | 0.45 | 0.11 |
| 95 | VP | 3 | 0.67 | 0.1 |
| 96 | VP | 3 | 0.68 | 0.15 |
| 97 | VP | 3 | 0.36 | 0.11 |
| 98 | VP | 3 | 0.79 | 0.09 |
| Control | VP | 0 | 1 | 0.20 |
| 159 | VP | 3 | 0.56 | 0.05 |
| 113 | VP | 3 | 0.45 | 0.11 |
| 114 | VP | 3 | 0.24 | 0.06 |
| 118 | VP | 3 | 0.39 | 0.14 |
| 120 | VP | 3 | 0.32 | 0.11 |
| 122 | VP | 3 | 0.47 | 0.09 |
| 125 | VP | 3 | 0.41 | 0.09 |
| 128 | VP | 3 | 0.40 | 0.12 |
| 138 | VP | 3 | 0.65 | 0.17 |
| 111 | VP | 3 | 0.33 | 0.10 |
| 124 | VP | 3 | 0.38 | 0.10 |
| 136 | VP | 3 | 0.46 | 0.13 |
| 134 | VP | 3 | 0.12 | 0.03 |
| 132 | VP | 3 | 0.23 | 0.14 |
| 131 | VP | 3 | 0.76 | 0.21 |
| 135 | VP | 3 | 0.57 | 0.11 |
| 133 | VP | 3 | 0.32 | 0.06 |
| 115 | VP | 3 | 0.66 | 0.09 |
| 139 | VP | 3 | 0.71 | 0.21 |
| 77 | VP | 3 | 0.18 | 0.03 |
| 85 | VP | 3 | 0.26 | 0.05 |
| 97 | VP | 3 | 0.34 | 0.06 |
| 94 | VP | 3 | 0.30 | 0.04 |
| 91 | VP | 3 | 0.40 | 0.08 |
| 100 | VP | 3 | 0.25 | 0.04 |
| 84 | VP | 3 | 0.39 | 0.09 |
| 79 | VP | 3 | 0.45 | 0.09 |
| 119 | VP | 3 | 0.27 | 0.06 |
| 123 | VP | 3 | 0.38 | 0.05 |
| 141 | VP | 3 | 0.32 | 0.05 |
| 137 | VP | 3 | 0.34 | 0.03 |
| 130 | VP | 3 | 0.51 | 0.14 |
| 140 | VP | 3 | 0.54 | 0.04 |
| 112 | VP | 3 | 0.37 | 0.04 |
| 127 | VP | 3 | 0.42 | 0.04 |
| 142 | VP | 3 | 0.34 | 0.10 |
| 145 | VP | 3 | 0.54 | 0.06 |
| 146 | VP | 3 | 0.51 | 0.13 |
| 148 | VP | 3 | 0.51 | 0.03 |
| 143 | VP | 3 | 0.60 | 0.04 |
| 144 | VP | 3 | 0.55 | 0.08 |
| 147 | VP | 3 | 0.63 | 0.08 |
| 149 | VP | 3 | 0.47 | 0.14 |
| 116 | VP | 3 | 0.25 | 0.03 |
| 117 | VP | 3 | 0.28 | 0.04 |
| 310 | VP | 3 | 0.43 | 0.13 |
| 121 | VP | 3 | 0.70 | 0.14 |
| 126 | VP | 3 | 1.18 | 0.25 |

n = 5 per group

### Example 16B: Efficacy of ALK7 siRNA with 3′-GalNAc Conjugates in Lean ALK7 AAV Mice

To assess the efficacy of the human active ALK7 siRNA sequences, the ALK7 siRNA molecules were evaluated for in vivo efficacy in an AAV human ALK7 mouse model. Female C57BL/6 mice greater than 10-weeks of age were intravenously injected with 1×10¹¹ genome copies per animal of adeno-associated virus (AAV) designed to express human ALK7 gene. At least 3-weeks after AAV injection, mice (n=5 per group) received a single subcutaneous injection of vehicle (PBS) or the ALK7 siRNA molecule conjugated to Gal-2 at a dose of 3 mg/kg. Liver was collected 2-weeks following siRNA administration. Hepatic mRNA expression of human ALK7 was determined using qPCR, with RPLP0 used as the reference gene. Average ALK7 expression for each compound was normalized relative to the control group (PBS, Table 26). Knockdown in Table 26 is expressed in the form of decimal fraction remaining ALK7 mRNA relative to control. dsRNAs used in Table 26 were bearing a 5′-vinyl phosphonate (VP) on their antisense and a Gal-2 conjugated via phosphate ester to the 3′-end of the sense strand as characterized in Table 19C.

TABLE 26
Knockdown of ALK7 mRNA Expression in Liver
Samples Collected from ALK7 AAV Mice Dosed
with ALK7 siRNA with GalNAc conjugates

| | 5′ Antisense | Dose | | |
|---|---|---|---|---|
| dsRNA NO | Modification | (mg/kg) | Mean | SEM |
| Control | | 0 | 1 | 0.20 |
| 280 | VP | 3 | 0.43 | 0.08 |
| 281 | VP | 3 | 0.51 | 0.08 |
| 324 | VP | 3 | 0.34 | 0.03 |

n = 5 per group

### Example 17A: Efficacy of ALK7 siRNA with 3′-NPRC-Binding Peptide Conjugates in Both Lean and Obese Mice

To evaluate the in vivo potency of the ALK7 siRNA sequence a dose response study was conducted (Table 27) in lean mice. Male C57BL/6 mice (n=8 per group) greater than 10-weeks of age received a single subcutaneous dose of vehicle (PBS) or the ALK7 siRNA molecule conjugated to an NPRC-binding peptide. Two weeks after siRNA administration, animals were euthanized and adipose tissue samples were collected. Adipose tissue mRNA expression of murine ALK7 was determined using qPCR, with RPLP0 used as the reference gene. Average ALK7 expression was normalized relative to the vehicle group (PBS, Tables 28 and 29, set to 1). Knockdown in Tables 28 and 29 was expressed in the form of decimal fraction remaining ALK7 mRNA relative to control.

TABLE 27
Knockdown of ALK7 mRNA Expression in Inguinal White
adipose Samples Collected from lean Mice Dosed
with ALK7 siRNA with 3′ ANP peptide conjugates

| | | |
|---|---|---|
| Conjugate | Dose | Inguinal White Adipose Tissue |

| NO | (mg/kg) | Mean | SEM |
|---|---|---|---|
| control | PBS | 1 | 0.09 |
| C3 | 1.8 | 0.39 | 0.04 |
| C3 | 4.4 | 0.26 | 0.02 |
| C3 | 8.8 | 0.34 | 0.06 |
| C7 | 1.7 | 0.44 | 0.05 |
| C7 | 3.4 | 0.32 | 0.03 |
| C7 | 6.8 | 0.49 | 0.05 |

n = 8 per group

TABLE 28
mRNA Expression in Epididymal White Adipose
Samples Collected from lean Mice Dosed with
ALK7 siRNA with 3′-ANP peptide conjugates

| | |
|---|---|
| Dose | Epididymal White Adipose |

| Conjugate NO | (mg/kg) | Mean | SEM |
|---|---|---|---|
| control | PBS | 1 | 0.12 |
| C3 | 1.8 | 0.30 | 0.04 |
| C3 | 4.4 | 0.29 | 0.04 |
| C3 | 8.8 | 0.42 | 0.07 |
| C7 | 1.7 | 0.36 | 0.06 |
| C7 | 3.4 | 0.28 | 0.02 |
| C7 | 6.8 | 0.34 | 0.02 |

n= 8 per group

TABLE 29
mRNA Expression in Interscapular Brown Adipose
Samples Collected from lean Mice Dosed with
ALK7 siRNA with 3′ ANP peptide conjugates

| | |
|---|---|
| Dose | iBrown Adipose Fat |

| Conjugate NO | (mg/kg) | Mean | SEM |
|---|---|---|---|
| control | PBS | 1 | 0.08 |
| C3 | 1.8 | 0.36 | 0.09 |
| C3 | 4.4 | 0.28 | 0.04 |
| C3 | 8.8 | 0.33 | 0.05 |
| C7 | 1.7 | 0.32 | 0.04 |
| C7 | 3.4 | 0.34 | 0.06 |
| C7 | 6.8 | 0.24 | 0.02 |

n = 8 per group

### Example 17B: Efficacy of ALK7 siRNA with X′-ANP Peptide Conjugates in Obese Mice

To evaluate the in vivo potency and efficacy of adipose tissue shuttles, obese mice were dosed with ALK7 siRNA conjugates (Table 30). Male C57BL/6 mice (n=6 per group) received a single subcutaneous dose of vehicle (PBS) or the ALK7 siRNA molecule conjugated to a ANP peptide (100 nmol/kg). Body weight was recorded at 4, and 8 weeks. 8 weeks after siRNA administration, animals were euthanized and adipose tissue samples were collected. Adipose tissue mRNA expression of murine ALK7 was determined using qPCR, with RPLP0 used as the reference gene. Average ALK7 expression was normalized relative to the vehicle group (PBS, Table 30—set to 1). Knockdown in Table 30 was expressed in the form of decimal fraction remaining ALK7 mRNA relative to control.

TABLE 30
Knockdown of ALK7 mRNA Expression in Inguinal White adipose
Samples Collected from Obese Mice and Body Weight Dosed
with ALK7 siRNA with 3′ ANP peptide conjugates

| | | Inguinal White | | | |
|---|---|---|---|---|---|
| | | Adipose Tissue | | Body Weigt Loss | |
| | | ALK7 KD 8-Weeks | | (% Vs Baseline) | |
| Conjugate | Dose | Post Dose | | 4 | 8 |
| NO | (nmol/kg) | Mean | SEM | weeks | weeks |
| control | PBS | 1 | 0.13 | 2.27 | 4.4 |
| C7 | 100 | 0.39 | 0.05 | −4.4 | −2.6 |
| C12 | 100 | 0.38 | 0.06 | −5.2 | 0.6 |
| C13 | 100 | 0.35 | 0.03 | −6.2 | −4.1 |
| C14 | 100 | 0.42 | 0.05 | −7.5 | −4.0 |
| C15 | 100 | 0.46 | 0.03 | −6.7 | −2.6 |
| C16 | 100 | 0.35 | 0.07 | −7.1 | −3.3 |
| C17 | 100 | 0.27 | 0.04 | −5.7 | −4.7 |
| C18 | 100 | 0.37 | 0.06 | −4.5 | −1.0 |
| C19 | 100 | 0.33 | 0.02 | −5.0 | −2.7 |
| C20 | 100 | 0.38 | 0.02 | −4.1 | −0.5 |
| C21 | 100 | 0.34 | 0.01 | −3.8 | −0.9 |

n = 5 per group

### Example 18: Efficacy of ALK7 siRNA with 3′-ANP Peptide Conjugates in Lean ALK7 AAV Mice

To evaluate the durability of ALK7 mRNA knockdown in adipose tissue, a time course study was conducted in lean mice. Male C57BL/6 mice (n=8 per group) greater than 10-weeks of age received a single subcutaneous dose of vehicle (PBS) or the ALK7 siRNA NPRC-binding-peptide conjugate. Adipose tissue samples were collected 2-, 4-, 8-weeks post siRNA administration. Adipose tissue mRNA expression of murine ALK7 was determined using qPCR, with RPLP0 used as the reference gene. Average ALK7 expression was normalized relative to the vehicle control group (PBS, Table 31—set to 1). Knockdown in Table 31 was expressed in the form of decimal fraction remaining ALK7 mRNA relative to control.

TABLE 31
Knockdown of ALK7 mRNA Expression in Epididymal White
adipose Samples Collected from lean Mice Dosed with ALK7
siRNA with 3′-NPRC-binding peptide conjugates.

| | In vivo_Lean CRS28 5 mg/kg KD % | | | | | |
|---|---|---|---|---|---|---|
| Conjugate | 2 weeks | | 4 weeks | | 8 weeks | |
| NO | Mean | SEM | Mean | SEM | Mean | SEM |
| control | 1 | 0.15 | 1 | 0.17 | 1 | 0.12 |
| C7 | 0.31 | 0.09 | 0.25 | 0.05 | 0.39 | 0.07 |
| C9 | 0.51 | 0.16 | 0.30 | 0.03 | 0.65 | 0.05 |
| C10 | 0.68 | 0.08 | 0.27 | 0.06 | 0.37 | 0.06 |
| C11 | 0.61 | 0.08 | 0.54 | 0.09 | 0.54 | 0.06 |
| C3 | 0.54 | 0.05 | 0.30 | 0.07 | 0.38 | 0.07 |

n = 8 per group

### Example 19: Efficacy of ALK7 siRNA with 3′-ANP Peptide Conjugates in ALK7 Humanized Lean Mice

To assess the efficacy of human ALK7 siRNA sequences, ALK7 siRNA ANP conjugates were evaluated in humanized ALK7 mice (Gem Pharmatech B6-hACVR1C mice, strain T066492). Male ALK7 humanized mice (n=6 per group) greater than 10-weeks of age received a single subcutaneous dose of vehicle (PBS) or the ALK7 siRNA ANP-peptide conjugate. Adipose tissue samples were collected 2-weeks post dose. Adipose tissue mRNA expression of human ALK7 was determined using qPCR, with RPLP0 used as the reference gene. Average ALK7 expression was normalized relative to the vehicle control group (PBS, Table 32—set to 1). Knockdown in Table 32 was expressed in the form of decimal fraction remaining ALK7 mRNA relative to control.

TABLE 32
In Vivo Knockdown (KD) of ALK7 mRNA Expression in Adipose Tissue Collected from ALK7
Humanized Mice 2 Weeks After Receiving a Single Subcutaneous Dose of ALK7 dsRNAconjugates

| | | | e WAT | | iWAT | | iBAT | |
|---|---|---|---|---|---|---|---|---|
| | | | Mean KD | | Mean KD | | Mean KD | |
| | | | relative | | relative | | relative | |
| dsRNA | Conjugate | Dose | to control | | to control | | to control | |
| NO | NO | (nmol/kg) | (%) | SEM | (%) | SEM | (%) | SEM |
| N/A | Control | 0 | 1 | 0.02 | 1 | 0.07 | 1 | 0.07 |
| 280 | C22 | 250 | 0.52 | 0.07 | 0.56 | 0.08 | 0.26 | 0.03 |
| 280 | C23 | 250 | 0.47 | 0.06 | 0.77 | 0.06 | 0.55 | 0.05 |
| 281 | C24 | 250 | 0.29 | 0.02 | 0.39 | 0.02 | 0.16 | 0.02 |
| 282 | C25 | 250 | 0.34 | 0.03 | 0.52 | 0.04 | 0.30 | 0.03 |
| 283 | C26 | 250 | 0.40 | 0.05 | 0.66 | 0.01 | 0.47 | 0.06 |
| 284 | C27 | 250 | 0.48 | 0.02 | 0.66 | 0.04 | 0.35 | 0.04 |
| 281 | C28 | 250 | 0.33 | 0.02 | 0.44 | 0.04 | 0.17 | 0.01 |
| 282 | C29 | 250 | 0.38 | 0.03 | 0.45 | 0.04 | 0.20 | 0.02 |
| 283 | C30 | 250 | 0.40 | 0.07 | 0.42 | 0.01 | 0.22 | 0.03 |
| 284 | C31 | 250 | 0.44 | 0.04 | 0.54 | 0.04 | 0.36 | 0.07 |

Epidydimal WAT (eWAT), inguinal WAT (iWAT) and interscapular BAT (iBAT).
n = 5 per treatment group.

### Example 20: In Vivo Adipose Shuttle Comparison Study with ALK7 Tool siRNA Sequence in Lean ALK7 Humanized Mice

To compare the in vivo efficacy of different adipose shuttles, an ALK7 tool sequence was conjugated to peptide and/or lipid-based shuttles and tested in ALK7 humanized mice (Gem Pharmatech B6-hACVR1C mice, strain T066492). Male ALK7 humanized mice (control, n=6 and test article, n=5 per group) greater than 10-weeks of age received a single subcutaneous dose of vehicle, or the ALK7 siRNA conjugates (100 nmol/kg). Adipose tissue samples were collected 4-weeks post administration of the ALK7 siRNA. Expression of human ALK7 was determined using qPCR, with RPLP0 used as the reference gene. Average ALK7 expression was normalized relative to the control group (PBS —Table 33, set to 1). Knockdown in Table 33 was expressed in the form of decimal fraction remaining ALK7 mRNA relative to control.

TABLE 33
In Vivo Knockdown (KD) of ALK7 mRNA Expression in Adipose Tissue Collected
from ALK7 Humanized Mice 4-Weeks After Receiving a Single Subcutaneous
Dose of Adipose Tissue Shuttles Conjugated to an ALK7 Tool Sequence

| | | | e WAT | | iWAT | |
|---|---|---|---|---|---|---|
| | | | Mean | | Mean | |
| | | | KD | | KD | |
| | | | relative | | relative | |
| | | | to | | to | |
| dsRNA | | Dose | control | | control | |
| NO | Conjugate NO | (nmol/kg) | (%) | SEM | (%) | SEM |
| | Control (PBS) | 0 | 1 | 0.07 | 1 | 0.05 |
| 291* | N/A | 100 | 0.40 | 0.02 | 0.48 | 0.04 |
| 292* | N/A | 100 | 0.44 | 0.06 | 0.60 | 0.10 |
| 293* | N/A | 100 | 0.29 | 0.06 | 0.34 | 0.08 |
| 294 | C42 | 100 | 0.62 | 0.07 | 0.55 | 0.08 |
| 294 | C43 | 100 | 0.65 | 0.02 | 0.68 | 0.06 |
| 294 | C44 | 100 | 0.70 | 0.06 | 0.70 | 0.04 |
| 294 | C45 | 100 | 0.59 | 0.04 | 0.57 | 0.07 |
| 294 | C46 | 100 | 0.47 | 0.01 | 0.50 | 0.06 |
| 294 | C47 | 100 | 0.49 | 0.09 | 0.53 | 0.09 |
| 294 | C48 | 100 | 0.49 | 0.04 | 0.55 | 0.06 |
| 295 | C49 | 100 | 0.50 | 0.07 | 0.60 | 0.07 |

*These dsRNAs were synthesized and characterized with chemistry configuration that is noted associated with Table 20B.

### Example 21: In Vivo Adipose Shuttle Comparison Study with ALK7 siRNA in Cynomolgus Monkeys

To compare the in vivo efficacy of different adipose shuttles, ALK7 siRNA duplexes were conjugated to peptide and/or lipid-based shuttles as described herein and tested in Cynomolgus monkeys. On day 1, animals (n=5 for control (PBS), n=6 and test article, n=4) received a single subcutaneous dose of vehicle, or the ALK7 siRNA conjugates (100 nmol/kg). Adipose tissue samples were collected 4-weeks post administration of the ALK7 siRNA. Expression of Cynomolgus monkey ALK7 mRNA was determined using qPCR, with PPIB, ELF1, RPL30 used as the reference genes. ALK7 mRNA expression for each compound was normalized relative to vehicle (PBS, Table 34—set to 1). Knockdown in Table 34 was expressed in the form of decimal fraction remaining ALK7 mRNA relative to control.

TABLE 34
In Vivo Knockdown (KD) of ALK7 mRNA Expression in Adipose Tissue Collected
from Cynomolgus Monkeys 4-Weeks After Receiving a Single Subcutaneous
Dose of Adipose Tissue Shuttles Conjugated to an ALK7 Tool Sequence

| | | | | | Gluteal | | | |
|---|---|---|---|---|---|---|---|---|
| | | | Omental | | Subcutaneous | | Abdominal | |
| | | | WAT | | WAT | | WAT | |
| | | | Mean | | Mean | | Mean | |
| | | | KD | | KD | | KD | |
| | | | relative | | relative | | relative | |
| | | | to | | to | | to | |
| dsRNA | Conjugate | Dose | control | | control | | control | |
| NO | NO | (nmol/kg) | (%) | SEM | (%) | SEM | (%) | SEM |
| N/A | Vehicle | 0 | 1 | 0.14 | 1 | 0.18 | 1 | 0.13 |
| 291* | N/A | 100 | 0.14 | 0.06 | 0.08 | 0.02 | 0.11 | 0.05 |
| 292* | N/A | 100 | 0.23 | 0.07 | 0.24 | 0.12 | 0.18 | 0.06 |
| 293* | N/A | 100 | 0.33 | 0.13 | 0.16 | 0.01 | 0.27 | 0.09 |
| 294 | C42 | 100 | 0.64 | 0.20 | 0.59 | 0.13 | 0.61 | 0.08 |
| 294 | C43 | 100 | 1.10 | 0.03 | 0.93 | 0.19 | 0.71 | 0.16 |
| 294 | C44 | 100 | 0.66 | 0.23 | 0.54 | 0.18 | 0.73 | 0.10 |
| 294 | C45 | 100 | 0.78 | 0.06 | 0.53 | 0.19 | 0.56 | 0.03 |
| 294 | C46 | 100 | 0.22 | 0.04 | 0.24 | 0.05 | 0.28 | 0.03 |
| 294 | C47 | 100 | 0.74 | 0.15 | 0.58 | 0.22 | 0.62 | 0.16 |
| 294 | C48 | 100 | 0.67 | 0.09 | 0.54 | 0.08 | 0.66 | 0.08 |
| 295 | C49 | 100 | 0.13 | 0.09 | 0.12 | 0.02 | 0.21 | 0.04 |
| | | | Mesenteric | | | | Perirenal | |
| | | | Adipose Tissue | | PC | | Adipose Tissue | |
| | | | Mean | | Mean | | Mean | |
| | | | KD | | KD | | KD | |
| | | | relative | | relative | | relative | |
| dsRNA | Conjugate | Dose | to | | to | | to | |
| NO | NO | (nmol/kg) | control | SEM | control | SEM | control | SEM |
| | Vh | 0 | 1 | 0.21 | 1 | 0.11 | 1 | 0.17 |
| 291* | N/A | 100 | 0.21 | 0.10 | 0.07 | 0.03 | 0.04 | 0.01 |
| 292* | N/A | 100 | 0.22 | 0.04 | 0.16 | 0.01 | 0.19 | 0.06 |
| 293* | N/A | 100 | 0.21 | 0.02 | 0.30 | 0.10 | 0.28 | 0.06 |
| 294 | C42 | 100 | 1.12 | 0.15 | 0.51 | 0.15 | 0.68 | 0.06 |
| 294 | C43 | 100 | 1.00 | 0.14 | 1.11 | 0.13 | 0.72 | 0.11 |
| 294 | C44 | 100 | 1.03 | 0.31 | 0.66 | 0.30 | 0.64 | 0.16 |
| 294 | C45 | 100 | 0.84 | 0.30 | 0.77 | 0.06 | 0.54 | 0.10 |
| 294 | C46 | 100 | 0.33 | 0.06 | 0.37 | 0.08 | 0.24 | 0.03 |
| 294 | C47 | 100 | 0.52 | 0.18 | 0.92 | 0.11 | 0.60 | 0.22 |
| 294 | C48 | 100 | 0.79 | 0.22 | 0.63 | 0.14 | 0.55 | 0.05 |
| 295 | C49 | 100 | 0.21 | 0.03 | 0.24 | 0.09 | 0.13 | 0.01 |

*These dsRNAs tested were those of chemistry configuration that is noted associated with Table 20B.

SEQUENCE LISTING

| SEQ | |
|---|---|
| ID | |
| NO | Sequence |
| 1 | GX11IDX14I where X11 is R, r, P, p Hyp or D-Hyp; X14 is R, r or Arg(Me) |
| 2 | GRIDRI |
| 3 | SX7X8X9GX11IDX14I where X7 is G, g, A, a, P, p, Hyp, D-Hyp S, s, C, or c; X8 is F, f or |
| | Cha, X9 is G, g, A, a or S, s; X11 is R, r, P, p Hyp or D-Hyp; X14 is R, r or Arg(Me) |
| 4 | SCFGGRIDRI |
| 5 | FGGRIDRIGA |
| 6 | RSSX7FGGRIDRI where X7 is S, s, C or c |
| 7 | RSSX7FGGRIDRIGA where X7 is S, s, C or c |
| 8 | X7-Cha-X9GX11IDX14I where X7 is P, p, Hyp, D-Hyp, |
| | G, g, C, c, A, or a; X9 is A, a, S, s, G |
| | or g; X11 is P, p, Hyp R or r; and X14 is R, r or Arg(Me) |
| 9 | fSp-Cha-aGPIDRI |
| 10 | SLRRSS[CFGGRIDRIGAQSGLGC]NSFRY-NH2 |
| 11 | RSS[CFGGRIDRIGAQSGLGC]-NH2 |
| 12 | RSSCFGGRIDRIGAQSGLGC-NH2 |
| 13 | RSSSFGGRIDRIGAQSGLGS-NH2 |
| 14 | RSS[CFGGRIDRIGAQSGLGC]-OH |
| 15 | RSSCFGGRIDRIGAQSGLGC-OH |
| 16 | RSSSFGGRIDRIGAQSGLGS-OH |
| 17 | RSSSFGGRIDRIGAQSGLGS-NH2 |
| 18 | RSS[CFGGRIDRIGAC]-NH2 |
| 19 | RSS[CFGGRIDRIGAQSGLGC]^{CH2}-OH |
| 20 | RSS[CFGGRIDRIGAQSGLGC]^{CH2}-NH2 |
| 21 | fSp-Cha-aGPIDRI-NH2 |
| 22 | fS-(D-Hyp)-Cha-sG-Hyp-ID-Arg(Me)-I-NHCH3 |
| 23 | fSp-Cha-aGPIDRIGSPSSGAPPPS-NH2 |
| 24 | fSp-Cha-aGPIDRI-OH |
| 25 | Orn-fSp-Cha-aGPIDSI-NH2 |
| 26 | fSp-Cha-aGPIDSI-Dap-NH2 |
| 27 | fS-(D-Pip)-Cha-aGPIDSI-NH2 |
| 28 | FSG-Cha-GGRIDRI-NH2 |
| 29 | FSG-Cha-Aib-GRIDRI-NH2 |
| 30 | fSp-Cha-aGPIERI-NH2 |
| 31 | fSp-Cha-aGPID-(NMe-Arg)-I-NH2 |
| 32 | RSFSGFGGRIDRI-NH2 |
| 33 | RSFSGFGGRIDRIGAQSGLGS-NH2 |
| 34 | RSFSGFGGRIDRIG-NH2 |
| 35 | C-Cha-GGRIDRIG-NH2 |
| 36 | Ac-C-Cha-GGRIDRIG-NH2 |
| 37 | S-Cha-GGRIDRIG-NH2 |
| 38 | A-Cha-GGRIDRIG-NH2 |
| 39 | Cha-GGRIDRIG-NH2 |
| 40 | Ac-Cha-GGRIDRIG-NH2 |
| 41 | G-Cha-GGRIDRIG-NH2 |
| 42 | SG-Cha-GGRIDRIG-NH2 |
| 43 | FSG-Cha-GGRIDRIG-NH2 |
| 44 | SFSG-Cha-GGRIDRIG-NH2 |
| 45 | RSFSG-Cha-GGRIDRIG-NH2 |
| 46 | RSSG-Cha-GGRIDRIG-NH2 |
| 47 | fSG-Cha-GGRIDRIG-NH2 |
| 48 | Ac-fSG-Cha-GGRIDRIG-NH2 |
| 49 | fSp-Cha-GGRIDRIG-NH2 |
| 50 | fSG-Cha-aGRIDRIG-NH2 |
| 51 | fSG-Cha-GGPIDRIG-NH2 |
| 52 | Ac-fSp-Cha-aGPIDRIG-NH2 |
| 53 | Ac-fSp-Cha-aGPIDRI-NH2 |
| 54 | Ac-fSp-Cha-aGPIDRI-NHCH3 |
| 55 | Ac-fSp-Cha-aGPID-Arg(Me)-I-NHCH3 |
| 56 | Ac-fS-(D-Hyp)-Cha-aG-Hyp-ID-Arg(Me)-I-NHCH3 |
| 57 | (HOCH2CO)-fS-(D-Hyp)-Cha-sG-Hyp-ID-Arg(Me-)I-NHCH3 |
| 58 | AEEA3-fSp-Cha-aGPIDRIGGGGSGGGGS |
| 59 | GGGGSGGGGSfSp-Cha-aGPIDRIGGGGSGGGGS |
| 60 | GGGGSGGGGSGGGGSGGGGS |
| 61 | AEEA3-GGGGSGGGGSGGGGSGGGGS |
| 62 | GGGGHGGGGHGGGGHGGGGH |
| 63 | GGGG-4Pal-GGGG-4Pal-GGGG-4Pal-GGGG-4Pal |
| 64 | GGGGSHHHGGGGSHHHGGGGS |
| 65 | AEEA3-GGGGSGGGGS |
| 66 | GGGGSGGGGSGGGGSGGGGSGGGGSGGGGS |
| 67 | GGGGSGGGGSGGGGSGGGGSP |
| 68 | GGGG-4Pal-GGGG-4Pal-GGGG-4Pal-GGGG-4Pal-P |
| 69 | GGGG-4Pal-GGGG-4Pal-GGGG-4Pal-GGGG-4Pal-GGGG-4Pal-GGGG-4Pal |
| 70 | GGGGEGGGGEGGGGSGGGGSGGGGSGGGGS |
| 71 | GGGGEGGGGSGGGGEGGGGSGGGGEGGGGS |
| 72 | GGGGEGGGGEGGGGEKEKEKGGGGSGGGGS |
| 73 | GSPSSGAPPPS |
| 74 | MPSLLVLTFSPCVLLGWALLAGGTGGGGVGGGGGGAGIGGGRQEREALPPQKIEVLV |
| | LLPQDDSYLFSLTRVRPAIEYALRSVEGNGTGRRLLPPGTRFQVAYEDSDCGNRALFSL |
| | VDRVAAARGAKPDLILGPVCEYAAAPVARLASHWDLPMLSAGALAAGFQHKDSEYS |
| | HLTRVAPAYAKMGEMMLALFRHHHWSRAALVYSDDKLERNCYFTLEGVHEVFQEE |
| | GLHTSIYSFDETKDLDLEDIVRNIQASERVVIMCASSDTIRSIMLVAHRHGMTSGDYAF |
| | FNIELFNSSSYGDGSWKRGDKHDFEAKQAYSSLQTVTLLRTVKPEFEKFSMEVKSSVE |
| | KQGLNMEDYVNMFVEGFHDAILLYVLALHEVLRAGYSKKDGGKIIQQTWNRTFEGIA |
| | GQVSIDANGDRYGDFSVIAMTDVEAGTQEVIGDYFGKEGRFEMRPNVKYPWGPLKLR |
| | IDENRIVEHTNSSPCKSSGGLEESAVTGIVVGALLGAGLLMAFYFFRKKYRITIERRTQQ |
| | EESNLGKHELREDSIRSHFSVA |
| 75 | MPSLLVLTFSPCVLLGWALLAGGTGGGGVGGGGGGAGIGGGRQEREALPPQKIEVLV |
| | LLPQDDSYLFSLTRVRPAIEYALRSVEGNGTGRRLLPPGTRFQVAYEDSDCGNRALFSL |
| | VDRVAAARGAKPDLILGPVCEYAAAPVARLASHWDLPMLSAGALAAGFQHKDSEYS |
| | HLTRVAPAYAKMGEMMLALFRHHHWSRAALVYSDDKLERNCYFTLEGVHEVFQEE |
| | GLHTSIYSFDETKDLDLEDIVRNIQASERVVIMCASSDTIRSIMLVAHRHGMTSGDYAF |
| | FNIELFNSSSYGDGSWKRGDKHDFEAKQAYSSLQTVTLLRTVKPEFEKFSMEVKSSVE |
| | KQGLNMEDYVNMFVEGFHDAILLYVLALHEVLRAGYSKKDGGKIIQQTWNRTFEGIA |
| | GQVSIDANGDRYGDFSVIAMTDVEAGTQEVIGDYFGKEGRFEMRPNVKYPWGPLKLR |
| | IDENRIVEHTNSSPCKSCGLEESAVTGIVVGALLGAGLLMAFYFFRKKYRITIERRTQQE |
| | ESNLGKHRELREDSIRSHFSVA |
| 76 | agtggcaggagcgccgcgcaccgccagccgcagggggcgtgggatgggggcggccgggga |
| | ggggggcgcccacactgactagagccaaccgcgcacttcaaaagggtgtcggtgccgcgc |
| | tcccctcccgcggcccgggaacttcaaagcgggccgtgctgccccggctgcctcgctctg |
| | ctctggggcctcgcagccccggcgcggccgcctggtggcgatgacccgggcgctctgctc |
| | agcgctccgccaggctctcctgctgctcgcagcggccgccgagctctcgccaggactgaa |
| | gtgtgtatgtcttttgtgtgattcttcaaacttcacctgccaaacagaaggagcatgttg |
| | ggcatcagtcatgctaaccaatggaaaagagcaggtgatcaaatcctgtgtctcccttcc |
| | agaactgaatgctcaagtcttctgtcatagttccaacaatgttaccaaaaccgaatgctg |
| | cttcacagatttttgcaacaacataacactgcaccttccaacagcatcaccaaatgcccc |
| | aaaacttggacccatggagctggccatcattattactgtgcctgtttgcctcctgtccat |
| | agctgcgatgctgacagtatgggcatgccagggtcgacagtgctcctacaggaagaaaaa |
| | gagaccaaatgtggaggaaccactctctgagtgcaatctggtaaatgctggaaaaactct |
| | gaaagatctgatttatgatgtgaccgcctctggatctggctctggtctacctctgttggt |
| | tcaaaggacaattgcaaggacgattgtgcttcaggaaatagtaggaaaaggtagatttgg |
| | tgaggtgtggcatggaagatggtgtggggaagatgtggctgtgaaaatattctcctccag |
| | agatgaaagatcttggtttcgtgaggcagaaatttaccagacggtcatgctgcgacatga |
| | aaacatccttggtttcattgctgctgacaacaaagataatggaacttggactcaactttg |
| | gctggtatctgaatatcatgaacagggctccttatatgactatttgaatagaaatatagt |
| | gaccgtggctggaatgatcaagctggcgctctcaattgctagtggtctggcacaccttca |
| | tatggagattgttggtacacaaggtaaacctgctattgctcatcgagacataaaatcaaa |
| | gaatatcttagtgaaaaagtgtgaaacttgtgccatagcggacttagggttggctgtgaa |
| | gcatgattcaatactgaacactatcgacatacctcagaatcctaaagtgggaaccaagag |
| | gtatatggctcctgaaatgcttgatgatacaatgaatgtgaatatctttgagtccttcaa |
| | acgagctgacatctattctgttggtctggtttactgggaaatagcccggaggtgttcagt |
| | cggaggaattgttgaggagtaccaattgccttattatgacatggtgccttcagatccctc |
| | gatagaggaaatgagaaaggttgtttgtgaccagaagtttcgaccaagtatcccaaacca |
| | gtggcaaagttgtgaagcactccgagtcatggggagaataatgcgtgagtgttggtatgc |
| | caacggagcggcccgcctaactgctcttcgtattaagaagactatatctcaactttgtgt |
| | caaagaagactgcaaagcctaatgatgataattatgttaaaaagaaatctctcatagctt |
| | tcttttccattttcccctttatgtgaatgtttttgccattttttttttgttctacctcaa |
| | agataagacagtacagtatttaagtgcccataaggcagcatgaaaagataactctaaagt |
| | taagcatgggcaggagttgacttcatccaatctctatgttatgtttaattttattttgaa |
| | agcaacacctcaactcatctttttatttaataaggaagaaatatattacaaaagtataaa |
| | ataagctctataaaaatgttatagtcattaagtttttattttacttgaaccaagagcaca |
| | tgaatgaacaggaaaagatgtaaaaacatttttttctgagatgaaaacatattaattaaa |
| | catgcaaattagagcatgctatctttaggtgatgcaatctatgtttccccctttttaagt |
| | tagcaggactttttaaaaataaatattgctctaaactttaatatatcgaacgtgagagtg |
| | gagctgcttagtggaagatgtaagtgaggtgggtgtcccatgtgcttggtctccccttct |
| | gctgttctcctgttcttcataatccactactgcagcagtccctgaaccactaaacttgtt |
| | cctttcatttacaaaagagatacctgacatcctgagacactgagaaatgtcctgaagtca |
| | cacagctaatggcagaactggcactaggtccaaatcttgtgataatgaacaccgtaaggt |
| | tagctagcttcctactttcccttgaatagtgcttttctccctatgtaatatcttttatta |
| | tgatatttgtggtttagaaggcatattgagttattttgcagaatcataatggacccgcac |
| | aaaatctcagaaccatatctgttgacattttttctcatagaaatatcatggttaccccat |
| | ttgttaatgagcattaatgttttctgaacacttccaaagattaatcaaacataaatattc |
| | attgtctgaaaatgtctttaagatacaattcagaggtccctatttcctttgtacatacac |
| | acttagaaagaaaagacagaaaaggaagaggaaggaaggaaatattttgagaatatattg |
| | agaagaattaagaaaactcttcaatgaagtgttaacaaccaaaccctacagacggtatca |
| | gaaacagcaaatagatattcctctaccctttcacagtgagtgagtgagtacagaagaatg |
| | ctcatgatagttttgccttcattctactttctgtggacacagagtaatgaatatttaatg |
| | ggacattaaatatgcccttcaaatctataattttactttggtaaacgagatttaacatga |
| | tgtcttttatgctcctaaaacatcttttttcaaactccattccttagaacattcttctac |
| | tgagatgatccaagaccaaaagtgttctttggtacttgcttataaagtgatagtacatgt |
| | tagcatataatgtattttgaagagtgaagtaaatgctattgataacagtaaaaaaaaaaa |
| | aaaaaactaataacagtaaagaaatgctacttgatttttttttaaactggattgctcaga |
| | ttacctgatcgtggtggaacccttttattaagaggaggggaaactttttactatcccata |
| | tttaactgttctataaagcaaagcacagcttgggtaatagcgttctgaaggatatacttc |
| | tgtattttctcatagagtacaatttagtgattatgcttcatttcactatggaaatatgtt |
| | actgaatctatcttcattttactgagttgaaataaggaaggcaaaaaactgacagctatg |
| | gagtttgcgtgtacttccatactcgttaatgctctcatccacttattaaataatcataga |
| | gcacccatatcttgcttgccacaatatcgggtacaagagggaatacaaagatagataggt |
| | cctgccctcagggattttaaagtctaatttgggaatgggaatagggatgtgagtgtgtgg |
| | gggaagagattaaattgacagataaaatacgaagtgggatgtcttgagttctgcatgaca |
| | gtgggtttctaggataggtctgaaaattgctttcatttgcaacacatttagaaagtagct |
| | ttatttggatattacagacaatctaaatatatcatcagtttttaaaagtgcctatgtgaa |
| | gtgatttttaaaaagagcctatgtgaaggggtaatcttgcttgttcttgttactaatttc |
| | tcatagattgtttttctgcatatataagaacgaaattatttatttactatggttgtacgt |
| | gcctcaaataaacaagaatgatatttcctgttttatttacttatgttgggtaaatatgct |
| | tattgaatttttaagagaggattttttaccatctccatttttcttgtcattatgttttgt |
| | agcttatttgagggtgtctaaatataatttcatattttattggttcaactttcactctga |
| | agaaatccgtatgttagtacattttgaggtatttttcttgttcttgtgttgtttaactat |
| | gactcctaactgagtagtcttatatttcaattacaaaatacattttttaagaaagggaat |
| | agagcagcaaaaatgataaggaaaatgttaaaagttgtaatatttcctttactcttaaca |
| | ggattatatatagaacatgctcacttacaaaaataggatgatgaagtttagagcataagg |
| | caggcttcttgtatatacttatgctgtcaaatgttatattgtttttaatggagtcccatt |
| | gtgtaatatttatttcttttacattttgttataagcaaaaaaaaaaaaaattctccttag |
| | gttatgttcagagtatcagtgttctttactccttacagatattttggctttggggtataa |
| | tacaagacttgggaaaacactattatgaattttcagtactgtataaagtggtgatgggat |
| | ttaaatgcagcatcactttctgaaaataagagaaacattatttgttgtcagtatttcagc |
| | atgaacttgttgccttgtaaattttgcctttaagtttgtaattggtacagattctgttgt |
| | atgctttcttctatgtctaaaatatttggcatgtcacatctagaattcttaatttatgtt |
| | ctgacttgagagttaagtgaaacatgactgtcgtgcactattttaggcatagcacttgct |
| | tttcatctttatactttcaattaactttgcattttaaatttccatgattgtatgaaaata |
| | gtaacctggttgcagtatctgaaaaaattaaaattagcttttatttaaaaatgaaaatct |
| | acaatagattcattaggttagaagttccagaataatttattattttattacacctactat |
| | tgtagaattactaataaaacaaaacaaaactactcctattttcctggaattttgccacca |
| | tgtgacttattggggcagagaaaactcagggttgtctttgagtctgcacaaaagcaccag |
| | ggaacctgcttagcaaatcgtctgaaaacagggagctgatgtttgccattatacaaagtt |
| | tgagtaaacaacttaaaattgcttgttagggcatagtctttgattgaaataagtatgaga |
| | atgtatttggctaaataaatgtatttaaaatatacaaattttatttcccactggaaaatt |
| | aagaaagtagcagtaccaaatgaataaaagctggcagttgatgtcttcaataatcattcc |
| | tttaaaataaattcacaaacacatcattacaagctacttagaaatgtttagtattcgtat |
| | cttaaaatggcaccattgtggttttcaaagatattttaaacatcttttgtgaaacatact |
| | atttctgtttgacaatgctttgttctagctctgtgtgctgatacctacatggagtttttc |
| | tgctattttagtgaaaacagtaattcttttatcttgagagctgcttctaaataacaaaaa |
| | agttaattggaatgtaagtttttaaaaaatgttaatattaaatagaatttttataatatg |
| | ggcatttttcaaaacattttaatgaaaatatatagatatttgactttctttttttcatct |
| | agccttgctttatatttcattatttttctcactttttttcttaatattccctcactatct |
| | ttcaacattatcattagttcctaattttaaaaataactcttatttaacttaaccgtctgg |
| | aatttagccttctaatgaaagagaatcccttagtactctcagagattatataaagttatt |
| | ccaattttgtgtagatgacgaaaccaaggatcagagattaaatgactactagttactagc |
| | agaactcttatgaaagcctggtggtgacacccagcactgttccctgccatatccccaact |
| | tttactgattaaaaattaattcgcatgcatctaaacctaccttgaatgcacactaacgta |
| | atgtgccattcaatgatgatgataaaatcccacttttctttgggtttctactaaaataat |
| | ttactcactcagagtgaggtcaatgagaaaaactaaactaggctaagaaagagttgtaga |
| | atggttgttgagctaagtaggcaaccactggggtgctgataaaattaatggataaaattt |
| | aggaactgtgagagtatagaatttcttagtgcaagtaatgattagagaagcttcctgaca |
| | tcccccaccctttctgtaaggactgttctttctctttgtaccttggaatgggggtgaaag |
| | gtgatttgatagctgaatttgggactatgtccagtgggatattatctaacttttctctct |
| | ttctcttttttttcccctcagtttctcattagtttgtctttggcattcatcttcttttag |
| | tgcattaaaaaatgtttggcagatctcatcaatcccaagtcactctataattcctgtatt |
| | tctttagttgtcgtttaacctgtccaaacttctacacaatgaacttcttaacaagatttt |
| | aagttctctgtataagaggtttcacctcataacttctccagtaatccttcatttggcact |
| | atagagtatttgttaatggcagagatgatttttcttttaaaacctaaaaagactagctgt |
| | tatttgtattctagcttttagctaacatataaagaatgtctacttttgctttaatgctaa |
| | attccgcttgagaaatagtaactgggaaagacaatttgaaatatatgccccataatgtga |
| | ttgttaaatttatttctgctgttccataccattgctttgttttgcttttgacaaactaag |
| | ccattatgctattagtttggaatataatactacagcaaaattaggtaacaatccctattt |
| | taaaattcccctaacaataatagaactgccagcatacttttctctttcagttgtagatga |
| | atacattcgagagaatatgagctgtatttcatcctagattttaatattttcagatgtgac |
| | tgtatttcctgatcattggtccaagttgtcctaaaagaaatttttctctccagacctaac |
| | agttttaactgcaagagtttactgtgggttatgttaatctgaattttaatagggccacta |
| | agaatctgagtgccttaggagattacccttatacccactgccatcacatccagtcaggcc |
| | tgttgtgctctatataaatcttcccagctgaggggcaggtgcgggctaaaatccaactgc |
| | aattggctcccagacataattttatattttacagagaagcatcttattggcttatatgtg |
| | tttaaagaatggtctggcttatacatcttcagaaaatgagaattaaaaagtcaaaataat |
| | tcttgacatctacagattgaacaaagaacttagaagaaataatactttatcttttcatcc |
| | tggcattcctgagagaagagaaattgattgtttatcatgttggtttaatttttcaaccca |
| | gacaatctgcagcaaggcacatggaccccaattttgatatcgtccatacagttttcattc |
| | tatgcatggagctaattactgactttgcctgtaaagagaggattgtgtgcctaaattttg |
| | tctaacaaatgcaagcgtagaatgacatttactaatatttctatttcttccataggctaa |
| | ataatagtaactaagtatttttaaggacacagccctttttttctctttatacaaaatgag |
| | agtatctgagccaaaatattaaattctagttcttttccgcaatgactagtgtcaagctca |
| | tgtactcttctgattctagactggagaagattattcaaacttgatctgtgtttcaggttt |
| | ttaaatgtcctaaaaacagaaaattagattcagatctcaaaaaaggaattttggattgac |
| | tttcaaagtactaatactaattatacttttcttttggtagcgtgactcttcttataccta |
| | agaacatattacaaatgtcaaaaccattgcattttgacattgcaaaacatgccttgaact |
| | cttgaactactgtgaaaagaatcaccgttgtaaagactttttgtaagctagctgatactc |
| | ttaagtatgtaaaaagattgtctttcagccgacaggcccaaaggaatgtatataaggaag |
| | gaatatgaaaaaataaattaggttttaaaataggaattgggcaataaactgtatcaaaaa |
| | tatgtagatggattttagtagttgtaatttaaatgtggaaggtgaagagaatttcaaact |
| | ccaaagagaaatgaatgatattcagatgtttcattaatttctagtctgtgaaaatatgca |
| | ttttatagtaatatgtatagacttattttatttagaaataatagtgttttagaatttatt |
| | aaaaactcagtgatagcctttataccaaaatgtttaactttaccaacagcaagtcataaa |
| | agtatttattttaaagctttttaatattatcgtgtaactttcatctgtcttcagatgtaa |
| | ataattatctgcctaaatgttatatttttatgtatgcattttctgaaaatgtattgtttt |
| | gtaaagtgggaaagataataaatcaagcacttcttgcacttgtttctgtgaagcatatag |
| | aactctattttaaataaaggaagatgtgtcgta |
| 77 | MTRALCSALRQALLLLAAAAELSPGLKCVCLLCDSSNFTCQTEG |
| | ACWASVMLTNGKEQVIKSCVSLPELNAQVFCHSSNNVTKTECCFTDFCNNITLHLPTA |
| | SPNAPKLGPMELAIIITVPVCLLSIAAMLTVWACQGRQCSYRKKKRPNVEEPLSECNL |
| | VNAGKTLKDLIYDVTASGSGSGLPLLVQRTIARTIVLQEIVGKGRFGEVWHGRWCGED |
| | VAVKIFSSRDERSWFREAEIYQTVMLRHENILGFIAADNKDNGTWTQLWLVSEYHEQG |
| | SLYDYLNRNIVTVAGMIKLALSIASGLAHLHMEIVGTQGKPAIAHRDIKSKNILVKKC |
| | ETCAIADLGLAVKHDSILNTIDIPQNPKVGTKRYMAPEMLDDTMNVNIFESFKRADIY |
| | SVGLVYWEIARRCSVGGIVEEYQLPYYDMVPSDPSIEEMRKVVCDQKFRPSIPNQWQS |
| | CEALRVMGRIMRECWYANGAARLTALRIKKTISQLCVKEDCKA |
| 78 | UUUUAUUUUACUUGAACCAAA |
| 79 | CCCGCCUAACUGCUCUUCGUA |
| 80 | CUAAUGAUGAUAAUUAUGUUA |
| 81 | AAGUAUAAAAUAAGCUCUAUA |
| 82 | UCCAUUUUCCCCUUUAUGUGA |
| 83 | AUGUUAAAAAGAAAUCUCUCA |
| 84 | AUAAGACAGUACAGUAUUUAA |
| 85 | AACUCUAAAGUUAAGCAUGGA |
| 86 | UUUAAUUUUAUUUUGAAAGCA |
| 87 | GUUAUAGUCAUUAAGUUUUUA |
| 88 | UUUAUGUGAAUGUUUUUGCCA |
| 89 | UUAUGACAUGGUGCCUUCAGA |
| 90 | UCAAAGAAGACUGCAAAGCCA |
| 91 | UGAAAAGAUAACUCUAAAGUA |
| 92 | UUUACUUGAACCAAGAGCACA |
| 93 | CAACUCAUCUUUUUAUUUAAA |
| 94 | AUGAUGAUAAUUAUGUUAAAA |
| 95 | UAUAUUACAAAAGUAUAAAAA |
| 96 | GUGAUAGUACAUGUUAGCAUA |
| 97 | UUGCCUUAUUAUGACAUGGUA |
| 98 | UAGCAUAUAAUGUAUUUUGAA |
| 99 | ACUUCUGUAUUUUCUCAUAGA |
| 100 | UUUUGUGUGAUUCUUCAAACA |
| 101 | UCUAAAGUUAAGCAUGGGCAA |
| 102 | GACAUGGUGCCUUCAGAUCCA |
| 103 | GCCCGCCUAACUGCUCUUCGA |
| 104 | UCAAAGAAUAUCUUAGUGAAA |
| 105 | GUAUCCCAAACCAGUGGCAAA |
| 106 | UUUUAUUAUGAUAUUUGUGGA |
| 107 | UUUUUUUAAACUGGAUUGCUA |
| 108 | UGUCUUUUGUGUGAUUCUUCA |
| 109 | CUCUAAAGUUAAGCAUGGGCA |
| 110 | UUUUACUUGAACCAAGAGCAA |
| 111 | UCCUGAAAUGCUUGAUGAUAA |
| 112 | UGAAAUGCUUGAUGAUACAAA |
| 113 | CAAGGUAAACCUGCUAUUGCA |
| 114 | GGGCUCCUUAUAUGACUAUUA |
| 115 | GGCUCCUGAAAUGCUUGAUGA |
| 116 | UUUAUUUUACUUGAACCAAGA |
| 117 | UUGAUGAUACAAUGAAUGUGA |
| 118 | UGAUUCAAUACUGAACACUAA |
| 119 | AUCUAUUCUGUUGGUCUGGUA |
| 120 | AGUUGACUUCAUCCAAUCUCA |
| 121 | CUCAUCGAGACAUAAAAUCAA |
| 122 | UACCUCAAAGAUAAGACAGUA |
| 123 | UAUAGUCAUUAAGUUUUUAUA |
| 124 | CAAAGAUAAGACAGUACAGUA |
| 125 | AAAGAAUAUCUUAGUGAAAAA |
| 126 | ACCAAUUGCCUUAUUAUGACA |
| 127 | UUUUUGUUCUACCUCAAAGAA |
| 128 | UUCCAUUUUCCCCUUUAUGUA |
| 129 | CUACCUCAAAGAUAAGACAGA |
| 130 | AAUCAAAGAAUAUCUUAGUGA |
| 131 | AUGCUUGAUGAUACAAUGAAA |
| 132 | UAGGUGAUGCAAUCUAUGUUA |
| 133 | AUAUCUCAACUUUGUGUCAAA |
| 134 | AUGAAUGUGAAUAUCUUUGAA |
| 135 | UUCAAACGAGCUGACAUCUAA |
| 136 | AAAAAUAAAUAUUGCUCUAAA |
| 137 | CUCUCAUAGCUUUCUUUUCCA |
| 138 | AUAGAGGAAAUGAGAAAGGUA |
| 139 | GUGAAAAAGUGUGAAACUUGA |
| 140 | UGGACUCAACUUUGGCUGGUA |
| 141 | UUAAGUUUUUAUUUUACUUGA |
| 142 | GAAGAUGUGGCUGUGAAAAUA |
| 143 | UAUGUUAAAAAGAAAUCUCUA |
| 144 | UAAUGAUGAUAAUUAUGUUAA |
| 145 | GAUAAUUAUGUUAAAAAGAAA |
| 146 | GACUGCAAAGCCUAAUGAUGA |
| 147 | CAUUAAGUUUUUAUUUUACUA |
| 148 | ACUGCUCUUCGUAUUAAGAAA |
| 149 | UUAUGUUAAAAAGAAAUCUCA |
| 150 | GUUGACUUCAUCCAAUCUCUA |
| 151 | CCUGAAAUGCUUGAUGAUACA |
| 152 | GAAAUGCUUGAUGAUACAAUA |
| 153 | UCCUUAUAUGACUAUUUGAAA |
| 154 | UUUGGUUCAAGUAAAAUAAAAAC |
| 155 | UACGAAGAGCAGUUAGGCGGGCC |
| 156 | UAACAUAAUUAUCAUCAUUAGGC |
| 157 | UAUAGAGCUUAUUUUAUACUUUU |
| 158 | UCACAUAAAGGGGAAAAUGGAAA |
| 159 | UGAGAGAUUUCUUUUUAACAUAA |
| 160 | UUAAAUACUGUACUGUCUUAUCU |
| 161 | UCCAUGCUUAACUUUAGAGUUAU |
| 162 | UGCUUUCAAAAUAAAAUUAAACA |
| 163 | UAAAAACUUAAUGACUAUAACAU |
| 164 | UGGCAAAAACAUUCACAUAAAGG |
| 165 | UCUGAAGGCACCAUGUCAUAAUA |
| 166 | UGGCUUUGCAGUCUUCUUUGACA |
| 167 | UACUUUAGAGUUAUCUUUUCAUG |
| 168 | UGUGCUCUUGGUUCAAGUAAAAU |
| 169 | UUUAAAUAAAAAGAUGAGUUGAG |
| 170 | UUUUAACAUAAUUAUCAUCAUUA |
| 171 | UUUUUAUACUUUUGUAAUAUAUU |
| 172 | UAUGCUAACAUGUACUAUCACUU |
| 173 | UACCAUGUCAUAAUAAGGCAAUU |
| 174 | UUCAAAAUACAUUAUAUGCUAAC |
| 175 | UCUAUGAGAAAAUACAGAAGUAU |
| 176 | UGUUUGAAGAAUCACACAAAAGA |
| 177 | UUGCCCAUGCUUAACUUUAGAGU |
| 178 | UGGAUCUGAAGGCACCAUGUCAU |
| 179 | UCGAAGAGCAGUUAGGCGGGCCG |
| 180 | UUUCACUAAGAUAUUCUUUGAUU |
| 181 | UUUGCCACUGGUUUGGGAUACUU |
| 182 | UCCACAAAUAUCAUAAUAAAAGA |
| 183 | UAGCAAUCCAGUUUAAAAAAAAA |
| 184 | UGAAGAAUCACACAAAAGACAUA |
| 185 | UGCCCAUGCUUAACUUUAGAGUU |
| 186 | UUGCUCUUGGUUCAAGUAAAAUA |
| 187 | UUAUCAUCAAGCAUUUCAGGAGC |
| 188 | UUUGUAUCAUCAAGCAUUUCAGG |
| 189 | UGCAAUAGCAGGUUUACCUUGUG |
| 190 | UAAUAGUCAUAUAAGGAGCCCUG |
| 191 | UCAUCAAGCAUUUCAGGAGCCAU |
| 192 | UCUUGGUUCAAGUAAAAUAAAAA |
| 193 | UCACAUUCAUUGUAUCAUCAAGC |
| 194 | UUAGUGUUCAGUAUUGAAUCAUG |
| 195 | UACCAGACCAACAGAAUAGAUGU |
| 196 | UGAGAUUGGAUGAAGUCAACUCC |
| 197 | UUGAUUUUAUGUCUCGAUGAGCA |
| 198 | UACUGUCUUAUCUUUGAGGUAGA |
| 199 | UAUAAAAACUUAAUGACUAUAAC |
| 200 | UACUGUACUGUCUUAUCUUUGAG |
| 201 | UUUUUCACUAAGAUAUUCUUUGA |
| 202 | UGUCAUAAUAAGGCAAUUGGUAC |
| 203 | UUCUUUGAGGUAGAACAAAAAAA |
| 204 | UACAUAAAGGGGAAAAUGGAAAA |
| 205 | UCUGUCUUAUCUUUGAGGUAGAA |
| 206 | UCACUAAGAUAUUCUUUGAUUUU |
| 207 | UUUCAUUGUAUCAUCAAGCAUUU |
| 208 | UAACAUAGAUUGCAUCACCUAAA |
| 209 | UUUGACACAAAGUUGAGAUAUAG |
| 210 | UUCAAAGAUAUUCACAUUCAUUG |
| 211 | UUAGAUGUCAGCUCGUUUGAAGG |
| 212 | UUUAGAGCAAUAUUUAUUUUUAA |
| 213 | UGGAAAAGAAAGCUAUGAGAGAU |
| 214 | UACCUUUCUCAUUUCCUCUAUCG |
| 215 | UCAAGUUUCACACUUUUUCACUA |
| 216 | UACCAGCCAAAGUUGAGUCCAAG |
| 217 | UCAAGUAAAAUAAAAACUUAAUG |
| 218 | UAUUUUCACAGCCACAUCUUCCC |
| 219 | UAGAGAUUUCUUUUUAACAUAAU |
| 220 | UUAACAUAAUUAUCAUCAUUAGG |
| 221 | UUUCUUUUUAACAUAAUUAUCAU |
| 222 | UCAUCAUUAGGCUUUGCAGUCUU |
| 223 | UAGUAAAAUAAAAACUUAAUGAC |
| 224 | UUUCUUAAUACGAAGAGCAGUUA |
| 225 | UGAGAUUUCUUUUUAACAUAAUU |
| 226 | UAGAGAUUGGAUGAAGUCAACUC |
| 227 | UGUAUCAUCAAGCAUUUCAGGAG |
| 228 | UAUUGUAUCAUCAAGCAUUUCAG |
| 229 | UUUCAAAUAGUCAUAUAAGGAGC |
| 230 | mU*mU*mUmUmAmUmUmUfUfAfCmUmUmGmAmAmCmCmA*mA*mA |
| 231 | mC*mC*mCmGmCmCmUmAfAfCfUmGmCmUmCmUmUmCmG*mU*mA |
| 232 | mC*mU*mAmAmUmGmAmUfGfAfUmAmAmUmUmAmUmGmU*mU*mA |
| 233 | mA*mA*mGmUmAmUmAmAfAfAfUmAmAmGmCmUmCmUmA*mU*mA |
| 234 | mU*mC*mCmAmUmUmUmUfCfCfCmCmUmUmUmAmUmGmU*mG*mA |
| 235 | mA*mU*mGmUmUmAmAmAfAfAfGmAmAmAmUmCmUmCmU*mC*mA |
| 236 | mA*mU*mAmAmGmAmCmAfGfUfAmCmAmGmUmAmUmUmU*mA*mA |
| 237 | mA*mA*mCmUmCmUmAmAfAfGfUmUmAmAmGmCmAmUmG*mG*mA |
| 238 | mU*mU*mUmAmAmUmUmUfUfAfUmUmUmUmGmAmAmAmG*mC*mA |
| 239 | mG*mU*mUmAmUmAmGmUfCfAfUmUmAmAmGmUmUmUmU*mU*mA |
| 240 | mU*mU*mUmAmUmGmUmGfAfAfUmGmUmUmUmUmUmGmC*mC*mA |
| 241 | mU*mU*mAmUmGmAmCmAfUfGfGmUmGmCmCmUmUmCmA*mG*mA |
| 242 | mU*mC*mAmAmAmGmAmAfGfAfCmUmGmCmAmAmAmGmC*mC*mA |
| 243 | mU*mG*mAmAmAmAmGmAfUfAfAmCmUmCmUmAmAmAmG*mU*mA |
| 244 | mU*mU*mUmAmCmUmUmGfAfAfCmCmAmAmGmAmGmCmA*mC*mA |
| 245 | mC*mA*mAmCmUmCmAmUfCfUfUmUmUmUmAmUmUmUmA*mA*mA |
| 246 | mA*mU*mGmAmUmGmAmUfAfAfUmUmAmUmGmUmUmAmA*mA*mA |
| 247 | mU*mA*mUmAmUmUmAmCfAfAfAmAmGmUmAmUmAmAmA*mA*mA |
| 248 | mG*mU*mGmAmUmAmGmUfAfCfAmUmGmUmUmAmGmCmA*mU*mA |
| 249 | mU*mU*mGmCmCmUmUmAfUfUfAmUmGmAmCmAmUmGmG*mU*mA |
| 250 | mU*mA*mGmCmAmUmAmUfAfAfUmGmUmAmUmUmUmUmG*mA*mA |
| 251 | mA*mC*mUmUmCmUmGmUfAfUfUmUmUmCmUmCmAmUmA*mG*mA |
| 252 | mU*mU*mUmUmGmUmGmUfGfAfUmUmCmUmUmCmAmAmA*mC*mA |
| 253 | mU*mC*mUmAmAmAmGmUfUfAfAmGmCmAmUmGmGmGmC*mA*mA |
| 254 | mG*mA*mCmAmUmGmGmUfGfCfCmUmUmCmAmGmAmUmC*mC*mA |
| 255 | mG*mC*mCmCmGmCmCmUfAfAfCmUmGmCmUmCmUmUmC*mG*mA |
| 256 | mU*mC*mAmAmAmGmAmAfUfAfUmCmUmUmAmGmUmGmA*mA*mA |
| 257 | mG*mU*mAmUmCmCmCmAfAfAfCmCmAmGmUmGmGmCmA*mA*mA |
| 258 | mU*mU*mUmUmAmUmUmAfUfGfAmUmAmUmUmUmGmUmG*mG*mA |
| 259 | mU*mU*mUmUmUmUmUmAfAfAfCmUmGmGmAmUmUmGmC*mU*mA |
| 260 | mU*mG*mUmCmUmUmUmUfGfUfGmUmGmAmUmUmCmUmU*mC*mA |
| 261 | mC*mU*mCmUmAmAmAmGfUfUfAmAmGmCmAmUmGmGmG*mC*mA |
| 262 | mU*mU*mUmUmAmCmUmUfGfAfAmCmCmAmAmGmAmGmC*mA*mA |
| 263 | mU*mC*mCmUmGmAmAmAfUfGfCmUmUmGmAmUmGmAmU*mA*mA |
| 264 | mU*mG*mAmAmAmUmGmCfUfUfGmAmUmGmAmUmAmCmA*mA*mA |
| 265 | mC*mA*mAmGmGmUmAmAfAfCfCmUmGmCmUmAmUmUmG*mC*mA |
| 266 | mG*mG*mGmCmUmCmCmUfUfAfUmAmUmGmAmCmUmAmU*mU*mA |
| 267 | mG*mG*mCmUmCmCmUmGfAfAfAmUmGmCmUmUmGmAmU*mG*mA |
| 268 | mU*mU*mUmAmUmUmUmUfAfCfUmUmGmAmAmCmCmAmA*mG*mA |
| 269 | mU*mU*mGmAmUmGmAmUfAfCfAmAmUmGmAmAmUmGmU*mG*mA |
| 270 | mU*mG*mAmUmUmCmAmAfUfAfCmUmGmAmAmCmAmCmU*mA*mA |
| 271 | mA*mU*mCmUmAmUmUmCfUfGfUmUmGmGmUmCmUmGmG*mU*mA |
| 272 | mA*mG*mUmUmGmAmCmUfUfCfAmUmCmCmAmAmUmCmU*mC*mA |
| 273 | mC*mU*mCmAmUmCmGmAfGfAfCmAmUmAmAmAmAmUmC*mA*mA |
| 274 | mU*mA*mCmCmUmCmAmAfAfGfAmUmAmAmGmAmCmAmG*mU*mA |
| 275 | mU*mA*mUmAmGmUmCmAfUfUfAmAmGmUmUmUmUmUmA*mU*mA |
| 276 | mC*mA*mAmAmGmAmUmAfAfGfAmCmAmGmUmAmCmAmG*mU*mA |
| 277 | mA*mA*mAmGmAmAmUmAfUfCfUmUmAmGmUmGmAmAmA*mA*mA |
| 278 | mA*mC*mCmAmAmUmUmGfCfCfUmUmAmUmUmAmUmGmA*mC*mA |
| 279 | mU*mU*mUmUmUmGmUmUfCfUfAmCmCmUmCmAmAmAmG*mA*mA |
| 280 | mU*mU*mCmCmAmUmUmUfUfCfCmCmCmUmUmUmAmUmG*mU*mA |
| 281 | mC*mU*mAmCmCmUmCmAfAfAfGmAmUmAmAmGmAmCmA*mG*mA |
| 282 | mA*mA*mUmCmAmAmAmGfAfAfUmAmUmCmUmUmAmGmU*mG*mA |
| 283 | mA*mU*mGmCmUmUmGmAfUfGfAmUmAmCmAmAmUmGmA*mA*mA |
| 284 | mU*mA*mGmGmUmGmAmUfGfCfAmAmUmCmUmAmUmGmU*mU*mA |
| 285 | mA*mU*mAmUmCmUmCmAfAfCfUmUmUmGmUmGmUmCmA*mA*mA |
| 286 | mA*mU*mGmAmAmUmGmUfGfAfAmUmAmUmCmUmUmUmG*mA*mA |
| 287 | mU*mU*mCmAmAmAmCmGfAfGfCmUmGmAmCmAmUmCmU*mA*mA |
| 288 | mA*mA*mAmAmAmUmAmAfAfUfAmUmUmGmCmUmCmUmA*mA*mA |
| 289 | mC*mU*mCmUmCmAmUmAfGfCfUmUmUmCmUmUmUmUmC*mC*mA |
| 290 | mA*mU*mAmGmAmGmGmAfAfAfUmGmAmGmAmAmAmGmG*mU*mA |
| 291 | mG*mU*mGmAmAmAmAmAfGfUfGmUmGmAmAmAmCmUmU*mG*mA |
| 292 | mU*mG*mGmAmCmUmCmAfAfCfUmUmUmGmGmCmUmGmG*mU*mA |
| 293 | mU*mU*mAmAmGmUmUmUfUfUfAmUmUmUmUmAmCmUmU*mG*mA |
| 294 | mG*mA*mAmGmAmUmGmUfGfGfCmUmGmUmGmAmAmAmA*mU*mA |
| 295 | mU*mA*mUmGmUmUmAmAfAfAfAmGmAmAmAmUmCmUmC*mU*mA |
| 296 | mU*mA*mAmUmGmAmUmGfAfUfAmAmUmUmAmUmGmUmU*mA*mA |
| 297 | mG*mA*mUmAmAmUmUmAfUfGfUmUmAmAmAmAmAmGmA*mA*mA |
| 298 | mG*mA*mCmUmGmCmAmAfAfGfCmCmUmAmAmUmGmAmU*mG*mA |
| 299 | mC*mA*mUmUmAmAmGmUfUfUfUmUmAmUmUmUmUmAmC*mU*mA |
| 300 | mA*mC*mUmGmCmUmCmUfUfCfGmUmAmUmUmAmAmGmA*mA*mA |
| 301 | mU*mU*mAmUmGmUmUmAfAfAfAmAmGmAmAmAmUmCmU*mC*mA |
| 302 | mG*mU*mUmGmAmCmUmUfCfAfUmCmCmAmAmUmCmUmC*mU*mA |
| 303 | mU*mU*mUmUmG[Uhd]mGmUfGfAfUmUmCmUmUmCmAmAmA*mC*mA |
| 304 | mU*mC*mUmAmA[Ahd]mGmUfUfAfAmGmCmAmUmGmGmGmC*mA*mA |
| 305 | mG*mA*mCmAmU[Ghd]mGmUfGfCfCmUmUmCmAmGmAmUmC*mC*mA |
| 306 | mU*mG*mUmCmU[Uhd]mUmUfGfUfGmUmGmAmUmUmCmUmUmC*mA |
| 307 | mC*mU*mCmUmA[Ahd]mAmGfUfUfAmAmGmCmAmUmGmGmG*mC*mA |
| 308 | mU*mU*mUmUmA[Chd]mUmUfGfAfAmCmCmAmAmGmAmGmC*mA*mA |
| 309 | mU*mC*mCmUmG[Ahd]mAmAfUfGfCmUmUmGmAmUmGmAmU*mA*mA |
| 310 | mC*mC*mUmGmAmAmAmUfGfCfUmUmGmAmUmGmAmUmA*mC*mA |
| 311 | mG*mA*mAmAmUmGmCmUfUfGfAmUmGmAmUmAmCmAmA*mU*mA |
| 312 | mU*mC*mCmUmUmAmUmAfUfGfAmCmUmAmUmUmUmGmA*mA*mA |
| 313 | mC*mC*mUmGmA[Ahd]mAmUfGfCfUmUmGmAmUmGmAmUmA*mC*mA |
| 314 | mG*mA*mAmAmU[Ghd]mCmUfUfGfAmUmGmAmUmAmCmAmA*mU*mA |
| 315 | mU*mC*mCmUmU[Ahd]mUmAfUfGfAmCmUmAmUmUmUmGmA*mA*mA |
| 316 | mU*fU*mUmGfGmUfUmCmAmAmGmUmAfAmAfAmUmAmAmAmA*mA*mC |
| 317 | mU*fA*mCmGfAmAfGmAmGmCmAmGmUfUmAfGmGmCmGmGmG*mC*mC |
| 318 | mU*fA*mAmCfAmUfAmAmUmUmAmUmCfAmUfCmAmUmUmAmG*mG*mC |
| 319 | mU*fA*mUmAfGmAfGmCmUmUmAmUmUfUmUfAmUmAmCmUmU*mU*mU |
| 320 | mU*fC*mAmCfAmUfAmAmAmGmGmGmGfAmAfAmAmUmGmGmA*mA*mA |
| 321 | mU*fG*mAmGfAmGfAmUmUmUmCmUmUfUmUfUmAmAmCmAmU*mA*mA |
| 322 | mU*fU*mAmAfAmUfAmCmUmGmUmAmCfUmGfUmCmUmUmAmU*mC*mU |
| 323 | mU*fC*mCmAfUmGfCmUmUmAmAmCmUfUmUfAmGmAmGmUmU*mA*mU |
| 324 | mU*fG*mCmUfUmUfCmAmAmAmAmUmAfAmAfAmUmUmAmAmA*mC*mA |
| 325 | mU*fA*mAmAfAmAfCmUmUmAmAmUmGfAmCfUmAmUmAmAmC*mA*mU |
| 326 | mU*fG*mGmCfAmAfAmAmAmCmAmUmUfCmAfCmAmUmAmAmA*mG*mG |
| 327 | mU*fC*mUmGfAmAfGmGmCmAmCmCmAfUmGfUmCmAmUmAmA*mU*mA |
| 328 | mU*fG*mGmCfUmUfUmGmCmAmGmUmCfUmUfCmUmUmUmGmA*mC*mA |
| 329 | mU*fA*mCmUfUmUfAmGmAmGmUmUmAfUmCfUmUmUmUmCmA*mU*mG |
| 330 | mU*fG*mUmGfCmUfCmUmUmGmGmUmUfCmAfAmGmUmAmAmA*mA*mU |
| 331 | mU*fUmUmAfAmAfUmAmAmAmAmAmGfAmUfGmAmGmUmUmG*mA*mG |
| 332 | mU*fU*mUmUfAmAfCmAmUmAmAmUmUfAmUfCmAmUmCmAmU*mU*mA |
| 333 | mU*fU*mUmUfUmAfUmAmCmUmUmUmUfGmUfAmAmUmAmUmA*mU*mU |
| 334 | mU*fA*mUmGfCmUfAmAmCmAmUmGmUfAmCfUmAmUmCmAmC*mU*mU |
| 335 | mU*fA*mCmCfAmUfGmUmCmAmUmAmAfUmAfAmGmGmCmAmA*mU*mU |
| 336 | mU*fU*mCmAfAmAfAmUmAmCmAmUmUfAmUfAmUmGmCmUmA*mA*mC |
| 337 | mU*fC*mUmAfUmGfAmGmAmAmAmAmUfAmCfAmGmAmAmGmU*mA*mU |
| 338 | mU*fG*mUmUfUmGfAmAmGmAmAmUmCfAmCfAmCmAmAmAmA*mG*mA |
| 339 | mU*fU*mGmCfCmCfAmUmGmCmUmUmAfAmCfUmUmUmAmGmA*mG*mU |
| 340 | mU*fG*mGmAfUmCfUmGmAmAmGmGmCfAmCfCmAmUmGmUmC*mA*mU |
| 341 | mU*fC*mGmAfAmGfAmGmCmAmGmUmUfAmGfGmCmGmGmGmC*mC*mG |
| 342 | mU*fU*mUmCfAmCfUmAmAmGmAmUmAfUmUfCmUmUmUmGmA*mU*mU |
| 343 | mU*fU*mUmGfCmCfAmCmUmGmGmUmUfUmGfGmGmAmUmAmC*mU*mU |
| 344 | mU*fC*mCmAfCmAfAmAmUmAmUmCmAfUmAfAmUmAmAmAmA*mG*mA |
| 345 | mU*fA*mGmCfAmAfUmCmCmAmGmUmUfUmAfAmAmAmAmAmA*mA*mA |
| 346 | mU*fG*mAmAfGmAfAmUmCmAmCmAmCfAmAfAmAmGmAmCmA*mU*mA |
| 347 | mU*fG*mCmCfCmAfUmGmCmUmUmAmAfCmUfUmUmAmGmAmG*mU*mU |
| 348 | mU*fU*mGmCfUmCfUmUmGmGmUmUmCfAmAfGmUmAmAmAmA*mU*mA |
| 349 | mU*fU*mAmUfCmAfUmCmAmAmGmCmAfUmUfUmCmAmGmGmA*mG*mC |
| 350 | mU*fU*mUmGfUmAfUmCmAmUmCmAmAfGmCfAmUmUmUmCmA*mG*mG |
| 351 | mU*fG*mCmAfAmUfAmGmCmAmGmGmUfUmUfAmCmCmUmUmG*mU*mG |
| 352 | mU*fA*mAmUfAmGfUmCmAmUmAmUmAfAmGfGmAmGmCmCmC*mU*mG |
| 353 | mU*fC*mAmUfCmAfAmGmCmAmUmUmUfCmAfGmGmAmGmCmC*mA*mU |
| 354 | mU*fC*mUmUfGmGfUmUmCmAmAmGmUfAmAfAmAmUmAmAmA*mA*mA |
| 355 | mU*fC*mAmCfAmUfUmCmAmUmUmGmUfAmUfCmAmUmCmAmA*mG*mC |
| 356 | mU*fU*mAmGfUmGfUmUmCmAmGmUmAfUmUfGmAmAmUmCmA*mU*mG |
| 357 | mU*fA*mCmCfAmGfAmCmCmAmAmCmAfGmAfAmUmAmGmAmU*mG*mU |
| 358 | mU*fG*mAmGfAmUfUmGmGmAmUmGmAfAmGfUmCmAmAmCmU*mC*mC |
| 359 | mU*fU*mGmAfUmUfUmUmAmUmGmUmCfUmCfGmAmUmGmAmG*mC*mA |
| 360 | mU*fA*mCmUfGmUfCmUmUmAmUmCmUfUmUfGmAmGmGmUmA*mG*mA |
| 361 | mU*fA*mUmAfAmAfAmAmCmUmUmAmAfUmGfAmCmUmAmUmA*mA*mC |
| 362 | mU*fA*mCmUfGmUfAmCmUmGmUmCmUfUmAfUmCmUmUmUmG*mA*mG |
| 363 | mU*fU*mUmUfUmCfAmCmUmAmAmGmAfUmAfUmUmCmUmUmU*mG*mA |
| 364 | mU*fG*mUmCfAmUfAmAmUmAmAmGmGfCmAfAmUmUmGmGmU*mA*mC |
| 365 | mU*fU*mCmUfUmUfGmAmGmGmUmAmGfAmAfCmAmAmAmAmA*mA*mA |
| 366 | mU*fA*mCmAfUmAfAmAmGmGmGmGmAfAmAfAmUmGmGmAmA*mA*mA |
| 36 | mU*fC*mUmGfUmCfUmUmAmUmCmUmUfUmGfAmGmGmUmAmG*mA*mA |
| 368 | mU*fC*mAmCfUmAfAmGmAmUmAmUmUfCmUfUmUmGmAmUmU*mU*mU |
| 369 | mU*fU*mUmCfAmUfUmGmUmAmUmCmAfUmCfAmAmGmCmAmU*mU*mU |
| 370 | mU*fA*mAmCfAmUfAmGmAmUmUmGmCfAmUfCmAmCmCmUmA*mA*mA |
| 371 | mU*fU*mUmGfAmCfAmCmAmAmAmGmUfUmGfAmGmAmUmAmU*mA*mG |
| 372 | mU*fU*mCmAfAmAfGmAmUmAmUmUmCfAmCfAmUmUmCmAmU*mU*mG |
| 373 | mU*fU*mAmGfAmUfGmUmCmAmGmCmUfCmGfUmUmUmGmAmA*mG*mG |
| 374 | mU*fU*mUmAfGmAfGmCmAmAmUmAmUfUmUfAmUmUmUmUmU*mA*mA |
| 375 | mU*fG*mGmAfAmAfAmGmAmAmAmGmCfUmAfUmGmAmGmAmG*mA*mU |
| 376 | mU*fA*mCmCfUmUfUmCmUmCmAmUmUfUmCfCmUmCmUmAmU*mC*mG |
| 377 | mU*fC*mAmAfGmUfUmUmCmAmCmAmCfUmUfUmUmUmCmAmC*mU*mA |
| 378 | mU*fA*mCmCfAmGfCmCmAmAmAmGmUfUmGfAmGmUmCmCmA*mA*mG |
| 379 | mU*fC*mAmAfGmUfAmAmAmAmUmAmAfAmAfAmCmUmUmAmA*mU*mG |
| 380 | mU*fA*mUmUfUmUfCmAmCmAmGmCmCfAmCfAmUmCmUmUmC*mC*mC |
| 381 | mU*fA*mGmAfGmAfUmUmUmCmUmUmUfUmUfAmAmCmAmUmA*mA*mU |
| 382 | mU*fU*mAmAfCmAfUmAmAmUmUmAmUfCmAfUmCmAmUmUmA*mG*mG |
| 383 | mU*fU*mUmCfUmUfUmUmUmAmAmCmAfUmAfAmUmUmAmUmC*mA*mU |
| 384 | mU*fC*mAmUfCmAfUmUmAmGmGmCmUfUmUfGmCmAmGmUmC*mU*mU |
| 385 | mU*fA*mGmUfAmAfAmAmUmAmAmAmAfAmCfUmUmAmAmUmG*mA*mC |
| 386 | mU*fU*mUmCfUmUfAmAmUmAmCmGmAfAmGfAmGmCmAmGmU*mU*mA |
| 387 | mU*fG*mAmGfAmUfUmUmCmUmUmUmUfUmAfAmCmAmUmAmA*mU*mU |
| 388 | mU*fA*mGmAfGmAfUmUmGmGmAmUmGfAmAfGmUmCmAmAmC*mU*mC |
| 389 | mU*fG*mUmAfUmCfAmUmCmAmAmGmCfAmUfUmUmCmAmGmG*mA*mG |
| 390 | mU*fA*mUmUfGmUfAmUmCmAmUmCmAfAmGfCmAmUmUmUmC*mA*mG |
| 391 | mU*fU*mUmCfAmAfAmUmAmGmUmCmAfUmAfUmAmAmGmGmA*mG*mC |
| 392 | H-Aib-EGTFTSDVSSYLEGQAAK((2-[2-(2-Amino-ethoxy)-ethoxy]-acetyl)₂-(γGlu)-CO- |
| | (CH₂)₁₆-CO₂H)EFIAWLVRGRG |
| 393 | agttccccttggctgggctctgaggactgaagtgtgtgtgtcttttgtgtgattcttcaa |
| | acttcacctgccaaacggaaggagcatgttgggcctccgtcatgctaaccaacgggaaag |
| | agcaggtgatcaaatcgtgtgtgtccctcccggaactaaatgctcaggtcttctgtcaca |
| | gttccaacaacgtgaccaaaaccgaatgttgcttcacagacttctgcaacaatatcacac |
| | tgcaccttcccacaggtctgcctctcttggttcaaagaacaatcgcaaggacaattgtac |
| | ttcaagaaatcgtaggaaaaggtcggtttggggaagtgtggcacggaagatggtgtggag |
| | aagatgtggctgtgaaaatattctcctccagggatgagagatcttggttccgtgaggcgg |
| | aaatttatcagacggtgatgctgagacacgagaacatcctcggtttcatcgcagctgaca |
| | acaaagataatggaacttggacacaactctggcttgtgtcagagtatcacgagcagggct |
| | ccttgtatgactatttgaacagaaacatagtgactgtggctggaatggtcaagctggcgc |
| | tttccatagcgagtggtctggctcacctgcacatggagatcgtgggtactcaaggtaagc |
| | ctgctattgctcaccgagatataaagtcaaagaatatcctagtcaaaaaatgtgacactt |
| | gtgccatagctgacttagggctggctgtgaagcacgattctatcatgaacactatagaca |
| | taccccagaatcctaaagtgggaaccaagaggtacatggctcccgaaatgcttgatgata |
| | caatgaacctgagcatctttgagtccttcaagcgagctgacatctattcggtggggctgg |
| | tttactgggaaatagctcgaaggtgttcagttggaggagttgttgaggagtaccagttgc |
| | cttactatgacatggtgccttcagatccttcgatagaagaaatgaggaaggttgtctgtg |
| | atcagaaactccgaccaaatctcccaaaccagtggcaaagctgtgaagcgctgcgggtca |
| | tgggaagaataatgcgtgagtgctggtacgccaacggggcagcacgcctgacagccctgc |
| | gcgtgaagaagaccatctcccagctgtgtgtcaaggaagactgtaaagcttagctctgcg |
| | tacaggcaaaggaagagtgctcacagctttccttcccgtctcccgctttgtgtaaatgtt |
| | tttgcttcttttctgctgtgttttattttagttctacctcaaggatgactcactacagtg |
| | ttgaagtgtcccaaaggcagcatgtaaagataacgctaaagttaagcatgggcaggtctt |
| | gactttcccagtttccatgttgtccttagttttatgtttaaaggggacacgactcgtttt |
| | ttctttttatttaaggaggaagatgttatgagtataagataagctacatacaatggtaaa |
| | aaaaatattaaatttttgttgtacttgaaccaagagcatccacaaatgagatgaagacat |
| | atttaaaatgtgaactactgtatattatggtgaaatggaactgtaaaaattaactgggtc |
| | cttaaaaagtaaattttgctcagaattttaacatctaaagagtggaaaggtggagtaaca |
| | aagtgacaccacgttctcaggacttgctttctctgcctatttgccactctgcatcactac |
| | gacagctgtccctaagtagttcgaaaaccacctgagccattactcctcctgatacataac |
| | agaggtccatatgtctcaaacactgtgaaatggcccagagtcacagagctcttgccagag |
| | ctggttacgaaccctgtgacaactagcactgtgaagtttgctggccattatactcccttg |
| | aattcccatagagcgtttcttcccaagtgggaagatcaatcatcagttataatattttaa |
| | atttagtcacatagtgaatgcattgagtattttgtaagaccataatagattttctagaat |
| | ttcagaaccaatctattagaatttcatcaaagaaatgtcatcattatcccattcgataat |
| | gaaaaaaaatgttttctgaacattgccaaatattgaccaattataaacatccattttcta |
| | aagatgtatgtcataagatgtcattgcttttgaggttcatggttggaaagaaaatgacag |
| | aaagataaacacggaaagaatttgaggagagttcagagaactctcccgaaggagtgtaac |
| | cccttactggtaacagaagatcacatcagtgattacaaggcattcctggccttttaacag |
| | ggaccatgcatggaagggtttggaggatctacctctattctccatttttgaagttcagaa |
| | tatttaacaggacaccagatatttctttcaaatgtcctataggctttagtgaagatggtt |
| | taactcaacgtctttcagtgttctgaaacagtttcccgaaccccattccttagaactttc |
| | atctacctagaagttgttagaatggatgtgcactctaacaaggtcttgaaaagtgatatg |
| | ttaacatattctattctttgatagttctagcagcaaagacacattccatggatattttta |
| | agggtaaatatttgattgtagcctttctattatgagtaaggtgaagttttactatctagc |
| | atcttctaaagcccaacatggtaaatgatgttgtgaagaatacgttatgtatcccataag |
| | agggtaacttgatgtctacatatcactatggagatatgctactcgatccatctcatttga |
| | ctaagtggagataagagaaacaaaagctgacagctgtggagtttgcatgtacttccattc |
| | tcagtcatgtcctcatccatttataagcacagggcaccccataacttacttgccacaata |
| | ttgggtacaagagggaataacaagatagataggtcctgccctcggggattttaaagtcta |
| | atttgggagtggggaaagacatgtgagtatgagggaagcgagtaaattgacagataaaat |
| | atgcagtgggatgcctttgagttctatgtgacaatggcttctagaataggtcttaaaatc |
| | acttccaatttgcaacacatttagaagggatctttatttagatattacagcctaattata |
| | tcatcagcatcaaaaggtgcgcctacgtgaatggatgttaagaagaaccgtgcgaaggga |
| | gagctggcttatgtctattatcttatttcacagtttctacatgacggcaatggaattatt |
| | tacttcaatgattgtatccagatatccaatgaaaaaaacctactctatcctgttccattc |
| | accactgttagggaagtatattttctagattttaaaagagggtgttgtaacatctttatt |
| | ttggtcattatgttttataatttatttcagggtatatacaaacataacttatattttctt |
| | ggtttagatttcacagtggagaaatctgcacattggtacattttgagacttttgctcatt |
| | attctgttgctgaactgtaatccctaagtgaataatcttaaagagcagagccacagaagt |
| | agtcagggaacaatagaagttgtagtttttgcttaactctcaacatagttctacatagat |
| | actgctcacttataaaaaatagagtaatttttagagcataaggcatatttactgtatata |
| | tctatgcagttcaattttgtattatgtgtttttgatggaatcttatgtaacacttggtcc |
| | ttgtaaattttgttacgagcaaaaatatcttcttaggttaggtgcaaagtgacaccattg |
| | ctttgctccttatggatattttggctctgggtataacatagagcttaggaatgctattat |
| | gaatttttagtactgtatgcaaaaaatggtggggtttatatacagcatcacttcttgaaa |
| | tacagaacgcattatttgttgtctacatttgagcatgaatttgttgccttataagtcctg |
| | cgttttgagtttgtaatcagtactgactctgttgtatgcatccttccacgtctaaaatat |
| | ttggcatgtcacatctagaattcttaatttatgttctgccatgagagttaagtgaaacat |
| | gactgtcatgctctattttaagcgcagcacttgcttttcatctttatacttttcaattaa |
| | ctttgcattttaaatttccataattgtatgaaaatagtaacctgatcgcaatgtctgaag |
| | agtttcaaatcggcgtttattttaaaatgaaaaaaaaaaaactacaacacattcattaga |
| | tcagaagttccacaatcacttattgttttgagtgcttcgtctattgcagaatgattaaat |
| | aaaagaaatgtctactgtcctggaattttgccaccatgggacttactgagacagagcaaa |
| | ctcagggcaatcttggagtggaagcaaaagtcccagggaagaccgcagtgcactgcctga |
| | aggcagggagcaggcatctgccatcatacaaggagcaagagaacaacttgaaattacttg |
| | gcaggcaatgatttttgactgattcttgaaaggaaatatgaacatttacatggctaaatt |
| | aattcatttaaaatgtggaaattttgatttctatcaagcagttagggaacaataacaaat |
| | gcatccaagttggttgctgaattcttcagtattcgtttcctttatgtaacttgaaaaact |
| | aatattatcaatcactcagaaatgtttatgcttagtgtcctaaaatggtgtcactgtagt |
| | ttttgaagctcccctctctcttttttcacttgtggaatgtattatctccatgaataacaa |
| | tactgtgttttaattccagatcctgatattcacttagattttattcactgtcttagtgaa |
| | gacagtcatcattttaagggcttttaattttttttttttttgcaggattctttaaaacta |
| | aaccaaaaaagatagagaaacagatagatggacagaaagaaagaaagagagaaagaaagg |
| | aagaaaggaagaaggaagaaagaggcaaaggaagaaaatctagaaggcaaaacattcatt |
| | aagtctagtattagtcagaatttctatagtgttcatgttcttctgaggatactgatgcaa |
| | actccctggtgcctggctttctctttaccctccaccttagcttcgtttcattatotttct |
| | tctactattaaacttgatgttctttcatcaccttccaaaattatagttacttcctaatta |
| | tttttaaacatagctattatttaacttaatcattatagaattgcacatttataatggaga |
| | agaaatcccttagtgttctaaattcttttatatgtgccacagcacaaaatgaaaatgaga |
| | atgtaaaagttaaagggactctgagcctcagatcttggttctaatattttgtagacgaag |
| | aaactgaagaccggtcattagatggcagctaatggttgtagaaacacagtggtgacacct |
| | acttctgcagcctcgcccacgtttacaaattgaaaagtcaatcagcatggatttaacact |
| | gcttcatgcacatattaacatcacatgccattcagtgctggtggtcagaccctacttgtt |
| | gatgcatttctattaaaatagatggcttaggccaaggtcactagggaaaaaatgaaaata |
| | gacaaagcaaaaacctaaaagttgttatcgagataggcagaaaagccatgaagcactgat |
| | gagtttggaaatgtaattgaagaaatttagtgtgtggagggttttagtgctggctggagg |
| | tgtagagcagaggccgagaggacttgcctcgaaagtgaaaagatttgggttcaattttca |
| | gcactggtaagagaaggaagataggaaggaggtaagaagggaaggaaggaagtgaggaag |
| | gaagaaaggaggaaaggaaggataaaagaaagcattcttagtatgaataatgattatcga |
| | agcctctggaccttccttgctcccttcctgaatgcattgttcctttcccttgtgtgttgg |
| | aatggtgaaaggtgatttggtacacgaactggagactatgtttagtgtcatttatagtct |
| | ctctcttgtttgttttgtctttgacatcattgtctcccctggtggcagatttcctgagcc |
| | ctgcccccaccactctactcagctctgtcctttcatgtatttgagccctgtccagactgt |
| | tacacacggaacgtcttaacaaggttttaatttccccactgtgtataagaggcctcggct |
| | caccagttcattagtgatcgttctcctgacactgggaagagggctctgttaatggcagac |
| | atgactttctttctcttccattttagtcagactgtctggttatcttgtagcttttggcta |
| | gtgcataaagaatgtccgcttctgctttcatgctgaactcagcttcaacagcgacttgaa |
| | caaagactgtgaaatacatatcccataatgtgattgctcatgttacgcctgtgataattg |
| | agccattggtttgttttattcctgccatcctttgaaatatttttatttatttatttttat |
| | gtgttatgggagttttgattgaatgtgggtctgtgtaccatatgcatatccggtgcttgg |
| | gtaggccaaaagagtctgatcccctgaaactagaattacaggtagttgtaagctgccatg |
| | tgggtgctggaaaccagaacttggtcctctggaagagcatcagccagtgcttctaactgc |
| | taagccatctctcttctgcttctgccatgtttttagactaagccatgtgccatgctgctt |
| | agaatgcagattatgctgaaattaggtaacgatctccactttaaaatttgatattgatag |
| | ctgccaggattctcctgcagttagtcatagatgaacacaacatttgggagaatatgagct |
| | ccagctcatcctcattttaataatgccagaaatgtatttttctccttgttggtttatgtt |
| | acactacaagaaaatttcctcctggacccaacactttcattggaatatgcaattcttccc |
| | cgggttttaggaatctgactgttcacacagcctgagtgtggtaggaaattcacctttctt |
| | tccagcatcccacatataaattctccacagggaactcaacagattggggcagctgaggac |
| | tggggtgagcgctggaactagctcttccctatagagacagttctagaccttacagagaaa |
| | gacattgtgtgtttctatatcaacataaaatgcccaggcttatccatcattatgaaatag |
| | atgctaaatttagaaatggttcttgacatatgcaaactgaacacagaatttggaagaata |
| | gaatactctgacttttcttctctgtattcccaagagtaacaagagtaacaaggtggactc |
| | tttatcctggtgatgggtgtatcttttccccaacctaagtcattggctgcaaggcacagt |
| | aacctcaatttctatacatccaccaagttttcattctgtggatgaacctactcatttgct |
| | tcacctgtaaagggaggcttgagtgtcaggctctaagaaagttaaccatactaagagatt |
| | tatcaatatgtttggtttttcattggcagaacagtagtatctaggcaattttaaagatgt |
| | agcagtttcctgttctctttaccaagtgagagaatctgagtaaaatattagactccagtt |
| | catttctagaatggcttatatcaagctcttgtactctcctgaccctgaactggataggat |
| | tgtttacacttgattgttgtttcaggttttaaaagggtctcaaaagaaaaaattggattc |
| | aggtctataaaaaaaaaaaaggattttgtattaattttcaaagcactaatactaattata |
| | cttttcttggggtagtgattctcttcttatacctaagaacatattacacatgtcaaaacc |
| | attgcattttgacattgcgaaacacaccttgaactcttgaactactgtgagcagaatcac |
| | tgttgtaaagatttttttttttttttttttttttggtaagcttgctggtattcttaagta |
| | tgtaaaaagatgtcggcttccacaggctcaaatgaatgtacataaggactgtgaaaaaaa |
| | aataaattgggttttaaaataagaactgggcgataaattgtatctaaactgcgcagatgg |
| | gcatcgtcgtgcggctgtgttttcattgttgaaggttgaacagagttctacagagtcgtg |
| | gatgacattcagatgtttcatgacttttggatctgcgaacatatgcattttatagtcatg |
| | tatacttatattgttaagaaataatcatattttagaatttagtaagaactcagtgatagc |
| | cttcataccaaaatgtttaactttgccaacagctagtcataaaaaagtatttattttaaa |
| | gctttttaatacaattgcataacttcttgccgcctccaggtgtacattcttctctgccta |
| | aatgttatatttttatgtatgcattttcagaaagcatattgttttggaaagtggcaaaga |
| | caatgaattaagcactcgttgcacttgtttctgtaaagcataaagagctctattttaaat |
| | aaaagaaaacgtgtcataaaaaaaaa |
| 394 | mltngkeqvi kscvslpeln aqvfchssnn vtkteccftd fonnitlhlp tglpllvqrt |
| | iartivlqei vgkgrfgevw hgrwcgedva vkifssrder swfreaeiyq tvmlrhenil |
| | gfiaadnkdn gtwtqlwlvs eyheqgslyd ylnrnivtva gmvklalsia sglahlhmei |
| | vgtqgkpaia hrdiksknil vkkcdtcaia dlglavkhds imntidipqn pkvgtkryma |
| | pemlddtmnl sifesfkrad iysvglvywe iarrcsvggv veeyqlpyyd mvpsdpsiee |
| | mrkvvcdqkl rpnlpnqwqs cealrvmgri mrecwyanga arltalrvkk tisqlcvked |
| | cka |
| 395 | GGGGEGGGGEGGGGEKEKEKGGGGSGGGGS-fSp-Cha-aGPIDRI-NH₂ |
| 396 | CUUGAUGAUACAAUGAAUGUA |
| 397 | UGCUUGAUGAUACAAUGAAUA |
| 398 | UGUUAGCAUAUAAUGUAUUUA |
| 399 | UCAUUAAGUUUUUAUUUUACA |
| 400 | AGACUGCAAAGCCUAAUGAUA |
| 401 | GGAAGAUGUGGCUGUGAAAAA |
| 402 | UAGAGGAAAUGAGAAAGGUUA |
| 403 | GGUGAUGCAAUCUAUGUUUCA |
| 404 | UUCUACCUCAAAGAUAAGACA |
| 405 | UAACUGCUCUUCGUAUUAAGA |
| 406 | AGGUAAACCUGCUAUUGCUCA |
| 407 | UUUCCAUUUUCCCCUUUAUGA |
| 408 | GCUGGUAUCUGAAUAUCAUGA |
| 409 | AAUAUAUUACAAAAGUAUAAA |
| 410 | AUUAUGUUAAAAAGAAAUCUA |
| 411 | ACUAUAUCUCAACUUUGUGUA |
| 412 | GGAGUUGACUUCAUCCAAUCA |
| 413 | AAAAUAAAUAUUGCUCUAAAA |
| 414 | CUGUCAUAGUUCCAACAAUGA |
| 415 | GCCUAAUGAUGAUAAUUAUGA |
| 416 | AUAACUCUAAAGUUAAGCAUA |
| 417 | UAAUUUUAUUUUGAAAGCAAA |
| 418 | AAGAUAAGACAGUACAGUAUA |
| 419 | AUGAUAAUUAUGUUAAAAAGA |
| 420 | UCUCAACUUUGUGUCAAAGAA |
| 421 | UUUUUUGUUCUACCUCAAAGA |
| 422 | AUUACAAAAGUAUAAAAUAAA |
| 423 | AAAGCCUAAUGAUGAUAAUUA |
| 424 | CUCAUAGCUUUCUUUUCCAUA |
| 425 | GCAUAUAAUGUAUUUUGAAGA |
| 426 | CUGCUCUUCGUAUUAAGAAGA |
| 427 | GUUUUUAUUUUACUUGAACCA |
| 428 | CCAUUUUCCCCUUUAUGUGAA |
| 429 | AUAUUACAAAAGUAUAAAAUA |
| 430 | GCUCCUGAAAUGCUUGAUGAA |
| 431 | AUAAUUAUGUUAAAAAGAAAA |
| 432 | UGAUGAUAAUUAUGUUAAAAA |
| 433 | ACUGCAAAGCCUAAUGAUGAA |
| 434 | ACACAAGGUAAACCUGCUAUA |
| 435 | UGAUAAUUAUGUUAAAAAGAA |
| 436 | AAGACUGCAAAGCCUAAUGAA |
| 437 | AAAUAUAUUACAAAAGUAUAA |
| 438 | AACUGCUCUUCGUAUUAAGAA |
| 439 | CCUCAAAGAUAAGACAGUACA |
| 440 | UUACUUGAACCAAGAGCACAA |
| 441 | UAUCUCAACUUUGUGUCAAAA |
| 442 | UGCAAAGCCUAAUGAUGAUAA |
| 443 | UAUUACAAAAGUAUAAAAUAA |
| 444 | UGGUAUCUGAAUAUCAUGAAA |
| 445 | GUCUGUCAUAGUUCCAACAAU |
| 446 | AUGAAUGUGAAUAUGUUUGAA |
| 447 | AUGAUUGUGAAUAUCUUUGAA |
| 448 | GGCUCCUGAAAUGCCUGAUGA |
| 449 | CAUAGCUUUCUUUUCCAUUUA |
| 450 | UUGAUGAUACAAUGCAUGUGA |
| 451 | UUCAAACGAGCUGAGAUCUAA |
| 452 | UUCAUACGAGCUGACAUCUAA |
| 453 | AUCUCAACUUUGUGUCAAA |
| 454 | UAUCUCAACUUUGUGUCAAA |
| 455 | UAUAUCUCAACUUUGUGUCAAA |
| 456 | CUAUAUCUCAACUUUGUGUCAAA |
| 457 | UACAUUCAUUGUAUCAUCAAGCA |
| 458 | UAUUCAUUGUAUCAUCAAGCAUU |
| 459 | UAAAUACAUUAUAUGCUAACAUG |
| 460 | UGUAAAAUAAAAACUUAAUGACU |
| 461 | UAUCAUUAGGCUUUGCAGUCUUC |
| 462 | UUUUUCACAGCCACAUCUUCCCC |
| 463 | UAACCUUUCUCAUUUCCUCUAUC |
| 464 | UGAAACAUAGAUUGCAUCACCUA |
| 465 | UGUCUUAUCUUUGAGGUAGAACA |
| 466 | UCUUAAUACGAAGAGCAGUUAGG |
| 467 | UGAGCAAUAGCAGGUUUACCUUG |
| 468 | UCAUAAAGGGGAAAAUGGAAAAG |
| 469 | UCAUGAUAUUCAGAUACCAGCCA |
| 470 | UUUAUACUUUUGUAAUAUAUUUC |
| 471 | UAGAUUUCUUUUUAACAUAAUUA |
| 472 | UACACAAAGUUGAGAUAUAGUCU |
| 473 | UGAUUGGAUGAAGUCAACUCCUG |
| 474 | UUUUAGAGCAAUAUUUAUUUUUA |
| 475 | UCAUUGUUGGAACUAUGACAGAA |
| 476 | UCAUAAUUAUCAUCAUUAGGCUU |
| 477 | UAUGCUUAACUUUAGAGUUAUCU |
| 478 | UUUGCUUUCAAAAUAAAAUUAAA |
| 479 | UAUACUGUACUGUCUUAUCUUUG |
| 480 | UCUUUUUAACAUAAUUAUCAUCA |
| 481 | UUCUUUGACACAAAGUUGAGAUA |
| 482 | UCUUUGAGGUAGAACAAAAAAAA |
| 483 | UUUAUUUUAUACUUUUGUAAUAU |
| 484 | UAAUUAUCAUCAUUAGGCUUUGC |
| 485 | UAUGGAAAAGAAAGCUAUGAGAG |
| 486 | UCUUCAAAAUACAUUAUAUGCUA |
| 487 | UCUUCUUAAUACGAAGAGCAGUU |
| 488 | UGGUUCAAGUAAAAUAAAAACUU |
| 489 | UUCACAUAAAGGGGAAAAUGGAA |
| 490 | UAUUUUAUACUUUUGUAAUAUAU |
| 491 | UUCAUCAAGCAUUUCAGGAGCCA |
| 492 | UUUUCUUUUUAACAUAAUUAUCA |
| 493 | UUUUUAACAUAAUUAUCAUCAUU |
| 494 | UUCAUCAUUAGGCUUUGCAGUCU |
| 495 | UAUAGCAGGUUUACCUUGUGUAC |
| 496 | UUCUUUUUAACAUAAUUAUCAUC |
| 497 | UUCAUUAGGCUUUGCAGUCUUCU |
| 498 | UUAUACUUUUGUAAUAUAUUUCU |
| 499 | UUCUUAAUACGAAGAGCAGUUAG |
| 500 | UGUACUGUCUUAUCUUUGAGGUA |
| 501 | UUGUGCUCUUGGUUCAAGUAAAA |
| 502 | UUUUGACACAAAGUUGAGAUAUA |
| 503 | UUAUCAUCAUUAGGCUUUGCAGU |
| 504 | UUAUUUUAUACUUUUGUAAUAUA |
| 505 | UUUCAUGAUAUUCAGAUACCAGC |
| 506 | AUUGUUGGAACUAUGACAGAC |
| 507 | UUCAAACAUAUUCACAUUCAUUG |
| 508 | UUCAAAIAUAUUCACAUUCAUUG |
| 507 | UUCAAACAUAUUCACAUUCAUUG |
| 509 | UUCAAAGAUAUUCACAAUCAUUG |
| 510 | UACAUAAAGGGGAAAAUGGAA |
| 511 | UCAUCAIGCAUUUCAGGAGCCAU |
| 512 | UAAAUGGAAAAGAAAGCUAUGAG |
| 513 | UCACAUICAUUGUAUCAUCAAGC |
| 514 | UUAGAUCUCAGCUCGUUUGAAGG |
| 515 | UUAGAUGUCAGCUCGUAUGAAGG |
| 516 | UUAGAUIUCAGCUCGUUUGAAGG |
| 517 | UUUGACACAAAGUUGAGAUAU |
| 518 | UUUGACACAAAGUUGAGAUAUA |
| 519 | mC*mU*mUmGmAmUmGmAfUfAfCmAmAmUmGmAmAmUmG*mU*mA |
| 520 | mU*mG*mCmUmUmGmAmUfGfAfUmAmCmAmAmUmGmAmA*mU*mA |
| 521 | mU*mG*mUmUmAmGmCmAfUfAfUmAmAmUmGmUmAmUmU*mU*mA |
| 522 | mU*mC*mAmUmUmAmAmGfUfUfUmUmUmAmUmUmUmUmA*mC*mA |
| 523 | mA*mG*mAmCmUmGmCmAfAfAfGmCmCmUmAmAmUmGmA*mU*mA |
| 524 | mG*mG*mAmAmGmAmUmGfUfGfGmCmUmGmUmGmAmAmA*mA*mA |
| 525 | mU*mA*mGmAmGmGmAmAfAfUfGmAmGmAmAmAmGmGmU*mU*mA |
| 526 | mG*mG*mUmGmAmUmGmCfAfAfUmCmUmAmUmGmUmUmU*mC*mA |
| 527 | mU*mU*mCmUmAmCmCmUfCfAfAmAmGmAmUmAmAmGmA*mC*mA |
| 528 | mU*mA*mAmCmUmGmCmUfCfUfUmCmGmUmAmUmUmAmA*mG*mA |
| 529 | mA*mG*mGmUmAmAmAmCfCfUfGmCmUmAmUmUmGmCmU*mC*mA |
| 530 | mU*mU*mUmCmCmAmUmUfUfUfCmCmCmCmUmUmUmAmU*mG*mA |
| 531 | mG*mC*mUmGmGmUmAmUfCfUfGmAmAmUmAmUmCmAmU*mG*mA |
| 532 | mA*mA*mUmAmUmAmUmUfAfCfAmAmAmAmGmUmAmUmA*mA*mA |
| 533 | mA*mU*mUmAmUmGmUmUfAfAfAmAmAmGmAmAmAmUmC*mU*mA |
| 534 | mA*mC*mUmAmUmAmUmCfUfCfAmAmCmUmUmUmGmUmG*mU*mA |
| 535 | mG*mG*mAmGmUmUmGmAfCfUfUmCmAmUmCmCmAmAmU*mC*mA |
| 536 | mA*mA*mAmAmUmAmAmAfUfAfUmUmGmCmUmCmUmAmA*mA*mA |
| 537 | mC*mU*mGmUmCmAmUmAfGfUfUmCmCmAmAmCmAmAmU*mG*mA |
| 538 | mG*mC*mCmUmAmAmUmGfAfUfGmAmUmAmAmUmUmAmU*mG*mA |
| 539 | mA*mU*mAmAmCmUmCmUfAfAfAmGmUmUmAmAmGmCmA*mU*mA |
| 540 | mU*mA*mAmUmUmUmUmAfUfUfUmUmGmAmAmAmGmCmA*mA*mA |
| 541 | mA*mA*mGmAmUmAmAmGfAfCfAmGmUmAmCmAmGmUmA*mU*mA |
| 542 | mA*mU*mGmAmUmAmAmUfUfAfUmGmUmUmAmAmAmAmA*mG*mA |
| 543 | mU*mC*mUmCmAmAmCmUfUfUfGmUmGmUmCmAmAmAmG*mA*mA |
| 544 | mU*mU*mUmUmUmUmGmUfUfCfUmAmCmCmUmCmAmAmA*mG*mA |
| 545 | mA*mU*mUmAmCmAmAmAfAfGfUmAmUmAmAmAmAmUmA*mA*mA |
| 546 | mA*mA*mAmGmCmCmUmAfAfUfGmAmUmGmAmUmAmAmU*mU*mA |
| 547 | mC*mU*mCmAmUmAmGmCfUfUfUmCmUmUmUmUmCmCmA*mU*mA |
| 548 | mG*mC*mAmUmAmUmAmAfUfGfUmAmUmUmUmUmGmAmA*mG*mA |
| 549 | mC*mU*mGmCmUmCmUmUfCfGfUmAmUmUmAmAmGmAmA*mG*mA |
| 550 | mG*mU*mUmUmUmUmAmUfUfUfUmAmCmUmUmGmAmAmC*mC*mA |
| 551 | mC*mC*mAmUmUmUmUmCfCfCfCmUmUmUmAmUmGmUmG*mA*mA |
| 552 | mA*mU*mAmUmUmAmCmAfAfAfAmGmUmAmUmAmAmAmA*mU*mA |
| 553 | mG*mC*mUmCmCmUmGmAfAfAfUmGmCmUmUmGmAmUmG*mA*mA |
| 554 | mA*mU*mAmAmUmUmAmUfGfUfUmAmAmAmAmAmGmAmA*mA*mA |
| 555 | mU*mG*mAmUmGmAmUmAfAfUfUmAmUmGmUmUmAmAmA*mA*mA |
| 556 | mA*mC*mUmGmCmAmAmAfGfCfCmUmAmAmUmGmAmUmG*mA*mA |
| 557 | mA*mC*mAmCmAmAmGmGfUfAfAmAmCmCmUmGmCmUmA*mU*mA |
| 558 | mU*mG*mAmUmAmAmUmUfAfUfGmUmUmAmAmAmAmAmG*mA*mA |
| 559 | mA*mA*mGmAmCmUmGmCfAfAfAmGmCmCmUmAmAmUmG*mA*mA |
| 560 | mA*mA*mAmUmAmUmAmUfUfAfCmAmAmAmAmGmUmAmU*mA*mA |
| 561 | mA*mA*mCmUmGmCmUmCfUfUfCmGmUmAmUmUmAmAmG*mA*mA |
| 562 | mC*mC*mUmCmAmAmAmGfAfUfAmAmGmAmCmAmGmUmA*mC*mA |
| 563 | mU*mU*mAmCmUmUmGmAfAfCfCmAmAmGmAmGmCmAmC*mA*mA |
| 564 | mU*mA*mUmCmUmCmAmAfCfUfUmUmGmUmGmUmCmAmA*mA*mA |
| 565 | mU*mG*mCmAmAmAmGmCfCfUfAmAmUmGmAmUmGmAmU*mA*mA |
| 566 | mU*mA*mUmUmAmCmAmAfAfAfGmUmAmUmAmAmAmAmU*mA*mA |
| 567 | mU*mG*mGmUmAmUmCmUfGfAfAmUmAmUmCmAmUmGmA*mA*mA |
| 568 | [iAb]*mU*mUmCmAmAmAmCmGfAfGfCmUmGmAmCmAmUmCmU*mA*mA |
| 569 | [iAb]*mA*mUmAmUmCmUmCmAfAfCfUmUmUmGmUmGmUmCmA*mA*mA |
| 570 | [iAb]*mU*mUmGmAmUmGmAmUfAfCfAmAmUmGmAmAmUmGmU*mG*mA |
| 571 | [iAb]*mA*mUmGmAmAmUmGmUfGfAfAmUmAmUmCmUmUmUmG*mA*mA |
| 572 | [iAb]*mG*mGmCmUmCmCmUmGfAfAfAmUmGmCmUmUmGmAmU*mG*mA |
| 573 | [iAb]*mA*mUmAmUmCmUmCmAfAfCfUmUmUmGmU[Ghd]mUmCmA*mA*mA |
| 574 | [iAb]*mU*mUmGmAmUmGmAmUfAfCfAmAmUmGmA[Ahd]mUmGmU*mG*m |
| | A |
| 575 | [iAb]*mG*mGmCmUmCmCmUmGfAfAfAmUmGmCmU[Uhd]mGmAmU*mG*mA |
| 576 | [iAb]*mU*mUmCmAmAmAmCmGfAfGfCmUmGmAmC[Ahd]mUmCmU*mA*mA |
| 577 | [iAb]*mA*mUmGmAmAmUmGmUfGfAfAmUmAmUmC[Uhd]mUmUmG*mA*m |
| | A |
| 578 | mC*mU*mAmAmUmGfAmUfGfAfUmAmAmUmU[Ada]mUmGmU*mU*mA |
| 579 | [iAb]*mGmUmCmUmGmUmCmAfUfAfGmUmUmCmCmAmAmCmAmAmU*[iAb] |
| 580 | [iAb]*mCmUmAmAmUmGfAmUfGfAfUmAmAmUmUmAmUmGmUmUmA*[iAb] |
| 581 | [iAb]*mC*mUmAmAmUmGfAmUfGfAfUmAmAmUmUmAmUmGmU*mU*mA |
| 582 | [iAb]*mC*mUmAmAmUmGfAmUfGfAfUmAmAmUmU[Ahd]mUmGmU*mU*mA |
| 583 | [iAb]*mC*mAmAmAmGmAmUmAfAfGfAmCmAmGmUmAmCmAmG*mU*mA |
| 584 | [iAb]*mU*mUmCmCmAmUmUmUfUfCfCmCmCmUmUmUmAmUmG*mU*mA |
| 585 | [iAb]*mA*mUmGmAmAmUmGmUfGfAfAmUmAmUmGmUmUmUmG*mA*mA |
| 586 | [iAb]*mA*mUmGmAmUmUmGmUfGfAfAmUmAmUmCmUmUmUmG*mA*mA |
| 587 | [iAb]*mG*mGmCmUmCmCmUmGfAfAfAmUmGmCmCmUmGmAmU*mG*mA |
| 588 | [iAb]*mU*mAmUmGmUmUmAmAfAfAfAmGmAmAmAmUmCmUmC*mU*mA |
| 589 | mC*mA*mUmAmGmCmUmUfUfCfUmUmUmUmCmCmAmUmU*mU*mA |
| 590 | [iAb]*mC*mAmUmAmGmCmUmUfUfCfUmUmUmUmCmCmAmUmU*mU*mA |
| 591 | [iAb]*mU*mUmGmAmUmGmAmUfAfCfAmAmUmGmCmAmUmGmU*mG*mA |
| 592 | [iAb]*mU*mUmCmAmAmAmCmGfAfGfCmUmGmAmGmAmUmCmU*mA*mA |
| 593 | [iAb]*mU*mUmCmAmUmAmCmGfAfGfCmUmGmAmCmAmUmCmU*mA*mA |
| 594 | [iAb]*mA*mUmCmUmCmAfAfCfUmUmUmGmUmGmUmCmA*mA*mA |
| 595 | [iAb]*mU*mAmUmCmUmCmAfAfCfUmUmUmGmUmGmUmCmA*mA*mA |
| 596 | [iAb]*mU*mAmUmAmUmCmUmCmAfAfCfUmUmUmGmUmGmUmCmA*mA*m |
| | A |
| 597 | [iAb]*mC*mUmAmUmAmUmCmUmCmAfAfCfUmUmUmGmUmGmUmCmA*mA |
| | *mA |
| 598 | [iAb]*mA*mAmUmCmAmAmAmGfAfAfUmAmUmCmUmUmAmGmU*mG*mA |
| 599 | mU*fA*mCmAfUmUfCmAmUmUmGmUmAfUmCfAmUmCmAmAmG*mC*mA |
| 600 | mU*fA*mUmUfCmAfUmUmGmUmAmUmCfAmUfCmAmAmGmCmA*mU*mU |
| 601 | mU*fA*mAmAfUmAfCmAmUmUmAmUmAfUmGfCmUmAmAmCmA*mU*mG |
| 602 | mU*fG*mUmAfAmAfAmUmAmAmAmAmAfCmUfUmAmAmUmGmA*mC*mU |
| 603 | mU*fA*mUmCfAmUfUmAmGmGmCmUmUfUmGfCmAmGmUmCmU*mU*mC |
| 604 | mU*fU*mUmUfUmCfAmCmAmGmCmCmAfCmAfUmCmUmUmCmC*mC*mC |
| 605 | mU*fA*mAmCfCmUfUmUmCmUmCmAmUfUmUfCmCmUmCmUmA*mU*mC |
| 606 | mU*fG*mAmAfAmCfAmUmAmGmAmUmUfGmCfAmUmCmAmCmC*mU*mA |
| 607 | mU*fG*mUmCfUmUfAmUmCmUmUmUmGfAmGfGmUmAmGmAmA*mC*mA |
| 608 | mU*fC*mUmUfAmAfUmAmCmGmAmAmGfAmGfCmAmGmUmUmA*mG*mG |
| 609 | mU*fG*mAmGfCmAfAmUmAmGmCmAmGfGmUfUmUmAmCmCmU*mU*mG |
| 610 | mU*fC*mAmUfAmAfAmGmGmGmGmAmAfAmAfUmGmGmAmAmA*mA*mG |
| 611 | mU*fC*mAmUfGmAfUmAmUmUmCmAmGfAmUfAmCmCmAmGmC*mC*mA |
| 612 | mU*fU*mUmAfUmAfCmUmUmUmUmGmUfAmAfUmAmUmAmUmU*mU*mC |
| 613 | mU*fA*mGmAfUmUfUmCmUmUmUmUmUfAmAfCmAmUmAmAmU*mU*mA |
| 614 | mU*fA*mCmAfCmAfAmAmGmUmUmGmAfGmAfUmAmUmAmGmU*mC*mU |
| 615 | mU*fG*mAmUfUmGfGmAmUmGmAmAmGfUmCfAmAmCmUmCmC*mU*mG |
| 616 | mU*fU*mUmUfAmGfAmGmCmAmAmUmAfUmUfUmAmUmUmUmU*mU*mA |
| 617 | mU*fC*mAmUfUmGfUmUmGmGmAmAmCfUmAfUmGmAmCmAmG*mA*mA |
| 618 | mU*fC*mAmUfAmAfUmUmAmUmCmAmUfCmAfUmUmAmGmGmC*mU*mU |
| 619 | mU*fA*mUmGfCmUfUmAmAmCmUmUmUfAmGfAmGmUmUmAmU*mC*mU |
| 620 | mU*fU*mUmGfCmUfUmUmCmAmAmAmAfUmAfAmAmAmUmUmA*mA*mA |
| 621 | mU*fA*mUmAfCmUfGmUmAmCmUmGmUfCmUfUmAmUmCmUmU*mU*mG |
| 622 | mU*fC*mUmUfUmUfUmAmAmCmAmUmAfAmUfUmAmUmCmAmU*mC*mA |
| 623 | mU*fU*mCmUfUmUfGmAmCmAmCmAmAfAmGfUmUmGmAmGmA*mU*mA |
| 624 | mU*fC*mUmUfUmGfAmGmGmUmAmGmAfAmCfAmAmAmAmAmA*mA*mA |
| 625 | mU*fU*mUmAfUmUfUmUmAmUmAmCmUfUmUfUmGmUmAmAmU*mA*mU |
| 626 | mU*fA*mAmUfUmAfUmCmAmUmCmAmUfUmAfGmGmCmUmUmU*mG*mC |
| 627 | mU*fA*mUmGfGmAfAmAmAmGmAmAmAfGmCfUmAmUmGmAmG*mA*mG |
| 628 | mU*fC*mUmUfCmAfAmAmAmUmAmCmAfUmUfAmUmAmUmGmC*mU*mA |
| 629 | mU*fC*mUmUfCmUfUmAmAmUmAmCmGfAmAfGmAmGmCmAmG*mU*mU |
| 630 | mU*fG*mGmUfUmCfAmAmGmUmAmAmAfAmUfAmAmAmAmAmC*mU*mU |
| 631 | mU*fU*mCmAfCmAfUmAmAmAmGmGmGfGmAfAmAmAmUmGmG*mA*mA |
| 632 | mU*fA*mUmUfUmUfAmUmAmCmUmUmUfUmGfUmAmAmUmAmU*mA*mU |
| 633 | mU*fU*mCmAfUmCfAmAmGmCmAmUmUfUmCfAmGmGmAmGmC*mC*mA |
| 634 | mU*fU*mUmUfCmUfUmUmUmUmAmAmCfAmUfAmAmUmUmAmU*mC*mA |
| 635 | mU*fU*mUmUfUmAfAmCmAmUmAmAmUfUmAfUmCmAmUmCmA*mU*mU |
| 636 | mU*fU*mCmAfUmCfAmUmUmAmGmGmCfUmUfUmGmCmAmGmU*mC*mU |
| 637 | mU*fA*mUmAfGmCfAmGmGmUmUmUmAfCmCfUmUmGmUmGmU*mA*mC |
| 638 | mU*fU*mCmUfUmUfUmUmAmAmCmAmUfAmAfUmUmAmUmCmA*mU*mC |
| 639 | mU*fU*mCmAfUmUfAmGmGmCmUmUmUfGmCfAmGmUmCmUmU*mC*mU |
| 640 | mU*fU*mAmUfAmCfUmUmUmUmGmUmAfAmUfAmUmAmUmUmU*mC*mU |
| 64 | mU*fU*mCmUfUmAfAmUmAmCmGmAmAfGmAfGmCmAmGmUmU*mA*mG |
| 642 | mU*fG*mUmAfCmUfGmUmCmUmUmAmUfCmUfUmUmGmAmGmG*mU*mA |
| 643 | mU*fU*mGmUfGmCfUmCmUmUmGmGmUfUmCfAmAmGmUmAmA*mA*mA |
| 644 | mU*fU*mUmUfGmAfCmAmCmAmAmAmGfUmUfGmAmGmAmUmA*mU*mA |
| 645 | mU*fU*mAmUfCmAfUmCmAmUmUmAmGfGmCfUmUmUmGmCmA*mG*mU |
| 646 | mU*fU*mAmUfUmUfUmAmUmAmCmUmUfUmUfGmUmAmAmUmA*mU*mA |
| 647 | mU*fU*mUmCfAmUfGmAmUmAmUmUmCfAmGfAmUmAmCmCmA*mG*mC |
| 648 | mU*fU*mAmGfAmUfGmUmCmAmGmCmUfCmGfUmUmUmGmA*mA*mG*mG |
| 649 | mU*fU*mUmGfAmCfAmCmAmAmAmGmUfUmGfAmGmAmUmA*mU*mA*mG |
| 650 | mU*fC*mAmCfAmUfUmCmAmUmUmGmUfAmUfCmAmUmCmA*mA*mG*mC |
| 651 | mU*fU*mCmAfAmAfGmAmUmAmUmUmCfAmCfAmUmUmCmA*mU*mU*mG |
| 652 | mU*fC*mAmUfCmAfAmGmCmAmUmUmUfCmAfGmGmAmGmC*mC*mA*mU |
| 653 | mU*fA*mAmCmAfUmAmAmUmUmAmUmCfAmUfCmAmUmUmAmG*mG*mC |
| 654 | [CpmA]*fU*mUmGmUmUfGmGmAmAmCfUmAfUmGfAmCmAmGmA*mC |
| 655 | mU*fG*mUmAfUmCfAmUmCmAmAmGmCfAmUfUmUmCmAmG*mG*mA*mG |
| 656 | mU*fA*mCmUfGmUfAmCmUmGmUmCmUfUmAfUmCmUmUmU*mG*mA*mG |
| 657 | mU*fA*mCmAfUmAfAmAmGmGmGmGmAfAmAfAmUmGmGmA*mA*mA*mA |
| 658 | mU*fU*mCmAfAmAfAmUmAmCmAmUmUfAmUfAmUmGmCmU*mA*mA*mC |
| 659 | mU*fU*mCmAfAmAfCmAmUmAmUmUmCfAmCfAmUmUmCmA*mU*mU*mG |
| 660 | mU*fU*mCmAfAmAfImAmUmAmUmUmCfAmCfAmUmUmCmA*mU*mU*mG |
| 661 | mU*fU*mCmAfAmAfGmAmUmAmUmUmCfAmCfAmAmUmCmA*mU*mU*mG |
| 662 | mU*fA*mCmAfUmAfAmAmGmGmGmGmAfAmAfAmUmG*mG*mA*mA |
| 663 | mU*fC*mAmUmCmAmAmGmCmAmUmUmUfCmAfGmGmAmGmC*mC*mA*m |
| | U |
| 664 | mU*fC*mAmUfC[UNA-A]fAmGmCmAmUmUmUfCmAfGmGmAmGm |
| | C*mC*mA*mU |
| 665 | mU*fC*mAmUfCmA[UNA-A]mGmCmAmUmUmUfCmAf |
| | GmGmAmGmC*mC*mA*mU |
| 666 | mU*fC*mAmUfCmAfImGmCmAmUmUmUfCmAfGmGmAmGmC*mC*mA*mU |
| 667 | mU*fA*mGmAfGmAfUmUmUmCmUmUmUfUmUfAmAmCmAmU*mA*mA*mU |
| 668 | mU*fA*mAmAfUmGfGmAmAmAmAmGmAfAmAfGmCmUmAmUmG*mA*mG |
| 669 | mU*fA*mAmAfUmGfGmAmAmAmAmGmAfAmAfGmCmUmAmU*mG*mA*mG |
| 670 | mU*fC*mAmCmAmUmUmCmAmUmUmGmUfAmUfCmAmUmCmA*mA*mG*m |
| | C |
| 671 | mU*fC*mAmCfA[UNA-U]fUmCmAmUmUmGmUfAmUfCmA |
| | mUmCmA*mA*mG*mC |
| 672 | mU*fC*mAmCfAmU[UNA-U]mCmAmUmUmGmUfAmUfCm |
| | AmUmCmA*mA*mG*mC |
| 673 | mU*fC*mAmCfAmUfImCmAmUmUmGmUfAmUfCmAmUmCmA*mA*mG*mC |
| 674 | mU*fU*mAmGfAmUfCmUmCmAmGmCmUfCmGfUmUmUmGmA*mA*mG*mG |
| 675 | mU*fU*mAmGfAmUfGmUmCmAmGmCmUfCmGfUmAmUmGmA*mA*mG*mG |
| 676 | mU*fU*mAmGfAmUfImUmCmAmGmCmUfCmGfUmUmUmGmA*mA*mG*mG |
| 677 | mU*fU*mUmGfAmCfAmCmAmAmAmGmUfUmGfAmGmA*mU*mA*mU |
| 678 | mU*fU*mUmGfAmCfAmCmAmAmAmGmUfUmGfAmGmAmU*mA*mU*mA |
| 679 | mU*fU*mUmGfAmCfAmCmAmAmAmGmUfUmGfAmGmAmU?mA?mU*mA*m |
| | G |
| 680 | mU*fC*mAmCfUmAfAmGmAmUmAmUmUfCmUfUmUmGmAmU*mU*mU*mU |
| 681 | mU*fC*mAmCfUmAfAmGmAmUmAmUmUfCmUfUmUmGmA?mU?mU*mU*mU |

1. An ALK7 RNAi agent comprising Formula: 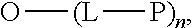
wherein O is a double stranded RNA (dsRNA) comprising a sense stand and an antisense strand, wherein the antisense strand is complementary to ALK7 mRNA;
wherein L is a linker or a bond;
wherein P is an NPR-C-binding peptide comprising SEQ ID NO: 1 (GX₁₁IDX₁₄I), wherein X₁₁ is arginine, proline, or hydroxyproline, and X₁₄ is arginine or N-methylarginine; and
wherein n is 1, 2, 3, or 4.

2. (canceled)

3. (canceled)

4. The ALK7 RNAi agent of claim 1, wherein P comprises one of SEQ ID NO: 2-7.

5. (canceled)

6. (canceled)

7. (canceled)

8. (canceled)

9. (canceled)

10. (canceled)

11. (canceled)

12. (canceled)

13. (canceled)

14. The ALK7 RNAi agent of claim 1, wherein P comprises SEQ ID NO: 8 (X₇-Cha-X₉GX₁₁IDX₁₄I), wherein:
X₇ is proline, hydroxyproline, glycine, cysteine, or alanine;
X₉ is alanine, serine, or glycine;
X₁₁ is proline, hydroxyproline, or arginine; and
X₁₄ is arginine or N-methylarginine.

15. The ALK7 RNAi agent of claim 14, wherein P comprises SEQ ID NO: 9 (fSp-Cha-aGPIDRI).

16. The ALK7 RNAi agent of claim 1, wherein P comprises a sequence selected from any one of SEQ ID NOs: 11-57.

17. The ALK7 RNAi agent of claim 1, wherein P is a linear peptide.

18. The ALK7 RNAi agent of claim 1, wherein P is a cyclic peptide.

19. (canceled)

20. (canceled)

21. (canceled)

22. The ALK7 RNAi agent of claim 1, wherein P comprises a C-terminal hydroxyl.

23. The ALK7 RNAi agent of claim 1, wherein P comprises a C-terminal amide.

24. The ALK7 RNAi agent of claim 1, wherein L comprises a linker core and one or more spacers.

25. The ALK7 RNAi agent of claim 24, wherein L comprises Spacer1-Linker Core-Spacer2.

26. The ALK7 RNAi agent of claim 24, wherein the Linker Core is selected from Table 7.

27. The ALK7 RNAi agent of claim 26, wherein the Spacer1 and Spacer 2 are selected from Table 8.

28. The ALK7 RNAi agent of claim 1, wherein L is selected from Table 9.

29. The ALK7 RNAi agent of claim 1, wherein n is 1 or 2.

30. The ALK7 RNAi agent of claim 1, wherein L is attached to the 5′ end of the sense strand and to the N-terminal end of the peptide.

31. The ALK7 RNAi agent of claim 1, wherein L is attached to the 5′ end of the sense strand and to the C-terminal end of the peptide.

32. The ALK7 RNAi agent of claim 1, wherein L is attached to the 3′ end of the sense strand and to the N-terminal end of the peptide.

33. The ALK7 RNAi agent of claim 1, wherein L is attached to the 3′ end of the sense strand and to the C-terminal end of the peptide.

34. The ALK7 RNAi agent of claim 1, wherein L comprises the formula:

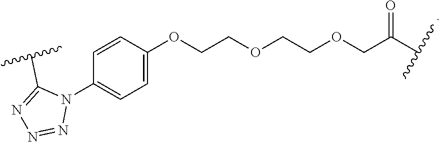 [SMILES: *C(=O)COCCOCCOc1ccc(-n2nnnc2*)cc1]

35. The ALK7 RNAi agent of claim 1, wherein L comprises the formula: 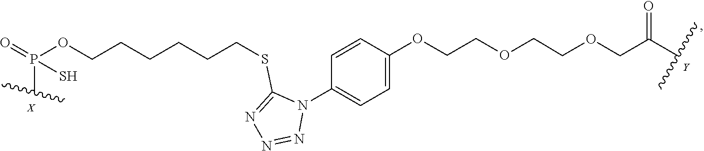
wherein X represents a position to which the 3′ end of the sense strand of O is conjugated, and
wherein Y represents a position to which an N-terminal end of P is conjugated.

36. The ALK7 RNAi agent of claim 1, further comprising a fatty acid (FA), wherein FA is attached to O or to P.

37. (canceled)

38. (canceled)

39. The ALK7 RNAi agent of claim 36, wherein the ALK7 RNAi agent comprises (FA)m-O-L-P or O-L-P-(FA)ₘ, wherein m is an integer of 1 to 4.

40. The ALK7 RNAi agent of claim 36, wherein the ALK7 RNAi agent further comprises a Spacer3, and wherein the ALK7 RNAi agent is of a formula selected from (FA-Spacer3)ₘ-O-L-P, O-L-P-(Spacer3-FA)ₘ, O-L-(FA-Spacer3)ₘ-P, and O-(Spacer3-FA)ₘ-L-P, wherein m is an integer of 1 to 4.

41. (canceled)

42. (canceled)

43. (canceled)

44. The ALK7 RNAi agent of claim 36, wherein the fatty acid is selected from the group consisting of FA1-FA27 of Table 10.

45. The ALK7 RNAi agent of claim 1, wherein the sense strand and the antisense strand form a duplex, and wherein the sense strand and the antisense strand are selected from the group consisting of:
the sense strand comprises a first nucleic acid sequence of SEQ ID NO: 78, and the antisense strand comprises a second nucleic acid sequence of SEQ ID NO: 154;
the sense strand comprises a first nucleic acid sequence of SEQ ID NO: 79, and the antisense strand comprises a second nucleic acid sequence of SEQ ID NO: 155;
the sense strand comprises a first nucleic acid sequence of SEQ ID NO: 80, and the antisense strand comprises a second nucleic acid sequence of SEQ ID NO: 156;
the sense strand comprises a first nucleic acid sequence of SEQ ID NO: 81, and the antisense strand comprises a second nucleic acid sequence of SEQ ID NO: 157;
the sense strand comprises a first nucleic acid sequence of SEQ ID NO: 82, and the antisense strand comprises a second nucleic acid sequence of SEQ ID NO: 158;
the sense strand comprises a first nucleic acid sequence of SEQ ID NO: 83, and the antisense strand comprises a second nucleic acid sequence of SEQ ID NO: 159;
the sense strand comprises a first nucleic acid sequence of SEQ ID NO: 84, and the antisense strand comprises a second nucleic acid sequence of SEQ ID NO: 160;
the sense strand comprises a first nucleic acid sequence of SEQ ID NO: 85, and the antisense strand comprises a second nucleic acid sequence of SEQ ID NO: 161;
the sense strand comprises a first nucleic acid sequence of SEQ ID NO: 86, and the antisense strand comprises a second nucleic acid sequence of SEQ ID NO: 162;
the sense strand comprises a first nucleic acid sequence of SEQ ID NO: 87, and the antisense strand comprises a second nucleic acid sequence of SEQ ID NO: 163;
the sense strand comprises a first nucleic acid sequence of SEQ ID NO: 88, and the antisense strand comprises a second nucleic acid sequence of SEQ ID NO: 164;
the sense strand comprises a first nucleic acid sequence of SEQ ID NO: 89, and the antisense strand comprises a second nucleic acid sequence of SEQ ID NO: 165;
the sense strand comprises a first nucleic acid sequence of SEQ ID NO: 90, and the antisense strand comprises a second nucleic acid sequence of SEQ ID NO: 166;
the sense strand comprises a first nucleic acid sequence of SEQ ID NO: 91, and the antisense strand comprises a second nucleic acid sequence of SEQ ID NO: 167;
the sense strand comprises a first nucleic acid sequence of SEQ ID NO: 92, and the antisense strand comprises a second nucleic acid sequence of SEQ ID NO: 168;
the sense strand comprises a first nucleic acid sequence of SEQ ID NO: 93, and the antisense strand comprises a second nucleic acid sequence of SEQ ID NO: 169;
the sense strand comprises a first nucleic acid sequence of SEQ ID NO: 94, and the antisense strand comprises a second nucleic acid sequence of SEQ ID NO: 170;
the sense strand comprises a first nucleic acid sequence of SEQ ID NO: 95, and the antisense strand comprises a second nucleic acid sequence of SEQ ID NO: 171;
the sense strand comprises a first nucleic acid sequence of SEQ ID NO: 96, and the antisense strand comprises a second nucleic acid sequence of SEQ ID NO: 172;
the sense strand comprises a first nucleic acid sequence of SEQ ID NO: 97, and the antisense strand comprises a second nucleic acid sequence of SEQ ID NO: 173;
the sense strand comprises a first nucleic acid sequence of SEQ ID NO: 98, and the antisense strand comprises a second nucleic acid sequence of SEQ ID NO: 174;
the sense strand comprises a first nucleic acid sequence of SEQ ID NO: 99, and the antisense strand comprises a second nucleic acid sequence of SEQ ID NO: 175;
the sense strand comprises a first nucleic acid sequence of SEQ ID NO: 100, and the antisense strand comprises a second nucleic acid sequence of SEQ ID NO: 176;
the sense strand comprises a first nucleic acid sequence of SEQ ID NO: 101, and the antisense strand comprises a second nucleic acid sequence of SEQ ID NO: 177;
the sense strand comprises a first nucleic acid sequence of SEQ ID NO: 102, and the antisense strand comprises a second nucleic acid sequence of SEQ ID NO: 178;
the sense strand comprises a first nucleic acid sequence of SEQ ID NO: 103, and the antisense strand comprises a second nucleic acid sequence of SEQ ID NO: 179;
the sense strand comprises a first nucleic acid sequence of SEQ ID NO: 104, and the antisense strand comprises a second nucleic acid sequence of SEQ ID NO: 180;
the sense strand comprises a first nucleic acid sequence of SEQ ID NO: 105, and the antisense strand comprises a second nucleic acid sequence of SEQ ID NO: 181;
the sense strand comprises a first nucleic acid sequence of SEQ ID NO: 106, and the antisense strand comprises a second nucleic acid sequence of SEQ ID NO: 182;
the sense strand comprises a first nucleic acid sequence of SEQ ID NO: 107, and the antisense strand comprises a second nucleic acid sequence of SEQ ID NO: 183;
the sense strand comprises a first nucleic acid sequence of SEQ ID NO: 108, and the antisense strand comprises a second nucleic acid sequence of SEQ ID NO: 184;
the sense strand comprises a first nucleic acid sequence of SEQ ID NO: 109, and the antisense strand comprises a second nucleic acid sequence of SEQ ID NO: 185;
the sense strand comprises a first nucleic acid sequence of SEQ ID NO: 110, and the antisense strand comprises a second nucleic acid sequence of SEQ ID NO: 186;
the sense strand comprises a first nucleic acid sequence of SEQ ID NO: 111, and the antisense strand comprises a second nucleic acid sequence of SEQ ID NO: 187;
the sense strand comprises a first nucleic acid sequence of SEQ ID NO: 112, and the antisense strand comprises a second nucleic acid sequence of SEQ ID NO: 188;
the sense strand comprises a first nucleic acid sequence of SEQ ID NO: 113, and the antisense strand comprises a second nucleic acid sequence of SEQ ID NO: 189;
the sense strand comprises a first nucleic acid sequence of SEQ ID NO: 114, and the antisense strand comprises a second nucleic acid sequence of SEQ ID NO: 190;
the sense strand comprises a first nucleic acid sequence of SEQ ID NO: 115, and the antisense strand comprises a second nucleic acid sequence of SEQ ID NO: 191;
the sense strand comprises a first nucleic acid sequence of SEQ ID NO: 116, and the antisense strand comprises a second nucleic acid sequence of SEQ ID NO: 192;
the sense strand comprises a first nucleic acid sequence of SEQ ID NO: 117, and the antisense strand comprises a second nucleic acid sequence of SEQ ID NO: 193;
the sense strand comprises a first nucleic acid sequence of SEQ ID NO: 118, and the antisense strand comprises a second nucleic acid sequence of SEQ ID NO: 194;
the sense strand comprises a first nucleic acid sequence of SEQ ID NO: 119, and the antisense strand comprises a second nucleic acid sequence of SEQ ID NO: 195;
the sense strand comprises a first nucleic acid sequence of SEQ ID NO: 120, and the antisense strand comprises a second nucleic acid sequence of SEQ ID NO: 196;
the sense strand comprises a first nucleic acid sequence of SEQ ID NO: 121, and the antisense strand comprises a second nucleic acid sequence of SEQ ID NO: 197;
the sense strand comprises a first nucleic acid sequence of SEQ ID NO: 122, and the antisense strand comprises a second nucleic acid sequence of SEQ ID NO: 198;
the sense strand comprises a first nucleic acid sequence of SEQ ID NO: 123, and the antisense strand comprises a second nucleic acid sequence of SEQ ID NO: 199;
the sense strand comprises a first nucleic acid sequence of SEQ ID NO: 124, and the antisense strand comprises a second nucleic acid sequence of SEQ ID NO: 200;
the sense strand comprises a first nucleic acid sequence of SEQ ID NO: 125, and the antisense strand comprises a second nucleic acid sequence of SEQ ID NO: 201;
the sense strand comprises a first nucleic acid sequence of SEQ ID NO: 126, and the antisense strand comprises a second nucleic acid sequence of SEQ ID NO: 202;
the sense strand comprises a first nucleic acid sequence of SEQ ID NO: 127, and the antisense strand comprises a second nucleic acid sequence of SEQ ID NO: 203;
the sense strand comprises a first nucleic acid sequence of SEQ ID NO: 128, and the antisense strand comprises a second nucleic acid sequence of SEQ ID NO: 204;
the sense strand comprises a first nucleic acid sequence of SEQ ID NO: 129, and the antisense strand comprises a second nucleic acid sequence of SEQ ID NO: 205;
the sense strand comprises a first nucleic acid sequence of SEQ ID NO: 130, and the antisense strand comprises a second nucleic acid sequence of SEQ ID NO: 206;
the sense strand comprises a first nucleic acid sequence of SEQ ID NO: 131, and the antisense strand comprises a second nucleic acid sequence of SEQ ID NO: 207;
the sense strand comprises a first nucleic acid sequence of SEQ ID NO: 132, and the antisense strand comprises a second nucleic acid sequence of SEQ ID NO: 208;
the sense strand comprises a first nucleic acid sequence of SEQ ID NO: 133, and the antisense strand comprises a second nucleic acid sequence of SEQ ID NO: 209;
the sense strand comprises a first nucleic acid sequence of SEQ ID NO: 134, and the antisense strand comprises a second nucleic acid sequence of SEQ ID NO: 210;
the sense strand comprises a first nucleic acid sequence of SEQ ID NO: 135, and the antisense strand comprises a second nucleic acid sequence of SEQ ID NO: 211;
the sense strand comprises a first nucleic acid sequence of SEQ ID NO: 136, and the antisense strand comprises a second nucleic acid sequence of SEQ ID NO: 212;
the sense strand comprises a first nucleic acid sequence of SEQ ID NO: 137, and the antisense strand comprises a second nucleic acid sequence of SEQ ID NO: 213;
the sense strand comprises a first nucleic acid sequence of SEQ ID NO: 138, and the antisense strand comprises a second nucleic acid sequence of SEQ ID NO: 214;
the sense strand comprises a first nucleic acid sequence of SEQ ID NO: 139, and the antisense strand comprises a second nucleic acid sequence of SEQ ID NO: 215;
the sense strand comprises a first nucleic acid sequence of SEQ ID NO: 140, and the antisense strand comprises a second nucleic acid sequence of SEQ ID NO: 216;
the sense strand comprises a first nucleic acid sequence of SEQ ID NO: 141, and the antisense strand comprises a second nucleic acid sequence of SEQ ID NO: 217;
the sense strand comprises a first nucleic acid sequence of SEQ ID NO: 142, and the antisense strand comprises a second nucleic acid sequence of SEQ ID NO: 218;
the sense strand comprises a first nucleic acid sequence of SEQ ID NO: 143, and the antisense strand comprises a second nucleic acid sequence of SEQ ID NO: 219;
the sense strand comprises a first nucleic acid sequence of SEQ ID NO: 144, and the antisense strand comprises a second nucleic acid sequence of SEQ ID NO: 220;
the sense strand comprises a first nucleic acid sequence of SEQ ID NO: 145, and the antisense strand comprises a second nucleic acid sequence of SEQ ID NO: 221;
the sense strand comprises a first nucleic acid sequence of SEQ ID NO: 146, and the antisense strand comprises a second nucleic acid sequence of SEQ ID NO: 222;
the sense strand comprises a first nucleic acid sequence of SEQ ID NO: 147, and the antisense strand comprises a second nucleic acid sequence of SEQ ID NO: 223;
the sense strand comprises a first nucleic acid sequence of SEQ ID NO: 148, and the antisense strand comprises a second nucleic acid sequence of SEQ ID NO: 224;
the sense strand comprises a first nucleic acid sequence of SEQ ID NO: 149, and the antisense strand comprises a second nucleic acid sequence of SEQ ID NO: 225;
the sense strand comprises a first nucleic acid sequence of SEQ ID NO: 150, and the antisense strand comprises a second nucleic acid sequence of SEQ ID NO: 226;
the sense strand comprises a first nucleic acid sequence of SEQ ID NO: 151, and the antisense strand comprises a second nucleic acid sequence of SEQ ID NO: 227;
the sense strand comprises a first nucleic acid sequence of SEQ ID NO: 152, and the antisense strand comprises a second nucleic acid sequence of SEQ ID NO: 228;
the sense strand comprises a first nucleic acid sequence of SEQ ID NO: 153, and the antisense strand comprises a second nucleic acid sequence of SEQ ID NO: 229;
the sense strand comprises a first nucleic acid sequence of SEQ ID NO: 396, and the antisense strand comprises a second nucleic acid sequence of SEQ ID NO: 457;
the sense strand comprises a first nucleic acid sequence of SEQ ID NO: 397, and the antisense strand comprises a second nucleic acid sequence of SEQ ID NO: 458;
the sense strand comprises a first nucleic acid sequence of SEQ ID NO: 398, and the antisense strand comprises a second nucleic acid sequence of SEQ ID NO: 459;
the sense strand comprises a first nucleic acid sequence of SEQ ID NO: 399, and the antisense strand comprises a second nucleic acid sequence of SEQ ID NO: 460;
the sense strand comprises a first nucleic acid sequence of SEQ ID NO: 400, and the antisense strand comprises a second nucleic acid sequence of SEQ ID NO: 461;
the sense strand comprises a first nucleic acid sequence of SEQ ID NO: 401, and the antisense strand comprises a second nucleic acid sequence of SEQ ID NO: 462;
the sense strand comprises a first nucleic acid sequence of SEQ ID NO: 402, and the antisense strand comprises a second nucleic acid sequence of SEQ ID NO: 463;
the sense strand comprises a first nucleic acid sequence of SEQ ID NO: 403, and the antisense strand comprises a second nucleic acid sequence of SEQ ID NO: 464;
the sense strand comprises a first nucleic acid sequence of SEQ ID NO: 404, and the antisense strand comprises a second nucleic acid sequence of SEQ ID NO: 465;
the sense strand comprises a first nucleic acid sequence of SEQ ID NO: 405, and the antisense strand comprises a second nucleic acid sequence of SEQ ID NO: 466;
the sense strand comprises a first nucleic acid sequence of SEQ ID NO: 406, and the antisense strand comprises a second nucleic acid sequence of SEQ ID NO: 467;
the sense strand comprises a first nucleic acid sequence of SEQ ID NO: 407, and the antisense strand comprises a second nucleic acid sequence of SEQ ID NO: 468;
the sense strand comprises a first nucleic acid sequence of SEQ ID NO: 408, and the antisense strand comprises a second nucleic acid sequence of SEQ ID NO: 469;
the sense strand comprises a first nucleic acid sequence of SEQ ID NO: 409, and the antisense strand comprises a second nucleic acid sequence of SEQ ID NO: 470;
the sense strand comprises a first nucleic acid sequence of SEQ ID NO: 410, and the antisense strand comprises a second nucleic acid sequence of SEQ ID NO: 471;
the sense strand comprises a first nucleic acid sequence of SEQ ID NO: 411, and the antisense strand comprises a second nucleic acid sequence of SEQ ID NO: 472;
the sense strand comprises a first nucleic acid sequence of SEQ ID NO: 412, and the antisense strand comprises a second nucleic acid sequence of SEQ ID NO: 473;
the sense strand comprises a first nucleic acid sequence of SEQ ID NO: 413, and the antisense strand comprises a second nucleic acid sequence of SEQ ID NO: 474;
the sense strand comprises a first nucleic acid sequence of SEQ ID NO: 414, and the antisense strand comprises a second nucleic acid sequence of SEQ ID NO: 475;
the sense strand comprises a first nucleic acid sequence of SEQ ID NO: 415, and the antisense strand comprises a second nucleic acid sequence of SEQ ID NO: 476;
the sense strand comprises a first nucleic acid sequence of SEQ ID NO: 416, and the antisense strand comprises a second nucleic acid sequence of SEQ ID NO: 477;
the sense strand comprises a first nucleic acid sequence of SEQ ID NO: 417, and the antisense strand comprises a second nucleic acid sequence of SEQ ID NO: 478;
the sense strand comprises a first nucleic acid sequence of SEQ ID NO: 418, and the antisense strand comprises a second nucleic acid sequence of SEQ ID NO: 479;
the sense strand comprises a first nucleic acid sequence of SEQ ID NO: 419, and the antisense strand comprises a second nucleic acid sequence of SEQ ID NO: 480;
the sense strand comprises a first nucleic acid sequence of SEQ ID NO: 420, and the antisense strand comprises a second nucleic acid sequence of SEQ ID NO: 481;
the sense strand comprises a first nucleic acid sequence of SEQ ID NO: 421, and the antisense strand comprises a second nucleic acid sequence of SEQ ID NO: 482;
the sense strand comprises a first nucleic acid sequence of SEQ ID NO: 422, and the antisense strand comprises a second nucleic acid sequence of SEQ ID NO: 483;
the sense strand comprises a first nucleic acid sequence of SEQ ID NO: 423, and the antisense strand comprises a second nucleic acid sequence of SEQ ID NO: 484;
the sense strand comprises a first nucleic acid sequence of SEQ ID NO: 424, and the antisense strand comprises a second nucleic acid sequence of SEQ ID NO: 485;
the sense strand comprises a first nucleic acid sequence of SEQ ID NO: 425, and the antisense strand comprises a second nucleic acid sequence of SEQ ID NO: 486;
the sense strand comprises a first nucleic acid sequence of SEQ ID NO: 426, and the antisense strand comprises a second nucleic acid sequence of SEQ ID NO: 487;
the sense strand comprises a first nucleic acid sequence of SEQ ID NO: 427, and the antisense strand comprises a second nucleic acid sequence of SEQ ID NO: 488;
the sense strand comprises a first nucleic acid sequence of SEQ ID NO: 428, and the antisense strand comprises a second nucleic acid sequence of SEQ ID NO: 489;
the sense strand comprises a first nucleic acid sequence of SEQ ID NO: 429, and the antisense strand comprises a second nucleic acid sequence of SEQ ID NO: 490;
the sense strand comprises a first nucleic acid sequence of SEQ ID NO: 430, and the antisense strand comprises a second nucleic acid sequence of SEQ ID NO: 491;
the sense strand comprises a first nucleic acid sequence of SEQ ID NO: 431, and the antisense strand comprises a second nucleic acid sequence of SEQ ID NO: 492;
the sense strand comprises a first nucleic acid sequence of SEQ ID NO: 432, and the antisense strand comprises a second nucleic acid sequence of SEQ ID NO: 493;
the sense strand comprises a first nucleic acid sequence of SEQ ID NO: 433, and the antisense strand comprises a second nucleic acid sequence of SEQ ID NO: 494;
the sense strand comprises a first nucleic acid sequence of SEQ ID NO: 434, and the antisense strand comprises a second nucleic acid sequence of SEQ ID NO: 495;
the sense strand comprises a first nucleic acid sequence of SEQ ID NO: 435, and the antisense strand comprises a second nucleic acid sequence of SEQ ID NO: 496;
the sense strand comprises a first nucleic acid sequence of SEQ ID NO: 436, and the antisense strand comprises a second nucleic acid sequence of SEQ ID NO: 497;
the sense strand comprises a first nucleic acid sequence of SEQ ID NO: 437, and the antisense strand comprises a second nucleic acid sequence of SEQ ID NO: 498;
the sense strand comprises a first nucleic acid sequence of SEQ ID NO: 438, and the antisense strand comprises a second nucleic acid sequence of SEQ ID NO: 499;
the sense strand comprises a first nucleic acid sequence of SEQ ID NO: 439, and the antisense strand comprises a second nucleic acid sequence of SEQ ID NO: 500;
the sense strand comprises a first nucleic acid sequence of SEQ ID NO: 440, and the antisense strand comprises a second nucleic acid sequence of SEQ ID NO: 501;
the sense strand comprises a first nucleic acid sequence of SEQ ID NO: 441, and the antisense strand comprises a second nucleic acid sequence of SEQ ID NO: 502;
the sense strand comprises a first nucleic acid sequence of SEQ ID NO: 442, and the antisense strand comprises a second nucleic acid sequence of SEQ ID NO: 503;
the sense strand comprises a first nucleic acid sequence of SEQ ID NO: 443, and the antisense strand comprises a second nucleic acid sequence of SEQ ID NO: 504;
the sense strand comprises a first nucleic acid sequence of SEQ ID NO: 444, and the antisense strand comprises a second nucleic acid sequence of SEQ ID NO: 505;
the sense strand comprises a first nucleic acid sequence of SEQ ID NO: 134, and the antisense strand comprises a second nucleic acid sequence of SEQ ID NO: 210;
the sense strand comprises a first nucleic acid sequence of SEQ ID NO: 115, and the antisense strand comprises a second nucleic acid sequence of SEQ ID NO: 191;
the sense strand comprises a first nucleic acid sequence of SEQ ID NO: 445, and the antisense strand comprises a second nucleic acid sequence of SEQ ID NO: 506;
the sense strand comprises a first nucleic acid sequence of SEQ ID NO: 134, and the antisense strand comprises a second nucleic acid sequence of SEQ ID NO: 507;
the sense strand comprises a first nucleic acid sequence of SEQ ID NO: 134, and the antisense strand comprises a second nucleic acid sequence of SEQ ID NO: 508;
the sense strand comprises a first nucleic acid sequence of SEQ ID NO: 446, and the antisense strand comprises a second nucleic acid sequence of SEQ ID NO: 507;
the sense strand comprises a first nucleic acid sequence of SEQ ID NO: 447, and the antisense strand comprises a second nucleic acid sequence of SEQ ID NO: 509;
the sense strand comprises a first nucleic acid sequence of SEQ ID NO: 128, and the antisense strand comprises a second nucleic acid sequence of SEQ ID NO: 510;
the sense strand comprises a first nucleic acid sequence of SEQ ID NO: 448, and the antisense strand comprises a second nucleic acid sequence of SEQ ID NO: 511;
the sense strand comprises a first nucleic acid sequence of SEQ ID NO: 449, and the antisense strand comprises a second nucleic acid sequence of SEQ ID NO: 512;
the sense strand comprises a first nucleic acid sequence of SEQ ID NO: 450, and the antisense strand comprises a second nucleic acid sequence of SEQ ID NO: 513;
the sense strand comprises a first nucleic acid sequence of SEQ ID NO: 451, and the antisense strand comprises a second nucleic acid sequence of SEQ ID NO: 514;
the sense strand comprises a first nucleic acid sequence of SEQ ID NO: 452, and the antisense strand comprises a second nucleic acid sequence of SEQ ID NO: 515;
the sense strand comprises a first nucleic acid sequence of SEQ ID NO: 135, and the antisense strand comprises a second nucleic acid sequence of SEQ ID NO: 516;
the sense strand comprises a first nucleic acid sequence of SEQ ID NO: 453, and the antisense strand comprises a second nucleic acid sequence of SEQ ID NO: 517;
the sense strand comprises a first nucleic acid sequence of SEQ ID NO: 454, and the antisense strand comprises a second nucleic acid sequence of SEQ ID NO: 518;
the sense strand comprises a first nucleic acid sequence of SEQ ID NO: 133, and the antisense strand comprises a second nucleic acid sequence of SEQ ID NO: 517;
the sense strand comprises a first nucleic acid sequence of SEQ ID NO: 455, and the antisense strand comprises a second nucleic acid sequence of SEQ ID NO: 518; or
the sense strand comprises a first nucleic acid sequence of SEQ ID NO: 456, and the antisense strand comprises a second nucleic acid sequence of SEQ ID NO: 209;
wherein optionally one or more nucleotides of the sense strand and the antisense strand are independently modified nucleotides, and
wherein optionally one or more internucleotide linkages of the sense strand and the antisense strand are modified internucleotide linkages.

46. The ALK7 RNAi agent of claim 1, wherein the sense strand and the antisense strand comprise a pair of nucleic acid sequences selected from the group consisting of:
the sense strand comprises a first nucleic acid sequence of SEQ ID NO: 230, and the antisense strand comprises a second nucleic acid sequence of SEQ ID NO: 316;
the sense strand comprises a first nucleic acid sequence of SEQ ID NO: 231, and the antisense strand comprises a second nucleic acid sequence of SEQ ID NO: 317;
the sense strand comprises a first nucleic acid sequence of SEQ ID NO: 232, and the antisense strand comprises a second nucleic acid sequence of SEQ ID NO: 318;
the sense strand comprises a first nucleic acid sequence of SEQ ID NO: 233, and the antisense strand comprises a second nucleic acid sequence of SEQ ID NO: 319;
the sense strand comprises a first nucleic acid sequence of SEQ ID NO: 234, and the antisense strand comprises a second nucleic acid sequence of SEQ ID NO: 320;
the sense strand comprises a first nucleic acid sequence of SEQ ID NO: 235, and the antisense strand comprises a second nucleic acid sequence of SEQ ID NO: 321;
the sense strand comprises a first nucleic acid sequence of SEQ ID NO: 236, and the antisense strand comprises a second nucleic acid sequence of SEQ ID NO: 322;
the sense strand comprises a first nucleic acid sequence of SEQ ID NO: 237, and the antisense strand comprises a second nucleic acid sequence of SEQ ID NO: 323;
the sense strand comprises a first nucleic acid sequence of SEQ ID NO: 238, and the antisense strand comprises a second nucleic acid sequence of SEQ ID NO: 324;
the sense strand comprises a first nucleic acid sequence of SEQ ID NO: 239, and the antisense strand comprises a second nucleic acid sequence of SEQ ID NO: 325;
the sense strand comprises a first nucleic acid sequence of SEQ ID NO: 240, and the antisense strand comprises a second nucleic acid sequence of SEQ ID NO: 326;
the sense strand comprises a first nucleic acid sequence of SEQ ID NO: 241, and the antisense strand comprises a second nucleic acid sequence of SEQ ID NO: 327;
the sense strand comprises a first nucleic acid sequence of SEQ ID NO: 242, and the antisense strand comprises a second nucleic acid sequence of SEQ ID NO: 328;
the sense strand comprises a first nucleic acid sequence of SEQ ID NO: 243, and the antisense strand comprises a second nucleic acid sequence of SEQ ID NO: 329;
the sense strand comprises a first nucleic acid sequence of SEQ ID NO: 244, and the antisense strand comprises a second nucleic acid sequence of SEQ ID NO: 330;
the sense strand comprises a first nucleic acid sequence of SEQ ID NO: 245, and the antisense strand comprises a second nucleic acid sequence of SEQ ID NO: 331;
the sense strand comprises a first nucleic acid sequence of SEQ ID NO: 246, and the antisense strand comprises a second nucleic acid sequence of SEQ ID NO: 332;
the sense strand comprises a first nucleic acid sequence of SEQ ID NO: 247, and the antisense strand comprises a second nucleic acid sequence of SEQ ID NO: 333;
the sense strand comprises a first nucleic acid sequence of SEQ ID NO: 248, and the antisense strand comprises a second nucleic acid sequence of SEQ ID NO: 334;
the sense strand comprises a first nucleic acid sequence of SEQ ID NO: 249, and the antisense strand comprises a second nucleic acid sequence of SEQ ID NO: 335;
the sense strand comprises a first nucleic acid sequence of SEQ ID NO: 250, and the antisense strand comprises a second nucleic acid sequence of SEQ ID NO: 336;
the sense strand comprises a first nucleic acid sequence of SEQ ID NO: 251, and the antisense strand comprises a second nucleic acid sequence of SEQ ID NO: 337;
the sense strand comprises a first nucleic acid sequence of SEQ ID NO: 252, and the antisense strand comprises a second nucleic acid sequence of SEQ ID NO: 338;
the sense strand comprises a first nucleic acid sequence of SEQ ID NO: 253, and the antisense strand comprises a second nucleic acid sequence of SEQ ID NO: 339;
the sense strand comprises a first nucleic acid sequence of SEQ ID NO: 254, and the antisense strand comprises a second nucleic acid sequence of SEQ ID NO: 340;
the sense strand comprises a first nucleic acid sequence of SEQ ID NO: 255, and the antisense strand comprises a second nucleic acid sequence of SEQ ID NO: 341;
the sense strand comprises a first nucleic acid sequence of SEQ ID NO: 256, and the antisense strand comprises a second nucleic acid sequence of SEQ ID NO: 342;
the sense strand comprises a first nucleic acid sequence of SEQ ID NO: 257, and the antisense strand comprises a second nucleic acid sequence of SEQ ID NO: 343;
the sense strand comprises a first nucleic acid sequence of SEQ ID NO: 258, and the antisense strand comprises a second nucleic acid sequence of SEQ ID NO: 344;
the sense strand comprises a first nucleic acid sequence of SEQ ID NO: 259, and the antisense strand comprises a second nucleic acid sequence of SEQ ID NO: 345;
the sense strand comprises a first nucleic acid sequence of SEQ ID NO: 260, and the antisense strand comprises a second nucleic acid sequence of SEQ ID NO: 346;
the sense strand comprises a first nucleic acid sequence of SEQ ID NO: 261, and the antisense strand comprises a second nucleic acid sequence of SEQ ID NO: 347;
the sense strand comprises a first nucleic acid sequence of SEQ ID NO: 262, and the antisense strand comprises a second nucleic acid sequence of SEQ ID NO: 348;
the sense strand comprises a first nucleic acid sequence of SEQ ID NO: 263, and the antisense strand comprises a second nucleic acid sequence of SEQ ID NO: 349;
the sense strand comprises a first nucleic acid sequence of SEQ ID NO: 264, and the antisense strand comprises a second nucleic acid sequence of SEQ ID NO: 350;
the sense strand comprises a first nucleic acid sequence of SEQ ID NO: 265, and the antisense strand comprises a second nucleic acid sequence of SEQ ID NO: 351;
the sense strand comprises a first nucleic acid sequence of SEQ ID NO: 266, and the antisense strand comprises a second nucleic acid sequence of SEQ ID NO: 352;
the sense strand comprises a first nucleic acid sequence of SEQ ID NO: 267, and the antisense strand comprises a second nucleic acid sequence of SEQ ID NO: 353;
the sense strand comprises a first nucleic acid sequence of SEQ ID NO: 268, and the antisense strand comprises a second nucleic acid sequence of SEQ ID NO: 354;
the sense strand comprises a first nucleic acid sequence of SEQ ID NO: 269, and the antisense strand comprises a second nucleic acid sequence of SEQ ID NO: 355;
the sense strand comprises a first nucleic acid sequence of SEQ ID NO: 270, and the antisense strand comprises a second nucleic acid sequence of SEQ ID NO: 356;
the sense strand comprises a first nucleic acid sequence of SEQ ID NO: 271, and the antisense strand comprises a second nucleic acid sequence of SEQ ID NO: 357;
the sense strand comprises a first nucleic acid sequence of SEQ ID NO: 272, and the antisense strand comprises a second nucleic acid sequence of SEQ ID NO: 358;
the sense strand comprises a first nucleic acid sequence of SEQ ID NO: 273, and the antisense strand comprises a second nucleic acid sequence of SEQ ID NO: 359;
the sense strand comprises a first nucleic acid sequence of SEQ ID NO: 274, and the antisense strand comprises a second nucleic acid sequence of SEQ ID NO: 360;
the sense strand comprises a first nucleic acid sequence of SEQ ID NO: 275, and the antisense strand comprises a second nucleic acid sequence of SEQ ID NO: 361;
the sense strand comprises a first nucleic acid sequence of SEQ ID NO: 276, and the antisense strand comprises a second nucleic acid sequence of SEQ ID NO: 362;
the sense strand comprises a first nucleic acid sequence of SEQ ID NO: 277, and the antisense strand comprises a second nucleic acid sequence of SEQ ID NO: 363;
the sense strand comprises a first nucleic acid sequence of SEQ ID NO: 278, and the antisense strand comprises a second nucleic acid sequence of SEQ ID NO: 364;
the sense strand comprises a first nucleic acid sequence of SEQ ID NO: 279, and the antisense strand comprises a second nucleic acid sequence of SEQ ID NO: 365;
the sense strand comprises a first nucleic acid sequence of SEQ ID NO: 280, and the antisense strand comprises a second nucleic acid sequence of SEQ ID NO: 366;
the sense strand comprises a first nucleic acid sequence of SEQ ID NO: 281, and the antisense strand comprises a second nucleic acid sequence of SEQ ID NO: 367;
the sense strand comprises a first nucleic acid sequence of SEQ ID NO: 282, and the antisense strand comprises a second nucleic acid sequence of SEQ ID NO: 368;
the sense strand comprises a first nucleic acid sequence of SEQ ID NO: 283, and the antisense strand comprises a second nucleic acid sequence of SEQ ID NO: 369;
the sense strand comprises a first nucleic acid sequence of SEQ ID NO: 284, and the antisense strand comprises a second nucleic acid sequence of SEQ ID NO: 370;
the sense strand comprises a first nucleic acid sequence of SEQ ID NO: 285, and the antisense strand comprises a second nucleic acid sequence of SEQ ID NO: 371;
the sense strand comprises a first nucleic acid sequence of SEQ ID NO: 286, and the antisense strand comprises a second nucleic acid sequence of SEQ ID NO: 372;
the sense strand comprises a first nucleic acid sequence of SEQ ID NO: 287, and the antisense strand comprises a second nucleic acid sequence of SEQ ID NO: 373;
the sense strand comprises a first nucleic acid sequence of SEQ ID NO: 288, and the antisense strand comprises a second nucleic acid sequence of SEQ ID NO: 374;
the sense strand comprises a first nucleic acid sequence of SEQ ID NO: 289, and the antisense strand comprises a second nucleic acid sequence of SEQ ID NO: 375;
the sense strand comprises a first nucleic acid sequence of SEQ ID NO: 290, and the antisense strand comprises a second nucleic acid sequence of SEQ ID NO: 376;
the sense strand comprises a first nucleic acid sequence of SEQ ID NO: 291, and the antisense strand comprises a second nucleic acid sequence of SEQ ID NO: 377;
the sense strand comprises a first nucleic acid sequence of SEQ ID NO: 292, and the antisense strand comprises a second nucleic acid sequence of SEQ ID NO: 378;
the sense strand comprises a first nucleic acid sequence of SEQ ID NO: 293, and the antisense strand comprises a second nucleic acid sequence of SEQ ID NO: 379;
the sense strand comprises a first nucleic acid sequence of SEQ ID NO: 294, and the antisense strand comprises a second nucleic acid sequence of SEQ ID NO: 380;
the sense strand comprises a first nucleic acid sequence of SEQ ID NO: 295, and the antisense strand comprises a second nucleic acid sequence of SEQ ID NO: 381;
the sense strand comprises a first nucleic acid sequence of SEQ ID NO: 296, and the antisense strand comprises a second nucleic acid sequence of SEQ ID NO: 382;
the sense strand comprises a first nucleic acid sequence of SEQ ID NO: 297, and the antisense strand comprises a second nucleic acid sequence of SEQ ID NO: 383;
the sense strand comprises a first nucleic acid sequence of SEQ ID NO: 298, and the antisense strand comprises a second nucleic acid sequence of SEQ ID NO: 384;
the sense strand comprises a first nucleic acid sequence of SEQ ID NO: 299, and the antisense strand comprises a second nucleic acid sequence of SEQ ID NO: 385;
the sense strand comprises a first nucleic acid sequence of SEQ ID NO: 300, and the antisense strand comprises a second nucleic acid sequence of SEQ ID NO: 386;
the sense strand comprises a first nucleic acid sequence of SEQ ID NO: 301, and the antisense strand comprises a second nucleic acid sequence of SEQ ID NO: 387;
the sense strand comprises a first nucleic acid sequence of SEQ ID NO: 302, and the antisense strand comprises a second nucleic acid sequence of SEQ ID NO: 388;
the sense strand comprises a first nucleic acid sequence of SEQ ID NO: 303, and the antisense strand comprises a second nucleic acid sequence of SEQ ID NO: 338;
the sense strand comprises a first nucleic acid sequence of SEQ ID NO: 304, and the antisense strand comprises a second nucleic acid sequence of SEQ ID NO: 339;
the sense strand comprises a first nucleic acid sequence of SEQ ID NO: 305, and the antisense strand comprises a second nucleic acid sequence of SEQ ID NO: 340;
the sense strand comprises a first nucleic acid sequence of SEQ ID NO: 306, and the antisense strand comprises a second nucleic acid sequence of SEQ ID NO: 346;
the sense strand comprises a first nucleic acid sequence of SEQ ID NO: 307, and the antisense strand comprises a second nucleic acid sequence of SEQ ID NO: 347;
the sense strand comprises a first nucleic acid sequence of SEQ ID NO: 308, and the antisense strand comprises a second nucleic acid sequence of SEQ ID NO: 348;
the sense strand comprises a first nucleic acid sequence of SEQ ID NO: 309, and the antisense strand comprises a second nucleic acid sequence of SEQ ID NO: 349;
the sense strand comprises a first nucleic acid sequence of SEQ ID NO: 310, and the antisense strand comprises a second nucleic acid sequence of SEQ ID NO: 389;
the sense strand comprises a first nucleic acid sequence of SEQ ID NO: 311, and the antisense strand comprises a second nucleic acid sequence of SEQ ID NO: 390;
the sense strand comprises a first nucleic acid sequence of SEQ ID NO: 312, and the antisense strand comprises a second nucleic acid sequence of SEQ ID NO: 391;
the sense strand comprises a first nucleic acid sequence of SEQ ID NO: 313, and the antisense strand comprises a second nucleic acid sequence of SEQ ID NO: 389;
the sense strand comprises a first nucleic acid sequence of SEQ ID NO: 314, and the antisense strand comprises a second nucleic acid sequence of SEQ ID NO: 390;
the sense strand comprises a first nucleic acid sequence of SEQ ID NO: 315, and the antisense strand comprises a second nucleic acid sequence of SEQ ID NO: 391;
the sense strand comprises a first nucleic acid sequence of SEQ ID NO: 519, and the antisense strand comprises a second nucleic acid sequence of SEQ ID NO: 599;
the sense strand comprises a first nucleic acid sequence of SEQ ID NO: 520, and the antisense strand comprises a second nucleic acid sequence of SEQ ID NO: 600;
the sense strand comprises a first nucleic acid sequence of SEQ ID NO: 521, and the antisense strand comprises a second nucleic acid sequence of SEQ ID NO: 601;
the sense strand comprises a first nucleic acid sequence of SEQ ID NO: 522, and the antisense strand comprises a second nucleic acid sequence of SEQ ID NO: 602;
the sense strand comprises a first nucleic acid sequence of SEQ ID NO: 523, and the antisense strand comprises a second nucleic acid sequence of SEQ ID NO: 603;
the sense strand comprises a first nucleic acid sequence of SEQ ID NO: 524, and the antisense strand comprises a second nucleic acid sequence of SEQ ID NO: 604;
the sense strand comprises a first nucleic acid sequence of SEQ ID NO: 525, and the antisense strand comprises a second nucleic acid sequence of SEQ ID NO: 605;
the sense strand comprises a first nucleic acid sequence of SEQ ID NO: 526, and the antisense strand comprises a second nucleic acid sequence of SEQ ID NO: 606;
the sense strand comprises a first nucleic acid sequence of SEQ ID NO: 527, and the antisense strand comprises a second nucleic acid sequence of SEQ ID NO: 607;
the sense strand comprises a first nucleic acid sequence of SEQ ID NO: 528, and the antisense strand comprises a second nucleic acid sequence of SEQ ID NO: 608;
the sense strand comprises a first nucleic acid sequence of SEQ ID NO: 529, and the antisense strand comprises a second nucleic acid sequence of SEQ ID NO: 609;
the sense strand comprises a first nucleic acid sequence of SEQ ID NO: 530, and the antisense strand comprises a second nucleic acid sequence of SEQ ID NO: 610;
the sense strand comprises a first nucleic acid sequence of SEQ ID NO: 531, and the antisense strand comprises a second nucleic acid sequence of SEQ ID NO: 611;
the sense strand comprises a first nucleic acid sequence of SEQ ID NO: 532, and the antisense strand comprises a second nucleic acid sequence of SEQ ID NO: 612;
the sense strand comprises a first nucleic acid sequence of SEQ ID NO: 533, and the antisense strand comprises a second nucleic acid sequence of SEQ ID NO: 613;
the sense strand comprises a first nucleic acid sequence of SEQ ID NO: 534, and the antisense strand comprises a second nucleic acid sequence of SEQ ID NO: 614;
the sense strand comprises a first nucleic acid sequence of SEQ ID NO: 535, and the antisense strand comprises a second nucleic acid sequence of SEQ ID NO: 615;
the sense strand comprises a first nucleic acid sequence of SEQ ID NO: 536, and the antisense strand comprises a second nucleic acid sequence of SEQ ID NO: 616;
the sense strand comprises a first nucleic acid sequence of SEQ ID NO: 537, and the antisense strand comprises a second nucleic acid sequence of SEQ ID NO: 617;
the sense strand comprises a first nucleic acid sequence of SEQ ID NO: 538, and the antisense strand comprises a second nucleic acid sequence of SEQ ID NO: 618;
the sense strand comprises a first nucleic acid sequence of SEQ ID NO: 539, and the antisense strand comprises a second nucleic acid sequence of SEQ ID NO: 619;
the sense strand comprises a first nucleic acid sequence of SEQ ID NO: 540, and the antisense strand comprises a second nucleic acid sequence of SEQ ID NO: 620;
the sense strand comprises a first nucleic acid sequence of SEQ ID NO: 541, and the antisense strand comprises a second nucleic acid sequence of SEQ ID NO: 621;
the sense strand comprises a first nucleic acid sequence of SEQ ID NO: 542, and the antisense strand comprises a second nucleic acid sequence of SEQ ID NO: 622;
the sense strand comprises a first nucleic acid sequence of SEQ ID NO: 543, and the antisense strand comprises a second nucleic acid sequence of SEQ ID NO: 623;
the sense strand comprises a first nucleic acid sequence of SEQ ID NO: 544, and the antisense strand comprises a second nucleic acid sequence of SEQ ID NO: 624;
the sense strand comprises a first nucleic acid sequence of SEQ ID NO: 545, and the antisense strand comprises a second nucleic acid sequence of SEQ ID NO: 625;
the sense strand comprises a first nucleic acid sequence of SEQ ID NO: 546, and the antisense strand comprises a second nucleic acid sequence of SEQ ID NO: 625;
the sense strand comprises a first nucleic acid sequence of SEQ ID NO: 547, and the antisense strand comprises a second nucleic acid sequence of SEQ ID NO: 627;
the sense strand comprises a first nucleic acid sequence of SEQ ID NO: 548, and the antisense strand comprises a second nucleic acid sequence of SEQ ID NO: 628;
the sense strand comprises a first nucleic acid sequence of SEQ ID NO: 549, and the antisense strand comprises a second nucleic acid sequence of SEQ ID NO: 629;
the sense strand comprises a first nucleic acid sequence of SEQ ID NO: 550, and the antisense strand comprises a second nucleic acid sequence of SEQ ID NO: 630;
the sense strand comprises a first nucleic acid sequence of SEQ ID NO: 551, and the antisense strand comprises a second nucleic acid sequence of SEQ ID NO: 631;
the sense strand comprises a first nucleic acid sequence of SEQ ID NO: 552, and the antisense strand comprises a second nucleic acid sequence of SEQ ID NO: 632;
the sense strand comprises a first nucleic acid sequence of SEQ ID NO: 553, and the antisense strand comprises a second nucleic acid sequence of SEQ ID NO: 633;
the sense strand comprises a first nucleic acid sequence of SEQ ID NO: 554, and the antisense strand comprises a second nucleic acid sequence of SEQ ID NO: 634;
the sense strand comprises a first nucleic acid sequence of SEQ ID NO: 555, and the antisense strand comprises a second nucleic acid sequence of SEQ ID NO: 635;
the sense strand comprises a first nucleic acid sequence of SEQ ID NO: 556, and the antisense strand comprises a second nucleic acid sequence of SEQ ID NO: 636;
the sense strand comprises a first nucleic acid sequence of SEQ ID NO: 557, and the antisense strand comprises a second nucleic acid sequence of SEQ ID NO: 636;
the sense strand comprises a first nucleic acid sequence of SEQ ID NO: 558, and the antisense strand comprises a second nucleic acid sequence of SEQ ID NO: 638;
the sense strand comprises a first nucleic acid sequence of SEQ ID NO: 559, and the antisense strand comprises a second nucleic acid sequence of SEQ ID NO: 639;
the sense strand comprises a first nucleic acid sequence of SEQ ID NO: 560, and the antisense strand comprises a second nucleic acid sequence of SEQ ID NO: 640;
the sense strand comprises a first nucleic acid sequence of SEQ ID NO: 561, and the antisense strand comprises a second nucleic acid sequence of SEQ ID NO: 641;
the sense strand comprises a first nucleic acid sequence of SEQ ID NO: 562, and the antisense strand comprises a second nucleic acid sequence of SEQ ID NO: 642;
the sense strand comprises a first nucleic acid sequence of SEQ ID NO: 563, and the antisense strand comprises a second nucleic acid sequence of SEQ ID NO: 643;
the sense strand comprises a first nucleic acid sequence of SEQ ID NO: 564, and the antisense strand comprises a second nucleic acid sequence of SEQ ID NO: 644;
the sense strand comprises a first nucleic acid sequence of SEQ ID NO: 565, and the antisense strand comprises a second nucleic acid sequence of SEQ ID NO: 645;
the sense strand comprises a first nucleic acid sequence of SEQ ID NO: 566, and the antisense strand comprises a second nucleic acid sequence of SEQ ID NO: 646;
the sense strand comprises a first nucleic acid sequence of SEQ ID NO: 567, and the antisense strand comprises a second nucleic acid sequence of SEQ ID NO: 647;
the sense strand comprises a first nucleic acid sequence of SEQ ID NO: 568, and the antisense strand comprises a second nucleic acid sequence of SEQ ID NO: 648;
the sense strand comprises a first nucleic acid sequence of SEQ ID NO: 569, and the antisense strand comprises a second nucleic acid sequence of SEQ ID NO: 649;
the sense strand comprises a first nucleic acid sequence of SEQ ID NO: 570, and the antisense strand comprises a second nucleic acid sequence of SEQ ID NO: 650;
the sense strand comprises a first nucleic acid sequence of SEQ ID NO: 571, and the antisense strand comprises a second nucleic acid sequence of SEQ ID NO: 651;
the sense strand comprises a first nucleic acid sequence of SEQ ID NO: 572, and the antisense strand comprises a second nucleic acid sequence of SEQ ID NO: 652;
the sense strand comprises a first nucleic acid sequence of SEQ ID NO: 573, and the antisense strand comprises a second nucleic acid sequence of SEQ ID NO: 649;
the sense strand comprises a first nucleic acid sequence of SEQ ID NO: 574, and the antisense strand comprises a second nucleic acid sequence of SEQ ID NO: 650;
the sense strand comprises a first nucleic acid sequence of SEQ ID NO: 575, and the antisense strand comprises a second nucleic acid sequence of SEQ ID NO: 652;
the sense strand comprises a first nucleic acid sequence of SEQ ID NO: 568, and the antisense strand comprises a second nucleic acid sequence of SEQ ID NO: 648;
the sense strand comprises a first nucleic acid sequence of SEQ ID NO: 576, and the antisense strand comprises a second nucleic acid sequence of SEQ ID NO: 648;
the sense strand comprises a first nucleic acid sequence of SEQ ID NO: 577, and the antisense strand comprises a second nucleic acid sequence of SEQ ID NO: 651;
the sense strand comprises a first nucleic acid sequence of SEQ ID NO: 578, and the antisense strand comprises a second nucleic acid sequence of SEQ ID NO: 653;
the sense strand comprises a first nucleic acid sequence of SEQ ID NO: 579, and the antisense strand comprises a second nucleic acid sequence of SEQ ID NO: 654;
the sense strand comprises a first nucleic acid sequence of SEQ ID NO: 580, and the antisense strand comprises a second nucleic acid sequence of SEQ ID NO: 653;
the sense strand comprises a first nucleic acid sequence of SEQ ID NO: 581, and the antisense strand comprises a second nucleic acid sequence of SEQ ID NO: 653;
the sense strand comprises a first nucleic acid sequence of SEQ ID NO: 582, and the antisense strand comprises a second nucleic acid sequence of SEQ ID NO: 653;
the sense strand comprises a first nucleic acid sequence of SEQ ID NO: 310, and the antisense strand comprises a second nucleic acid sequence of SEQ ID NO: 655;
the sense strand comprises a first nucleic acid sequence of SEQ ID NO: 583, and the antisense strand comprises a second nucleic acid sequence of SEQ ID NO: 656;
the sense strand comprises a first nucleic acid sequence of SEQ ID NO: 584, and the antisense strand comprises a second nucleic acid sequence of SEQ ID NO: 657;
the sense strand comprises a first nucleic acid sequence of SEQ ID NO: 250, and the antisense strand comprises a second nucleic acid sequence of SEQ ID NO: 658;
the sense strand comprises a first nucleic acid sequence of SEQ ID NO: 571, and the antisense strand comprises a second nucleic acid sequence of SEQ ID NO: 659;
the sense strand comprises a first nucleic acid sequence of SEQ ID NO: 571, and the antisense strand comprises a second nucleic acid sequence of SEQ ID NO: 660;
the sense strand comprises a first nucleic acid sequence of SEQ ID NO: 585, and the antisense strand comprises a second nucleic acid sequence of SEQ ID NO: 659;
the sense strand comprises a first nucleic acid sequence of SEQ ID NO: 586, and the antisense strand comprises a second nucleic acid sequence of SEQ ID NO: 661;
the sense strand comprises a first nucleic acid sequence of SEQ ID NO: 584, and the antisense strand comprises a second nucleic acid sequence of SEQ ID NO: 662;
the sense strand comprises a first nucleic acid sequence of SEQ ID NO: 572, and the antisense strand comprises a second nucleic acid sequence of SEQ ID NO: 663;
the sense strand comprises a first nucleic acid sequence of SEQ ID NO: 572, and the antisense strand comprises a second nucleic acid sequence of SEQ ID NO: 664;
the sense strand comprises a first nucleic acid sequence of SEQ ID NO: 572, and the antisense strand comprises a second nucleic acid sequence of SEQ ID NO: 665;
the sense strand comprises a first nucleic acid sequence of SEQ ID NO: 587, and the antisense strand comprises a second nucleic acid sequence of SEQ ID NO: 666;
the sense strand comprises a first nucleic acid sequence of SEQ ID NO: 588, and the antisense strand comprises a second nucleic acid sequence of SEQ ID NO: 667;
the sense strand comprises a first nucleic acid sequence of SEQ ID NO: 589, and the antisense strand comprises a second nucleic acid sequence of SEQ ID NO: 668;
the sense strand comprises a first nucleic acid sequence of SEQ ID NO: 590, and the antisense strand comprises a second nucleic acid sequence of SEQ ID NO: 669;
the sense strand comprises a first nucleic acid sequence of SEQ ID NO: 570, and the antisense strand comprises a second nucleic acid sequence of SEQ ID NO: 670;
the sense strand comprises a first nucleic acid sequence of SEQ ID NO: 570, and the antisense strand comprises a second nucleic acid sequence of SEQ ID NO: 671;
the sense strand comprises a first nucleic acid sequence of SEQ ID NO: 570, and the antisense strand comprises a second nucleic acid sequence of SEQ ID NO: 672;
the sense strand comprises a first nucleic acid sequence of SEQ ID NO: 591, and the antisense strand comprises a second nucleic acid sequence of SEQ ID NO: 673;
the sense strand comprises a first nucleic acid sequence of SEQ ID NO: 592, and the antisense strand comprises a second nucleic acid sequence of SEQ ID NO: 674;
the sense strand comprises a first nucleic acid sequence of SEQ ID NO: 593, and the antisense strand comprises a second nucleic acid sequence of SEQ ID NO: 675;
the sense strand comprises a first nucleic acid sequence of SEQ ID NO: 568, and the antisense strand comprises a second nucleic acid sequence of SEQ ID NO: 676;
the sense strand comprises a first nucleic acid sequence of SEQ ID NO: 594, and the antisense strand comprises a second nucleic acid sequence of SEQ ID NO: 677;
the sense strand comprises a first nucleic acid sequence of SEQ ID NO: 595, and the antisense strand comprises a second nucleic acid sequence of SEQ ID NO: 678;
the sense strand comprises a first nucleic acid sequence of SEQ ID NO: 569, and the antisense strand comprises a second nucleic acid sequence of SEQ ID NO: 677;
the sense strand comprises a first nucleic acid sequence of SEQ ID NO: 596, and the antisense strand comprises a second nucleic acid sequence of SEQ ID NO: 678;
the sense strand comprises a first nucleic acid sequence of SEQ ID NO: 597, and the antisense strand comprises a second nucleic acid sequence of SEQ ID NO: 678;
the sense strand comprises a first nucleic acid sequence of SEQ ID NO: 569, and the antisense strand comprises a second nucleic acid sequence of SEQ ID NO: 679;
the sense strand comprises a first nucleic acid sequence of SEQ ID NO: 598, and the antisense strand comprises a second nucleic acid sequence of SEQ ID NO: 680; and
the sense strand comprises a first nucleic acid sequence of SEQ ID NO: 598, and the antisense strand comprises a second nucleic acid sequence of SEQ ID NO: 681.

47. An ALK7 RNAi agent of Formula: 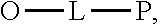
wherein O comprises a sense stand and an antisense strand, wherein the antisense strand is complementary to ALK7 mRNA;
wherein L is a linker comprising the formula: 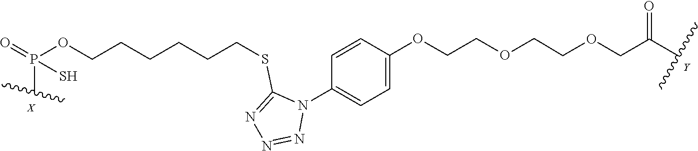
wherein P is an NPR-C-binding peptide comprising SEQ ID NO: 395.

48. The ALK7 RNAi agent of claim 47, wherein:
the sense strand comprises a first nucleic acid sequence of SEQ ID NO: 115, and the antisense strand comprises a second nucleic acid sequence of SEQ ID NO: 191;
the sense strand comprises a first nucleic acid sequence of SEQ ID NO: 117, and the antisense strand comprises a second nucleic acid sequence of SEQ ID NO: 193;
the sense strand comprises a first nucleic acid sequence of SEQ ID NO: 133, and the antisense strand comprises a second nucleic acid sequence of SEQ ID NO: 209;
the sense strand comprises a first nucleic acid sequence of SEQ ID NO: 134, and the antisense strand comprises a second nucleic acid sequence of SEQ ID NO: 210; or
the sense strand comprises a first nucleic acid sequence of SEQ ID NO: 135, and the antisense strand comprises a second nucleic acid sequence of SEQ ID NO: 211.

49. The ALK7 RNAi agent of claim 47, wherein the antisense strand is modified with 2′-fluoro at each of positions 2, 5, 7, 14, and 16; and wherein all other positions of the antisense strand are modified with 2′-OMe.

50. (canceled)

51. The ALK7 RNAi agent of claim 47, wherein the antisense strand comprises phosphorothioate linkages between positions 1 and 2, 2 and 3, 20 and 21, 21 and 22, and 22 and 23.

52. The ALK7 RNAi agent of claim 47, wherein the antisense comprises a vinylphosphonate moiety conjugated to the 5′ end.

53. The ALK7 RNAi agent of claim 47, wherein the sense strand comprises 2′-fluoro modified nucleotides at each of positions 9, 10, and 11; and wherein all other positions of the sense strand are modified with 2′-OMe.

54. (canceled)

55. The ALK7 RNAi agent of claim 47, wherein the sense strand comprises phosphorothioate linkages between positions 1 and 2, 19 and 20, and 20 and 21.

56. The ALK7 RNAi agent of claim 55, wherein the sense strand comprises the inverted abasic moiety conjugated at the 5′ end, wherein the inverted abasic moiety is conjugated to the sense strand by a phosphorothioate linkage.

57. The ALK7 RNAi agent of claim 47, wherein the linker L is attached to a 3′ end of the sense strand.

58. The ALK7 RNAi agent of claim 47, wherein:
the sense strand comprises a first nucleic acid sequence of SEQ ID NO: 568, and the antisense strand comprises a second nucleic acid sequence of SEQ ID NO: 648;
the sense strand comprises a first nucleic acid sequence of SEQ ID NO: 569, and the antisense strand comprises a second nucleic acid sequence of SEQ ID NO: 649;
the sense strand comprises a first nucleic acid sequence of SEQ ID NO: 570, and the antisense strand comprises a second nucleic acid sequence of SEQ ID NO: 650;
the sense strand comprises a first nucleic acid sequence of SEQ ID NO: 571, and the antisense strand comprises a second nucleic acid sequence of SEQ ID NO: 651;
the sense strand comprises a first nucleic acid sequence of SEQ ID NO: 572, and the antisense strand comprises a second nucleic acid sequence of SEQ ID NO: 652;
the sense strand comprises a first nucleic acid sequence of SEQ ID NO: 573, and the antisense strand comprises a second nucleic acid sequence of SEQ ID NO: 649;
the sense strand comprises a first nucleic acid sequence of SEQ ID NO: 574, and the antisense strand comprises a second nucleic acid sequence of SEQ ID NO: 650;
the sense strand comprises a first nucleic acid sequence of SEQ ID NO: 575, and the antisense strand comprises a second nucleic acid sequence of SEQ ID NO: 652;
the sense strand comprises a first nucleic acid sequence of SEQ ID NO: 568, and the antisense strand comprises a second nucleic acid sequence of SEQ ID NO: 648;
the sense strand comprises a first nucleic acid sequence of SEQ ID NO: 576, and the antisense strand comprises a second nucleic acid sequence of SEQ ID NO: 648; or
the sense strand comprises a first nucleic acid sequence of SEQ ID NO: 577, and the antisense strand comprises a second nucleic acid sequence of SEQ ID NO: 651.

59.-87. (canceled)

88. A pharmaceutical composition comprising the ALK7 RNAi agent of claim 1, and a pharmaceutically acceptable carrier.

89. A method of treating a disease or condition of adipose tissue in a patient in need thereof, comprising administering to the patient an effective amount of the ALK7 RNAi agent of claim 1.

90. The method of claim 89, wherein the disease or condition is obesity or obesity-related comorbidity.

91. The method of claim 89, wherein the ALK7 RNAi agent or pharmaceutical composition is administered intravenously or subcutaneously.

92.-96. (canceled)

97. A method of delivering an oligonucleotide to an adipose tissue, comprising administering to a subject the ALK7 RNAi agent of claim 1.
